(19) United States
(12) Patent Application Publication
(10) Pub. No.: US 2005/0032747 A1
(43) Pub. Date: Feb. 10, 2005

(54) COX-2 and FAAH inhibitors

(76) Inventors: Wilmin Bartolini, Amesbury, MA (US); Brian M. Cali, Arlington, MA (US); Barbara Chen, Watertown, MA (US); Yueh-Tyng Chien, Newton, MA (US); Mark G. Currie, Sterling, MA (US); G. Todd Milne, Brookline, MA (US); James Philip Pearson, Cambridge, MA (US); John Jeffrey Talley, Somerville, MA (US); Craig Zimmerman, Topsfield, MA (US)

Correspondence Address: FISH & RICHARDSON PC, 225 FRANKLIN ST, BOSTON, MA, 02110, US

(21) Appl. No.: 10/883,900

(22) Filed: Jul. 1, 2004

Related U.S. Application Data
(60) Provisional application No. 60/483,935, filed on Jul. 1, 2003.
(60) Provisional application No. 60/511,792, filed on Oct. 16, 2003.
(60) Provisional application No. 60/563,589, filed on Apr. 20, 2004.
(60) Provisional application No. 60/570,620, filed on May 13, 2004.

(51) Int. Cl. A61K 31/675; A61K 31/405; C07D 209/42

(52) U.S. Cl. 514/80; 514/419; 548/413; 548/492

(57) ABSTRACT

The invention features compounds that inhibit COX-2 and/or FAAH. The COX-2 inhibitors are selective COX-2 inhibitors in that they are selective for COX-2 compared to COX-1. Certain of the FAAH inhibitors are selective for FAAH relative to COX-2. Certain of the COX-2 inhibitors, in addition for being selective for COX-2 relative to COX-1, are selective for COX-2 relative to FAAH.

Under 35 USC § 119(e)(1), this application claims the benefit of prior U.S. Provisional Application Ser. No. 60/483,935, filed on Jul. 1, 2003; Provisional Application Ser. No. 60/511,792 filed on Oct. 16, 2003; Provisional Application Ser. No. 60/563,589 filed on Apr. 20, 2004; and Provisional Application Ser. No. 60/570,620 filed on May 13, 2004.

This invention relates to inhibitors of cyclooxygenase and to inhibitors of fatty acid amide hydrolase.

### BACKGROUND

Cyclooxygenases play an essential role in prostaglandin synthesis. Cyclooxygenase-1 (COX-1) is constitutive and relatively long-lived, whereas cyclooxygenase-2 (COX-2) is inducible and relatively short-lived. COX-1 is thought to be responsible for maintaining basal level prostaglandin production, which is important for normal gastrointestinal and renal function. COX-2 is induced by certain inflammatory agents, hormones, growth factors, cytokines, and other agents. COX-2 plays a significant role in prostaglandin synthesis within inflammatory cells such as macrophages and monocytes, and prostaglandin production associated with COX-2 induction can have a deleterious effect on the body. Thus, to reduce unwanted inflammation and to treat certain other conditions, it can be desirable to inhibit COX-2 activity without significantly inhibiting COX-1 activity.

Many non-steroidal anti-inflammatory drugs (NSAIDs) inhibit both COX-1 and COX-2. These non-selective inhibitors include indomethacin (Shen et al. 1963 J Am Chem Soc 85:4881; 4-chlorobenzoyl-5-methoxy-2-methyl-1H-indole-3-acetic acid). It is desirable to identify NSAIDs that inhibit COX-2 activity, but do not significantly inhibit COX-1 activity at physiological levels where COX-2 activity is significantly inhibited. Such selective inhibitors are expected to have the desirable anti-inflammatory, anti-pyretic, and analgesic properties associated with NSAIDs, while having reduced or no gastrointestinal or renal toxicity.

Subsequent to indomethacin administration, the unchanged parent compound, the desmethyl metabolite (O-desmethylindomethacin), the desbenzoyl metabolite (N-deschlorobenzoylindomethacin) and the desmethy-desbenzoyl metabolite (O-desmethy-N-deschlorobenzoylindomethacin) can be found in plasma in significant amounts (Strachman et al. 1964 J Am Chem Soc 8:799; Helleberg 1981 Clin Pharmacokinet 6:245), all in an unconjugated form (Harman et al. 1964 J Pharmocol Exp Therap 143:215). It has been reported that all three metabolites are devoid of anti-inflammatory activity (Helleberg 1981 Clin Pharmacokine. 6:245 and Duggan et al. 1972 Pharmacol and Exp Ther 181:562), although it has also been reported that the desmethyl metabolite has some ability to inhibit prostaglandin synthesis (Shen et al.1977 Adv Drug Res 12:90).

Indomethacin derivatives in which the benzoyl group has been replaced by a 4-bromobenzyl group or the acetic acid side chain has been extended exhibit greater selectivity for inhibition of COX-2 relative to COX-1 (Black et al. 1996 Bioorganic & Medicinal Chem Lett 6:725 and Black et al. 1997 Advances in Experimental Medicine and Biology 407:73). In addition, synthesis methodology has been demonstrated for the preparation of indomethacin analogues, some of which do not inhibit cyclooxygenases (Touhey et al. 2002 Eur J Cancer 38:1661).

Many fatty acid amides are known to have analgesic activity. A number of fatty acid amides (e.g., arachidonyl amino acids and anandamide) induce analgesia in animal models of pain (see, for example, Walker et al 1999 Proc Natl Acad Sci 96:12198, Fride and Mechoulam 1993 Eur J Pharmacol 231:313). Anandamide and certain other fatty acid amides (e.g., N-palmitoyl ethanolamine, N-oleoyl ethanolamide, oleamide, 2-arachidonoylglycerol) are cleaved and inactivated by fatty acid amide hydrolase (FAAH) (Deutsch et al. 2003 Prostaglandins Leukot Essent Fatty Acids 66:201; and Cravatt and Lichtman 2003 Current Opinion in Chemical Biology 7:469).

Inhibition of FAAH is expected to lead to an increase in the level of anandamide and other fatty acid amides. This increase in fatty acid amides may lead to an increase in the nociceptive threshold. Thus, inhibitors of FAAH are useful in the treatment of pain. Such inhibitors might also be useful in the treatment of other disorders that can be treated using fatty acid amides or modulators of cannabinoid receptors (e.g., anxiety, eating disorders, and cardiovascular disorders). NPAA (N-palmitoylethanolamine acid anhydrolase) is a hydrolase that breaks down N-palmitoyl ethanolamine (PEA), a fatty acid amide. PEA is a naturally occurring substrate for the cannabinoid receptor 2 (CB2 receptor). Inhibition of NPAA may lead to increased PEA levels. Accordingly, NPAA inhibitors may be useful in the treatment of inflammation and nociceptive pain control.

In addition, there is evidence (see, e.g., Weber et al. 2004 J. Lipid Res. 45:757) that when FAAH activity is reduced or absent, one of its substrates, anandamide, acts as a substrate for COX-2 that can be converted to a prostamide. Thus, certain prostamides may be elevated in the presence of an FAAH inhibitor. Given that certain prostamides are associated with reduced intraocular pressure and ocular hypotensivity, FAAH inhibitors may be useful agents for treating glaucoma.

### SUMMARY

The invention features compounds having Formula I or a pharmaceutically acceptable salt thereof, pharmaceutical compositions comprising such compounds and methods for treating a patient by administering such pharmaceutical compositions alone or in combination with one or more other therapeutic agents. Thus, the invention features compounds having the formula:

The invention features a compound (e.g., a purified compound) having the formula: 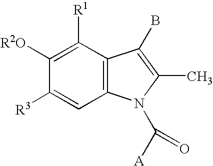
wherein:
R¹ is H or a halogen (Cl, F, Br, I);
R² is H; a C₁ to C₆ alkyl (C₁, C₂, C₃, C₄, C₅, C₆), alkenyl, alkynyl optionally independently substituted with one or more halogen (Cl, F, Br, I), —OH, —C(O)OH, —NH₃; 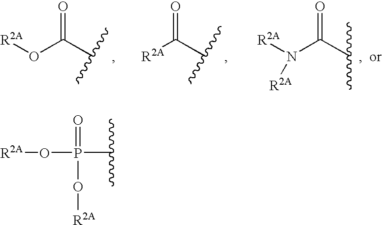
   wherein each R^{2A} is independently: H or a C₁ to C₆ (C₁, C₂, C₃, C₄, C₅, C₆) alkyl, alkenyl, alkynyl, aryl, cycloalkyl, or arylalkyl optionally independently substituted with one or more halogen —OH, —C(O)OH, —NH₃;
R³ is H or a halogen (Cl, F, Br, I);
A is 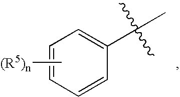
   wherein
   each R⁵ is independently H, a halogen, —CH₃, —CN, —OCH₃, —SCH₃, or —CH₂CH₃, optionally independently substituted with one or more halogen (e.g., —OCF₂, —OCF₃)OH, —C(O)OH, —NH₃; n=1, 2, 3, 4, or 5;
B is 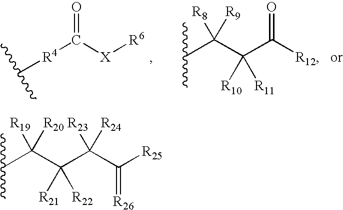
   wherein
   R⁴ is an optionally independently (singly or multiply) substituted C₁ to C₁₀ (C₃, C₄, C₅, C₆, C₇, C₈, C₁ to C₈, C₁ to C₆, C₁ to C₄, or C₁ to C₃ alkyl, —CH₂CH₂—, —CH₂—), alkenyl, alkynyl, wherein the substituents are selected from halogen, —OH, —C(O)OH, —CH₃, —OCH₃, —SCH₃, and —CH₂CH₃, optionally independently substituted with one or more halogen;
   X is —O—, —S—, —N(H)— or —N(H)S(O₂)—;
   R⁶ is: H; an optionally independently substituted C₁ to C₁₀ alkyl; an optionally independently substituted C₄ to C₈ cycloalkyl; a C₁ to C₆ hydroxyalkyl; a hydroxyl substituted C₄ to C₈ aryl; a primary, secondary or tertiary C₁ to C₆ alkylamino; a primary, secondary or tertiary branched C₁ to C₆ alkylamino; primary, secondary or tertiary C₄ to C₈ arylamino; C₂ to C₆ alkylcarboxylic acid; a branched C₃ to C₆ alkylcarboxylic acid; a C₁ to C₆ alkylester; a branched C₁ to C₆ alkylester; a C₄ to C₈ aryl; a C₄ to C₈ arylcarboxylic acid; a C₄ to C₈ arylester; a C₄ to C₈ aryl substituted C₁ to C₆ alkyl; a C₄ to C₈ heterocyclic alkyl or aryl; an alkyl-substituted or aryl-substituted C₄ to C₈ heterocyclic alkyl or aryl, wherein one or more H can be independently substituted by halogen, —OH, and —C(O)OH, —OCH₃, —CH₃, SCH₃ (wherein one or more H can be substituted by a halogen, e.g., F);
   R⁸, R⁹, R¹⁰, and R¹¹ are independently selected from:
      (a) H,
      (b) F,
      (c) methyl or ethyl,
      (d) —CF₃, —CF₂H or —CFH₂,
      (e) hydroxy, —OR¹⁴, SR¹⁴, S(O)R¹⁴, or S(O)₂R¹⁴,
      (f) naphthylmethyl, and
      (g) mono-substituted or di-substituted benzyl, wherein the substituents are selected from:
         (i) H,
         (ii) —CF₃,
         (iii) —CN,
         (iv) F, Cl, Br, or I,
         (v) C₁ to C₆ alkyl, and
         (vi) SR¹⁴, S(O)R¹⁴, or S(O)₂R¹⁴;
   or R⁸ and R⁹ together form an oxo group,
   or R¹⁰ and R¹¹ together form an oxo group,
   or R⁸ and R⁹ are joined so that together with the carbon atom to which they are attached there is formed a cycloalkyl having 3, 4, 5, 6 or 7 ring carbons;
   or R⁸ and R¹⁰ are joined so that together with the carbon atom to which they are attached there is formed a cycloalkyl having 3, 4, 5, 6 or 7 ring carbons;
   or R⁸ and R¹¹ are joined so that together with the carbon atom to which they are attached there is formed a cycloalkyl having 3, 4, 5, 6 or 7 ring carbons;
   or R⁹ and R¹⁰ are joined so that together with the carbon atom to which they are attached there is formed a cycloalkyl having 3, 4, 5, 6 or 7 ring carbons;
   or R⁹ and R¹¹ are joined so that together with the carbon atom to which they are attached there is formed a cycloalkyl having 3, 4, 5, 6 or 7 ring carbons;
   or R¹⁰ and R¹¹ are joined so that together with the carbon atom to which they are attached there is formed a cycloalkyl having 3, 4, 5, 6 or 7 ring carbons;
   R¹² is selected from:
      (a) —OR₁₃, and
      (b) —NR₁₅R₁₆;
   R¹³ is selected from:
      (a) H, and
      (b) C₁ to C₄ alkyl;
   R¹⁴ is selected from methyl, ethyl, mono-substituted benzyl or di-substituted benzyl, wherein the substituents are selected from:
      (a) H,
      (b) CF₃,
      (c) CN,
      (d) F, Cl, Br, I, and
      (e) C₁ to C₆ alkyl;
   R¹⁵ and R¹⁶ are independently selected from:
      (a) H,
      (b) C₁ to C₃ alkyl,
      (c) —OR₁₃,
      (d) —C(O)R₁₇
      (e) —S(O)₂R₁₈,
      (f) a mono-substituted C₂ to C₄ alkyl, wherein the substituent is selected from:
         (i) hydroxy,
         (ii) amino,
         (iii) methylamino,
         (iv) dimethyl amino, and
      (g) an unsubstituted, mono-substituted, or disubstituted phenyl, benzyl or pyridyl group, wherein the substituents are selected from:
         (i) H,
         (ii) CF₃,
         (iii) CN,
         (iv) F, Cl, Br, I, and
         (v) C₁₋₆ alkyl;
   or R¹⁵ and R¹⁶ are joined so that together with the nitrogen atom to which they are attached there is formed a 3, 4, 5, 6, or 7-membered ring, optionally including one or two additional heteroatoms, wherein the additional heteroatoms are selected from N, O, and S, the ring optionally including one or two carbonyl or sulfonyl groups;
   R¹⁷ is selected from:
      (a) H,
      (b) C₁ to C₄ alkyl,
      (c) CF₃, and
      (d) an unsubstituted, mono-substituted, or disubstituted phenyl or benzyl group, wherein the substituents are selected from:
         (i) H,
         (ii) CF₃,
         (iii) CN,
         (iv) F, Cl, Br, I, and
         (v) C₁ to C₆ alkyl;
   R¹⁸ is selected from:
      (a) C₁ to C₄ alkyl,
      (b) CF₃, and
      (c) an unsubstituted, mono-substituted, or disubstituted phenyl or benzyl group, wherein the substituents are selected from:
         (i) H,
         (ii) CF₃,
         (iii) CN,
         (iv) F, Cl, Br, I, and
         (v) C₁ to C₆ alkyl;
   R¹⁹R²⁰, R²¹, R²², R²³ and R²⁴ are independently selected from:
      (a) H,
      (b) F or Cl,
      (c) C₁ to C₅ alkyl or haloalkyl,
      (d) C₃ to C₆ cycloalkyl,
      (e) —CF₃, —CF₂H or —CFH₂,
      (f) hydroxy, —OR₂₇, SR₂₇, S(O)R₂₇, or S(O)₂R₂₇,
      (g) mono-substituted or di-substituted benzyl, wherein the substituents are selected from:
         (i) —CF₃,
         (ii) —CN,
         (iii) F, Cl, Br, or I,
         (iv) C₁ to C₆ alkyl, and
         (v) SR₂₇, S(O)R₂₇, or S(O)₂R₂₇,
      (h) mono-substituted or di-substituted phenyl, wherein the substituents are selected from:
         (i) —CF₃,
         (ii) —CN,
         (iii) F, Cl, Br, or I,
         (iv) C₁ to C₆ alkyl, and
         (v) SR₂₇, S(O)R₂₇, or S(O)₂R₂₇,
   or R¹⁹ and R²⁰ together form an oxo group,
   or R²¹ and R²² together form an oxo group,
   or R²³ and R²⁴ together form an oxo group,
   or R¹⁹ and R²⁰ are joined so that together with the carbon atom to which they are attached there is formed a cycloalkyl having saturated 3, 4, 5, 6 or 7 ring carbons;
   or R²⁰ and R21 are joined so that together with the carbon atom to which they are attached there is formed a cycloalkyl having saturated 3, 4, 5, 6 or 7 ring carbons;
   or R²⁰ and R²³ are joined so that together with the carbon atom to which they are attached there is formed cycloalkyl having saturated 3, 4, 5, 6 or 7 ring carbons;
   or R²¹ and R²² are joined so that together with the carbon atom to which they are attached there is formed a cycloalkyl having saturated 3, 4, 5, 6 or 7 ring carbons;
   or R²¹ and R²³ are joined so that together with the carbon atom to which they are attached there is formed a cycloalkyl having saturated 3, 4, 5, 6 or 7 ring carbons;
   or R²⁴ and R²⁵ are joined so that together with the carbon atom to which they are attached there is formed a cycloalkyl having saturated 3, 4, 5, 6 or 7 ring carbons;
   R²⁶ is O or S;
   R²⁷ is methyl, ethyl, mono-substituted benzyl or di-substituted benzyl, wherein the substituents are selected from:
      (a) CF₃,
      (b) CN,
      (c) F, Cl, Br, I, and
      (d) C₁ to C₆ alkyl;
   R²⁸ and R²⁹ are independently selected from:
      (a) H,
      (b) C₁₋₃ alkyl,
      (c) C₃₋₆ cycloalkyl
      (d) —OR³²,
      (e) —C(O)R³⁰
      (f) —S(O)₂R³¹,
      (g) a mono-substituted C₂ to C₄ alkyl, wherein the substituent is selected from:
         (i) hydroxy,
         (ii) amino,
         (iii) methylamino,
         (iv) dimethyl amino
      (h) an unsubstituted, mono-substituted, or disubstituted phenyl, benzyl or pyridyl group, wherein the substituents are selected from:
         (i) H,
         (ii) CF₃,
         (iii) CN,
         (iv) F, Cl, Br, I, and
         (v) C₁ to C₆ alkyl,
   or R²⁸ and R²⁹ are joined so that together with the nitrogen atom to which they are attached there is formed a 3, 4, 5, 6, or 7-membered ring, optionally including one or two additional heteroatoms, wherein the additional heteroatoms are selected from N, O, and S, wherein a carbon atom may optionally be substituted with an oxo group and a sulfur atom of the ring may optionally substituted with two oxo groups;
   R³⁰ is selected from:
      (a) H,
      (b) C₁ to C₄ alkyl,
      (c) CF₃,
      (d) an unsubstituted, mono-substituted, or disubstituted phenyl or benzyl group, wherein the substituents are selected from:
         (i) CF₃,
         (ii) CN,
         (iii) F, Cl, Br, I, and
         (iv) C₁ to C₆ alkyl;
   R³¹ is selected from:
      (a) C₁ to C₄ alkyl,
      (b) CF₃,
      (c) an unsubstituted, mono-substituted, or disubstituted phenyl or benzyl group, wherein the substituents are selected from:
         (i) CF₃,
         (ii) CN,
         (iii) F, Cl, Br, I, and
         (iv) C₁ to C₆ alkyl;
   or a pharmaceutically acceptable salt thereof;
   provided that:
   (a) when R¹ is H, R² is CH₃, R³ is H, and A is 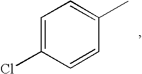
      then B is other than —CH₂C(O)NHCH₂C(O)OCH₂CH₃, —CH₂C(O)NHCH₂CH₂OH, —CH₂C(O)CH₂CH₃, CH₂CH₂C(O)OH, —CH₂C(O)OCH₂CH₃ and —CH(CH₃)C(O)OCH₂CH₃;
   (b) when R¹ is H, R² is CH₃, R³ is H, B is —CH₂C(O)OH and A is 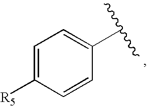
      then R⁵ is other than H, F or Cl;
   (c) when R¹ is H, R² is H, R³ is H, and A is 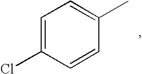
      then B is other than —CH₂C(O)OH, —CH₂C(O)OCH₂CH₃;
   (d) when R¹ is F, R² is —CH₃, R³ is H, A is 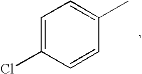
      B is other than —CH₂C(O)OH;
   (e) when R¹ is H, R² is —CH₃, R³ is H, A is 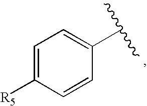
      R⁵ is —CF₃, then B is other than —CH₂C(O)OH;
   (f) when R¹ is H, R² is —CH₃, R³ is H, A is 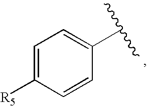
      R⁵ is H, then B is other than —CH₂C(O)OH;
   (g) when R¹ is H, R² is —CH₃, R³ is H, A is 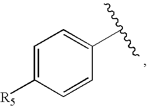
      R⁵ is H, then B is other than —CH₂C(O)OCH₃ or CH₂C(O)OC(CH₃)₃; and
   (h) when R¹ is H, R² is —CH₃, R³ is H, A is 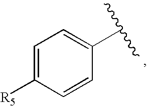
      R⁵ is F, then B is other than —CH₂C(O)OCH₃.

In various embodiments, R² is: (a) a C₁ to C₆ alkyl, (b) a C₁ to C₅ alkyl, (c) a C₁ to C₄ alkyl, (d) propyl, (e) ethyl, (f) methyl.

In certain embodiments where A is 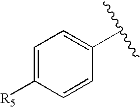
R⁵ is not —CF₃.

In other embodiments: R³ is F, Cl or Br; R³ is F or Cl; R³ is Cl; A is

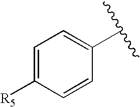

In other embodiments: R¹ is H; R⁵ is Cl; R⁵ is Cl, R³ is Cl, and R¹ is H; A is 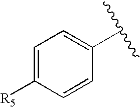
R⁵ is Cl, R³ is Cl, and R¹ is H; A is 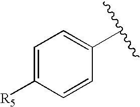
R⁵ is Cl, R³ is Cl, and R¹ is H, and R² is a C₁ to C₆ optionally substituted alkyl.

In other embodiments,
B is

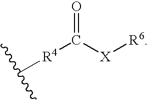

In other embodiments: the compound is in the form of a TRIS salt.

The invention features a pharmaceutical composition comprising the compound (or a salt thereof, e.g., a TRIS or other salt thereof) and a pharmaceutically acceptable carrier.

In certain embodiments: wherein X is —O—; X is —N—; R⁴ is an optionally independently substituted C₁ to C₆ alkyl; R⁴ is substituted; R⁴ is not substituted; X is —N— and R⁴ is an optionally independently substituted C₁ to C₆ alkyl; R⁴ is an optionally independently substituted C₁ to C₆ alkyl; R² is H or a C₁ to C₃ alkyl optionally substituted with a halogen; R² is H or a C₁ to C₃ alkyl optionally substituted with F; R² is selected from —CH₃, —CF₂H, —CF₃; R^{2A} is an unsubstituted C₁ to C₃ alkyl; R^{2A} is a substituted C₁ to C₃ alkyl; R^{2A} is an optionally independently substituted C₁ to C₆ alkyl, alkenyl, alkynyl or an optionally independently substituted C₆ to C₁₀ aryl or cycloalkyl; R⁶ is an optionally independently substituted C₁ to C₆ alkyl, alkenyl, alkynyl or an optionally independently substituted C₆ to C₁₀ aryl or cycloalkyl; R⁶ is substituted; and R⁶ is not substituted.

In certain embodiments: the compound exhibits an IC₅₀ for COX-1 that is at least 10-fold greater than for COX-2; the compound exhibits an IC₅₀ for COX-2 that is less than 1.0 μM; and the compound exhibits an IC₅₀ for FAAH that is less than 40 μM.

The invention also features: a method for treating pain and inflammation comprising administering the compound; a method for treating anxiety comprising administering the compound; a method for treating pain and inflammation comprising administering a pharmaceutical composition comprising the compound; a method for treating anxiety comprising administering a pharmaceutical composition comprising the compound.

A compound having the formula: 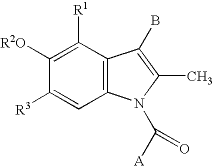
wherein:
R¹ is H or a halogen;
R² is H; a C₁ to C₆ alkyl, alkenyl, alkynyl optionally independently substituted with one or more halogen, —OH, —C(O)OH, —NH₃; 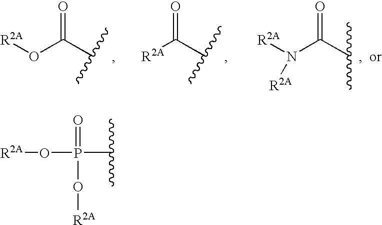
   wherein each R^{2A} is independently: H or a C₁ to C₆ alkyl, alkenyl, alkynyl, aryl, cycloalkyl, or arylalkyl optionally independently substituted with one or more halogen —OH, —C(O)OH, —NH₃;
R³ is H or a halogen;
A is 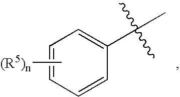
   wherein
   each R⁵ is independently H, a halogen, —CH₃, —CN, —OCH₃, —SCH₃, —OCF₂H, —OCF₃, or —CH₂CH₃, optionally independently substituted with one or more halogen, —OH, —C(O)OH, —NH₃; n=1, 2, 3, 4, or 5;
   provided that R³ is H only when R⁵ is —OCH₃, optionally independently substituted with one or more halogen;
B is 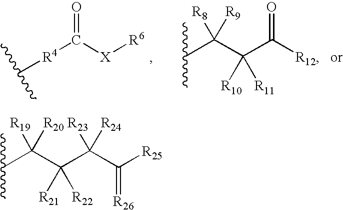
   wherein
   R⁴ is an optionally independently (singly or multiply) substituted C₁ to C₁₀ (C₁ to C₈, C₁ to C₆, C₁ to C₄, or C₁ to C₃ alkyl, —CH₂CH₂—, —CH₂—), alkenyl, alkynyl, wherein the substituents are selected from halogen, —OH, —C(O)OH, —CH₃, —OCH₃, —SCH₃, and —CH₂CH₃, optionally independently substituted with one or more halogen;
   X is —O—, —S—, —N(H)— or —N(H)S(O₂)—;
   R⁶ is: H; an optionally independently substituted C₁ to C₁₀ alkyl; an optionally independently substituted C₄ to C₈ cycloalkyl; a C₁ to C₆ hydroxyalkyl; a hydroxyl substituted C₄ to C₈ aryl; a primary, secondary or tertiary C₁ to C₆ alkylamino; a primary, secondary or tertiary branched C₁ to C₆ alkylamino; primary, secondary or tertiary C₄ to C₈ arylamino; C₂ to C₆ alkylcarboxylic acid; a branched C₃ to C₆ alkylcarboxylic acid; a C₁ to C₆ alkylester; a branched C₁ to C₆ alkylester; a C₄ to C₈ aryl; a C₄ to C₈ arylcarboxylic acid; a C₄ to C₈ arylester; a C₄ to C₈ aryl substituted C₁ to C₆ alkyl; a C₄ to C₈ heterocyclic alkyl or aryl; an alkyl-substituted or aryl-substituted C₄ to C₈ heterocyclic alkyl or aryl, wherein one or more H can be independently substituted by halogen, —OH, and —C(O)OH, —OCH₃, —CH₃, SCH₃ (wherein one or more H can be substituted by a halogen, e.g., F);
   R⁸, R⁹, R¹⁰, and R¹¹ are independently selected from:
      (a) H,
      (b) F,
      (c) methyl or ethyl,
      (d) —CF₃, —CF₂H or —CFH₂,
      (e) hydroxy, —OR¹⁴, SR¹⁴, S(O)R¹⁴, or S(O)₂R¹⁴,
      (f) naphthylmethyl, and
      (g) mono-substituted or di-substituted benzyl, wherein the substituents are selected from:
         (i) H,
         (ii) —CF₃,
         (iii) —CN,
         (iv) F, Cl, Br, or I,
         (v) C₁ to C₆ alkyl, and
         (vi) SR¹⁴, S(O)R¹⁴, or S(O)₂R¹⁴;
   or R⁸ and R⁹ together form an oxo group,
   or R¹⁰ and R¹¹ together form an oxo group,
   or R⁸ and R⁹ are joined so that together with the carbon atom to which they are attached there is formed a cycloalkyl having 3, 4, 5, 6 or 7 ring carbons;
   or R⁸ and R¹⁰ are joined so that together with the carbon atom to which they are attached there is formed a cycloalkyl having 3, 4, 5, 6 or 7 ring carbons;
   or R⁸ and R¹¹ are joined so that together with the carbon atom to which they are attached there is formed a cycloalkyl having 3, 4, 5, 6 or 7 ring carbons;
   or R⁹ and R¹⁰ are joined so that together with the carbon atom to which they are attached there is formed a cycloalkyl having 3, 4, 5, 6 or 7 ring carbons;
   or R⁹ and R¹¹ are joined so that together with the carbon atom to which they are attached there is formed a cycloalkyl having 3, 4, 5, 6 or 7 ring carbons;
   or R¹⁰ and R¹¹ are joined so that together with the carbon atom to which they are attached there is formed a cycloalkyl having 3, 4, 5, 6 or 7 ring carbons;
   R¹² is selected from:
      (a) —OR₁₃, and
      (b) —NR₁₅R₁₆;
   R¹³ is selected from:
      (a) H, and
      (b) C₁ to C₄ alkyl;
   R¹⁴ is selected from methyl, ethyl, mono-substituted benzyl or di-substituted benzyl, wherein the substituents are selected from:
      (a) H,
      (b) CF₃,
      (c) CN,
      (d) F, Cl, Br, I, and
      (e) C₁ to C₆ alkyl;
   R¹⁵ and R¹⁶ are independently selected from:
      (a) H,
      (b) C₁ to C₃ alkyl,
      (c) —OR₁₃,
      (d) —C(O)R₁₇
      (e) —S(O)₂R₁₈,
      (f) a mono-substituted C₂ to C₄ alkyl, wherein the substituent is selected from:
         (i) hydroxy,
         (ii) amino,
         (iii) methylamino,
         (iv) dimethyl amino, and
      (g) an unsubstituted, mono-substituted, or disubstituted phenyl, benzyl or pyridyl group, wherein the substituents are selected from:
         (i) H,
         (ii) CF₃,
         (iii) CN,
         (iv) F, Cl, Br, I, and
         (v) C₁₋₆ alkyl;
   or R¹⁵ and R¹⁶ are joined so that together with the nitrogen atom to which they are attached there is formed a 3, 4, 5, 6, or 7-membered ring, optionally including one or two additional heteroatoms, wherein the additional heteroatoms are selected from N, O, and S, the ring optionally including one or two carbonyl or sulfonyl groups;
   R¹⁷ is selected from:
      (a) H,
      (b) C₁ to C₄ alkyl,
      (c) CF₃, and
      (d) an unsubstituted, mono-substituted, or disubstituted phenyl or benzyl group, wherein the substituents are selected from:
         (i) H,
         (ii) CF₃,
         (iii) CN,
         (iv) F, Cl, Br, I, and
         (v) C₁ to C₆ alkyl;
   R¹⁸ is selected from:
      (a) C₁ to C₄ alkyl,
      (b) CF₃, and
      (c) an unsubstituted, mono-substituted, or disubstituted phenyl or benzyl group, wherein the substituents are selected from:
         (i) H,
         (ii) CF₃,
         (iii) CN,
         (iv) F, Cl, Br, I, and
         (v) C₁ to C₆ alkyl;
   R¹⁹, R²⁰, R²¹, R²², R²³ and R²⁴ are independently selected from:
      (a) H,
      (b) F or Cl,
      (c) C₁ to C₅ alkyl or haloalkyl,
      (d) C₃ to C₆ cycloalkyl,
      (e) —CF₃, —CF₂H or —CFH₂,
      (f) hydroxy, —OR₂₇, SR₂₇, S(O)R₂₇, or S(O)₂R₂₇,
      (g) mono-substituted or di-substituted benzyl, wherein the substituents are selected from:
         (i) —CF₃,
         (ii) —CN,
         (iii) F, Cl, Br, or I,
         (iv) C₁ to C₆ alkyl, and
         (v) SR₂₇, S(O)R₂₇, or S(O)₂R₂₇,
      (h) mono-substituted or di-substituted phenyl, wherein the substituents are selected from:
         (i) —CF₃,
         (ii) —CN,
         (iii) F, Cl, Br, or I,
         (iv) C₁ to C₆ alkyl, and
         (v) SR₂₇, S(O)R₂₇, or S(O)₂R₂₇,
   or R¹⁹ and R²⁰ together form an oxo group,
   or R²¹ and R²² together form an oxo group,
   or R²³ and R²⁴ together form an oxo group,
   or R¹⁹ and R²⁰ are joined so that together with the carbon atom to which they are attached there is formed a cycloalkyl having saturated 3, 4, 5, 6 or 7 ring carbons;
   or R²⁰ and R21 are joined so that together with the carbon atom to which they are attached there is formed a cycloalkyl having saturated 3, 4, 5, 6 or 7 ring carbons;
   or R²⁰ and R²³ are joined so that together with the carbon atom to which they are attached there is formed cycloalkyl having saturated 3, 4, 5, 6 or 7 ring carbons;
   or R²¹ and R²² are joined so that together with the carbon atom to which they are attached there is formed a cycloalkyl having saturated 3, 4, 5, 6 or 7 ring carbons;
   or R²¹ and R²³ are joined so that together with the carbon atom to which they are attached there is formed a cycloalkyl having saturated 3, 4, 5, 6 or 7 ring carbons;
   or R²⁴ and R²⁵ are joined so that together with the carbon atom to which they are attached there is formed a cycloalkyl having saturated 3, 4, 5, 6 or 7 ring carbons;
   R²⁶ is O or S;
   R²⁷ is methyl, ethyl, mono-substituted benzyl or di-substituted benzyl, wherein the substituents are selected from:
      (a) CF₃,
      (b) CN,
      (c) F, Cl, Br, I, and
      (d) C₁ to C₆ alkyl;
   R²⁸ and R²⁹ are independently selected from:
      (a) H,
      (b) C₁₋₃ alkyl,
      (c) C₃₋₆ cycloalkyl
      (d) —OR³²,
      (e) —C(O)R³⁰
      (f) —S(O)₂R³¹,
      (g) a mono-substituted C₂ to C₄ alkyl, wherein the substituent is selected from:
         (i) hydroxy,
         (ii) amino,
         (iii) methylamino,
         (iv) dimethyl amino
      (h) an unsubstituted, mono-substituted, or disubstituted phenyl, benzyl or pyridyl group, wherein the substituents are selected from:
         (i) H,
         (ii) CF₃,
         (iii) CN,
         (iv) F, Cl, Br, I, and
         (v) C₁ to C₆ alkyl,
   or R²⁸ and R²⁹ are joined so that together with the nitrogen atom to which they are attached there is formed a 3, 4, 5, 6, or 7-membered ring, optionally including one or two additional heteroatoms, wherein the additional heteroatoms are selected from N, O, and S, wherein a carbon atom may optionally be substituted with an oxo group and a sulfur atom of the ring may optionally substituted with two oxo groups;
   R³⁰ is selected from:
      (a) H,
      (b) C₁ to C₄ alkyl,
      (c) CF₃,
      (d) an unsubstituted, mono-substituted, or disubstituted phenyl or benzyl group, wherein the substituents are selected from:
         (i) CF₃,
         (ii) CN,
         (iii) F, Cl, Br, I, and
         (iv) C₁ to C₆ alkyl;
   R³¹ is selected from:
      (a) C₁ to C₄ alkyl,
      (b) CF₃,
      (c) an unsubstituted, mono-substituted, or disubstituted phenyl or benzyl group, wherein the substituents are selected from:
         (i) CF₃,
         (ii) CN,
         (iii) F, Cl, Br, I, and
         (iv) C₁ to C₆ alkyl;
   or a pharmaceutically acceptable salt thereof.

The compounds of the invention inhibit COX-2 or fatty acid amide hydrolase (FAAH) or both COX-2 and FAAH. In addition, certain of the compounds of the invention may inhibit NPAA. The compounds of the invention are useful in treating pain, inflammation and autoimmune diseases as well as other disorders. The compounds of the invention that inhibit COX-2 activity are relatively selective for COX-2 relative to COX-1. Thus, the COX-2 inhibitors of the invention do not substantially inhibit COX-1 at pharmacologically relevant concentrations at which COX-2 is substantially inhibited. Compounds of the invention that are relatively selective for FAAH do not substantially inhibit COX-2 at concentrations at which FAAH is substantially inhibited. Some compounds are relatively selective for COX-2 as compared to FAAH. These compounds do not substantially inhibit FAAH at concentrations at which COX-2 is substantially inhibited. Other compounds inhibit both COX-2 and FAAH at similar concentrations. These compounds are not particularly selective for COX-2 versus FAAH.

Certain compounds when administered to a patient exhibit FAAH inhibition and are then metabolized to a compound that does not significantly inhibit FAAH yet gain or retains the ability to selectively inhibit COX-2 over COX-1 at physiological concentrations.

COX-2 Inhibitors

Certain compounds of Formula I are COX-2 inhibitors that are selective for inhibition of COX-2 over COX-1 and do not substantially inhibit FAAH.

FAAH Inhibitors

Certain compounds within Formula I are FAAH inhibitors and are selective for inhibition of FAAH over both COX-2 and COX-1. Such compounds can include those in which B is 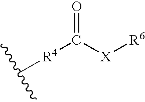
X is —O— or —N— and R⁶ is an alkyl (for example, B is an ester or amide) as well as other compounds.
FAAH and COX-2 Inhibitors

Certain compounds within Formula I inhibit both FAAH and COX-2 and are selective for inhibition of COX-2 over COX-1. Certain compounds have the ability to initially inhibit both FAAH and COX-2 and are metabolized to compounds that inhibit COX-2, but do not significantly inhibit FAAH.

Exemplary Compounds

Among the compounds of Formula I are:
[1-(4-chlorobenzoyl)-5-hydroxy-2-methyl-1H-indol-3-yl]acetic acid,
methyl [1-(4-chlorobenzoyl)-5-hydroxy-2-methyl-1H-indol-3-yl]acetate
ethyl [1-(4-chlorobenzoyl)-5-hydroxy-2-methyl-1H-indol-3-yl]acetate
propyl [1-(4-chlorobenzoyl)-5-hydroxy-2-methyl-1H-indol-3-yl]acetate
butyl [1-(4-chlorobenzoyl)-5-hydroxy-2-methyl-1H-indol-3-yl]acetate
sec-butyl [1-(4-chlorobenzoyl)-5-hydroxy-2-methyl-1H-indol-3-yl]acetate
(R) sec-butyl [1-(4-chlorobenzoyl)-5-hydroxy-2-methyl-1H-indol-3-yl]acetate
(S) sec-butyl [1-(4-chlorobenzoyl)-5-hydroxy-2-methyl-1H-indol-3-yl]acetate
isopropyl [1-(4-chlorobenzoyl)-5-hydroxy-2-methyl-1H-indol-3-yl]acetate
tert-butyl [1-(4-chlorobenzoyl)-5-hydroxy-2-methyl-1H-indol-3-yl]acetate
cyclobutyl [1-(4-chlorobenzoyl)-5-hydroxy-2-methyl-1H-indol-3-yl]acetate
cyclopentyl [1-(4-chlorobenzoyl)-5-hydroxy-2-methyl-1H-indol-3-yl]acetate
2-[1-(4-chlorobenzoyl)-5-hydroxy-2-methyl-1H-indol-3-yl]acetamide
2-[1-(4-chlorobenzoyl)-5-hydroxy-2-methyl-1H-indol-3-yl]-N-methylacetamide
2-[1-(4-chlorobenzoyl)-5-hydroxy-2-methyl-1H-indol-3-yl]-N-ethylacetamide
2-[1-(4-chlorobenzoyl)-5-hydroxy-2-methyl-1H-indol-3-yl]-N-propylacetamide
2-[1-(4-chlorobenzoyl)-5-hydroxy-2-methyl-1H-indol-3-yl]-N-isopropylacetamide
2-[1-(4-chlorobenzoyl)-5-hydroxy-2-methyl-1H-indol-3-yl]-N-butylacetamide
2-[1-(4-chlorobenzoyl)-5-hydroxy-2-methyl-1H-indol-3-yl]-N-sec-butylacetamide
2-[1-(4-chlorobenzoyl)-5-hydroxy-2-methyl-1H-indol-3-yl]-N-(2-hydroxyethyl)acetamide
({[1-(4-chlorobenzoyl)-5-hydroxy-2-methyl-1H-indol-3-yl]acetyl}amino)acetic acid
3-({[1-(4-chlorobenzoyl)-5-hydroxy-2-methyl-1H-indol-3-yl]acetyl}amino)propanoic acid
3-({[1-(4-chlorobenzoyl)-5-hydroxy-2-methyl-1H-indol-3-yl]acetyl}amino)-2-methylpropanoic acid
3-({[1-(4-chlorobenzoyl)-5-hydroxy-2-methyl-1H-indol-3-yl]acetyl}amino)-3-methylpropanoic acid
2-({[1-(4-chlorobenzoyl)-5-hydroxy-2-methyl-1H-indol-3-yl]acetyl}amino)propanoic acid
2-({[1-(4-chlorobenzoyl)-5-hydroxy-2-methyl-1H-indol-3-yl]acetyl}amino)-2-methylpropanoic acid
(2R)-2-[1-(4-chlorobenzoyl)-5-hydroxy-2-methyl-1H-indol-3-yl]propionic acid,
methyl (2R)-2-[1-(4-chlorobenzoyl)-5-hydroxy-2-methyl-1H-indol-3-yl]propanoate
ethyl (2R)-2-[1-(4-chlorobenzoyl)-5-hydroxy-2-methyl-1H-indol-3-yl]propanoate
propyl (2R)-2-[1-(4-chlorobenzoyl)-5-hydroxy-2-methyl-1H-indol-3-yl]propanoate
isopropyl (2R)-2-[1-(4-chlorobenzoyl)-5-hydroxy-2-methyl-1H-indol-3-yl]propanoate
butyl (2R)-2-[1-(4-chlorobenzoyl)-5-hydroxy-2-methyl-1H-indol-3-yl]propanoate
sec-butyl (2R)-2-[1-(4-chlorobenzoyl)-5-hydroxy-2-methyl-1H-indol-3-yl]propanoate
(R) sec-butyl (2R)-2-[1-(4-chlorobenzoyl)-5-hydroxy-2-methyl-1H-indol-3-yl]propanoate
(S) sec-butyl (2R)-2-[1-(4-chlorobenzoyl)-5-hydroxy-2-methyl-1H-indol-3-yl]propanoate
tert-butyl (2R)-2-[1-(4-chlorobenzoyl)-5-hydroxy-2-methyl-1H-indol-3-yl]propanoate
cyclobutyl (2R)-2-[1-(4-chlorobenzoyl)-5-hydroxy-2-methyl-1H-indol-3-yl]propanoate
cyclopentyl (2R)-2-[1-(4-chlorobenzoyl)-5-hydroxy-2-methyl-1H-indol-3-yl]propanoate
(2R)-2-[1-(4-chlorobenzoyl)-5-hydroxy-2-methyl-1H-indol-3-yl]propanamide
(2R)-2-[1-(4-chlorobenzoyl)-5-hydroxy-2-methyl-1H-indol-3-yl]-N-methylpropanamide
(2R)-2-[1-(4-chlorobenzoyl)-5-hydroxy-2-methyl-1H-indol-3-yl]-N-ethylacetamide
(2R)-2-[1-(4-chlorobenzoyl)-5-hydroxy-2-methyl-1H-indol-3-yl]-N-propylacetamide
(2R)-2-[1-(4-chlorobenzoyl)-5-hydroxy-2-methyl-1H-indol-3-yl]-N-isopropylacetamide
(2R)-2-[1-(4-chlorobenzoyl)-5-hydroxy-2-methyl-1H-indol-3-yl]-N-butylacetamide
(2R)-2-[1-(4-chlorobenzoyl)-5-hydroxy-2-methyl-1H-indol-3-yl]-N-sec-butylacetamide
(2R)-2-[1-(4-chlorobenzoyl)-5-hydroxy-2-methyl-1H-indol-3-yl]-N-(hydroxymethyl)propanamide
({(2R)-2-[1-(4-chlorobenzoyl)-5-hydroxy-2-methyl-1H-indol-3-yl]propanoyl}amino)acetic acid
({(2R)-2-[1-(4-chlorobenzoyl)-5-hydroxy-2-methyl-1H-indol-3-yl]propanoyl}amino)-2-methylpropionic acid
({(2R)-2-[1-(4-chlorobenzoyl)-5-hydroxy-2-methyl-1H-indol-3-yl]propanoyl}amino)-2,2-methylbutyric acid
(2R)-3-({[1-(4-chlorobenzoyl)-5-hydroxy-2-methyl-1H-indol-3-yl]propanoyl}amino)propanoic acid
(2R)-3-({[1-(4-chlorobenzoyl)-5-hydroxy-2-methyl-1H-indol-3-yl]propanoyl}amino)-3-methylpropanic acid
(2R)-3-({[1-(4-chlorobenzoyl)-5-hydroxy-2-methyl-1H-indol-3-yl]propanoyl}amino)-2-methylpropanic acid
(2S)-2-[1-(4-chlorobenzoyl)-5-hydroxy-2-methyl-1H-indol-3-yl]propionic acid,
methyl (2S)-2-[1-(4-chlorobenzoyl)-5-hydroxy-2-methyl-1H-indol-3-yl]propanoate
ethyl (2S)-2-[1-(4-chlorobenzoyl)-5-hydroxy-2-methyl-1H-indol-3-yl]propanoate
propyl (2S)-2-[1-(4-chlorobenzoyl)-5-hydroxy-2-methyl-1H-indol-3-yl]propanoate
isopropyl (2S)-2-[1-(4-chlorobenzoyl)-5-hydroxy-2-methyl-1H-indol-3-yl]propanoate
butyl (2S)-2-[1-(4-chlorobenzoyl)-5-hydroxy-2-methyl-1H-indol-3-yl]propanoate
sec-butyl (2S)-2-[1-(4-chlorobenzoyl)-5-hydroxy-2-methyl-1H-indol-3-yl]propanoate
(R) sec-butyl (2S)-2-[1-(4-chlorobenzoyl)-5-hydroxy-2-methyl-1H-indol-3-yl]propanoate
(S) sec-butyl (2S)-2-[1-(4-chlorobenzoyl)-5-hydroxy-2-methyl-1H-indol-3-yl]propanoate
tert-butyl (2S)-2-[1-(4-chlorobenzoyl)-5-hydroxy-2-methyl-1H-indol-3-yl]propanoate
cyclobutyl (2S)-2-[1-(4-chlorobenzoyl)-5-hydroxy-2-methyl-1H-indol-3-yl]propanoate
cyclopentyl (2S)-2-[1-(4-chlorobenzoyl)-5-hydroxy-2-methyl-1H-indol-3-yl]propanoate
(2S)-2-[1-(4-chlorobenzoyl)-5-hydroxy-2-methyl-1H-indol-3-yl]propanamide
(2S)-2-[1-(4-chlorobenzoyl)-5-hydroxy-2-methyl-1H-indol-3-yl]-N-methylpropanamide
(2S)-2-[1-(4-chlorobenzoyl)-5-hydroxy-2-methyl-1H-indol-3-yl]-N-ethylacetamide
(2S)-2-[1-(4-chlorobenzoyl)-5-hydroxy-2-methyl-1H-indol-3-yl]-N-propylacetamide
(2S)-2-[1-(4-chlorobenzoyl)-5-hydroxy-2-methyl-1H-indol-3-yl]-N-isopropylacetamide
(2S)-2-[1-(4-chlorobenzoyl)-5-hydroxy-2-methyl-1H-indol-3-yl]-N-butylacetamide
(2S)-2-[1-(4-chlorobenzoyl)-5-hydroxy-2-methyl-1H-indol-3-yl]-N-sec-butylacetamide
(2S)-2-[1-(4-chlorobenzoyl)-5-hydroxy-2-methyl-1H-indol-3-yl]-N-(hydroxymethyl)propanamide
({(2S)-2-[1-(4-chlorobenzoyl)-5-hydroxy-2-methyl-1H-indol-3-yl]propanoyl}amino)acetic acid
({(2S)-2-[1-(4-chlorobenzoyl)-5-hydroxy-2-methyl-1H-indol-3-yl]propanoyl}amino)-2-methylpropionic acid
({(2S)-2-[1-(4-chlorobenzoyl)-5-hydroxy-2-methyl-1H-indol-3-yl]propanoyl}amino)-2,2-methylbutyric acid
(2S)-3-({[1-(4-chlorobenzoyl)-5-hydroxy-2-methyl-1H-indol-3-yl]propanoyl}amino)propanoic acid
(2S)-3-({[1-(4-chlorobenzoyl)-5-hydroxy-2-methyl-1H-indol-3-yl]propanoyl}amino)-3-methylpropanic acid
(2S)-3-({[1-(4-chlorobenzoyl)-5-hydroxy-2-methyl-1H-indol-3-yl]propanoyl}amino)-2-methylpropanic acid
(2R,S)-2[1-(4-chlorobenzoyl)-5-hydroxy-2-methyl-1H-indol-3-yl]propionic acid,
methyl (2R,S)-2-[1-(4-chlorobenzoyl)-5-hydroxy-2-methyl-1H-indol-3-yl]propanoate
ethyl (2R,S)-2-[1-(4-chlorobenzoyl)-5-hydroxy-2-methyl-1H-indol-3-yl]propanoate
propyl (2R,S)-2-[1-(4-chlorobenzoyl)-5-hydroxy-2-methyl-1H-indol-3-yl]propanoate
isopropyl (2R,S)-2-[1-(4-chlorobenzoyl)-5-hydroxy-2-methyl-1H-indol-3-yl]propanoate
butyl (2R,S)-2-[1-(4-chlorobenzoyl)-5-hydroxy-2-methyl-1H-indol-3-yl]propanoate
sec-butyl (2R,S)-2-[1-(4-chlorobenzoyl)-5-hydroxy-2-methyl-1H-indol-3-yl]propanoate
(R) sec-butyl (2R,S)-2-[1-(4-chlorobenzoyl)-5-hydroxy-2-methyl-1H-indol-3-yl]propanoate
(S) sec-butyl (2R,S)-2-[1-(4-chlorobenzoyl)-5-hydroxy-2-methyl-1H-indol-3-yl]propanoate
tert-butyl (2R,S)-2-[1-(4-chlorobenzoyl)-5-hydroxy-2-methyl-1H-indol-3-yl]propanoate
cyclobutyl (2R,S)-2-[1-(4-chlorobenzoyl)-5-hydroxy-2-methyl-1H-indol-3-yl]propanoate
cyclopentyl (2R,S)-2-[1-(4-chlorobenzoyl)-5-hydroxy-2-methyl-1H-indol-3-yl]propanoate
(2R,S)-2-[1-(4-chlorobenzoyl)-5-hydroxy-2-methyl-1H-indol-3-yl]propanamide
(2R,S)-2-[1-(4-chlorobenzoyl)-5-hydroxy-2-methyl-1H-indol-3-yl]-N-methylpropanamide
(2R,S)-2-[1-(4-chlorobenzoyl)-5-hydroxy-2-methyl-1H-indol-3-yl]-N-ethylacetamide
(2R,S)-2-[1-(4-chlorobenzoyl)-5-hydroxy-2-methyl-1H-indol-3-yl]-N-propylacetamide
(2R,S)-2-[1-(4-chlorobenzoyl)-5-hydroxy-2-methyl-1H-indol-3-yl]-N-isopropylacetamide
(2R,S)-2-[1-(4-chlorobenzoyl)-5-hydroxy-2-methyl-1H-indol-3-yl]-N-butylacetamide
(2R,S)-2-[1-(4-chlorobenzoyl)-5-hydroxy-2-methyl-1H-indol-3-yl]-N-sec-butylacetamide
(2R,S)-2-[1-(4-chlorobenzoyl)-5-hydroxy-2-methyl-1H-indol-3-yl]-N-(hydroxymethyl)propanamide
({(2R,S)-2-[1-(4-chlorobenzoyl)-5-hydroxy-2-methyl-1H-indol-3-yl]propanoyl}amino)acetic acid
({(2R,S)-2-[1-(4-chlorobenzoyl)-5-hydroxy-2-methyl-1H-indol-3-yl]propanoyl}amino)-2-methylpropionic acid
({(2R,S)-2-[1-(4-chlorobenzoyl)-5-hydroxy-2-methyl-1H-indol-3-yl]propanoyl}amino)-2,2-methylbutyric acid
(2R,S)-3-({[1-(4-chlorobenzoyl)-5-hydroxy-2-methyl-1H-indol-3-yl]propanoyl}amino)propanoic acid
(2R,S)-3-({[1-(4-chlorobenzoyl)-5-hydroxy-2-methyl-1H-indol-3-yl]propanoyl}amino)-3-methylpropanic acid
(2R,S)-3-({[1-(4-chlorobenzoyl)-5-hydroxy-2-methyl-1H-indol-3-yl]propanoyl}amino)-2-methylpropanic acid
[1-(4-chlorobenzoyl)-6-fluoro-5-hydroxy-2-methyl-1H-indol-3-yl]acetic acid,
methyl [1-(4-chlorobenzoyl)-6-fluoro-5-hydroxy-2-methyl-1H-indol-3-yl]acetate
ethyl [1-(4-chlorobenzoyl)-6-fluoro-5-hydroxy-2-methyl-1H-indol-3-yl]acetate
propyl [1-(4-chlorobenzoyl)-6-fluoro-5-hydroxy-2-methyl-1H-indol-3-yl]acetate
butyl [1-(4-chlorobenzoyl)-6-fluoro-5-hydroxy-2-methyl-1H-indol-3-yl]acetate
sec-butyl [1-(4-chlorobenzoyl)-6-fluoro-5-hydroxy-2-methyl-1H-indol-3-yl]acetate
(R) sec-butyl [1-(4-chlorobenzoyl)-6-fluoro-5-hydroxy-2-methyl-1H-indol-3-yl]acetate
(S) sec-butyl [1-(4-chlorobenzoyl)-6-fluoro-5-hydroxy-2-methyl-1H-indol-3-yl]acetate
isopropyl [1-(4-chlorobenzoyl)-6-fluoro-5-hydroxy-2-methyl-1H-indol-3-yl]acetate
tert-butyl [1-(4-chlorobenzoyl)-6-fluoro-5-hydroxy-2-methyl-1H-indol-3-yl]acetate
cyclobutyl [1-(4-chlorobenzoyl)-6-fluoro-5-hydroxy-2-methyl-1H-indol-3-yl]acetate
cyclopentyl [1-(4-chlorobenzoyl)-6-fluoro-5-hydroxy-2-methyl-1H-indol-3-yl]acetate
2-[1-(4-chlorobenzoyl)-6-fluoro-5-hydroxy-2-methyl-1H-indol-3-yl]acetamide
2-[1-(4-chlorobenzoyl)-6-fluoro-5-hydroxy-2-methyl-1H-indol-3-yl]-N-methylacetamide
2-[1-(4-chlorobenzoyl)-6-fluoro-5-hydroxy-2-methyl-1H-indol-3-yl]-N-ethylacetamide
2-[1-(4-chlorobenzoyl)-6-fluoro-5-hydroxy-2-methyl-1H-indol-3-yl]-N-propylacetamide
2-[1-(4-chlorobenzoyl)-6-fluoro-5-hydroxy-2-methyl-1H-indol-3-yl]-N-isopropylacetamide
2-[1-(4-chlorobenzoyl)-6-fluoro-5-hydroxy-2-methyl-1H-indol-3-yl]-N-butylacetamide
2-[1-(4-chlorobenzoyl)-6-fluoro-5-hydroxy-2-methyl-1H-indol-3-yl]-N-sec-butylacetamide
2-[1-(4-chlorobenzoyl)-6-fluoro-5-hydroxy-2-methyl-1H-indol-3-yl]-N-(2-hydroxyethyl)acetamide
({[1-(4-chlorobenzoyl)-6-fluoro-5-hydroxy-2-methyl-1H-indol-3-yl]acetyl}amino)acetic acid
3-({[1-(4-chlorobenzoyl)-6-fluoro-5-hydroxy-2-methyl-1H-indol-3-yl]acetyl}amino)propanoic acid
3-({[1-(4-chlorobenzoyl)-6-fluoro-5-hydroxy-2-methyl-1H-indol-3-yl]acetyl}amino)-2-methylpropanoic acid
3-({[1-(4-chlorobenzoyl)-6-fluoro-5-hydroxy-2-methyl-1H-indol-3-yl]acetyl}amino)-3-methylpropanoic acid
2-({[1-(4-chlorobenzoyl)-6-fluoro-5-hydroxy-2-methyl-1H-indol-3-yl]acetyl}amino)propanoic acid
2-({[1-(4-chlorobenzoyl)-6-fluoro-5-hydroxy-2-methyl-1H-indol-3-yl]acetyl}amino)-2-methylpropanoic acid
[1-(4-chlorobenzoyl)-4-fluoro-5-hydroxy-2-methyl-1H-indol-3-yl]acetic acid,
methyl [1-(4-chlorobenzoyl)-4-fluoro-5-hydroxy-2-methyl-1H-indol-3-yl]acetate
ethyl [1-(4-chlorobenzoyl)-4-fluoro-5-hydroxy-2-methyl-1H-indol-3-yl]acetate
propyl [1-(4-chlorobenzoyl)-4-fluoro-5-hydroxy-2-methyl-1H-indol-3-yl]acetate
butyl [1-(4-chlorobenzoyl)-4-fluoro-5-hydroxy-2-methyl-1H-indol-3-yl]acetate
sec-butyl [1-(4-chlorobenzoyl)-4-fluoro-5-hydroxy-2-methyl-1H-indol-3-yl]acetate
(R) sec-butyl [1-(4-chlorobenzoyl)-4-fluoro-5-hydroxy-2-methyl-1H-indol-3-yl]acetate
(S) sec-butyl [1-(4-chlorobenzoyl)-4-fluoro-5-hydroxy-2-methyl-1H-indol-3-yl]acetate
isopropyl [1-(4-chlorobenzoyl)-4-fluoro-5-hydroxy-2-methyl-1H-indol-3-yl]acetate
tert-butyl [1-(4-chlorobenzoyl)-4-fluoro-5-hydroxy-2-methyl-1H-indol-3-yl]acetate
cyclobutyl [1-(4-chlorobenzoyl)-4-fluoro-5-hydroxy-2-methyl-1H-indol-3-yl]acetate
cyclopentyl [1-(4-chlorobenzoyl)-4-fluoro-5-hydroxy-2-methyl-1H-indol-3-yl]acetate
2-[1-(4-chlorobenzoyl)-4-fluoro-5-hydroxy-2-methyl-1H-indol-3-yl]acetamide
2-[1-(4-chlorobenzoyl)-4-fluoro-5-hydroxy-2-methyl-1H-indol-3-yl]-N-methylacetamide
2-[1-(4-chlorobenzoyl)-4-fluoro-5-hydroxy-2-methyl-1H-indol-3-yl]-N-ethylacetamide
2-[1-(4-chlorobenzoyl)-4-fluoro-5-hydroxy-2-methyl-1H-indol-3-yl]-N-propylacetamide
2-[1-(4-chlorobenzoyl)-4-fluoro-5-hydroxy-2-methyl-1H-indol-3-yl]-N-isopropylacetamide
2-[1-(4-chlorobenzoyl)-4-fluoro-5-hydroxy-2-methyl-1H-indol-3-yl]-N-butylacetamide
2-[1-(4-chlorobenzoyl)-4-fluoro-5-hydroxy-2-methyl-1H-indol-3-yl]-N-sec-butylacetamide
2-[1-(4-chlorobenzoyl)-4-fluoro-5-hydroxy-2-methyl-1H-indol-3-yl]-N-(2-hydroxyethyl)acetamide
({[1-(4-chlorobenzoyl)-4-fluoro-5-hydroxy-2-methyl-1H-indol-3-yl]acetyl}amino)acetic acid
3-({[1-(4-chlorobenzoyl)-4-fluoro-5-hydroxy-2-methyl-1H-indol-3-yl]acetyl}amino)propanoic acid
3-({[1-(4-chlorobenzoyl)-4-fluoro-5-hydroxy-2-methyl-1H-indol-3-yl]acetyl}amino)-2-methylpropanoic acid
3-({[1-(4-chlorobenzoyl)-4-fluoro-5-hydroxy-2-methyl-1H-indol-3-yl]acetyl}amino)-3-methylpropanoic acid
2-({[1-(4-chlorobenzoyl)-4-fluoro-5-hydroxy-2-methyl-1H-indol-3-yl]acetyl}amino)propanoic acid
2-({[1-(4-chlorobenzoyl)-4-fluoro-5-hydroxy-2-methyl-1H-indol-3-yl]acetyl}amino)-2-methylpropanoic acid
[1-(4-chlorobenzoyl)-4,6-difluoro-5-hydroxy-2-methyl-1H-indol-3-yl]acetic acid,
methyl [1-(4-chlorobenzoyl)-4,6-difluoro-5-hydroxy-2-methyl-1H-indol-3-yl]acetate
ethyl [1-(4-chlorobenzoyl)-4,6-difluoro-5-hydroxy-2-methyl-1H-indol-3-yl]acetate
propyl [1-(4-chlorobenzoyl)-4,6-difluoro-5-hydroxy-2-methyl-1H-indol-3-yl]acetate
butyl [1-(4-chlorobenzoyl)-4,6-difluoro-5-hydroxy-2-methyl-1H-indol-3-yl]acetate
sec-butyl [1-(4-chlorobenzoyl)-4,6-difluoro-5-hydroxy-2-methyl-1H-indol-3-yl]acetate
(R) sec-butyl [1-(4-chlorobenzoyl)-4,6-difluoro-5-hydroxy-2-methyl-1H-indol-3-yl]acetate
(S) sec-butyl [1-(4-chlorobenzoyl)-4,6-difluoro-5-hydroxy-2-methyl-1H-indol-3-yl]acetate
isopropyl [1-(4-chlorobenzoyl)-4,6-difluoro-5-hydroxy-2-methyl-1H-indol-3-yl]acetate
tert-butyl [1-(4-chlorobenzoyl)-4,6-difluoro-5-hydroxy-2-methyl-1H-indol-3-yl]acetate
cyclobutyl [1-(4-chlorobenzoyl)-4,6-difluoro-5-hydroxy-2-methyl-1H-indol-3-yl]acetate
cyclopentyl [1-(4-chlorobenzoyl)-4,6-difluoro-5-hydroxy-2-methyl-1H-indol-3-yl]acetate
2-[1-(4-chlorobenzoyl)-4,6-difluoro-5-hydroxy-2-methyl-1H-indol-3-yl]acetamide
2-[1-(4-chlorobenzoyl)-4,6-difluoro-5-hydroxy-2-methyl-1H-indol-3-yl]-N-methylacetamide
2-[1-(4-chlorobenzoyl)-4,6-difluoro-5-hydroxy-2-methyl-1H-indol-3-yl]-N-ethylacetamide
2-[1-(4-chlorobenzoyl)-4,6-difluoro-5-hydroxy-2-methyl-1H-indol-3-yl]-N-propylacetamide
2-[1-(4-chlorobenzoyl)-4,6-difluoro-5-hydroxy-2-methyl-1H-indol-3-yl]-N-isopropylacetamide
2-[1-(4-chlorobenzoyl)-4,6-difluoro-5-hydroxy-2-methyl-1H-indol-3-yl]-N-butylacetamide
2-[1-(4-chlorobenzoyl)-4,6-difluoro-5-hydroxy-2-methyl-1H-indol-3-yl]-N-sec-butylacetamide
2-[1-(4-chlorobenzoyl)-4,6-difluoro-5-hydroxy-2-methyl-1H-indol-3-yl]-N-(2-hydroxyethyl)acetamide
({[1-(4-chlorobenzoyl)-4,6-difluoro-5-hydroxy-2-methyl-1H-indol-3-yl]acetyl}amino)acetic acid
3-({[1-(4-chlorobenzoyl)-4,6-difluoro-5-hydroxy-2-methyl-1H-indol-3-yl]acetyl}amino)propanoic acid
3-({[1-(4-chlorobenzoyl)-4,6-difluoro-5-hydroxy-2-methyl-1H-indol-3-yl]acetyl}amino)-2-methylpropanoic acid
3-({[1-(4-chlorobenzoyl)-4,6-difluoro-5-hydroxy-2-methyl-1H-indol-3-yl]acetyl}amino)-3-methylpropanoic acid
2-({[1-(4-chlorobenzoyl)-4,6-difluoro-5-hydroxy-2-methyl-1H-indol-3-yl]acetyl}amino)propanoic acid
2-({[1-(4-chlorobenzoyl)-4,6-difluoro-5-hydroxy-2-methyl-1H-indol-3-yl]acetyl}amino)-2-methylpropanoic acid
(2R,S)-2[1-(4-chlorobenzoyl)-4-fluoro-5-hydroxy-2-methyl-1H-indol-3-yl]propionic acid,
methyl (2R,S)-2-[1-(4-chlorobenzoyl)-4-fluoro-5-hydroxy-2-methyl-1H-indol-3-yl]propanoate
ethyl (2R,S)-2-[1-(4-chlorobenzoyl)-4-fluoro-5-hydroxy-2-methyl-1H-indol-3-yl]propanoate
propyl (2R,S)-2-[1-(4-chlorobenzoyl)-4-fluoro-5-hydroxy-2-methyl-1H-indol-3-yl]propanoate
isopropyl (2R,S)-2-[1-(4-chlorobenzoyl)-4-fluoro-5-hydroxy-2-methyl-1H-indol-3-yl]propanoate
butyl (2R,S)-2-[1-(4-chlorobenzoyl)-4-fluoro-5-hydroxy-2-methyl-1H-indol-3-yl]propanoate
sec-butyl (2R,S)-2-[1-(4-chlorobenzoyl)-4-fluoro-5-hydroxy-2-methyl-1H-indol-3-yl]propanoate
(R) sec-butyl (2R,S)-2-[1-(4-chlorobenzoyl)-4-fluoro-5-hydroxy-2-methyl-1H-indol-3-yl]propanoate
(S) sec-butyl (2R,S)-2-[1-(4-chlorobenzoyl)-4-fluoro-5-hydroxy-2-methyl-1H-indol-3-yl]propanoate
tert-butyl (2R,S)-2-[1-(4-chlorobenzoyl)-4-fluoro-5-hydroxy-2-methyl-1H-indol-3-yl]propanoate
cyclobutyl (2R,S)-2-[1-(4-chlorobenzoyl)-4-fluoro-5-hydroxy-2-methyl-1H-indol-3-yl]propanoate
cyclopentyl (2R,S)-2-[1-(4-chlorobenzoyl)-4-fluoro-5-hydroxy-2-methyl-1H-indol-3-yl]propanoate
(2R,S)-2-[1-(4-chlorobenzoyl)-4-fluoro-5-hydroxy-2-methyl-1H-indol-3-yl]propanamide
(2R,S)-2-[1-(4-chlorobenzoyl)-4-fluoro-5-hydroxy-2-methyl-1H-indol-3-yl]-N-methylpropanamide
(2R,S)-2-[1-(4-chlorobenzoyl)-4-fluoro-5-hydroxy-2-methyl-1H-indol-3-yl]-N-ethylacetamide
(2R,S)-2-[1-(4-chlorobenzoyl)-4-fluoro-5-hydroxy-2-methyl-1H-indol-3-yl]-N-propylacetamide
(2R,S)-2-[1-(4-chlorobenzoyl)-4-fluoro-5-hydroxy-2-methyl-1H-indol-3-yl]-N-isopropylacetamide
(2R,S)-2-[1-(4-chlorobenzoyl)-4-fluoro-5-hydroxy-2-methyl-1H-indol-3-yl]-N-butylacetamide
(2R,S)-2-[1-(4-chlorobenzoyl)-4-fluoro-5-hydroxy-2-methyl-1H-indol-3-yl]-N-sec-butylacetamide
(2R,S)-2-[1-(4-chlorobenzoyl)-4-fluoro-5-hydroxy-2-methyl-1H-indol-3-yl]-N-(hydroxymethyl)propanamide
({(2R,S)-2-[1-(4-chlorobenzoyl)-4-fluoro-5-hydroxy-2-methyl-1H-indol-3-yl]propanoyl}amino)acetic acid
({(2R,S)-2-[1-(4-chlorobenzoyl)-4-fluoro-5-hydroxy-2-methyl-1H-indol-3-yl]propanoyl}amino)-2-methylpropionic acid
({(2R,S)-2-[1-(4-chlorobenzoyl)-4-fluoro-5-hydroxy-2-methyl-1H-indol-3-yl]propanoyl}amino)-2,2-methylbutyric acid
(2R,S)-3-({[1-(4-chlorobenzoyl)-4-fluoro-5-hydroxy-2-methyl-1H-indol-3-yl]propanoyl}amino)propanoic acid
(2R,S)-3-({[1-(4-chlorobenzoyl)-4-fluoro-5-hydroxy-2-methyl-1H-indol-3-yl]propanoyl}amino)-3-methylpropanic acid
(2R,S)-3-({[1-(4-chlorobenzoyl)-4-fluoro-5-hydroxy-2-methyl-1H-indol-3-yl]propanoyl}amino)-2-methylpropanic acid
(2R,S)-2[1-(4-chlorobenzoyl)-6-fluoro-5-hydroxy-2-methyl-1H-indol-3-yl]propionic acid,
methyl (2R,S)-2-[1-(4-chlorobenzoyl)-6-fluoro-5-hydroxy-2-methyl-1H-indol-3-yl]propanoate
ethyl (2R,S)-2-[1-(4-chlorobenzoyl)-6-fluoro-5-hydroxy-2-methyl-1H-indol-3-yl]propanoate
propyl (2R,S)-2-[1-(4-chlorobenzoyl)-6-fluoro-5-hydroxy-2-methyl-1H-indol-3-yl]propanoate
isopropyl (2R,S)-2-[1-(4-chlorobenzoyl)-6-fluoro-5-hydroxy-2-methyl-1H-indol-3-yl]propanoate
butyl (2R,S)-2-[1-(4-chlorobenzoyl)-6-fluoro-5-hydroxy-2-methyl-1H-indol-3-yl]propanoate
sec-butyl (2R,S)-2-[1-(4-chlorobenzoyl)-6-fluoro-5-hydroxy-2-methyl-1H-indol-3-yl]propanoate
(R) sec-butyl (2R,S)-2-[1-(4-chlorobenzoyl)-6-fluoro-5-hydroxy-2-methyl-1H-indol-3-yl]propanoate
(S) sec-butyl (2R,S)-2-[1-(4-chlorobenzoyl)-6-fluoro-5-hydroxy-2-methyl-1H-indol-3-yl]propanoate
tert-butyl (2R,S)-2-[1-(4-chlorobenzoyl)-6-fluoro-5-hydroxy-2-methyl-1H-indol-3-yl]propanoate
cyclobutyl (2R,S)-2-[1-(4-chlorobenzoyl)-6-fluoro-5-hydroxy-2-methyl-1H-indol-3-yl]propanoate
cyclopentyl (2R,S)-2-[1-(4-chlorobenzoyl)-6-fluoro-5-hydroxy-2-methyl-1H-indol-3-yl]propanoate
(2R,S)-2-[1-(4-chlorobenzoyl)-6-fluoro-5-hydroxy-2-methyl-1H-indol-3-yl]propanamide
(2R,S)-2-[1-(4-chlorobenzoyl)-6-fluoro-5-hydroxy-2-methyl-1H-indol-3-yl]-N-methylpropanamide
(2R,S)-2-[1-(4-chlorobenzoyl)-6-fluoro-5-hydroxy-2-methyl-1H-indol-3-yl]-N-ethylacetamide
(2R,S)-2-[1-(4-chlorobenzoyl)-6-fluoro-5-hydroxy-2-methyl-1H-indol-3-yl]-N-propylacetamide
(2R,S)-2-[1-(4-chlorobenzoyl)-6-fluoro-5-hydroxy-2-methyl-1H-indol-3-yl]-N-isopropylacetamide
(2R,S)-2-[1-(4-chlorobenzoyl)-6-fluoro-5-hydroxy-2-methyl-1H-indol-3-yl]-N-butylacetamide
(2R,S)-2-[1-(4-chlorobenzoyl)-6-fluoro-5-hydroxy-2-methyl-1H-indol-3-yl]-N-sec-butylacetamide
(2R,S)-2-[1-(4-chlorobenzoyl)-6-fluoro-5-hydroxy-2-methyl-1H-indol-3-yl]-N-(hydroxymethyl)propanamide
({(2R,S)-2-[1-(4-chlorobenzoyl)-6-fluoro-5-hydroxy-2-methyl-1H-indol-3-yl]propanoyl}amino)acetic acid
({(2R,S)-2-[1-(4-chlorobenzoyl)-6-fluoro-5-hydroxy-2-methyl-1H-indol-3-yl]propanoyl}amino)-2-methylpropionic acid
({(2R,S)-2-[1-(4-chlorobenzoyl)-6-fluoro-5-hydroxy-2-methyl-1H-indol-3-yl]propanoyl}amino)-2,2-methylbutyric acid
(2R,S)-3-({[1-(4-chlorobenzoyl)-6-fluoro-5-hydroxy-2-methyl-1H-indol-3-yl]propanoyl}amino)propanoic acid
(2R,S)-3-({[1-(4-chlorobenzoyl)-6-fluoro-5-hydroxy-2-methyl-1H-indol-3-yl]propanoyl}amino)-3-methylpropanic acid
(2R,S)-3-({[1-(4-chlorobenzoyl)-6-fluoro-5-hydroxy-2-methyl-1H-indol-3-yl]propanoyl}amino)-2-methylpropanic acid
(2R,S)-2[1-(4-chlorobenzoyl)-4,6-difluoro-5-hydroxy-2-methyl-1H-indol-3-yl]propionic acid,
methyl (2R,S)-2-[1-(4-chlorobenzoyl)-4,6-difluoro-5-hydroxy-2-methyl-1H-indol-3-yl]propanoate
ethyl (2R,S)-2-[1-(4-chlorobenzoyl)-4,6-difluoro-5-hydroxy-2-methyl-1H-indol-3-yl]propanoate
propyl (2R,S)-2-[1-(4-chlorobenzoyl)-4,6-difluoro-5-hydroxy-2-methyl-1H-indol-3-yl]propanoate
isopropyl (2R,S)-2-[1-(4-chlorobenzoyl)-4,6-difluoro-5-hydroxy-2-methyl-1H-indol-3-yl]propanoate
butyl (2R,S)-2-[1′-(4-chlorobenzoyl)-4,6-difluoro-5-hydroxy-2-methyl-1H-indol-3-yl]propanoate
sec-butyl (2R,S)-2-[1-(4-chlorobenzoyl)-4,6-difluoro-5-hydroxy-2-methyl-1H-indol-3-yl]propanoate
(R) sec-butyl (2R,S)-2-[1-(4-chlorobenzoyl)-4,6-difluoro-5-hydroxy-2-methyl-1H-indol-3-yl]propanoate
(S) sec-butyl (2R,S)-2-[1-(4-chlorobenzoyl)-4,6-difluoro-5-hydroxy-2-methyl-1H-indol-3-yl]propanoate
tert-butyl (2R,S)-2-[1-(4-chlorobenzoyl)-4,6-difluoro-5-hydroxy-2-methyl-1H-indol-3-yl]propanoate
cyclobutyl (2R,S)-2-[1-(4-chlorobenzoyl)-4,6-difluoro-5-hydroxy-2-methyl-1H-indol-3-yl]propanoate
cyclopentyl (2R,S)-2-[1-(4-chlorobenzoyl)-4,6-difluoro-5-hydroxy-2-methyl-1H-indol-3-yl]propanoate
(2R,S)-2-[1-(4-chlorobenzoyl)-4,6-difluoro-5-hydroxy-2-methyl-1H-indol-3-yl]propanamide
(2R,S)-2-[1-(4-chlorobenzoyl)-4,6-difluoro-5-hydroxy-2-methyl-1H-indol-3-yl]-N-methylpropanamide
(2R,S)-2-[1-(4-chlorobenzoyl)-4,6-difluoro-5-hydroxy-2-methyl-1H-indol-3-yl]-N-ethylacetamide
(2R,S)-2-[1-(4-chlorobenzoyl)-4,6-difluoro-5-hydroxy-2-methyl-1H-indol-3-yl]-N-propylacetamide
(2R,S)-2-[1-(4-chlorobenzoyl)-4,6-difluoro-5-hydroxy-2-methyl-1H-indol-3-yl]-N-isopropylacetamide
(2R,S)-2-[1-(4-chlorobenzoyl)-4,6-difluoro-5-hydroxy-2-methyl-1H-indol-3-yl]-N-butylacetamide
(2R,S)-2-[1-(4-chlorobenzoyl)-4,6-difluoro-5-hydroxy-2-methyl-1H-indol-3-yl]-N-sec-butylacetamide
(2R,S)-2-[1-(4-chlorobenzoyl)-4,6-difluoro-5-hydroxy-2-methyl-1H-indol-3-yl]-N-(hydroxymethyl)propanamide
({(2R,S)-2-[1-(4-chlorobenzoyl)-4,6-difluoro-5-hydroxy-2-methyl-1H-indol-3-yl]propanoyl}amino)acetic acid
({(2R,S)-2-[1-(4-chlorobenzoyl)-4,6-difluoro-5-hydroxy-2-methyl-1H-indol-3-yl]propanoyl}amino)-2-methylpropionic acid
({(2R,S)-2-[1-(4-chlorobenzoyl)-4,6-difluoro-5-hydroxy-2-methyl-1H-indol-3-yl]propanoyl}amino)-2,2-methylbutyric acid
(2R,S)-3-({[1-(4-chlorobenzoyl)-4,6-difluoro-5-hydroxy-2-methyl-1H-indol-3-yl]propanoyl}amino)propanoic acid
(2R,S)-3-({[1-(4-chlorobenzoyl)-4,6-difluoro-5-hydroxy-2-methyl-1H-indol-3-yl]propanoyl}amino)-3-methylpropanic acid
(2R,S)-3-({[1-(4-chlorobenzoyl)-4,6-difluoro-5-hydroxy-2-methyl-1H-indol-3-yl]propanoyl}amino)-2-methylpropanic acid
[1-(3-chlorobenzoyl)-4,6-difluoro-5-hydroxy-2-methyl-1H-indol-3-yl]acetic acid,
methyl [1-(3-chlorobenzoyl)-4,6-difluoro-5-hydroxy-2-methyl-1H-indol-3-yl]acetate
ethyl [1-(3-chlorobenzoyl)-4,6-difluoro-5-hydroxy-2-methyl-1H-indol-3-yl]acetate
propyl [1-(3-chlorobenzoyl)-4,6-difluoro-5-hydroxy-2-methyl-1H-indol-3-yl]acetate
butyl [1-(3-chlorobenzoyl)-4,6-difluoro-5-hydroxy-2-methyl-1H-indol-3-yl]acetate
sec-butyl [1-(3-chlorobenzoyl)-4,6-difluoro-5-hydroxy-2-methyl-1H-indol-3-yl]acetate
(R) sec-butyl [1-(3-chlorobenzoyl)-4,6-difluoro-5-hydroxy-2-methyl-1H-indol-3-yl]acetate
(S) sec-butyl [1-(3-chlorobenzoyl)-4,6-difluoro-5-hydroxy-2-methyl-1H-indol-3-yl]acetate
isopropyl [1-(3-chlorobenzoyl)-4,6-difluoro-5-hydroxy-2-methyl-1H-indol-3-yl]acetate
tert-butyl [1-(3-chlorobenzoyl)-4,6-difluoro-5-hydroxy-2-methyl-1H-indol-3-yl]acetate
cyclobutyl [1-(3-chlorobenzoyl)-4,6-difluoro-5-hydroxy-2-methyl-1H-indol-3-yl]acetate
cyclopentyl [1-(3-chlorobenzoyl)-4,6-difluoro-5-hydroxy-2-methyl-1H-indol-3-yl]acetate
2-[1-(3-chlorobenzoyl)-4,6-difluoro-5-hydroxy-2-methyl-1H-indol-3-yl]acetamide
2-[1-(3-chlorobenzoyl)-4,6-difluoro-5-hydroxy-2-methyl-1H-indol-3-yl]-N-methylacetamide
2-[1-(3-chlorobenzoyl)-4,6-difluoro-5-hydroxy-2-methyl-1H-indol-3-yl]-N-ethylacetamide
2-[1-(3-chlorobenzoyl)-4,6-difluoro-5-hydroxy-2-methyl-1H-indol-3-yl]-N-propylacetamide
2-[1-(3-chlorobenzoyl)-4,6-difluoro-5-hydroxy-2-methyl-1H-indol-3-yl]-N-isopropylacetamide
2-[1-(3-chlorobenzoyl)-4,6-difluoro-5-hydroxy-2-methyl-1H-indol-3-yl]-N-butylacetamide
2-[1-(3-chlorobenzoyl)-4,6-difluoro-5-hydroxy-2-methyl-1H-indol-3-yl]-N-sec-butylacetamide
2-[1-(3-chlorobenzoyl)-4,6-difluoro-5-hydroxy-2-methyl-1H-indol-3-yl]-N-(2-hydroxyethyl)acetamide
({[1-(3-chlorobenzoyl)-4,6-difluoro-5-hydroxy-2-methyl-1H-indol-3-yl]acetyl}amino)acetic acid
3-({[1-(3-chlorobenzoyl)-4,6-difluoro-5-hydroxy-2-methyl-1H-indol-3-yl]acetyl}amino)propanoic acid
3-({[1-(3-chlorobenzoyl)-4,6-difluoro-5-hydroxy-2-methyl-1H-indol-3-yl]acetyl}amino)-2-methylpropanoic acid
3-({[1-(3-chlorobenzoyl)-4,6-difluoro-5-hydroxy-2-methyl-1H-indol-3-yl]acetyl}amino)-3-methylpropanoic acid
2-({[1-(3-chlorobenzoyl)-4,6-difluoro-5-hydroxy-2-methyl-1H-indol-3-yl]acetyl}amino)propanoic acid
2-({[1-(3-chlorobenzoyl)-4,6-difluoro-5-hydroxy-2-methyl-1H-indol-3-yl]acetyl}amino)-2-methylpropanoic acid
[1-(3-chlorobenzoyl)-6-fluoro-5-hydroxy-2-methyl-1H-indol-3-yl]acetic acid,
methyl [1-(3-chlorobenzoyl)-6-fluoro-5-hydroxy-2-methyl-1H-indol-3-yl]acetate
ethyl [1-(3-chlorobenzoyl)-6-fluoro-5-hydroxy-2-methyl-1H-indol-3-yl]acetate
propyl [1-(3-chlorobenzoyl)-6-fluoro-5-hydroxy-2-methyl-1H-indol-3-yl]acetate
butyl [1-(3-chlorobenzoyl)-6-fluoro-5-hydroxy-2-methyl-1H-indol-3-yl]acetate
sec-butyl [1-(3-chlorobenzoyl)-6-fluoro-5-hydroxy-2-methyl-1H-indol-3-yl]acetate
(R) sec-butyl [1-(3-chlorobenzoyl)-6-fluoro-5-hydroxy-2-methyl-1H-indol-3-yl]acetate
(S) sec-butyl [1-(3-chlorobenzoyl)-6-fluoro-5-hydroxy-2-methyl-1H-indol-3-yl]acetate
isopropyl [1-(3-chlorobenzoyl)-6-fluoro-5-hydroxy-2-methyl-1H-indol-3-yl]acetate
tert-butyl [1-(3-chlorobenzoyl)-6-fluoro-5-hydroxy-2-methyl-1H-indol-3-yl]acetate
cyclobutyl [1-(3-chlorobenzoyl)-6-fluoro-5-hydroxy-2-methyl-1H-indol-3-yl]acetate
cyclopentyl [1-(3-chlorobenzoyl)-6-fluoro-5-hydroxy-2-methyl-1H-indol-3-yl]acetate
2-[1-(3-chlorobenzoyl)-6-fluoro-5-hydroxy-2-methyl-1H-indol-3-yl]acetamide
2-[1-(3-chlorobenzoyl)-6-fluoro-5-hydroxy-2-methyl-1H-indol-3-yl]-N-methylacetamide
2-[1-(3-chlorobenzoyl)-6-fluoro-5-hydroxy-2-methyl-1H-indol-3-yl]-N-ethylacetamide
2-[1-(3-chlorobenzoyl)-6-fluoro-5-hydroxy-2-methyl-1H-indol-3-yl]-N-propylacetamide
2-[1-(3-chlorobenzoyl)-6-fluoro-5-hydroxy-2-methyl-1H-indol-3-yl]-N-isopropylacetamide
2-[1-(3-chlorobenzoyl)-6-fluoro-5-hydroxy-2-methyl-1H-indol-3-yl]-N-butylacetamide
2-[1-(3-chlorobenzoyl)-6-fluoro-5-hydroxy-2-methyl-1H-indol-3-yl]-N-sec-butylacetamide
2-[1-(3-chlorobenzoyl)-6-fluoro-5-hydroxy-2-methyl-1H-indol-3-yl]-N-(2-hydroxyethyl)acetamide
({[1-(3-chlorobenzoyl)-6-fluoro-5-hydroxy-2-methyl-1H-indol-3-yl]acetyl}amino)acetic acid
3-({[1-(3-chlorobenzoyl)-6-fluoro-5-hydroxy-2-methyl 1H-indol-3-yl]acetyl}amino)propanoic acid
3-({[1-(3-chlorobenzoyl)-6-fluoro-5-hydroxy-2-methyl-1H-indol-3-yl]acetyl}amino)-2-methylpropanoic acid
3-({[1-(3-chlorobenzoyl)-6-fluoro-5-hydroxy-2-methyl-1H-indol-3-yl]acetyl}amino)-3-methylpropanoic acid
2-({[1-(3-chlorobenzoyl)-6-fluoro-5-hydroxy-2-methyl-1H-indol-3-yl]acetyl}amino)propanoic acid
2-({[1-(3-chlorobenzoyl)-6-fluoro-5-hydroxy-2-methyl-1H-indol-3-yl]acetyl}amino)-2-methylpropanoic acid
[1-(3-chlorobenzoyl)-4-fluoro-5-hydroxy-2-methyl-1H-indol-3-yl]acetic acid,
methyl [1-(3-chlorobenzoyl)-4-fluoro-5-hydroxy-2-methyl-1H-indol-3-yl]acetate
ethyl [1-(3-chlorobenzoyl)-4-fluoro-5-hydroxy-2-methyl-1H-indol-3-yl]acetate
propyl [1-(3-chlorobenzoyl)-4-fluoro-5-hydroxy-2-methyl-1H-indol-3-yl]acetate
butyl [1-(3-chlorobenzoyl)-4-fluoro-5-hydroxy-2-methyl-1H-indol-3-yl]acetate
sec-butyl [1-(3-chlorobenzoyl)-4-fluoro-5-hydroxy-2-methyl-1H-indol-3-yl]acetate
(R) sec-butyl [1-(3-chlorobenzoyl)-4-fluoro-5-hydroxy-2-methyl-1H-indol-3-yl]acetate
(S) sec-butyl [1-(3-chlorobenzoyl)-4-fluoro-5-hydroxy-2-methyl-1H-indol-3-yl]acetate
isopropyl [1-(3-chlorobenzoyl)-4-fluoro-5-hydroxy-2-methyl-1H-indol-3-yl]acetate
tert-butyl [1-(3-chlorobenzoyl)-4-fluoro-5-hydroxy-2-methyl-1H-indol-3-yl]acetate
cyclobutyl [1-(3-chlorobenzoyl)-4-fluoro-5-hydroxy-2-methyl-1H-indol-3-yl]acetate
cyclopentyl [1-(3-chlorobenzoyl)-4-fluoro-5-hydroxy-2-methyl-1H-indol-3-yl]acetate
2-[1-(3-chlorobenzoyl)-4-fluoro-5-hydroxy-2-methyl-1H-indol-3-yl]acetamide
2-[1-(3-chlorobenzoyl)-4-fluoro-5-hydroxy-2-methyl-1H-indol-3-yl]-N-methylacetamide
2-[1-(3-chlorobenzoyl)-4-fluoro-5-hydroxy-2-methyl-1H-indol-3-yl]-N-ethylacetamide
2-[1-(3-chlorobenzoyl)-4-fluoro-5-hydroxy-2-methyl-1H-indol-3-yl]-N-propylacetamide
2-[1-(3-chlorobenzoyl)-4-fluoro-5-hydroxy-2-methyl-1H-indol-3-yl]-N-isopropylacetamide
2-[1-(3-chlorobenzoyl)-4-fluoro-5-hydroxy-2-methyl-1H-indol-3-yl]-N-butylacetamide
2-[1-(3-chlorobenzoyl)-4-fluoro-5-hydroxy-2-methyl-1H-indol-3-yl]-N-sec-butylacetamide
2-[1-(3-chlorobenzoyl)-4-fluoro-5-hydroxy-2-methyl-1H-indol-3-yl]-N-(2-hydroxyethyl)acetamide
({[1-(3-chlorobenzoyl)-4-fluoro-5-hydroxy-2-methyl-1H-indol-3-yl]acetyl}amino)acetic acid
3-({[1-(3-chlorobenzoyl)-4-fluoro-5-hydroxy-2-methyl-1H-indol-3-yl]acetyl}amino)propanoic acid
3-({[1-(3-chlorobenzoyl)-4-fluoro-5-hydroxy-2-methyl-1H-indol-3-yl]acetyl}amino)-2-methylpropanoic acid
3-({[1-(3-chlorobenzoyl)-4-fluoro-5-hydroxy-2-methyl-1H-indol-3-yl]acetyl}amino)-3-methylpropanoic acid
2-({[1-(3-chlorobenzoyl)-4-fluoro-5-hydroxy-2-methyl-1H-indol-3-yl]acetyl}amino)propanoic acid
2-({[1-(3-chlorobenzoyl)-4-fluoro-5-hydroxy-2-methyl-1H-indol-3-yl]acetyl}amino)-2-methylpropanoic acid
[1-(3-chlorobenzoyl)-5-hydroxy-2-methyl-1H-indol-3-yl]acetic acid,
methyl [1-(3-chlorobenzoyl)-5-hydroxy-2-methyl-1H-indol-3-yl]acetate
ethyl [1-(3-chlorobenzoyl)-5-hydroxy-2-methyl-1H-indol-3-yl]acetate
propyl [1-(3-chlorobenzoyl)-5-hydroxy-2-methyl-1H-indol-3-yl]acetate
butyl [1-(3-chlorobenzoyl)-5-hydroxy-2-methyl-1H-indol-3-yl]acetate
sec-butyl [1-(3-chlorobenzoyl)-5-hydroxy-2-methyl-1H-indol-3-yl]acetate
(R) sec-butyl [1-(3-chlorobenzoyl)-5-hydroxy-2-methyl-1H-indol-3-yl]acetate
(S) sec-butyl [1-(3-chlorobenzoyl)-5-hydroxy-2-methyl-1H-indol-3-yl]acetate
isopropyl [1-(3-chlorobenzoyl)-5-hydroxy-2-methyl-1H-indol-3-yl]acetate
tert-butyl [1-(3-chlorobenzoyl)-5-hydroxy-2-methyl-1H-indol-3-yl]acetate
cyclobutyl [1-(3-chlorobenzoyl)-5-hydroxy-2-methyl-1H-indol-3-yl]acetate
cyclopentyl [1-(3-chlorobenzoyl)-5-hydroxy-2-methyl-1H-indol-3-yl]acetate
2-[1-(3-chlorobenzoyl)-5-hydroxy-2-methyl-1H-indol-3-yl]acetamide
2-[1-(3-chlorobenzoyl)-5-hydroxy-2-methyl-1H-indol-3-yl]-N-methylacetamide
2-[1-(3-chlorobenzoyl)-5-hydroxy-2-methyl-1H-indol-3-yl]-N-ethylacetamide
2-[1-(3-chlorobenzoyl)-5-hydroxy-2-methyl-1H-indol-3-yl]-N-propylacetamide
2-[1-(3-chlorobenzoyl)-5-hydroxy-2-methyl-1H-indol-3-yl]-N-isopropylacetamide
2-[1-(3-chlorobenzoyl)-5-hydroxy-2-methyl-1H-indol-3-yl]-N-butylacetamide
2-[1-(3-chlorobenzoyl)-5-hydroxy-2-methyl-1H-indol-3-yl]-N-sec-butylacetamide
2-[1-(3-chlorobenzoyl)-5-hydroxy-2-methyl-1H-indol-3-yl]-N-(2-hydroxyethyl)acetamide
({[1-(3-chlorobenzoyl)-5-hydroxy-2-methyl-1H-indol-3-yl]acetyl}amino)acetic acid
3-({[1-(3-chlorobenzoyl)-5-hydroxy-2-methyl-1H-indol-3-yl]acetyl}amino)propanoic acid
3-({[1-(3-chlorobenzoyl)-5-hydroxy-2-methyl-1H-indol-3-yl]acetyl}amino)-2-methylpropanoic acid
3-({[1-(3-chlorobenzoyl)-5-hydroxy-2-methyl-1H-indol-3-yl]acetyl}amino)-3-methylpropanoic acid
2-({[1-(3-chlorobenzoyl)-5-hydroxy-2-methyl-1H-indol-3-yl]acetyl}amino)propanoic acid
2-({[1-(3-chlorobenzoyl)-5-hydroxy-2-methyl-1H-indol-3-yl]acetyl}amino)-2-methylpropanoic acid
[1-(3,4-dichlorobenzoyl)-5-hydroxy-2-methyl-1H-indol-3-yl]acetic acid,
methyl [1-(3,4-dichlorobenzoyl)-5-hydroxy-2-methyl-1H-indol-3-yl]acetate
ethyl [1-(3,4-dichlorobenzoyl)-5-hydroxy-2-methyl-1H-indol-3-yl]acetate
propyl [1-(3,4-dichlorobenzoyl)-5-hydroxy-2-methyl-1H-indol-3-yl]acetate
butyl [1-(3,4-dichlorobenzoyl)-5-hydroxy-2-methyl-1H-indol-3-yl]acetate
sec-butyl [1-(3,4-dichlorobenzoyl)-5-hydroxy-2-methyl-1H-indol-3-yl]acetate
(R) sec-butyl [1-(3,4-dichlorobenzoyl)-5-hydroxy-2-methyl-1H-indol-3-yl]acetate
(S) sec-butyl [1-(3,4-dichlorobenzoyl)-5-hydroxy-2-methyl-1H-indol-3-yl]acetate
isopropyl [1-(3,4-dichlorobenzoyl)-5-hydroxy-2-methyl-1H-indol-3-yl]acetate
tert-butyl [1-(3,4-dichlorobenzoyl)-5-hydroxy-2-methyl-1H-indol-3-yl]acetate
cyclobutyl [1-(3,4-dichlorobenzoyl)-5-hydroxy-2-methyl-1H-indol-3-yl]acetate
cyclopentyl [1-(3,4-dichlorobenzoyl)-5-hydroxy-2-methyl-1H-indol-3-yl]acetate
2-[1-(3,4-dichlorobenzoyl)-5-hydroxy-2-methyl-1H-indol-3-yl]acetamide
2-[1-(3,4-dichlorobenzoyl)-5-hydroxy-2-methyl-1H-indol-3-yl]-N-methylacetamide
2-[1-(3,4-dichlorobenzoyl)-5-hydroxy-2-methyl-1H-indol-3-yl]-N-ethylacetamide
2-[1-(3,4-dichlorobenzoyl)-5-hydroxy-2-methyl-1H-indol-3-yl]-N-propylacetamide
2-[1-(3,4-dichlorobenzoyl)-5-hydroxy-2-methyl-1H-indol-3-yl]-N-isopropylacetamide
2-[1-(3,4-dichlorobenzoyl)-5-hydroxy-2-methyl-1H-indol-3-yl]-N-butylacetamide
2-[1-(3,4-dichlorobenzoyl)-5-hydroxy-2-methyl-1H-indol-3-yl]-N-sec-butylacetamide
2-[1-(3,4-dichlorobenzoyl)-5-hydroxy-2-methyl-1H-indol-3-yl]-N-(2-hydroxyethyl)acetamide
({[1-(3,4-dichlorobenzoyl)-5-hydroxy-2-methyl-1H-indol-3-yl]acetyl}amino)acetic acid
3-({[1-(3,4-dichlorobenzoyl)-5-hydroxy-2-methyl-1H-indol-3-yl]acetyl}amino)propanoic acid
3-({[1-(3,4-dichlorobenzoyl)-5-hydroxy-2-methyl-1H-indol-3-yl]acetyl}amino)-2-methylpropanoic acid
3-({[1-(3,4-dichlorobenzoyl)-5-hydroxy-2-methyl-1H-indol-3-yl]acetyl}amino)-3-methylpropanoic acid
2-({[1-(3,4-dichlorobenzoyl)-5-hydroxy-2-methyl-1H-indol-3-yl]acetyl}amino)propanoic acid
2-({[1-(3,4-dichlorobenzoyl)-5-hydroxy-2-methyl-1H-indol-3-yl]acetyl}amino)-2-methylpropanoic acid
[1-(3,4-dichlorobenzoyl)-6-fluoro-5-hydroxy-2-methyl-1H-indol-3-yl]acetic acid,
methyl [1-(3,4-dichlorobenzoyl)-6-fluoro-5-hydroxy-2-methyl-1H-indol-3-yl]acetate
ethyl [1-(3,4-dichlorobenzoyl)-6-fluoro-5-hydroxy-2-methyl-1H-indol-3-yl]acetate
propyl [1-(3,4-dichlorobenzoyl)-6-fluoro-5-hydroxy-2-methyl-1H-indol-3-yl]acetate
butyl [1-(3,4-dichlorobenzoyl)-6-fluoro-5-hydroxy-2-methyl-1H-indol-3-yl]acetate
sec-butyl [1-(3,4-dichlorobenzoyl)-6-fluoro-5-hydroxy-2-methyl-1H-indol-3-yl]acetate
(R) sec-butyl [1-(3,4-dichlorobenzoyl)-6-fluoro-5-hydroxy-2-methyl-1H-indol-3-yl]acetate
(S) sec-butyl [1-(3,4-dichlorobenzoyl)-6-fluoro-5-hydroxy-2-methyl-1H-indol-3-yl]acetate
isopropyl [1-(3,4-dichlorobenzoyl)-6-fluoro-5-hydroxy-2-methyl-1H-indol-3-yl]acetate
tert-butyl [1-(3,4-dichlorobenzoyl)-6-fluoro-5-hydroxy-2-methyl-1H-indol-3-yl]acetate
cyclobutyl [1-(3,4-dichlorobenzoyl)-6-fluoro-5-hydroxy-2-methyl-1H-indol-3-yl]acetate
cyclopentyl [1-(3,4-dichlorobenzoyl)-6-fluoro-5-hydroxy-2-methyl-1H-indol-3-yl]acetate
2-[1-(3,4-dichlorobenzoyl)-6-fluoro-5-hydroxy-2-methyl-1H-indol-3-yl]acetamide
2-[1-(3,4-dichlorobenzoyl)-6-fluoro-5-hydroxy-2-methyl-1H-indol-3-yl]-N-methylacetamide
2-[1-(3,4-dichlorobenzoyl)-6-fluoro-5-hydroxy-2-methyl-1H-indol-3-yl]-N-ethylacetamide
2-[1-(3,4-dichlorobenzoyl)-6-fluoro-5-hydroxy-2-methyl-1H-indol-3-yl]-N-propylacetamide
2-[1-(3,4-dichlorobenzoyl)-6-fluoro-5-hydroxy-2-methyl-1H-indol-3-yl]-N-isopropylacetamide
2-[1-(3,4-dichlorobenzoyl)-6-fluoro-5-hydroxy-2-methyl-1H-indol-3-yl]-N-butylacetamide
2-[1-(3,4-dichlorobenzoyl)-6-fluoro-5-hydroxy-2-methyl-1H-indol-3-yl]-N-sec-butylacetamide
2-[1-(3,4-dichlorobenzoyl)-6-fluoro-5-hydroxy-2-methyl-1H-indol-3-yl]-N-(2-hydroxyethyl)acetamide
({[1-(3,4-dichlorobenzoyl)-6-fluoro-5-hydroxy-2-methyl-1H-indol-3-yl]acetyl}amino)acetic acid
3-({[1-(3,4-dichlorobenzoyl)-6-fluoro-5-hydroxy-2-methyl-1H-indol-3-yl]acetyl}amino)propanoic acid
3-({[1-(3,4-dichlorobenzoyl)-6-fluoro-5-hydroxy-2-methyl-1H-indol-3-yl]acetyl}amino)-2-methylpropanoic acid
3-({[1-(3,4-dichlorobenzoyl)-6-fluoro-5-hydroxy-2-methyl-1H-indol-3-yl]acetyl}amino)-3-methylpropanoic acid
2-({[1-(3,4-dichlorobenzoyl)-6-fluoro-5-hydroxy-2-methyl-1H-indol-3-yl]acetyl}amino)propanoic acid
2-({[1-(3,4-dichlorobenzoyl)-6-fluoro-5-hydroxy-2-methyl-1H-indol-3-yl]acetyl}amino)-2-methylpropanoic acid
[1-(3,4-dichlorobenzoyl)-4-fluoro-5-hydroxy-2-methyl-1H-indol-3-yl]acetic acid,
methyl [1-(3,4-dichlorobenzoyl)-4-fluoro-5-hydroxy-2-methyl-1H-indol-3-yl]acetate
ethyl [1-(3,4-dichlorobenzoyl)-4-fluoro-5-hydroxy-2-methyl-1H-indol-3-yl]acetate
propyl [1-(3,4-dichlorobenzoyl)-4-fluoro-5-hydroxy-2-methyl-1H-indol-3-yl]acetate
butyl [1-(3,4-dichlorobenzoyl)-4-fluoro-5-hydroxy-2-methyl-1H-indol-3-yl]acetate
sec-butyl [1-(3,4-dichlorobenzoyl)-4-fluoro-5-hydroxy-2-methyl-1H-indol-3-yl]acetate
(R) sec-butyl [1-(3,4-dichlorobenzoyl)-4-fluoro-5-hydroxy-2-methyl-1H-indol-3-yl]acetate
(S) sec-butyl [1-(3,4-dichlorobenzoyl)-4-fluoro-5-hydroxy-2-methyl-1H-indol-3-yl]acetate
isopropyl [1-(3,4-dichlorobenzoyl)-4-fluoro-5-hydroxy-2-methyl-1H-indol-3-yl]acetate
tert-butyl [1-(3,4-dichlorobenzoyl)-4-fluoro-5-hydroxy-2-methyl-1H-indol-3-yl]acetate
cyclobutyl [1-(3,4-dichlorobenzoyl)-4-fluoro-5-hydroxy-2-methyl-1H-indol-3-yl]acetate
cyclopentyl [1-(3,4-dichlorobenzoyl)-4-fluoro-5-hydroxy-2-methyl-1H-indol-3-yl]acetate
2-[1-(3,4-dichlorobenzoyl)-4-fluoro-5-hydroxy-2-methyl-1H-indol-3-yl]acetamide
2-[1-(3,4-dichlorobenzoyl)-4-fluoro-5-hydroxy-2-methyl-1H-indol-3-yl]-N-methylacetamide
2-[1-(3,4-dichlorobenzoyl)-4-fluoro-5-hydroxy-2-methyl-1H-indol-3-yl]-N-ethylacetamide
2-[1-(3,4-dichlorobenzoyl)-4-fluoro-5-hydroxy-2-methyl-1H-indol-3-yl]-N-propylacetamide
2-[1-(3,4-dichlorobenzoyl)-4-fluoro-5-hydroxy-2-methyl-1H-indol-3-yl]-N-isopropylacetamide
2-[1-(3,4-dichlorobenzoyl)-4-fluoro-5-hydroxy-2-methyl-1H-indol-3-yl]-N-butylacetamide
2-[1-(3,4-dichlorobenzoyl)-4-fluoro-5-hydroxy-2-methyl-1H-indol-3-yl]-N-sec-butylacetamide
2-[1-(3,4-dichlorobenzoyl)-4-fluoro-5-hydroxy-2-methyl-1H-indol-3-yl]-N-(2-hydroxyethyl)acetamide
({[1-(3,4-dichlorobenzoyl)-4-fluoro-5-hydroxy-2-methyl-1H-indol-3-yl]acetyl}amino)acetic acid
3-({[1-(3,4-dichlorobenzoyl)-4-fluoro-5-hydroxy-2-methyl-1H-indol-3-yl]acetyl}amino)propanoic acid
3-({[1-(3,4-dichlorobenzoyl)-4-fluoro-5-hydroxy-2-methyl-1H-indol-3-yl]acetyl}amino)-2-methylpropanoic acid
3-({[1-(3,4-dichlorobenzoyl)-4-fluoro-5-hydroxy-2-methyl-1H-indol-3-yl]acetyl}amino)-3-methylpropanoic acid
2-({[1-(3,4-dichlorobenzoyl)-4-fluoro-5-hydroxy-2-methyl-1H-indol-3-yl]acetyl}amino)propanoic acid
2-({[1-(3,4-dichlorobenzoyl)-4-fluoro-5-hydroxy-2-methyl-1H-indol-3-yl]acetyl}amino)-2-methylpropanoic acid
[1-(3,4-dichlorobenzoyl)-4,6-difluoro-5-hydroxy-2-methyl-1H-indol-3-yl]acetic acid,
methyl [1-(3,4-dichlorobenzoyl)-4,6-difluoro-5-hydroxy-2-methyl-1H-indol-3-yl]acetate
ethyl [1-(3,4-dichlorobenzoyl)-4,6-difluoro-5-hydroxy-2-methyl-1H-indol-3-yl]acetate
propyl [1-(3,4-dichlorobenzoyl)-4,6-difluoro-5-hydroxy-2-methyl-1H-indol-3-yl]acetate
butyl [1-(3,4-dichlorobenzoyl)-4,6-difluoro-5-hydroxy-2-methyl-1H-indol-3-yl]acetate
sec-butyl [1-(3,4-dichlorobenzoyl)-4,6-difluoro-5-hydroxy-2-methyl-1H-indol-3-yl]acetate
(R) sec-butyl [1-(3,4-dichlorobenzoyl)-4,6-difluoro-5-hydroxy-2-methyl-1H-indol-3-yl]acetate
(S) sec-butyl [1-(3,4-dichlorobenzoyl)-4,6-difluoro-5-hydroxy-2-methyl-1H-indol-3-yl]acetate
isopropyl [1-(3,4-dichlorobenzoyl)-4,6-difluoro-5-hydroxy-2-methyl-1H-indol-3-yl]acetate
tert-butyl [1-(3,4-dichlorobenzoyl)-4,6-difluoro-5-hydroxy-2-methyl-1H-indol-3-yl]acetate
cyclobutyl [1-(3,4-dichlorobenzoyl)-4,6-difluoro-5-hydroxy-2-methyl-1H-indol-3-yl]acetate
cyclopentyl [1-(3,4-dichlorobenzoyl)-4,6-difluoro-5-hydroxy-2-methyl-1H-indol-3-yl]acetate
2-[1-(3,4-dichlorobenzoyl)-4,6-difluoro-5-hydroxy-2-methyl-1H-indol-3-yl]acetamide
2-[1-(3,4-dichlorobenzoyl)-4,6-difluoro-5-hydroxy-2-methyl-1H-indol-3-yl]-N-methylacetamide
2-[1-(3,4-dichlorobenzoyl)-4,6-difluoro-5-hydroxy-2-methyl-1H-indol-3-yl]-N-ethylacetamide
2-[1-(3,4-dichlorobenzoyl)-4,6-difluoro-5-hydroxy-2-methyl-1H-indol-3-yl]-N-propylacetamide
2-[1-(3,4-dichlorobenzoyl)-4,6-difluoro-5-hydroxy-2-methyl-1H-indol-3-yl]-N-isopropylacetamide
2-[1-(3,4-dichlorobenzoyl)-4,6-difluoro-5-hydroxy-2-methyl-1H-indol-3-yl]-N-butylacetamide
2-[1-(3,4-dichlorobenzoyl)-4,6-difluoro-5-hydroxy-2-methyl-1H-indol-3-yl]-N-sec-butylacetamide
2-[1-(3,4-dichlorobenzoyl)-4,6-difluoro-5-hydroxy-2-methyl-1H-indol-3-yl]-N-(2-hydroxyethyl)acetamide
({[1-(3,4-dichlorobenzoyl)-4,6-difluoro-5-hydroxy-2-methyl-1H-indol-3-yl]acetyl}amino)acetic acid
3-({[1-(3,4-dichlorobenzoyl)-4,6-difluoro-5-hydroxy-2-methyl-1H-indol-3-yl]acetyl}amino)propanoic acid
3-({[1-(3,4-dichlorobenzoyl)-4,6-difluoro-5-hydroxy-2-methyl-1H-indol-3-yl]acetyl}amino)-2-methylpropanoic acid
3-({[1-(3,4-dichlorobenzoyl)-4,6-difluoro-5-hydroxy-2-methyl-1H-indol-3-yl]acetyl}amino)-3-methylpropanoic acid
2-({[1-(3,4-dichlorobenzoyl)-4,6-difluoro-5-hydroxy-2-methyl-1H-indol-3-yl]acetyl}amino)propanoic acid
2-({[1-(3,4-dichlorobenzoyl)-4,6-difluoro-5-hydroxy-2-methyl-1H-indol-3-yl]acetyl}amino)-2-methylpropanoic acid
[1-(3,4-difluorobenzoyl)-5-hydroxy-2-methyl-1H-indol-3-yl]acetic acid,
methyl [1-(3,4-difluorobenzoyl)-5-hydroxy-2-methyl-1H-indol-3-yl]acetate
ethyl [1-(3,4-difluorobenzoyl)-5-hydroxy-2-methyl-1H-indol-3-yl]acetate
propyl [1-(3,4-difluorobenzoyl)-5-hydroxy-2-methyl-1H-indol-3-yl]acetate
butyl [1-(3,4-difluorobenzoyl)-5-hydroxy-2-methyl-1H-indol-3-yl]acetate
sec-butyl [1-(3,4-difluorobenzoyl)-5-hydroxy-2-methyl-1H-indol-3-yl]acetate
(R) sec-butyl [1-(3,4-difluorobenzoyl)-5-hydroxy-2-methyl-1H-indol-3-yl]acetate
(S) sec-butyl [1-(3,4-difluorobenzoyl)-5-hydroxy-2-methyl-1H-indol-3-yl]acetate
isopropyl [1-(3,4-difluorobenzoyl)-5-hydroxy-2-methyl-1H-indol-3-yl]acetate
tert-butyl [1-(3,4-difluorobenzoyl)-5-hydroxy-2-methyl-1H-indol-3-yl]acetate
cyclobutyl [1-(3,4-difluorobenzoyl)-5-hydroxy-2-methyl-1H-indol-3-yl]acetate
cyclopentyl [1-(3,4-difluorobenzoyl)-5-hydroxy-2-methyl-1H-indol-3-yl]acetate
2-[1-(3,4-difluorobenzoyl)-5-hydroxy-2-methyl-1H-indol-3-yl]acetamide
2-[1-(3,4-difluorobenzoyl)-5-hydroxy-2-methyl-1H-indol-3-yl]-N-methylacetamide
2-[1-(3,4-difluorobenzoyl)-5-hydroxy-2-methyl-1H-indol-3-yl]-N-ethylacetamide
2-[1-(3,4-difluorobenzoyl)-5-hydroxy-2-methyl-1H-indol-3-yl]-N-propylacetamide
2-[1-(3,4-difluorobenzoyl)-5-hydroxy-2-methyl-1H-indol-3-yl]-N-isopropylacetamide
2-[1-(3,4-difluorobenzoyl)-5-hydroxy-2-methyl-1H-indol-3-yl]-N-butylacetamide
2-[1-(3,4-difluorobenzoyl)-5-hydroxy-2-methyl-1H-indol-3-yl]-N-sec-butylacetamide
2-[1-(3,4-difluorobenzoyl)-5-hydroxy-2-methyl-1H-indol-3-yl]-N-(2-hydroxyethyl)acetamide
({[1-(3,4-difluorobenzoyl)-5-hydroxy-2-methyl-1H-indol-3-yl]acetyl}amino)acetic acid
3-({[1-(3,4-difluorobenzoyl)-5-hydroxy-2-methyl-1H-indol-3-yl]acetyl}amino)propanoic acid
3-({[1-(3,4-difluorobenzoyl)-5-hydroxy-2-methyl-1H-indol-3-yl]acetyl}amino)-2-methylpropanoic acid
3-({[1-(3,4-difluorobenzoyl)-5-hydroxy-2-methyl-1H-indol-3-yl]acetyl}amino)-3-methylpropanoic acid
2-({[1-(3,4-difluorobenzoyl)-5-hydroxy-2-methyl-1H-indol-3-yl]acetyl}amino)propanoic acid
2-({[1-(3,4-difluorobenzoyl)-5-hydroxy-2-methyl-1H-indol-3-yl]acetyl}amino)-2-methylpropanoic acid
[1-(3,4-difluorobenzoyl)-6-fluoro-5-hydroxy-2-methyl-1H-indol-3-yl]acetic acid,
methyl [1-(3,4-difluorobenzoyl)-6-fluoro-5-hydroxy-2-methyl-1H-indol-3-yl]acetate
ethyl [1-(3,4-difluorobenzoyl)-6-fluoro-5-hydroxy-2-methyl-1H-indol-3-yl]acetate
propyl [1-(3,4-difluorobenzoyl)-6-fluoro-5-hydroxy-2-methyl-1H-indol-3-yl]acetate
butyl [1-(3,4-difluorobenzoyl)-6-fluoro-5-hydroxy-2-methyl-1H-indol-3-yl]acetate
sec-butyl [1-(3,4-difluorobenzoyl)-6-fluoro-5-hydroxy-2-methyl-1H-indol-3-yl]acetate
(R) sec-butyl [1-(3,4-difluorobenzoyl)-6-fluoro-5-hydroxy-2-methyl-1H-indol-3-yl]acetate
(S) sec-butyl [1-(3,4-difluorobenzoyl)-6-fluoro-5-hydroxy-2-methyl-1H-indol-3-yl]acetate
isopropyl [1-(3,4-difluorobenzoyl)-6-fluoro-5-hydroxy-2-methyl-1H-indol-3-yl]acetate
tert-butyl [1-(3,4-difluorobenzoyl)-6-fluoro-5-hydroxy-2-methyl-1H-indol-3-yl]acetate
cyclobutyl [1-(3,4-difluorobenzoyl)-6-fluoro-5-hydroxy-2-methyl-1H-indol-3-yl]acetate
cyclopentyl [1-(3,4-difluorobenzoyl)-6-fluoro-5-hydroxy-2-methyl-1H-indol-3-yl]acetate
2-[1-(3,4-difluorobenzoyl)-6-fluoro-5-hydroxy-2-methyl-1H-indol-3-yl]acetamide
2-[1-(3,4-difluorobenzoyl)-6-fluoro-5-hydroxy-2-methyl-1H-indol-3-yl]-N-methylacetamide
2-[1-(3,4-difluorobenzoyl)-6-fluoro-5-hydroxy-2-methyl-1H-indol-3-yl]-N-ethylacetamide
2-[1-(3,4-difluorobenzoyl)-6-fluoro-5-hydroxy-2-methyl-1H-indol-3-yl]-N-propylacetamide
2-[1-(3,4-difluorobenzoyl)-6-fluoro-5-hydroxy-2-methyl-1H-indol-3-yl]-N-isopropylacetamide
2-[1-(3,4-difluorobenzoyl)-6-fluoro-5-hydroxy-2-methyl-1H-indol-3-yl]-N-butylacetamide
2-[1-(3,4-difluorobenzoyl)-6-fluoro-5-hydroxy-2-methyl-1H-indol-3-yl]-N-sec-butylacetamide
2-[1-(3,4-difluorobenzoyl)-6-fluoro-5-hydroxy-2-methyl-1H-indol-3-yl]-N-(2-hydroxyethyl)acetamide
({[1-(3,4-difluorobenzoyl)-6-fluoro-5-hydroxy-2-methyl-1H-indol-3-yl]acetyl}amino)acetic acid
3-({[1-(3,4-difluorobenzoyl)-6-fluoro-5-hydroxy-2-methyl-1H-indol-3-yl]acetyl}amino)propanoic acid
3-({[1-(3,4-difluorobenzoyl)-6-fluoro-5-hydroxy-2-methyl-1H-indol-3-yl]acetyl}amino)-2-methylpropanoic acid
3-({[1-(3,4-difluorobenzoyl)-6-fluoro-5-hydroxy-2-methyl-1H-indol-3-yl]acetyl}amino)-3-methylpropanoic acid
2-({[1-(3,4-difluorobenzoyl)-6-fluoro-5-hydroxy-2-methyl-1H-indol-3-yl]acetyl}amino)propanoic acid
2-({[1-(3,4-difluorobenzoyl)-6-fluoro-5-hydroxy-2-methyl-1H-indol-3-yl]acetyl}amino)-2-methylpropanoic acid
[1-(3,4-difluorobenzoyl)-4-fluoro-5-hydroxy-2-methyl-1H-indol-3-yl]acetic acid,
methyl [1-(3,4-difluorobenzoyl)-4-fluoro-5-hydroxy-2-methyl-1H-indol-3-yl]acetate
ethyl [1-(3,4-difluorobenzoyl)-4-fluoro-5-hydroxy-2-methyl-1H-indol-3-yl]acetate
propyl [1-(3,4-difluorobenzoyl)-4-fluoro-5-hydroxy-2-methyl-1H-indol-3-yl]acetate
butyl [1-(3,4-difluorobenzoyl)-4-fluoro-5-hydroxy-2-methyl-1H-indol-3-yl]acetate
sec-butyl [1-(3,4-difluorobenzoyl)-4-fluoro-5-hydroxy-2-methyl-1H-indol-3-yl]acetate
(R) sec-butyl [1-(3,4-difluorobenzoyl)-4-fluoro-5-hydroxy-2-methyl-1H-indol-3-yl]acetate
(S) sec-butyl [1-(3,4-difluorobenzoyl)-4-fluoro-5-hydroxy-2-methyl-1H-indol-3-yl]acetate
isopropyl [1-(3,4-difluorobenzoyl)-4-fluoro-5-hydroxy-2-methyl-1H-indol-3-yl]acetate
tert-butyl [1-(3,4-difluorobenzoyl)-4-fluoro-5-hydroxy-2-methyl-1H-indol-3-yl]acetate
cyclobutyl [1-(3,4-difluorobenzoyl)-4-fluoro-5-hydroxy-2-methyl-1H-indol-3-yl]acetate
cyclopentyl [1-(3,4-difluorobenzoyl)-4-fluoro-5-hydroxy-2-methyl-1H-indol-3-yl]acetate
2-[1-(3,4-difluorobenzoyl)-4-fluoro-5-hydroxy-2-methyl-1H-indol-3-yl]acetamide
2-[1-(3,4-difluorobenzoyl)-4-fluoro-5-hydroxy-2-methyl-1H-indol-3-yl]-N-methylacetamide
2-[1-(3,4-difluorobenzoyl)-4-fluoro-5-hydroxy-2-methyl-1H-indol-3-yl]-N-ethylacetamide
2-[1-(3,4-difluorobenzoyl)-4-fluoro-5-hydroxy-2-methyl-1H-indol-3-yl]-N-propylacetamide
2-[1-(3,4-difluorobenzoyl)-4-fluoro-5-hydroxy-2-methyl-1H-indol-3-yl]-N-isopropylacetamide
2-[1-(3,4-difluorobenzoyl)-4-fluoro-5-hydroxy-2-methyl-1H-indol-3-yl]-N-butylacetamide
2-[1-(3,4-difluorobenzoyl)-4-fluoro-5-hydroxy-2-methyl-1H-indol-3-yl]-N-sec-butylacetamide
2-[1-(3,4-difluorobenzoyl)-4-fluoro-5-hydroxy-2-methyl-1H-indol-3-yl]-N-(2-hydroxyethyl)acetamide
({[1-(3,4-difluorobenzoyl)-4-fluoro-5-hydroxy-2-methyl-1H-indol-3-yl]acetyl}amino)acetic acid
3-({[1-(3,4-difluorobenzoyl)-4-fluoro-5-hydroxy-2-methyl-1H-indol-3-yl]acetyl}amino)propanoic acid
3-({[1-(3,4-difluorobenzoyl)-4-fluoro-5-hydroxy-2-methyl-1H-indol-3-yl]acetyl}amino)-2-methylpropanoic acid
3-({[1-(3,4-difluorobenzoyl)-4-fluoro-5-hydroxy-2-methyl-1H-indol-3-yl]acetyl}amino)-3-methylpropanoic acid
2-({[1-(3,4-difluorobenzoyl)-4-fluoro-5-hydroxy-2-methyl-1H-indol-3-yl]acetyl}amino)propanoic acid
2-({[1-(3,4-difluorobenzoyl)-4-fluoro-5-hydroxy-2-methyl-1H-indol-3-yl]acetyl}amino)-2-methylpropanoic acid
[1-(3,4-difluorobenzoyl)-4,6-difluoro-5-hydroxy-2-methyl-1H-indol-3-yl]acetic acid,
methyl [1-(3,4-difluorobenzoyl)-4,6-difluoro-5-hydroxy-2-methyl-1H-indol-3-yl]acetate
ethyl [1-(3,4-difluorobenzoyl)-4,6-difluoro-5-hydroxy-2-methyl-1H-indol-3-yl]acetate
propyl [1-(3,4-difluorobenzoyl)-4,6-difluoro-5-hydroxy-2-methyl-1H-indol-3-yl]acetate
butyl [1-(3,4-difluorobenzoyl)-4,6-difluoro-5-hydroxy-2-methyl-1H-indol-3-yl]acetate
sec-butyl [1-(3,4-difluorobenzoyl)-4,6-difluoro-5-hydroxy-2-methyl-1H-indol-3-yl]acetate
(R) sec-butyl [1-(3,4-difluorobenzoyl)-4,6-difluoro-5-hydroxy-2-methyl-1H-indol-3-yl]acetate
(S) sec-butyl [1-(3,4-difluorobenzoyl)-4,6-difluoro-5-hydroxy-2-methyl-1H-indol-3-yl]acetate
isopropyl [1-(3,4-difluorobenzoyl)-4,6-difluoro-5-hydroxy-2-methyl-1H-indol-3-yl]acetate
tert-butyl [1-(3,4-difluorobenzoyl)-4,6-difluoro-5-hydroxy-2-methyl-1H-indol-3-yl]acetate
cyclobutyl [1-(3,4-difluorobenzoyl)-4,6-difluoro-5-hydroxy-2-methyl-1H-indol-3-yl]acetate
cyclopentyl [1-(3,4-difluorobenzoyl)-4,6-difluoro-5-hydroxy-2-methyl-1H-indol-3-yl]acetate
2-[1-(3,4-difluorobenzoyl)-4,6-difluoro-5-hydroxy-2-methyl-1H-indol-3-yl]acetamide
2-[1-(3,4-difluorobenzoyl)-4,6-difluoro-5-hydroxy-2-methyl-1H-indol-3-yl]-N-methylacetamide
2-[1-(3,4-difluorobenzoyl)-4,6-difluoro-5-hydroxy-2-methyl-1H-indol-3-yl]-N-ethylacetamide
2-[1-(3,4-difluorobenzoyl)-4,6-difluoro-5-hydroxy-2-methyl-1H-indol-3-yl]-N-propylacetamide
2-[1-(3,4-difluorobenzoyl)-4,6-difluoro-5-hydroxy-2-methyl-1H-indol-3-yl]-N-isopropylacetamide
2-[1-(3,4-difluorobenzoyl)-4,6-difluoro-5-hydroxy-2-methyl-1H-indol-3-yl]-N-butylacetamide
2-[1-(3,4-difluorobenzoyl)-4,6-difluoro-5-hydroxy-2-methyl-1H-indol-3-yl]-N-sec-butylacetamide
2-[1-(3,4-difluorobenzoyl)-4,6-difluoro-5-hydroxy-2-methyl-1H-indol-3-yl]-N-(2-hydroxyethyl)acetamide
({[1-(3,4-difluorobenzoyl)-4,6-difluoro-5-hydroxy-2-methyl-1H-indol-3-yl]acetyl}amino)acetic acid
3-({[1-(3,4-difluorobenzoyl)-4,6-difluoro-5-hydroxy-2-methyl-1H-indol-3-yl]acetyl}amino)propanoic acid
3-({[1-(3,4-difluorobenzoyl)-4,6-difluoro-5-hydroxy-2-methyl-1H-indol-3-yl]acetyl}amino)-2-methylpropanoic acid
3-({[1-(3,4-difluorobenzoyl)-4,6-difluoro-5-hydroxy-2-methyl-1H-indol-3-yl]acetyl}amino)-3-methylpropanoic acid
2-({[1-(3,4-difluorobenzoyl)-4,6-difluoro-5-hydroxy-2-methyl-1H-indol-3-yl]acetyl}amino)propanoic acid
2-({[1-(3,4-difluorobenzoyl)-4,6-difluoro-5-hydroxy-2-methyl-1H-indol-3-yl]acetyl}amino)-2-methylpropanoic acid
[1-(4-chloro-3-fluorobenzoyl)-5-hydroxy-2-methyl-1H-indol-3-yl]acetic acid,
methyl [1-(4-chloro-3-fluorobenzoyl)-5-hydroxy-2-methyl-1H-indol-3-yl]acetate
ethyl [1-(4-chloro-3-fluorobenzoyl)-5-hydroxy-2-methyl-1H-indol-3-yl]acetate
propyl [1-(4-chloro-3-fluorobenzoyl)-5-hydroxy-2-methyl-1H-indol-3-yl]acetate
butyl [1-(4-chloro-3-fluorobenzoyl)-5-hydroxy-2-methyl-1H-indol-3-yl]acetate
sec-butyl [1-(4-chloro-3-fluorobenzoyl)-5-hydroxy-2-methyl-1H-indol-3-yl]acetate
(R) sec-butyl [1-(4-chloro-3-fluorobenzoyl)-5′-hydroxy-2-methyl-1H-indol-3-yl]acetate
(S) sec-butyl [1-(4-chloro-3-fluorobenzoyl)-5-hydroxy-2-methyl-1H-indol-3-yl]acetate
isopropyl [1-(4-chloro-3-fluorobenzoyl)-5-hydroxy-2-methyl-1H-indol-3-yl]acetate
tert-butyl [1-(4-chloro-3-fluorobenzoyl)-5-hydroxy-2-methyl-1H-indol-3-yl]acetate
cyclobutyl [1-(4-chloro-3-fluorobenzoyl)-5-hydroxy-2-methyl-1H-indol-3-yl]acetate
cyclopentyl [1-(4-chloro-3-fluorobenzoyl)-5-hydroxy-2-methyl-1H-indol-3-yl]acetate
2-[1-(4-chloro-3-fluorobenzoyl)-5-hydroxy-2-methyl-1H-indol-3-yl]acetamide
2-[1-(4-chloro-3-fluorobenzoyl)-5-hydroxy-2-methyl-1H-indol-3-yl]-N-methylacetamide
2-[1-(4-chloro-3-fluorobenzoyl)-5-hydroxy-2-methyl-1H-indol-3-yl]-N-ethylacetamide
2-[1-(4-chloro-3-fluorobenzoyl)-5-hydroxy-2-methyl-1H-indol-3-yl]-N-propylacetamide
2-[1-(4-chloro-3-fluorobenzoyl)-5-hydroxy-2-methyl-1H-indol-3-yl]-N-isopropylacetamide
2-[1-(4-chloro-3-fluorobenzoyl)-5-hydroxy-2-methyl-1H-indol-3-yl]-N-butylacetamide
2-[1-(4-chloro-3-fluorobenzoyl)-5-hydroxy-2-methyl-1H-indol-3-yl]-N-sec-butylacetamide
2-[1-(4-chloro-3-fluorobenzoyl)-5-hydroxy-2-methyl-1H-indol-3-yl]-N-(2-hydroxyethyl)acetamide
({[1-(4-chloro-3-fluorobenzoyl)-5-hydroxy-2-methyl-1H-indol-3-yl]acetyl}amino)acetic acid
3-({[1-(4-chloro-3-fluorobenzoyl)-5-hydroxy-2-methyl-1H-indol-3-yl]acetyl}amino)propanoic acid
3-({[1-(4-chloro-3-fluorobenzoyl)-5-hydroxy-2-methyl-1H-indol-3-yl]acetyl}amino)-2-methylpropanoic acid
3-({[1-(4-chloro-3-fluorobenzoyl)-5-hydroxy-2-methyl-1H-indol-3-yl]acetyl}amino)-3-methylpropanoic acid
2-({[1-(4-chloro-3-fluorobenzoyl)-5-hydroxy-2-methyl-1H-indol-3-yl]acetyl}amino)propanoic acid
2-({[1-(4-chloro-3-fluorobenzoyl)-5-hydroxy-2-methyl-1H-indol-3-yl]acetyl}amino)-2-methylpropanoic acid
[1-(4-chloro-3-fluorobenzoyl)-6-fluoro-5-hydroxy-2-methyl-1H-indol-3-yl]acetic acid,
methyl [1-(4-chloro-3-fluorobenzoyl)-6-fluoro-5-hydroxy-2-methyl-1H-indol-3-yl]acetate
ethyl [1-(4-chloro-3-fluorobenzoyl)-6-fluoro-5-hydroxy-2-methyl-1H-indol-3-yl]acetate
propyl [1-(4-chloro-3-fluorobenzoyl)-6-fluoro-5-hydroxy-2-methyl-1H-indol-3-yl]acetate
butyl [1-(4-chloro-3-fluorobenzoyl)-6-fluoro-5-hydroxy-2-methyl-1H-indol-3-yl]acetate
sec-butyl [1-(4-chloro-3-fluorobenzoyl)-6-fluoro-5-hydroxy-2-methyl-1H-indol-3-yl]acetate
(R) sec-butyl [1-(4-chloro-3-fluorobenzoyl)-6-fluoro-5-hydroxy-2-methyl-1H-indol-3-yl]acetate
(S) sec-butyl [1-(4-chloro-3-fluorobenzoyl)-6-fluoro-5-hydroxy-2-methyl-1H-indol-3-yl]acetate
isopropyl [1-(4-chloro-3-fluorobenzoyl)-6-fluoro-5-hydroxy-2-methyl-1H-indol-3-yl]acetate
tert-butyl [1-(4-chloro-3-fluorobenzoyl)-6-fluoro-5-hydroxy-2-methyl-1H-indol-3-yl]acetate
cyclobutyl [1-(4-chloro-3-fluorobenzoyl)-6-fluoro-5-hydroxy-2-methyl-1H-indol-3-yl]acetate
cyclopentyl [1-(4-chloro-3-fluorobenzoyl)-6-fluoro-5-hydroxy-2-methyl-1H-indol-3-yl]acetate
2-[1-(4-chloro-3-fluorobenzoyl)-6-fluoro-5-hydroxy-2-methyl-1H-indol-3-yl]acetamide
2-[1-(4-chloro-3-fluorobenzoyl)-6-fluoro-5-hydroxy-2-methyl-1H-indol-3-yl]-N-methylacetamide
2-[1-(4-chloro-3-fluorobenzoyl)-6-fluoro-5-hydroxy-2-methyl-1H-indol-3-yl]-N-ethylacetamide
2-[1-(4-chloro-3-fluorobenzoyl)-6-fluoro-5-hydroxy-2-methyl-1H-indol-3-yl]-N-propylacetamide
2-[1-(4-chloro-3-fluorobenzoyl)-6-fluoro-5-hydroxy-2-methyl-1H-indol-3-yl]-N-isopropylacetamide
2-[1-(4-chloro-3-fluorobenzoyl)-6-fluoro-5-hydroxy-2-methyl-1H-indol-3-yl]-N-butylacetamide
2-[1-(4-chloro-3-fluorobenzoyl)-6-fluoro-5-hydroxy-2-methyl-1H-indol-3-yl]-N-sec-butylacetamide
2-[1-(4-chloro-3-fluorobenzoyl)-6-fluoro-5-hydroxy-2-methyl-1H-indol-3-yl]-N-(2-hydroxyethyl)acetamide
({[1-(4-chloro-3-fluorobenzoyl)-6-fluoro-5-hydroxy-2-methyl-1H-indol-3-yl]acetyl}amino)acetic acid
3-({[1-(4-chloro-3-fluorobenzoyl)-6-fluoro-5-hydroxy-2-methyl-1H-indol-3-yl]acetyl}amino)propanoic acid
3-({[1-(4-chloro-3-fluorobenzoyl)-6-fluoro-5-hydroxy-2-methyl-1H-indol-3-yl]acetyl}amino)-2-methylpropanoic acid
3-({[1-(4-chloro-3-fluorobenzoyl)-6-fluoro-5-hydroxy-2-methyl-1H-indol-3-yl]acetyl}amino)-3-methylpropanoic acid
2-({[1-(4-chloro-3-fluorobenzoyl)-6-fluoro-5-hydroxy-2-methyl-1H-indol-3-yl]acetyl}amino)propanoic acid
2-({[1-(4-chloro-3-fluorobenzoyl)-6-fluoro-5-hydroxy-2-methyl-1H-indol-3-yl]acetyl}amino)-2-methylpropanoic acid
[1-(4-chloro-3-fluorobenzoyl)-4-fluoro-5-hydroxy-2-methyl-1H-indol-3-yl]acetic acid,
methyl [1-(4-chloro-3-fluorobenzoyl)-4-fluoro-5-hydroxy-2-methyl-1H-indol-3-yl]acetate
ethyl [1-(4-chloro-3-fluorobenzoyl)-4-fluoro-5-hydroxy-2-methyl-1H-indol-3-yl]acetate
propyl [1-(4-chloro-3-fluorobenzoyl)-4-fluoro-5-hydroxy-2-methyl-1H-indol-3-yl]acetate
butyl [1-(4-chloro-3-fluorobenzoyl)-4-fluoro-5-hydroxy-2-methyl-1H-indol-3-yl]acetate
sec-butyl [1-(4-chloro-3-fluorobenzoyl)-4-fluoro-5-hydroxy-2-methyl-1H-indol-3-yl]acetate
(R) sec-butyl [1-(4-chloro-3-fluorobenzoyl)-4-fluoro-5-hydroxy-2-methyl-1H-indol-3-yl]acetate
(S) sec-butyl [1-(4-chloro-3-fluorobenzoyl)-4-fluoro-5-hydroxy-2-methyl-1H-indol-3-yl]acetate
isopropyl [1-(4-chloro-3-fluorobenzoyl)-4-fluoro-5-hydroxy-2-methyl-1H-indol-3-yl]acetate
tert-butyl [1-(4-chloro-3-fluorobenzoyl)-4-fluoro-5-hydroxy-2-methyl-1H-indol-3-yl]acetate
cyclobutyl [1-(4-chloro-3-fluorobenzoyl)-4-fluoro-5-hydroxy-2-methyl-1H-indol-3-yl]acetate
cyclopentyl [1-(4-chloro-3-fluorobenzoyl)-4-fluoro-5-hydroxy-2-methyl-1H-indol-3-yl]acetate
2-[1-(4-chloro-3-fluorobenzoyl)-4-fluoro-5-hydroxy-2-methyl-1H-indol-3-yl]acetamide
2-[1-(4-chloro-3-fluorobenzoyl)-4-fluoro-5-hydroxy-2-methyl-1H-indol-3-yl]-N-methylacetamide
2-[1-(4-chloro-3-fluorobenzoyl)-4-fluoro-5-hydroxy-2-methyl-1H-indol-3-yl]-N-ethylacetamide
2-[1-(4-chloro-3-fluorobenzoyl)-4-fluoro-5-hydroxy-2-methyl-1H-indol-3-yl]-N-propylacetamide
2-[1-(4-chloro-3-fluorobenzoyl)-4-fluoro-5-hydroxy-2-methyl-1H-indol-3-yl]-N-isopropylacetamide
2-[1-(4-chloro-3-fluorobenzoyl)-4-fluoro-5-hydroxy-2-methyl-1H-indol-3-yl]-N-butylacetamide
2-[1-(4-chloro-3-fluorobenzoyl)-4-fluoro-5-hydroxy-2-methyl-1H-indol-3-yl]-N-sec-butylacetamide
2-[1-(4-chloro-3-fluorobenzoyl)-4-fluoro-5-hydroxy-2-methyl-1H-indol-3-yl]-N-(2-hydroxyethyl)acetamide
({[1-(4-chloro-3-fluorobenzoyl)-4-fluoro-5-hydroxy-2-methyl-1H-indol-3-yl]acetyl}amino)acetic acid
3-({[1-(4-chloro-3-fluorobenzoyl)-4-fluoro-5-hydroxy-2-methyl-1H-indol-3-yl]acetyl}amino)propanoic acid
3-({[1-(4-chloro-3-fluorobenzoyl)-4-fluoro-5-hydroxy-2-methyl-1H-indol-3-yl]acetyl}amino)-2-methylpropanoic acid
3-({[1-(4-chloro-3-fluorobenzoyl)-4-fluoro-5-hydroxy-2-methyl-1H-indol-3-yl]acetyl}amino)-3-methylpropanoic acid
2-({[1-(4-chloro-3-fluorobenzoyl)-4-fluoro-5-hydroxy-2-methyl-1H-indol-3-yl]acetyl}amino)propanoic acid
2-({[1-(4-chloro-3-fluorobenzoyl)-4-fluoro-5-hydroxy-2-methyl-1H-indol-3-yl]acetyl}amino)-2-methylpropanoic acid
[1-(4-chloro-3-fluorobenzoyl)-4,6-difluoro-5-hydroxy-2-methyl-1H-indol-3-yl]acetic acid,
methyl [1-(4-chloro-3-fluorobenzoyl)-4,6-difluoro-5-hydroxy-2-methyl-1H-indol-3-yl]acetate
ethyl [1-(4-chloro-3-fluorobenzoyl)-4,6-difluoro-5-hydroxy-2-methyl-1H-indol-3-yl]acetate
propyl [1-(4-chloro-3-fluorobenzoyl)-4,6-difluoro-5-hydroxy-2-methyl-1H-indol-3-yl]acetate
butyl [1-(4-chloro-3-fluorobenzoyl)-4,6-difluoro-5-hydroxy-2-methyl-1H-indol-3-yl]acetate
sec-butyl [1-(4-chloro-3-fluorobenzoyl)-4,6-difluoro-5-hydroxy-2-methyl-1H-indol-3-yl]acetate
(R) sec-butyl [1-(4-chloro-3-fluorobenzoyl)-4,6-difluoro-5-hydroxy-2-methyl-1H-indol-3-yl]acetate
(S) sec-butyl [1-(4-chloro-3-fluorobenzoyl)-4,6-difluoro-5-hydroxy-2-methyl-1H-indol-3-yl]acetate
isopropyl [1-(4-chloro-3-fluorobenzoyl)-4,6-difluoro-5-hydroxy-2-methyl-1H-indol-3-yl]acetate
tert-butyl [1-(4-chloro-3-fluorobenzoyl)-4,6-difluoro-5-hydroxy-2-methyl-1H-indol-3-yl]acetate
cyclobutyl [1-(4-chloro-3-fluorobenzoyl)-4,6-difluoro-5-hydroxy-2-methyl-1H-indol-3-yl]acetate
cyclopentyl [1-(4-chloro-3-fluorobenzoyl)-4,6-difluoro-5-hydroxy-2-methyl-1H-indol-3-yl]acetate
2-[1-(4-chloro-3-fluorobenzoyl)-4,6-difluoro-5-hydroxy-2-methyl-1H-indol-3-yl]acetamide
2-[1-(4-chloro-3-fluorobenzoyl)-4,6-difluoro-5-hydroxy-2-methyl-1H-indol-3-yl]-N-methylacetamide
2-[1-(4-chloro-3-fluorobenzoyl)-4,6-difluoro-5-hydroxy-2-methyl-1H-indol-3-yl]-N-ethylacetamide
2-[1-(4-chloro-3-fluorobenzoyl)-4,6-difluoro-5-hydroxy-2-methyl-1H-indol-3-yl]-N-propylacetamide
2-[1-(4-chloro-3-fluorobenzoyl)-4,6-difluoro-5-hydroxy-2-methyl-1H-indol-3-yl]-N-isopropylacetamide
2-[1-(4-chloro-3-fluorobenzoyl)-4,6-difluoro-5-hydroxy-2-methyl-1H-indol-3-yl]-N-butylacetamide
2-[1-(4-chloro-3-fluorobenzoyl)-4,6-difluoro-5-hydroxy-2-methyl-1H-indol-3-yl]-N-sec-butylacetamide
2-[1-(4-chloro-3-fluorobenzoyl)-4,6-difluoro-5-hydroxy-2-methyl-1H-indol-3-yl]-N-(2-hydroxyethyl)acetamide
({[1-(4-chloro-3-fluorobenzoyl)-4,6-difluoro-5-hydroxy-2-methyl-1H-indol-3-yl]acetyl}amino)acetic acid
3-({[1-(4-chloro-3-fluorobenzoyl)-4,6-difluoro-5-hydroxy-2-methyl-1H-indol-3-yl]acetyl}amino)propanoic acid
3-({[1-(4-chloro-3-fluorobenzoyl)-4,6-difluoro-5-hydroxy-2-methyl-1H-indol-3-yl]acetyl}amino)-2-methylpropanoic acid
3-({[1-(4-chloro-3-fluorobenzoyl)-4,6-difluoro-5-hydroxy-2-methyl-1H-indol-3-yl]acetyl}amino)-3-methylpropanoic acid
2-({[1-(4-chloro-3-fluorobenzoyl)-4,6-difluoro-5-hydroxy-2-methyl-1H-indol-3-yl]acetyl}amino)propanoic acid
2-({[1-(4-chloro-3-fluorobenzoyl)-4,6-difluoro-5-hydroxy-2-methyl-1H-indol-3-yl]acetyl}amino)-2-methylpropanoic acid
[1-(3-fluorobenzoyl)-5-hydroxy-2-methyl-1H-indol-3-yl]acetic acid,
methyl [1-(3-fluorobenzoyl)-5-hydroxy-2-methyl-1H-indol-3-yl]acetate
ethyl [1-(3-fluorobenzoyl)-5-hydroxy-2-methyl-1H-indol-3-yl]acetate
propyl [1-(3-fluorobenzoyl)-5-hydroxy-2-methyl-1H-indol-3-yl]acetate
butyl [1-(3-fluorobenzoyl)-5-hydroxy-2-methyl-1H-indol-3-yl]acetate
sec-butyl [1-(3-fluorobenzoyl)-5-hydroxy-2-methyl-1H-indol-3-yl]acetate
(R) sec-butyl [1-(3-fluorobenzoyl)-5-hydroxy-2-methyl-1H-indol-3-yl]acetate
(S) sec-butyl [1-(3-fluorobenzoyl)-5-hydroxy-2-methyl-1H-indol-3-yl]acetate
isopropyl [1-(3-fluorobenzoyl)-5-hydroxy-2-methyl-1H-indol-3-yl]acetate
tert-butyl [1-(3-fluorobenzoyl)-5-hydroxy-2-methyl-1H-indol-3-yl]acetate
cyclobutyl [1-(3-fluorobenzoyl)-5-hydroxy-2-methyl-1H-indol-3-yl]acetate
cyclopentyl [1-(3-fluorobenzoyl)-5-hydroxy-2-methyl-1H-indol-3-yl]acetate
2-[1-(3-fluorobenzoyl)-5-hydroxy-2-methyl-1H-indol-3-yl]acetamide
2-[1-(3-fluorobenzoyl)-5-hydroxy-2-methyl-1H-indol-3-yl]-N-methylacetamide
2-[1-(3-fluorobenzoyl)-5-hydroxy-2-methyl-1H-indol-3-yl]-N-ethylacetamide
2-[1-(3-fluorobenzoyl)-5-hydroxy-2-methyl-1H-indol-3-yl]-N-propylacetamide
2-[1-(3-fluorobenzoyl)-5-hydroxy-2-methyl-1H-indol-3-yl]-N-isopropylacetamide
2-[1-(3-fluorobenzoyl)-5-hydroxy-2-methyl-1H-indol-3-yl]-N-butylacetamide
2-[1-(3-fluorobenzoyl)-5-hydroxy-2-methyl-1H-indol-3-yl]-N-sec-butylacetamide
2-[1-(3-fluorobenzoyl)-5-hydroxy-2-methyl-1H-indol-3-yl]-N-(2-hydroxyethyl)acetamide
({[1-(3-fluorobenzoyl)-5-hydroxy-2-methyl-1H-indol-3-yl]acetyl}amino)acetic acid
3-({[1-(3-fluorobenzoyl)-5-hydroxy-2-methyl-1H-indol-3-yl]acetyl}amino)propanoic acid
3-({[1-(3-fluorobenzoyl)-5-hydroxy-2-methyl-1H-indol-3-yl]acetyl}amino)-2-methylpropanoic acid
3-({[1-(3-fluorobenzoyl)-5-hydroxy-2-methyl-1H-indol-3-yl]acetyl}amino)-3-methylpropanoic acid
2-({[1-(3-fluorobenzoyl)-5-hydroxy-2-methyl-1H-indol-3-yl]acetyl}amino)propanoic acid
2-({[1-(3-fluorobenzoyl)-5-hydroxy-2-methyl-1H-indol-3-yl]acetyl}amino)-2-methylpropanoic acid
[1-(3-fluorobenzoyl)-6-fluoro-5-hydroxy-2-methyl-1H-indol-3-yl]acetic acid,
methyl [1-(3-fluorobenzoyl)-6-fluoro-5-hydroxy-2-methyl-1H-indol-3-yl]acetate
ethyl [1-(3-fluorobenzoyl)-6-fluoro-5-hydroxy-2-methyl-1H-indol-3-yl]acetate
propyl [1-(3-fluorobenzoyl)-6-fluoro-5-hydroxy-2-methyl-1H-indol-3-yl]acetate
butyl [1-(3-fluorobenzoyl)-6-fluoro-5-hydroxy-2-methyl-1H-indol-3-yl]acetate
sec-butyl [1-(3-fluorobenzoyl)-6-fluoro-5-hydroxy-2-methyl-1H-indol-3-yl]acetate
(R) sec-butyl [1-(3-fluorobenzoyl)-6-fluoro-5-hydroxy-2-methyl-1H-indol-3-yl]acetate
(S) sec-butyl [1-(3-fluorobenzoyl)-6-fluoro-5-hydroxy-2-methyl-1H-indol-3-yl]acetate
isopropyl [1-(3-fluorobenzoyl)-6-fluoro-5-hydroxy-2-methyl-1H-indol-3-yl]acetate
tert-butyl [1-(3-fluorobenzoyl)-6-fluoro-5-hydroxy-2-methyl-1H-indol-3-yl]acetate
cyclobutyl [1-(3-fluorobenzoyl)-6-fluoro-5-hydroxy-2-methyl-1H-indol-3-yl]acetate
cyclopentyl [1-(3-fluorobenzoyl)-6-fluoro-5-hydroxy-2-methyl-1H-indol-3-yl]acetate
2-[1-(3-fluorobenzoyl)-6-fluoro-5-hydroxy-2-methyl-1H-indol-3-yl]acetamide
2-[1-(3-fluorobenzoyl)-6-fluoro-5-hydroxy-2-methyl-1H-indol-3-yl]-N-methylacetamide
2-[1-(3-fluorobenzoyl)-6-fluoro-5-hydroxy-2-methyl-1H-indol-3-yl]-N-ethylacetamide
2-[1-(3-fluorobenzoyl)-6-fluoro-5-hydroxy-2-methyl-1H-indol-3-yl]-N-propylacetamide
2-[1-(3-fluorobenzoyl)-6-fluoro-5-hydroxy-2-methyl-1H-indol-3-yl]-N-isopropylacetamide
2-[1-(3-fluorobenzoyl)-6-fluoro-5-hydroxy-2-methyl-1H-indol-3-yl]-N-butylacetamide
2-[1-(3-fluorobenzoyl)-6-fluoro-5-hydroxy-2-methyl-1H-indol-3-yl]-N-sec-butylacetamide
2-[1-(3-fluorobenzoyl)-6-fluoro-5-hydroxy-2-methyl-1H-indol-3-yl]-N-(2-hydroxyethyl)acetamide
({[1-(3-fluorobenzoyl)-6-fluoro-5-hydroxy-2-methyl-1H-indol-3-yl]acetyl}amino)acetic acid
3-({[1-(3-fluorobenzoyl)-6-fluoro-5-hydroxy-2-methyl-1H-indol-3-yl]acetyl}amino)propanoic acid
3-({[1-(3-fluorobenzoyl)-6-fluoro-5-hydroxy-2-methyl-1H-indol-3-yl]acetyl}amino)-2-methylpropanoic acid
3-({[1-(3-fluorobenzoyl)-6-fluoro-5-hydroxy-2-methyl-1H-indol-3-yl]acetyl}amino)-3-methylpropanoic acid
2-({[1-(3-fluorobenzoyl)-6-fluoro-5-hydroxy-2-methyl-1H-indol-3-yl]acetyl}amino)propanoic acid
2-({[1-(3-fluorobenzoyl)-6-fluoro-5-hydroxy-2-methyl-1H-indol-3-yl]acetyl}amino)-2-methylpropanoic acid
[1-(3-fluorobenzoyl)-4-fluoro-5-hydroxy-2-methyl-1H-indol-3-yl]acetic acid,
methyl [1-(3-fluorobenzoyl)-4-fluoro-5-hydroxy-2-methyl-1H-indol-3-yl]acetate
ethyl [1-(3-fluorobenzoyl)-4-fluoro-5-hydroxy-2-methyl-1H-indol-3-yl]acetate
propyl [1-(3-fluorobenzoyl)-4-fluoro-5-hydroxy-2-methyl-1H-indol-3-yl]acetate
butyl [1-(3-fluorobenzoyl)-4-fluoro-5-hydroxy-2-methyl-1H-indol-3-yl]acetate
sec-butyl [1-(3-fluorobenzoyl)-4-fluoro-5-hydroxy-2-methyl-1H-indol-3-yl]acetate
(R) sec-butyl [1-(3-fluorobenzoyl)-4-fluoro-5-hydroxy-2-methyl-1H-indol-3-yl]acetate
(S) sec-butyl [1-(3-fluorobenzoyl)-4-fluoro-5-hydroxy-2-methyl-1H-indol-3-yl]acetate
isopropyl [1-(3-fluorobenzoyl)-4-fluoro-5-hydroxy-2-methyl-1H-indol-3-yl]acetate
tert-butyl [1-(3-fluorobenzoyl)-4-fluoro-5-hydroxy-2-methyl-1H-indol-3-yl]acetate
cyclobutyl [1-(3-fluorobenzoyl)-4-fluoro-5-hydroxy-2-methyl-1H-indol-3-yl]acetate
cyclopentyl [1-(3-fluorobenzoyl)-4-fluoro-5-hydroxy-2-methyl-1H-indol-3-yl]acetate
2-[1-(3-fluorobenzoyl)-4-fluoro-5-hydroxy-2-methyl-1H-indol-3-yl]acetamide
2-[1-(3-fluorobenzoyl)-4-fluoro-5-hydroxy-2-methyl-1H-indol-3-yl]-N-methylacetamide
2-[1-(3-fluorobenzoyl)-4-fluoro-5-hydroxy-2-methyl-1H-indol-3-yl]-N-ethylacetamide
2-[1-(3-fluorobenzoyl)-4-fluoro-5-hydroxy-2-methyl-1H-indol-3-yl]-N-propylacetamide
2-[1-(3-fluorobenzoyl)-4-fluoro-5-hydroxy-2-methyl-1H-indol-3-yl]-N-isopropylacetamide
2-[1-(3-fluorobenzoyl)-4-fluoro-5-hydroxy-2-methyl-1H-indol-3-yl]-N-butylacetamide
2-[1-(3-fluorobenzoyl)-4-fluoro-5-hydroxy-2-methyl-1H-indol-3-yl]-N-sec-butylacetamide
2-[1-(3-fluorobenzoyl)-4-fluoro-5-hydroxy-2-methyl-1H-indol-3-yl]-N-(2-hydroxyethyl)acetamide
({[1-(3-fluorobenzoyl)-4-fluoro-5-hydroxy-2-methyl-1H-indol-3-yl]acetyl}amino)acetic acid
3-({[1-(3-fluorobenzoyl)-4-fluoro-5-hydroxy-2-methyl-1H-indol-3-yl]acetyl}amino)propanoic acid
3-({[1-(3-fluorobenzoyl)-4-fluoro-5-hydroxy-2-methyl-1H-indol-3-yl]acetyl}amino)-2-methylpropanoic acid
3-({[1-(3-fluorobenzoyl)-4-fluoro-5-hydroxy-2-methyl-1H-indol-3-yl]acetyl}amino)-3-methylpropanoic acid
2-({[1-(3-fluorobenzoyl)-4-fluoro-5-hydroxy-2-methyl-1H-indol-3-yl]acetyl}amino)propanoic acid
2-({[1-(3-fluorobenzoyl)-4-fluoro-5-hydroxy-2-methyl-1H-indol-3-yl]acetyl}amino)-2-methylpropanoic acid
[1-(3-fluorobenzoyl)-4,6-difluoro-5-hydroxy-2-methyl-1H-indol-3-yl]acetic acid,
methyl [1-(3-fluorobenzoyl)-4,6-difluoro-5-hydroxy-2-methyl-1H-indol-3-yl]acetate
ethyl [1-(3-fluorobenzoyl)-4,6-difluoro-5-hydroxy-2-methyl-1H-indol-3-yl]acetate
propyl [1-(3-fluorobenzoyl)-4,6-difluoro-5-hydroxy-2-methyl-1H-indol-3-yl]acetate
butyl [1-(3-fluorobenzoyl)-4,6-difluoro-5-hydroxy-2-methyl-1H-indol-3-yl]acetate
sec-butyl [1-(3-fluorobenzoyl)-4,6-difluoro-5-hydroxy-2-methyl-1H-indol-3-yl]acetate
(R) sec-butyl [1-(3-fluorobenzoyl)-4,6-difluoro-5-hydroxy-2-methyl-1H-indol-3-yl]acetate
(S) sec-butyl [1-(3-fluorobenzoyl)-4,6-difluoro-5-hydroxy-2-methyl-1H-indol-3-yl]acetate
isopropyl [1-(3-fluorobenzoyl)-4,6-difluoro-5-hydroxy-2-methyl-1H-indol-3-yl]acetate
tert-butyl [1-(3-fluorobenzoyl)-4,6-difluoro-5-hydroxy-2-methyl-1H-indol-3-yl]acetate
cyclobutyl [1-(3-fluorobenzoyl)-4,6-difluoro-5-hydroxy-2-methyl-1H-indol-3-yl]acetate
cyclopentyl [1-(3-fluorobenzoyl)-4,6-difluoro-5-hydroxy-2-methyl-1H-indol-3-yl]acetate
2-[1-(3-fluorobenzoyl)-4,6-difluoro-5-hydroxy-2-methyl-1H-indol-3-yl]acetamide
2-[1-(3-fluorobenzoyl)-4,6-difluoro-5-hydroxy-2-methyl-1H-indol-3-yl]-N-methylacetamide
2-[1-(3-fluorobenzoyl)-4,6-difluoro-5-hydroxy-2-methyl-1H-indol-3-yl]-N-ethylacetamide
2-[1-(3-fluorobenzoyl)-4,6-difluoro-5-hydroxy-2-methyl-1H-indol-3-yl]-N-propylacetamide
2-[1-(3-fluorobenzoyl)-4,6-difluoro-5-hydroxy-2-methyl-1H-indol-3-yl]-N-isopropylacetamide
2-[1-(3-fluorobenzoyl)-4,6-difluoro-5-hydroxy-2-methyl-1H-indol-3-yl]-N-butylacetamide
2-[1-(3-fluorobenzoyl)-4,6-difluoro-5-hydroxy-2-methyl-1H-indol-3-yl]-N-sec-butylacetamide
2-[1-(3-fluorobenzoyl)-4,6-difluoro-5-hydroxy-2-methyl-1H-indol-3-yl]-N-(2-hydroxyethyl)acetamide
({[1-(3-fluorobenzoyl)-4,6-difluoro-5-hydroxy-2-methyl-1H-indol-3-yl]acetyl}amino)acetic acid
3-({[1-(3-fluorobenzoyl)-4,6-difluoro-5-hydroxy-2-methyl-1H-indol-3-yl]acetyl}amino)propanoic acid
3-({[1-(3-fluorobenzoyl)-4,6-difluoro-5-hydroxy-2-methyl-1H-indol-3-yl]acetyl}amino)-2-methylpropanoic acid
3-({[1-(3-fluorobenzoyl)-4,6-difluoro-5-hydroxy-2-methyl-1H-indol-3-yl]acetyl}amino)-3-methylpropanoic acid
2-({[1-(3-fluorobenzoyl)-4,6-difluoro-5-hydroxy-2-methyl-1H-indol-3-yl]acetyl}amino)propanoic acid
2-({[1-(3-fluorobenzoyl)-4,6-difluoro-5-hydroxy-2-methyl-1H-indol-3-yl]acetyl}amino)-2-methylpropanoic acid
(2R,S)-2[1-(3-chlorobenzoyl)-4,6-difluoro-5-hydroxy-2-methyl-1H-indol-3-yl]propionic acid,
methyl (2R,S)-2-[1-(3-chlorobenzoyl)-4,6-difluoro-5-hydroxy-2-methyl-1H-indol-3-yl]propanoate
ethyl (2R,S)-2-[1-(3-chlorobenzoyl)-4,6-difluoro-5-hydroxy-2-methyl-1H-indol-3-yl]propanoate
propyl (2R,S)-2-[1-(3-chlorobenzoyl)-4,6-difluoro-5-hydroxy-2-methyl-1H-indol-3-yl]propanoate
isopropyl (2R,S)-2-[1-(3-chlorobenzoyl)-4,6-difluoro-5-hydroxy-2-methyl-1H-indol-3-yl]propanoate
butyl (2R,S)-2-[1-(3-chlorobenzoyl)-4,6-difluoro-5-hydroxy-2-methyl-1H-indol-3-yl]propanoate
sec-butyl (2R,S)-2-[1-(3-chlorobenzoyl)-4,6-difluoro-5-hydroxy-2-methyl-1H-indol-3-yl]propanoate
(R) sec-butyl (2R,S)-2-[1-(3-chlorobenzoyl)-4,6-difluoro-5-hydroxy-2-methyl-1H-indol-3-yl]propanoate
(S) sec-butyl (2R,S)-2-[1-(3-chlorobenzoyl)-4,6-difluoro-5-hydroxy-2-methyl-1H-indol-3-yl]propanoate
tert-butyl (2R,S)-2-[1-(3-chlorobenzoyl)-4,6-difluoro-5-hydroxy-2-methyl-1H-indol-3-yl]propanoate
cyclobutyl (2R,S)-2-[1-(3-chlorobenzoyl)-4,6-difluoro-5-hydroxy-2-methyl-1H-indol-3-yl]propanoate
cyclopentyl (2R,S)-2-[1-(3-chlorobenzoyl)-4,6-difluoro-5-hydroxy-2-methyl-1H-indol-3-yl]propanoate
(2R,S)-2-[1-(3-chlorobenzoyl)-4,6-difluoro-5-hydroxy-2-methyl-1H-indol-3-yl]propanamide
(2R,S)-2-[1-(3-chlorobenzoyl)-4,6-difluoro-5-hydroxy-2-methyl-1H-indol-3-yl]-N-methylpropanamide
(2R,S)-2-[1-(3-chlorobenzoyl)-4,6-difluoro-5-hydroxy-2-methyl-1H-indol-3-yl]-N-ethylacetamide
(2R,S)-2-[1-(3-chlorobenzoyl)-4,6-difluoro-5-hydroxy-2-methyl-1H-indol-3-yl]-N-propylacetamide
(2R,S)-2-[1-(3-chlorobenzoyl)-4,6-difluoro-5-hydroxy-2-methyl-1H-indol-3-yl]-N-isopropylacetamide
(2R,S)-2-[1-(3-chlorobenzoyl)-4,6-difluoro-5-hydroxy-2-methyl-1H-indol-3-yl]-N-butylacetamide
(2R,S)-2-[1-(3-chlorobenzoyl)-4,6-difluoro-5-hydroxy-2-methyl-1H-indol-3-yl]-N-sec-butylacetamide
(2R,S)-2-[1-(3-chlorobenzoyl)-4,6-difluoro-5-hydroxy-2-methyl-1H-indol-3-yl]-N-(hydroxymethyl)propanamide
({(2R,S)-2-[1-(3-chlorobenzoyl)-4,6-difluoro-5-hydroxy-2-methyl-1H-indol-3-yl]propanoyl}amino)acetic acid
({(2R,S)-2-[1-(3-chlorobenzoyl)-4,6-difluoro-5-hydroxy-2-methyl-1H-indol-3-yl]propanoyl}amino)-2-methylpropionic acid
({(2R,S)-2-[1-(3-chlorobenzoyl)-4,6-difluoro-5-hydroxy-2-methyl-1H-indol-3-yl]propanoyl}amino)-2,2-methylbutyric acid
(2R,S)-3-({[1-(3-chlorobenzoyl)-4,6-difluoro-5-hydroxy-2-methyl-1H-indol-3-yl]propanoyl}amino)propanoic acid
(2R,S)-3-({[1-(3-chlorobenzoyl)-4,6-difluoro-5-hydroxy-2-methyl-1H-indol-3-yl]propanoyl}amino)-3-methylpropanic acid
(2R,S)-3-({[1-(3-chlorobenzoyl)-4,6-difluoro-5-hydroxy-2-methyl-1H-indol-3-yl]propanoyl}amino)-2-methylpropanic acid
(2R,S)-2[1-(3-chlorobenzoyl)-6-fluoro-5-hydroxy-2-methyl-1H-indol-3-yl]propionic acid,
methyl (2R,S)-2-[1-(3-chlorobenzoyl)-6-fluoro-5-hydroxy-2-methyl-1H-indol-3-yl]propanoate
ethyl (2R,S)-2-[1-(3-chlorobenzoyl)-6-fluoro-5-hydroxy-2-methyl-1H-indol-3-yl]propanoate
propyl (2R,S)-2-[1-(3-chlorobenzoyl)-6-fluoro-5-hydroxy-2-methyl-1H-indol-3-yl]propanoate
isopropyl (2R,S)-2-[1-(3-chlorobenzoyl)-6-fluoro-5-hydroxy-2-methyl-1H-indol-3-yl]propanoate
butyl (2R,S)-2-[1-(3-chlorobenzoyl)-6-fluoro-5-hydroxy-2-methyl-1H-indol-3-yl]propanoate
sec-butyl (2R,S)-2-[1-(3-chlorobenzoyl)-6-fluoro-5-hydroxy-2-methyl-1H-indol-3-yl]propanoate
(R) sec-butyl (2R,S)-2-[1-(3-chlorobenzoyl)-6-fluoro-5-hydroxy-2-methyl-1H-indol-3-yl]propanoate
(S) sec-butyl (2R,S)-2-[1-(3-chlorobenzoyl)-6-fluoro-5-hydroxy-2-methyl-1H-indol-3-yl]propanoate
tert-butyl (2R,S)-2-[1-(3-chlorobenzoyl)-6-fluoro-5-hydroxy-2-methyl-1H-indol-3-yl]propanoate
cyclobutyl (2R,S)-2-[1-(3-chlorobenzoyl)-6-fluoro-5-hydroxy-2-methyl-1H-indol-3-yl]propanoate
cyclopentyl (2R,S)-2-[1-(3-chlorobenzoyl)-6-fluoro-5-hydroxy-2-methyl-1H-indol-3-yl]propanoate
(2R,S)-2-[1-(3-chlorobenzoyl)-6-fluoro-5-hydroxy-2-methyl-1H-indol-3-yl]propanamide
(2R,S)-2-[1-(3-chlorobenzoyl)-6-fluoro-5-hydroxy-2-methyl-1H-indol-3-yl]-N-methylpropanamide
(2R,S)-2-[1-(3-chlorobenzoyl)-6-fluoro-5-hydroxy-2-methyl-1H-indol-3-yl]-N-ethylacetamide
(2R,S)-2-[1-(3-chlorobenzoyl)-6-fluoro-5-hydroxy-2-methyl-1H-indol-3-yl]-N-propylacetamide
(2R,S)-2-[1-(3-chlorobenzoyl)-6-fluoro-5-hydroxy-2-methyl-1H-indol-3-yl]-N-isopropylacetamide
(2R,S)-2-[1-(3-chlorobenzoyl)-6-fluoro-5-hydroxy-2-methyl-1H-indol-3-yl]-N-butylacetamide
(2R,S)-2-[1-(3-chlorobenzoyl)-6-fluoro-5-hydroxy-2-methyl-1H-indol-3-yl]-N-sec-butylacetamide
(2R,S)-2-[1-(3-chlorobenzoyl)-6-fluoro-5-hydroxy-2-methyl-1H-indol-3-yl]-N-(hydroxymethyl)propanamide
({(2R,S)-2-[1-(3-chlorobenzoyl)-6-fluoro-5-hydroxy-2-methyl-1H-indol-3-yl]propanoyl}amino)acetic acid
({(2R,S)-2-[1-(3-chlorobenzoyl)-6-fluoro-5-hydroxy-2-methyl-1H-indol-3-yl]propanoyl}amino)-2-methylpropionic acid
({(2R,S)-2-[1-(3-chlorobenzoyl)-6-fluoro-5-hydroxy-2-methyl-1H-indol-3-yl]propanoyl}amino)-2,2-methylbutyric acid
(2R,S)-3-({[1-(3-chlorobenzoyl)-6-fluoro-5-hydroxy-2-methyl-1H-indol-3-yl]propanoyl}amino)propanoic acid
(2R,S)-3-({[1-(3-chlorobenzoyl)-6-fluoro-5-hydroxy-2-methyl-1H-indol-3-yl]propanoyl}amino)-3-methylpropanic acid
(2R,S)-3-({[1-(3-chlorobenzoyl)-6-fluoro-5-hydroxy-2-methyl-1H-indol-3-yl]propanoyl}amino)-2-methylpropanic acid
(2R,S)-2[1-(3-chlorobenzoyl)-4-fluoro-5-hydroxy-2-methyl-1H-indol-3-yl]propionic acid,
methyl (2R,S)-2-[1-(3-chlorobenzoyl)-4-fluoro-5-hydroxy-2-methyl-1H-indol-3-yl]propanoate
ethyl (2R,S)-2-[1-(3-chlorobenzoyl)-4-fluoro-5-hydroxy-2-methyl-1H-indol-3-yl]propanoate
propyl (2R,S)-2-[1-(3-chlorobenzoyl)-4-fluoro-5-hydroxy-2-methyl-1H-indol-3-yl]propanoate
isopropyl (2R,S)-2-[1-(3-chlorobenzoyl)-4-fluoro-5-hydroxy-2-methyl-1H-indol-3-yl]propanoate
butyl (2R,S)-2-[1-(3-chlorobenzoyl)-4-fluoro-5-hydroxy-2-methyl-1H-indol-3-yl]propanoate
sec-butyl (2R,S)-2-[1-(3-chlorobenzoyl)-4-fluoro-5-hydroxy-2-methyl-1H-indol-3-yl]propanoate
(R) sec-butyl (2R,S)-2-[1-(3-chlorobenzoyl)-4-fluoro-5-hydroxy-2-methyl-1H-indol-3-yl]propanoate
(S) sec-butyl (2R,S)-2-[1-(3-chlorobenzoyl)-4-fluoro-5-hydroxy-2-methyl-1H-indol-3-yl]propanoate
tert-butyl (2R,S)-2-[1-(3-chlorobenzoyl)-4-fluoro-5-hydroxy-2-methyl-1H-indol-3-yl]propanoate
cyclobutyl (2R,S)-2-[1-(3-chlorobenzoyl)-4-fluoro-5-hydroxy-2-methyl-1H-indol-3-yl]propanoate
cyclopentyl (2R,S)-2-[1-(3-chlorobenzoyl)-4-fluoro-5-hydroxy-2-methyl-1H-indol-3-yl]propanoate
(2R,S)-2-[1-(3-chlorobenzoyl)-4-fluoro-5-hydroxy-2-methyl-1H-indol-3-yl]propanamide
(2R,S)-2-[1-(3-chlorobenzoyl)-4-fluoro-5-hydroxy-2-methyl-1H-indol-3-yl]-N-methylpropanamide
(2R,S)-2-[1-(3-chlorobenzoyl)-4-fluoro-5-hydroxy-2-methyl-1H-indol-3-yl]-N-ethylacetamide
(2R,S)-2-[1-(3-chlorobenzoyl)-4-fluoro-5-hydroxy-2-methyl-1H-indol-3-yl]-N-propylacetamide
(2R,S)-2-[1-(3-chlorobenzoyl)-4-fluoro-5-hydroxy-2-methyl-1H-indol-3-yl]-N-isopropylacetamide
(2R,S)-2-[1-(3-chlorobenzoyl)-4-fluoro-5-hydroxy-2-methyl-1H-indol-3-yl]-N-butylacetamide
(2R,S)-2-[1-(3-chlorobenzoyl)-4-fluoro-5-hydroxy-2-methyl-1H-indol-3-yl]-N-sec-butylacetamide
(2R,S)-2-[1-(3-chlorobenzoyl)-4-fluoro-5-hydroxy-2-methyl-1H-indol-3-yl]-N-(hydroxymethyl)propanamide
({(2R,S)-2-[1-(3-chlorobenzoyl)-4-fluoro-5-hydroxy-2-methyl-1H-indol-3-yl]propanoyl}amino)acetic acid
({(2R,S)-2-[1-(3-chlorobenzoyl)-4-fluoro-5-hydroxy-2-methyl-1H-indol-3-yl]propanoyl}amino)-2-methylpropionic acid
({(2R,S)-2-[1-(3-chlorobenzoyl)-4-fluoro-5-hydroxy-2-methyl-1H-indol-3-yl]propanoyl}amino)-2,2-methylbutyric acid
(2R,S)-3-({[1-(3-chlorobenzoyl)-4-fluoro-5-hydroxy-2-methyl-1H-indol-3-yl]propanoyl}amino)propanoic acid
(2R,S)-3-({[1-(3-chlorobenzoyl)-4-fluoro-5-hydroxy-2-methyl-1H-indol-3-yl]propanoyl}amino)-3-methylpropanic acid
(2R,S)-3-({[1-(3-chlorobenzoyl)-4-fluoro-5-hydroxy-2-methyl-1H-indol-3-yl]propanoyl}amino)-2-methylpropanic acid
(2R,S)-2[1-(3-chlorobenzoyl)-5-hydroxy-2-methyl-1H-indol-3-yl]propionic acid,
methyl (2R,S)-2-[1-(3-chlorobenzoyl)-5-hydroxy-2-methyl-1H-indol-3-yl]propanoate
ethyl (2R,S)-2-[1-(3-chlorobenzoyl)-5-hydroxy-2-methyl-1H-indol-3-yl]propanoate
propyl (2R,S)-2-[1-(3-chlorobenzoyl)-5-hydroxy-2-methyl-1H-indol-3-yl]propanoate
isopropyl (2R,S)-2-[1-(3-chlorobenzoyl)-5-hydroxy-2-methyl-1H-indol-3-yl]propanoate
butyl (2R,S)-2-[1-(3-chlorobenzoyl)-5-hydroxy-2-methyl-1H-indol-3-yl]propanoate
sec-butyl (2R,S)-2-[1-(3-chlorobenzoyl)-5-hydroxy-2-methyl-1H-indol-3-yl]propanoate
(R) sec-butyl (2R,S)-2-[1-(3-chlorobenzoyl)-5-hydroxy-2-methyl-1H-indol-3-yl]propanoate
(S) sec-butyl (2R,S)-2-[1-(3-chlorobenzoyl)-5-hydroxy-2-methyl-1H-indol-3-yl]propanoate
tert-butyl (2R,S)-2-[1-(3-chlorobenzoyl)-5-hydroxy-2-methyl-1H-indol-3-yl]propanoate
cyclobutyl (2R,S)-2-[1-(3-chlorobenzoyl)-5-hydroxy-2-methyl-1H-indol-3-yl]propanoate
cyclopentyl (2R,S)-2-[1-(3-chlorobenzoyl)-5-hydroxy-2-methyl-1H-indol-3-yl]propanoate
(2R,S)-2-[1-(3-chlorobenzoyl)-5-hydroxy-2-methyl-1H-indol-3-yl]propanamide
(2R,S)-2-[1-(3-chlorobenzoyl)-5-hydroxy-2-methyl-1H-indol-3-yl]-N-methylpropanamide
(2R,S)-2-[1-(3-chlorobenzoyl)-5-hydroxy-2-methyl-1H-indol-3-yl]-N-ethylacetamide
(2R,S)-2-[1-(3-chlorobenzoyl)-5-hydroxy-2-methyl-1H-indol-3-yl]-N-propylacetamide
(2R,S)-2-[1-(3-chlorobenzoyl)-5-hydroxy-2-methyl-1H-indol-3-yl]-N-isopropylacetamide
(2R,S)-2-[1-(3-chlorobenzoyl)-5-hydroxy-2-methyl-1H-indol-3-yl]-N-butylacetamide
(2R,S)-2-[1-(3-chlorobenzoyl)-5-hydroxy-2-methyl-1H-indol-3-yl]-N-sec-butylacetamide
(2R,S)-2-[1-(3-chlorobenzoyl)-5-hydroxy-2-methyl-1H-indol-3-yl]-N-(hydroxymethyl)propanamide
({(2R,S)-2-[1-(3-chlorobenzoyl)-5-hydroxy-2-methyl-1H-indol-3-yl]propanoyl}amino)acetic acid
({(2R,S)-2-[1-(3-chlorobenzoyl)-5-hydroxy-2-methyl-1H-indol-3-yl]propanoyl}amino)-2-methylpropionic acid
({(2R,S)-2-[1-(3-chlorobenzoyl)-5-hydroxy-2-methyl-1H-indol-3-yl]propanoyl}amino)-2,2-methylbutyric acid
(2R,S)-3-({[1-(3-chlorobenzoyl)-5-hydroxy-2-methyl-1H-indol-3-yl]propanoyl}amino)propanoic acid
(2R,S)-3-({[1-(3-chlorobenzoyl)-5-hydroxy-2-methyl-1H-indol-3-yl]propanoyl}amino)-3-methylpropanic acid
(2R,S)-3-({[1-(3-chlorobenzoyl)-5-hydroxy-2-methyl-1H-indol-3-yl]propanoyl}amino)-2-methylpropanic acid
(2R,S)-2[1-(3,4-dichlorobenzoyl)-5-hydroxy-2-methyl-1H-indol-3-yl]propionic acid,
methyl (2R,S)-2-[1-(3,4-dichlorobenzoyl)-5-hydroxy-2-methyl-1H-indol-3-yl]propanoate
ethyl (2R,S)-2-[1-(3,4-dichlorobenzoyl)-5-hydroxy-2-methyl-1H-indol-3-yl]propanoate
propyl (2R,S)-2-[1-(3,4-dichlorobenzoyl)-5-hydroxy-2-methyl-1H-indol-3-yl]propanoate
isopropyl (2R,S)-2-[1-(3,4-dichlorobenzoyl)-5-hydroxy-2-methyl-1H-indol-3-yl]propanoate
butyl (2R,S)-2-[1-(3,4-dichlorobenzoyl)-5-hydroxy-2-methyl-1H-indol-3-yl]propanoate
sec-butyl (2R,S)-2-[1-(3,4-dichlorobenzoyl)-5-hydroxy-2-methyl-1H-indol-3-yl]propanoate
(R) sec-butyl (2R,S)-2-[1-(3,4-dichlorobenzoyl)-5-hydroxy-2-methyl-1H-indol-3-yl]propanoate
(S) sec-butyl (2R,S)-2-[1-(3,4-dichlorobenzoyl)-5-hydroxy-2-methyl-1H-indol-3-yl]propanoate
tert-butyl (2R,S)-2-[1-(3,4-dichlorobenzoyl)-5-hydroxy-2-methyl-1H-indol-3-yl]propanoate
cyclobutyl (2R,S)-2-[1-(3,4-dichlorobenzoyl)-5-hydroxy-2-methyl-1H-indol-3-yl]propanoate
cyclopentyl (2R,S)-2-[1-(3,4-dichlorobenzoyl)-5-hydroxy-2-methyl-1H-indol-3-yl]propanoate
(2R,S)-2-[1-(3,4-dichlorobenzoyl)-5-hydroxy-2-methyl-1H-indol-3-yl]propanamide
(2R,S)-2-[1-(3,4-dichlorobenzoyl)-5-hydroxy-2-methyl-1H-indol-3-yl]-N-methylpropanamide
(2R,S)-2-[1-(3,4-dichlorobenzoyl)-5-hydroxy-2-methyl-1H-indol-3-yl]-N-ethylacetamide
(2R,S)-2-[1-(3,4-dichlorobenzoyl)-5-hydroxy-2-methyl-1H-indol-3-yl]-N-propylacetamide
(2R,S)-2-[1-(3,4-dichlorobenzoyl)-5-hydroxy-2-methyl-1H-indol-3-yl]-N-isopropylacetamide
(2R,S)-2-[1-(3,4-dichlorobenzoyl)-5-hydroxy-2-methyl-1H-indol-3-yl]-N-butylacetamide
(2R,S)-2-[1-(3,4-dichlorobenzoyl)-5-hydroxy-2-methyl-1H-indol-3-yl]-N-sec-butylacetamide
(2R,S)-2-[1-(3,4-dichlorobenzoyl)-5-hydroxy-2-methyl-1H-indol-3-yl]-N-(hydroxymethyl)propanamide
({(2R,S)-2-[1-(3,4-dichlorobenzoyl)-5-hydroxy-2-methyl-1H-indol-3-yl]propanoyl}amino)acetic acid
({(2R,S)-2-[1-(3,4-dichlorobenzoyl)-5-hydroxy-2-methyl-1H-indol-3-yl]propanoyl}amino)-2-methylpropionic acid
({(2R,S)-2-[1-(3,4-dichlorobenzoyl)-5-hydroxy-2-methyl-1H-indol-3-yl]propanoyl}amino)-2,2-methylbutyric acid
(2R,S)-3-({[1-(3,4-dichlorobenzoyl)-5-hydroxy-2-methyl-1H-indol-3-yl]propanoyl}amino)propanoic acid
(2R,S)-3-({[1-(3,4-dichlorobenzoyl)-5-hydroxy-2-methyl-1H-indol-3-yl]propanoyl}amino)-3-methylpropanic acid
(2R,S)-3-({[1-(3,4-dichlorobenzoyl)-5-hydroxy-2-methyl-1H-indol-3-yl]propanoyl}amino)-2-methylpropanic acid
(2R,S)-2[1-(3,4-dichlorobenzoyl)-6-fluoro-5-hydroxy-2-methyl-1H-indol-3-yl]propionic acid,
methyl (2R,S)-2-[1-(3,4-dichlorobenzoyl)-6-fluoro-5-hydroxy-2-methyl-1H-indol-3-yl]propanoate
ethyl (2R,S)-2-[1-(3,4-dichlorobenzoyl)-6-fluoro-5-hydroxy-2-methyl-1H-indol-3-yl]propanoate
propyl (2R,S)-2-[1-(3,4-dichlorobenzoyl)-6-fluoro-5-hydroxy-2-methyl-1H-indol-3-yl]propanoate
isopropyl (2R,S)-2-[1-(3,4-dichlorobenzoyl)-6-fluoro-5-hydroxy-2-methyl-1H-indol-3-yl]propanoate
butyl (2R,S)-2-[1-(3,4-dichlorobenzoyl)-6-fluoro-5-hydroxy-2-methyl-1H-indol-3-yl]propanoate
sec-butyl (2R,S)-2-[1-(3,4-dichlorobenzoyl)-6-fluoro-5-hydroxy-2-methyl-1H-indol-3-yl]propanoate
(R) sec-butyl (2R,S)-2-[1-(3,4-dichlorobenzoyl)-6-fluoro-5-hydroxy-2-methyl-1H-indol-3-yl]propanoate
(S) sec-butyl (2R,S)-2-[1-(3,4-dichlorobenzoyl)-6-fluoro-5-hydroxy-2-methyl-1H-indol-3-yl]propanoate
tert-butyl (2R,S)-2-[1-(3,4-dichlorobenzoyl)-6-fluoro-5-hydroxy-2-methyl-1H-indol-3-yl]propanoate
cyclobutyl (2R,S)-2-[1-(3,4-dichlorobenzoyl)-6-fluoro-5-hydroxy-2-methyl-1H-indol-3-yl]propanoate
cyclopentyl (2R,S)-2-[1-(3,4-dichlorobenzoyl)-6-fluoro-5-hydroxy-2-methyl-1H-indol-3-yl]propanoate
(2R,S)-2-[1-(3,4-dichlorobenzoyl)-6-fluoro-5-hydroxy-2-methyl-1H-indol-3-yl]propanamide
(2R,S)-2-[1-(3,4-dichlorobenzoyl)-6-fluoro-5-hydroxy-2-methyl-1H-indol-3-yl]-N-methylpropanamide
(2R,S)-2-[1-(3,4-dichlorobenzoyl)-6-fluoro-5-hydroxy-2-methyl-1H-indol-3-yl]-N-ethylacetamide
(2R,S)-2-[1-(3,4-dichlorobenzoyl)-6-fluoro-5-hydroxy-2-methyl-1H-indol-3-yl]-N-propylacetamide
(2R,S)-2-[1-(3,4-dichlorobenzoyl)-6-fluoro-5-hydroxy-2-methyl-1H-indol-3-yl]-N-isopropylacetamide
(2R,S)-2-[1-(3,4-dichlorobenzoyl)-6-fluoro-5-hydroxy-2-methyl-1H-indol-3-yl]-N-butylacetamide
(2R,S)-2-[1-(3,4-dichlorobenzoyl)-6-fluoro-5-hydroxy-2-methyl-1H-indol-3-yl]-N-sec-butylacetamide
(2R,S)-2-[1-(3,4-dichlorobenzoyl)-6-fluoro-5-hydroxy-2-methyl-1H-indol-3-yl]-N-(hydroxymethyl)propanamide
({(2R,S)-2-[1-(3,4-dichlorobenzoyl)-6-fluoro-5-hydroxy-2-methyl-1H-indol-3-yl]propanoyl}amino)acetic acid
({(2R,S)-2-[1-(3,4-dichlorobenzoyl)-6-fluoro-5-hydroxy-2-methyl-1H-indol-3-yl]propanoyl}amino)-2-methylpropionic acid
({(2R,S)-2-[1-(3,4-dichlorobenzoyl)-6-fluoro-5-hydroxy-2-methyl-1H-indol-3-yl]propanoyl}amino)-2,2-methylbutyric acid
(2R,S)-3-({[1-(3,4-dichlorobenzoyl)-6-fluoro-5-hydroxy-2-methyl-1H-indol-3-yl]propanoyl}amino)propanoic acid
(2R,S)-3-({[1-(3,4-dichlorobenzoyl)-6-fluoro-5-hydroxy-2-methyl-1H-indol-3-yl]propanoyl}amino)-3-methylpropanic acid
(2R,S)-3-({[1-(3,4-dichlorobenzoyl)-6-fluoro-5-hydroxy-2-methyl-1H-indol-3-yl]propanoyl}amino)-2-methylpropanic acid
(2R,S)-2[1-(3,4-dichlorobenzoyl)-4-fluoro-5-hydroxy-2-methyl-1H-indol-3-yl]propionic acid,
methyl (2R,S)-2-[1-(3,4-dichlorobenzoyl)-4-fluoro-5-hydroxy-2-methyl-1H-indol-3-yl]propanoate
ethyl (2R,S)-2-[1-(3,4-dichlorobenzoyl)-4-fluoro-5-hydroxy-2-methyl-1H-indol-3-yl]propanoate
propyl (2R,S)-2-[1-(3,4-dichlorobenzoyl)-4-fluoro-5-hydroxy-2-methyl-1H-indol-3-yl]propanoate
isopropyl (2R,S)-2-[1-(3,4-dichlorobenzoyl)-4-fluoro-5-hydroxy-2-methyl-1H-indol-3-yl]propanoate
butyl (2R,S)-2-[1-(3,4-dichlorobenzoyl)-4-fluoro-5-hydroxy-2-methyl-1H-indol-3-yl]propanoate
sec-butyl (2R,S)-2-[1-(3,4-dichlorobenzoyl)-4-fluoro-5-hydroxy-2-methyl-1H-indol-3-yl]propanoate
(R) sec-butyl (2R,S)-2-[1-(3,4-dichlorobenzoyl)-4-fluoro-5-hydroxy-2-methyl-1H-indol-3-yl]propanoate
(S) sec-butyl (2R,S)-2-[1-(3,4-dichlorobenzoyl)-4-fluoro-5-hydroxy-2-methyl-1H-indol-3-yl]propanoate
tert-butyl (2R,S)-2-[1-(3,4-dichlorobenzoyl)-4-fluoro-5-hydroxy-2-methyl-1H-indol-3-yl]propanoate
cyclobutyl (2R,S)-2-[1-(3,4-dichlorobenzoyl)-4-fluoro-5-hydroxy-2-methyl-1H-indol-3-yl]propanoate
cyclopentyl (2R,S)-2-[1-(3,4-dichlorobenzoyl)-4-fluoro-5-hydroxy-2-methyl-1H-indol-3-yl]propanoate
(2R,S)-2-[1-(3,4-dichlorobenzoyl)-4-fluoro-5-hydroxy-2-methyl-1H-indol-3-yl]propanamide
(2R,S)-2-[1-(3,4-dichlorobenzoyl)-4-fluoro-5-hydroxy-2-methyl-1H-indol-3-yl]-N-methylpropanamide
(2R,S)-2-[1-(3,4-dichlorobenzoyl)-4-fluoro-5-hydroxy-2-methyl-1H-indol-3-yl]-N-ethylacetamide
(2R,S)-2-[1-(3,4-dichlorobenzoyl)-4-fluoro-5-hydroxy-2-methyl-1H-indol-3-yl]-N-propylacetamide
(2R,S)-2-[1-(3,4-dichlorobenzoyl)-4-fluoro-5-hydroxy-2-methyl-1H-indol-3-yl]-N-isopropylacetamide
(2R,S)-2-[1-(3,4-dichlorobenzoyl)-4-fluoro-5-hydroxy-2-methyl-1H-indol-3-yl]-N-butylacetamide
(2R,S)-2-[1-(3,4-dichlorobenzoyl)-4-fluoro-5-hydroxy-2-methyl-1H-indol-3-yl]-N-sec-butylacetamide
(2R,S)-2-[1-(3,4-dichlorobenzoyl)-4-fluoro-5-hydroxy-2-methyl-1H-indol-3-yl]-N-(hydroxymethyl)propanamide
({(2R,S)-2-[1-(3,4-dichlorobenzoyl)-4-fluoro-5-hydroxy-2-methyl-1H-indol-3-yl]propanoyl}amino)acetic acid
({(2R,S)-2-[1-(3,4-dichlorobenzoyl)-4-fluoro-5-hydroxy-2-methyl-1H-indol-3-yl]propanoyl}amino)-2-methylpropionic acid
({(2R,S)-2-[1-(3,4-dichlorobenzoyl)-4-fluoro-5-hydroxy-2-methyl-1H-indol-3-yl]propanoyl}amino)-2,2-methylbutyric acid
(2R,S)-3-({[1-(3,4-dichlorobenzoyl)-4-fluoro-5-hydroxy-2-methyl-1H-indol-3-yl]propanoyl}amino)propanoic acid
(2R,S)-3-({[1-(3,4-dichlorobenzoyl)-4-fluoro-5-hydroxy-2-methyl-1H-indol-3-yl]propanoyl}amino)-3-methylpropanic acid
(2R,S)-3-({[1-(3,4-dichlorobenzoyl)-4-fluoro-5-hydroxy-2-methyl-1H-indol-3-yl]propanoyl}amino)-2-methylpropanic acid
(2R,S)-2[1-(3,4-dichlorobenzoyl)-4,6-difluoro-5-hydroxy-2-methyl-1H-indol-3-yl)propionic acid,
methyl (2R,S)-2-[1-(3,4-dichlorobenzoyl)-4,6-difluoro-5-hydroxy-2-methyl-1H-indol-3-yl]propanoate
ethyl (2R,S)-2-[1-(3,4-dichlorobenzoyl)-4,6-difluoro-5-hydroxy-2-methyl-1H-indol-3-yl]propanoate
propyl (2R,S)-2-[1-(3,4-dichlorobenzoyl)-4,6-difluoro-5-hydroxy-2-methyl-1H-indol-3-yl]propanoate
isopropyl (2R,S)-2-[1-(3,4-dichlorobenzoyl)-4,6-difluoro-5-hydroxy-2-methyl-1H-indol-3-yl]propanoate
butyl (2R,S)-2-[1-(3,4-dichlorobenzoyl)-4,6-difluoro-5-hydroxy-2-methyl-1H-indol-3-yl]propanoate
sec-butyl (2R,S)-2-[1-(3,4-dichlorobenzoyl)-4,6-difluoro-5-hydroxy-2-methyl-1H-indol-3-yl]propanoate
(R) sec-butyl (2R,S)-2-[1-(3,4-dichlorobenzoyl)-4,6-difluoro-5-hydroxy-2-methyl-1H-indol-3-yl]propanoate
(S) sec-butyl (2R,S)-2-[1-(3,4-dichlorobenzoyl)-4,6-difluoro-5-hydroxy-2-methyl-1H-indol-3-yl]propanoate
tert-butyl (2R,S)-2-[1-(3,4-dichlorobenzoyl)-4,6-difluoro-5-hydroxy-2-methyl-1H-indol-3-yl]propanoate
cyclobutyl (2R,S)-2-[1-(3,4-dichlorobenzoyl)-4,6-difluoro-5-hydroxy-2-methyl-1H-indol-3-yl]propanoate
cyclopentyl (2R,S)-2-[1-(3,4-dichlorobenzoyl)-4,6-difluoro-5-hydroxy-2-methyl-1H-indol-3-yl]propanoate
(2R,S)-2-[1-(3,4-dichlorobenzoyl)-4,6-difluoro-5-hydroxy-2-methyl-1H-indol-3-yl]propanamide
(2R,S)-2-[1-(3,4-dichlorobenzoyl)-4,6-difluoro-5-hydroxy-2-methyl-1H-indol-3-yl]-N-methylpropanamide
(2R,S)-2-[1-(3,4-dichlorobenzoyl)-4,6-difluoro-5-hydroxy-2-methyl-1H-indol-3-yl]-N-ethylacetamide
(2R,S)-2-[1-(3,4-dichlorobenzoyl)-4,6-difluoro-5-hydroxy-2-methyl-1H-indol-3-yl]-N-propylacetamide
(2R,S)-2-[1-(3,4-dichlorobenzoyl)-4,6-difluoro-5-hydroxy-2-methyl-1H-indol-3-yl]-N-isopropylacetamide
(2R,S)-2-[1-(3,4-dichlorobenzoyl)-4,6-difluoro-5-hydroxy-2-methyl-1H-indol-3-yl]-N-butylacetamide
(2R,S)-2-[1-(3,4-dichlorobenzoyl)-4,6-difluoro-5-hydroxy-2-methyl-1H-indol-3-yl]-N-sec-butylacetamide
(2R,S)-2-[1-(3,4-dichlorobenzoyl)-4,6-difluoro-5-hydroxy-2-methyl-1H-indol-3-yl]-N-(hydroxymethyl)propanamide
({(2R,S)-2-[1-(3,4-dichlorobenzoyl)-4,6-difluoro-5-hydroxy-2-methyl-1H-indol-3-yl]propanoyl}amino)acetic acid
({(2R,S)-2-[1-(3,4-dichlorobenzoyl)-4,6-difluoro-5-hydroxy-2-methyl-1H-indol-3-yl]propanoyl}amino)-2-methylpropionic acid
({(2R,S)-2-[1-(3,4-dichlorobenzoyl)-4,6-difluoro-5-hydroxy-2-methyl-1H-indol-3-yl]propanoyl}amino)-2,2-methylbutyric acid
(2R,S)-3-({[1-(3,4-dichlorobenzoyl)-4,6-difluoro-5-hydroxy-2-methyl-1H-indol-3-yl]propanoyl}amino)propanoic acid
(2R,S)-3-({[1-(3,4-dichlorobenzoyl)-4,6-difluoro-5-hydroxy-2-methyl-1H-indol-3-yl]propanoyl}amino)-3-methylpropanic acid
(2R,S)-3-({[1-(3,4-dichlorobenzoyl)-4,6-difluoro-5-hydroxy-2-methyl-1H-indol-3-yl]propanoyl}amino)-2-methylpropanic acid
(2R,S)-2[1-(3,4-difluorobenzoyl)-5-hydroxy-2-methyl-1H-indol-3-yl]propionic acid,
methyl (2R,S)-2-[1-(3,4-difluorobenzoyl)-5-hydroxy-2-methyl-1H-indol-3-yl]propanoate
ethyl (2R,S)-2-[1-(3,4-difluorobenzoyl)-5-hydroxy-2-methyl-1H-indol-3-yl]propanoate
propyl (2R,S)-2-[1-(3,4-difluorobenzoyl)-5-hydroxy-2-methyl-1H-indol-3-yl]propanoate
isopropyl (2R,S)-2-[1-(3,4-difluorobenzoyl)-5-hydroxy-2-methyl-1H-indol-3-yl]propanoate
butyl (2R,S)-2-[1-(3,4-difluorobenzoyl)-5-hydroxy-2-methyl-1H-indol-3-yl]propanoate
sec-butyl (2R,S)-2-[1-(3,4-difluorobenzoyl)-5-hydroxy-2-methyl-1H-indol-3-yl]propanoate
(R) sec-butyl (2R,S)-2-[1-(3,4-difluorobenzoyl)-5-hydroxy-2-methyl-1H-indol-3-yl]propanoate
(S) sec-butyl (2R,S)-2-[1-(3,4-difluorobenzoyl)-5-hydroxy-2-methyl-1H-indol-3-yl]propanoate
tert-butyl (2R,S)-2-[1-(3,4-difluorobenzoyl)-5-hydroxy-2-methyl-1H-indol-3-yl]propanoate
cyclobutyl (2R,S)-2-[1-(3,4-difluorobenzoyl)-5-hydroxy-2-methyl-1H-indol-3-yl]propanoate
cyclopentyl (2R,S)-2-[1-(3,4-difluorobenzoyl)-5-hydroxy-2-methyl-1H-indol-3-yl]propanoate
(2R,S)-2-[1-(3,4-difluorobenzoyl)-5-hydroxy-2-methyl-1H-indol-3-yl]propanamide
(2R,S)-2-[1-(3,4-difluorobenzoyl)-5-hydroxy-2-methyl-1H-indol-3-yl]-N-methylpropanamide
(2R,S)-2-[1-(3,4-difluorobenzoyl)-5-hydroxy-2-methyl-1H-indol-3-yl]-N-ethylacetamide
(2R,S)-2-[1-(3,4-difluorobenzoyl)-5-hydroxy-2-methyl-1H-indol-3-yl]-N-propylacetamide
(2R,S)-2-[1-(3,4-difluorobenzoyl)-5-hydroxy-2-methyl-1H-indol-3-yl]-N-isopropylacetamide
(2R,S)-2-[1-(3,4-difluorobenzoyl)-5-hydroxy-2-methyl-1H-indol-3-yl]-N-butylacetamide
(2R,S)-2-[1-(3,4-difluorobenzoyl)-5-hydroxy-2-methyl-1H-indol-3-yl]-N-sec-butylacetamide
(2R,S)-2-[1-(3,4-difluorobenzoyl)-5-hydroxy-2-methyl-1H-indol-3-yl]-N-(hydroxymethyl)propanamide
({(2R,S)-2-[1-(3,4-difluorobenzoyl)-5-hydroxy-2-methyl-1H-indol-3-yl]propanoyl}amino)acetic acid
({(2R,S)-2-[1-(3,4-difluorobenzoyl)-5-hydroxy-2-methyl-1H-indol-3-yl]propanoyl}amino)-2-methylpropionic acid
({(2R,S)-2-[1-(3,4-difluorobenzoyl)-5-hydroxy-2-methyl-1H-indol-3-yl]propanoyl}amino)-2,2-methylbutyric acid
(2R,S)-3-({[1-(3,4-difluorobenzoyl)-5-hydroxy-2-methyl-1H-indol-3-yl]propanoyl}amino)propanoic acid
(2R,S)-3-({[1-(3,4-difluorobenzoyl)-5-hydroxy-2-methyl-1H-indol-3-yl]propanoyl}amino)-3-methylpropanic acid
(2R,S)-3-({[1-(3,4-difluorobenzoyl)-5-hydroxy-2-methyl-1H-indol-3-yl]propanoyl}amino)-2-methylpropanic acid
(2R,S)-2[1-(3,4-difluorobenzoyl)-6-fluoro-5-hydroxy-2-methyl-1H-indol-3-yl]propionic acid,
methyl (2R,S)-2-[1-(3,4-difluorobenzoyl)-6-fluoro-5-hydroxy-2-methyl-1H-indol-3-yl]propanoate
ethyl (2R,S)-2-[1-(3,4-difluorobenzoyl)-6-fluoro-5-hydroxy-2-methyl-1H-indol-3-yl]propanoate
propyl (2R,S)-2-[1-(3,4-difluorobenzoyl)-6-fluoro-5-hydroxy-2-methyl-1H-indol-3-yl]propanoate
isopropyl (2R,S)-2-[1-(3,4-difluorobenzoyl)-6-fluoro-5-hydroxy-2-methyl-1H-indol-3-yl]propanoate
butyl (2R,S)-2-[1-(3,4-difluorobenzoyl)-6-fluoro-5-hydroxy-2-methyl-1H-indol-3-yl]propanoate
sec-butyl (2R,S)-2-[1-(3,4-difluorobenzoyl)-6-fluoro-5-hydroxy-2-methyl-1H-indol-3-yl]propanoate
(R) sec-butyl (2R,S)-2-[1-(3,4-difluorobenzoyl)-6-fluoro-5-hydroxy-2-methyl-1H-indol-3-yl]propanoate
(S) sec-butyl (2R,S)-2-[1-(3,4-difluorobenzoyl)-6-fluoro-5-hydroxy-2-methyl-1H-indol-3-yl]propanoate
tert-butyl (2R,S)-2-[1-(3,4-difluorobenzoyl)-6-fluoro-5-hydroxy-2-methyl-1H-indol-3-yl]propanoate
cyclobutyl (2R,S)-2-[1-(3,4-difluorobenzoyl)-6-fluoro-5-hydroxy-2-methyl-1H-indol-3-yl]propanoate
cyclopentyl (2R,S)-2-[1-(3,4-difluorobenzoyl)-6-fluoro-5-hydroxy-2-methyl-1H-indol-3-yl]propanoate
(2R,S)-2-[1-(3,4-difluorobenzoyl)-6-fluoro-5-hydroxy-2-methyl-1H-indol-3-yl]propanamide
(2R,S)-2-[1-(3,4-difluorobenzoyl)-6-fluoro-5-hydroxy-2-methyl-1H-indol-3-yl]-N-methylpropanamide
(2R,S)-2-[1-(3,4-difluorobenzoyl)-6-fluoro-5-hydroxy-2-methyl-1H-indol-3-yl]-N-ethylacetamide
(2R,S)-2-[1-(3,4-difluorobenzoyl)-6-fluoro-5-hydroxy-2-methyl-1H-indol-3-yl]-N-propylacetamide
(2R,S)-2-[1-(3,4-difluorobenzoyl)-6-fluoro-5-hydroxy-2-methyl-1H-indol-3-yl]-N-isopropylacetamide
(2R,S)-2-[1-(3,4-difluorobenzoyl)-6-fluoro-5-hydroxy-2-methyl-1H-indol-3-yl]-N-butylacetamide
(2R,S)-2-[1-(3,4-difluorobenzoyl)-6-fluoro-5-hydroxy-2-methyl-1H-indol-3-yl]-N-sec-butylacetamide
(2R,S)-2-[1-(3,4-difluorobenzoyl)-6-fluoro-5-hydroxy-2-methyl-1H-indol-3-yl]-N-(hydroxymethyl)propanamide
({(2R,S)-2-[1-(3,4-difluorobenzoyl)-6-fluoro-5-hydroxy-2-methyl-1H-indol-3-yl]propanoyl}amino)acetic acid
({(2R,S)-2-[1-(3,4-difluorobenzoyl)-6-fluoro-5-hydroxy-2-methyl-1H-indol-3-yl]propanoyl}amino)-2-methylpropionic acid
({(2R,S)-2-[1-(3,4-difluorobenzoyl)-6-fluoro-5-hydroxy-2-methyl-1H-indol-3-yl]propanoyl}amino)-2,2-methylbutyric acid
(2R,S)-3-({[1-(3,4-difluorobenzoyl)-6-fluoro-5-hydroxy-2-methyl-1H-indol-3-yl]propanoyl}amino)propanoic acid
(2R,S)-3-({[1-(3,4-difluorobenzoyl)-6-fluoro-5-hydroxy-2-methyl-1H-indol-3-yl]propanoyl}amino)-3-methylpropanic acid
(2R,S)-3-({[1-(3,4-difluorobenzoyl)-6-fluoro-5-hydroxy-2-methyl-1H-indol-3-yl]propanoyl}amino)-2-methylpropanic acid
(2R,S)-2[1-(3,4-difluorobenzoyl)-4-fluoro-5-hydroxy-2-methyl-1H-indol-3-yl]propionic acid,
methyl (2R,S)-2-[1-(3,4-difluorobenzoyl)-4-fluoro-5-hydroxy-2-methyl-1H-indol-3-yl]propanoate
ethyl (2R,S)-2-[1-(3,4-difluorobenzoyl)-4-fluoro-5-hydroxy-2-methyl-1H-indol-3-yl]propanoate
propyl (2R,S)-2-[1-(3,4-difluorobenzoyl)-4-fluoro-5-hydroxy-2-methyl-1H-indol-3-yl]propanoate
isopropyl (2R,S)-2-[1-(3,4-difluorobenzoyl)-4-fluoro-5-hydroxy-2-methyl-1H-indol-3-yl]propanoate
butyl (2R,S)-2-[1-(3,4-difluorobenzoyl)-4-fluoro-5-hydroxy-2-methyl-1H-indol-3-yl]propanoate
sec-butyl (2R,S)-2-[1-(3,4-difluorobenzoyl)-4-fluoro-5-hydroxy-2-methyl-1H-indol-3-yl]propanoate
(R) sec-butyl (2R,S)-2-[1-(3,4-difluorobenzoyl)-4-fluoro-5-hydroxy-2-methyl-1H-indol-3-yl]propanoate
(S) sec-butyl (2R,S)-2-[1-(3,4-difluorobenzoyl)-4-fluoro-5-hydroxy-2-methyl-1H-indol-3-yl]propanoate
tert-butyl (2R,S)-2-[1-(3,4-difluorobenzoyl)-4-fluoro-5-hydroxy-2-methyl-1H-indol-3-yl]propanoate
cyclobutyl (2R,S)-2-[1-(3,4-difluorobenzoyl)-4-fluoro-5-hydroxy-2-methyl-1H-indol-3-yl]propanoate
cyclopentyl (2R,S)-2-[1-(3,4-difluorobenzoyl)-4-fluoro-5-hydroxy-2-methyl-1H-indol-3-yl]propanoate
(2R,S)-2-[1-(3,4-difluorobenzoyl)-4-fluoro-5-hydroxy-2-methyl-1H-indol-3-yl]propanamide
(2R,S)-2-[1-(3,4-difluorobenzoyl)-4-fluoro-5-hydroxy-2-methyl-1H-indol-3-yl]-N-methylpropanamide
(2R,S)-2-[1-(3,4-difluorobenzoyl)-4-fluoro-5-hydroxy-2-methyl-1H-indol-3-yl]-N-ethylacetamide
(2R,S)-2-[1-(3,4-difluorobenzoyl)-4-fluoro-5-hydroxy-2-methyl-1H-indol-3-yl]-N-propylacetamide
(2R,S)-2-[1-(3,4-difluorobenzoyl)-4-fluoro-5-hydroxy-2-methyl-1H-indol-3-yl]-N-isopropylacetamide
(2R,S)-2-[1-(3,4-difluorobenzoyl)-4-fluoro-5-hydroxy-2-methyl-1H-indol-3-yl]-N-butylacetamide
(2R,S)-2-[1-(3,4-difluorobenzoyl)-4-fluoro-5-hydroxy-2-methyl-1H-indol-3-yl]-N-sec-butylacetamide
(2R,S)-2-[1-(3,4-difluorobenzoyl)-4-fluoro-5-hydroxy-2-methyl-1H-indol-3-yl]-N-(hydroxymethyl)propanamide
({(2R,S)-2-[1-(3,4-difluorobenzoyl)-4-fluoro-5-hydroxy-2-methyl-1H-indol-3-yl]propanoyl}amino)acetic acid
({(2R,S)-2-[1-(3,4-difluorobenzoyl)-4-fluoro-5-hydroxy-2-methyl-1H-indol-3-yl]propanoyl}amino)-2-methylpropionic acid
({(2R,S)-2-[1-(3,4-difluorobenzoyl)-4-fluoro-5-hydroxy-2-methyl-1H-indol-3-yl]propanoyl}amino)-2,2-methylbutyric acid
(2R,S)-3-({[1-(3,4-difluorobenzoyl)-4-fluoro-5-hydroxy-2-methyl-1H-indol-3-yl]propanoyl}amino)propanoic acid
(2R,S)-3-({[1-(3,4-difluorobenzoyl)-4-fluoro-5-hydroxy-2-methyl-1H-indol-3-yl]propanoyl}amino)-3-methylpropanic acid
(2R,S)-3-({[1-(3,4-difluorobenzoyl)-4-fluoro-5-hydroxy-2-methyl-1H-indol-3-yl]propanoyl}amino)-2-methylpropanic acid
(2R,S)-2[1-(3,4-difluorobenzoyl)-4,6-difluoro-5-hydroxy-2-methyl-1H-indol-3-yl]propionic acid,
methyl (2R,S)-2-[1-(3,4-difluorobenzoyl)-4,6-difluoro-5-hydroxy-2-methyl-1H-indol-3-yl]propanoate
ethyl (2R,S)-2-[1-(3,4-difluorobenzoyl)-4,6-difluoro-5-hydroxy-2-methyl-1H-indol-3-yl]propanoate
propyl (2R,S)-2-[1-(3,4-difluorobenzoyl)-4,6-difluoro-5-hydroxy-2-methyl-1H-indol-3-yl]propanoate
isopropyl (2R,S)-2-[1-(3,4-difluorobenzoyl)-4,6-difluoro-5-hydroxy-2-methyl-1H-indol-3-yl]propanoate
butyl (2R,S)-2-[1-(3,4-difluorobenzoyl)-4,6-difluoro-5-hydroxy-2-methyl-1H-indol-3-yl]propanoate
sec-butyl (2R,S)-2-[1-(3,4-difluorobenzoyl)-4,6-difluoro-5-hydroxy-2-methyl-1H-indol-3-yl]propanoate
(R) sec-butyl (2R,S)-2-[1-(3,4-difluorobenzoyl)-4,6-difluoro-5-hydroxy-2-methyl-1H-indol-3-yl]propanoate
(S) sec-butyl (2R,S)-2-[1-(3,4-difluorobenzoyl)-4,6-difluoro-5-hydroxy-2-methyl-1H-indol-3-yl]propanoate
tert-butyl (2R,S)-2-[1-(3,4-difluorobenzoyl)-4,6-difluoro-5-hydroxy-2-methyl-1H-indol-3-yl]propanoate
cyclobutyl (2R,S)-2-[1-(3,4-difluorobenzoyl)-4,6-difluoro-5-hydroxy-2-methyl-1H-indol-3-yl]propanoate
cyclopentyl (2R,S)-2-[1-(3,4-difluorobenzoyl)-4,6-difluoro-5-hydroxy-2-methyl-1H-indol-3-yl]propanoate
(2R,S)-2-[1-(3,4-difluorobenzoyl)-4,6-difluoro-5-hydroxy-2-methyl-1H-indol-3-yl]propanamide
(2R,S)-2-[1-(3,4-difluorobenzoyl)-4,6-difluoro-5-hydroxy-2-methyl-1H-indol-3-yl]-N-methylpropanamide
(2R,S)-2-[1-(3,4-difluorobenzoyl)-4,6-difluoro-5-hydroxy-2-methyl-1H-indol-3-yl]-N-ethylacetamide
(2R,S)-2-[1-(3,4-difluorobenzoyl)-4,6-difluoro-5-hydroxy-2-methyl-1H-indol-3-yl]-N-propylacetamide
(2R,S)-2-[1-(3,4-difluorobenzoyl)-4,6-difluoro-5-hydroxy-2-methyl-1H-indol-3-yl]-N-isopropylacetamide
(2R,S)-2-[1-(3,4-difluorobenzoyl)-4,6-difluoro-5-hydroxy-2-methyl-1H-indol-3-yl]-N-butylacetamide
(2R,S)-2-[1-(3,4-difluorobenzoyl)-4,6-difluoro-5-hydroxy-2-methyl-1H-indol-3-yl]-N-sec-butylacetamide
(2R,S)-2-[1-(3,4-difluorobenzoyl)-4,6-difluoro-5-hydroxy-2-methyl-1H-indol-3-yl]-N-(hydroxymethyl)propanamide
({(2R,S)-2-[1-(3,4-difluorobenzoyl)-4,6-difluoro-5-hydroxy-2-methyl-1H-indol-3-yl]propanoyl}amino)acetic acid
({(2R,S)-2-[1-(3,4-difluorobenzoyl)-4,6-difluoro-5-hydroxy-2-methyl-1H-indol-3-yl]propanoyl}amino)-2-methylpropionic acid
({(2R,S)-2-[1-(3,4-difluorobenzoyl)-4,6-difluoro-5-hydroxy-2-methyl-1H-indol-3-yl]propanoyl}amino)-2,2-methylbutyric acid
(2R,S)-3-({[1-(3,4-difluorobenzoyl)-4,6-difluoro-5-hydroxy-2-methyl-1H-indol-3-yl]propanoyl}amino)propanoic acid
(2R,S)-3-({[1-(3,4-difluorobenzoyl)-4,6-difluoro-5-hydroxy-2-methyl-1H-indol-3-yl]propanoyl}amino)-3-methylpropanic acid
(2R,S)-3-({[1-(3,4-difluorobenzoyl)-4,6-difluoro-5-hydroxy-2-methyl-1H-indol-3-yl]propanoyl}amino)-2-methylpropanic acid
(2R,S)-2[1-(4-choro-3-fluorobenzoyl)-5-hydroxy-2-methyl-1H-indol-3-yl]propionic acid,
methyl (2R,S)-2-[1-(4-choro-3-fluorobenzoyl)-5-hydroxy-2-methyl-1H-indol-3-yl]propanoate
ethyl (2R,S)-2-[1-(4-choro-3-fluorobenzoyl)-5-hydroxy-2-methyl-1H-indol-3-yl]propanoate
propyl (2R,S)-2-[1-(4-choro-3-fluorobenzoyl)-5-hydroxy-2-methyl-1H-indol-3-yl]propanoate
isopropyl (2R,S)-2-[1-(4-choro-3-fluorobenzoyl)-5-hydroxy-2-methyl-1H-indol-3-yl]propanoate
butyl (2R,S)-2-[1-(4-choro-3-fluorobenzoyl)-5-hydroxy-2-methyl-1H-indol-3-yl]propanoate
sec-butyl (2R,S)-2-[1-(4-choro-3-fluorobenzoyl)-5-hydroxy-2-methyl-1H-indol-3-yl]propanoate
(R) sec-butyl (2R,S)-2-[1-(4-choro-3-fluorobenzoyl)-5-hydroxy-2-methyl-1H-indol-3-yl]propanoate
(S) sec-butyl (2R,S)-2-[1-(4-choro-3-fluorobenzoyl)-5-hydroxy-2-methyl-1H-indol-3-yl]propanoate
tert-butyl (2R,S)-2-[1-(4-choro-3-fluorobenzoyl)-5-hydroxy-2-methyl-1H-indol-3-yl]propanoate
cyclobutyl (2R,S)-2-[1-(4-choro-3-fluorobenzoyl)-5-hydroxy-2-methyl-1H-indol-3-yl]propanoate
cyclopentyl (2R,S)-2-[1-(4-choro-3-fluorobenzoyl)-5-hydroxy-2-methyl-1H-indol-3-yl]propanoate
(2R,S)-2-[1-(4-choro-3-fluorobenzoyl)-5-hydroxy-2-methyl-1H-indol-3-yl]propanamide
(2R,S)-2-[1-(4-choro-3-fluorobenzoyl)-5-hydroxy-2-methyl-1H-indol-3-yl]-N-methylpropanamide
(2R,S)-2-[1-(4-choro-3-fluorobenzoyl)-5-hydroxy-2-methyl-1H-indol-3-yl]-N-ethylacetamide
(2R,S)-2-[1-(4-choro-3-fluorobenzoyl)-5-hydroxy-2-methyl-1H-indol-3-yl]-N-propylacetamide
(2R,S)-2-[1-(4-choro-3-fluorobenzoyl)-5-hydroxy-2-methyl-1H-indol-3-yl]-N-isopropylacetamide
(2R,S)-2-[1-(4-choro-3-fluorobenzoyl)-5-hydroxy-2-methyl-1H-indol-3-yl]-N-butylacetamide
(2R,S)-2-[1-(4-choro-3-fluorobenzoyl)-5-hydroxy-2-methyl-1H-indol-3-yl]-N-sec-butylacetamide
(2R,S)-2-[1-(4-choro-3-fluorobenzoyl)-5-hydroxy-2-methyl-1H-indol-3-yl]-N-(hydroxymethyl)propanamide
({(2R,S)-2-[1-(4-choro-3-fluorobenzoyl)-5-hydroxy-2-methyl-1H-indol-3-yl]propanoyl}amino)acetic acid
({(2R,S)-2-[1-(4-choro-3-fluorobenzoyl)-5-hydroxy-2-methyl-1H-indol-3-yl]propanoyl}amino)-2-methylpropionic acid
({(2R,S)-2-[1-(4-choro-3-fluorobenzoyl)-5-hydroxy-2-methyl-1H-indol-3-yl]propanoyl}amino)-2,2-methylbutyric acid
(2R,S)-3-({[1-(4-choro-3-fluorobenzoyl)-5-hydroxy-2-methyl-1H-indol-3-yl]propanoyl}amino)propanoic acid
(2R,S)-3-({[1-(4-choro-3-fluorobenzoyl)-5-hydroxy-2-methyl-1H-indol-3-yl]propanoyl}amino)-3-methylpropanic acid
(2R,S)-3-({[1-(4-choro-3-fluorobenzoyl)-5-hydroxy-2-methyl-1H-indol-3-yl]propanoyl}amino)-2-methylpropanic acid
(2R,S)-2[1-(4-choro-3-fluorobenzoyl)-6-fluoro-5-hydroxy-2-methyl-1H-indol-3-yl]propionic acid,
methyl (2R,S)-2-[1-(4-choro-3-fluorobenzoyl)-6-fluoro-5-hydroxy-2-methyl-1H-indol-3-yl]propanoate
ethyl (2R,S)-2-[1-(4-choro-3-fluorobenzoyl)-6-fluoro-5-hydroxy-2-methyl-1H-indol-3-yl]propanoate
propyl (2R,S)-2-[1-(4-choro-3-fluorobenzoyl)-6-fluoro-5-hydroxy-2-methyl-1H-indol-3-yl]propanoate
isopropyl (2R,S)-2-[1-(4-choro-3-fluorobenzoyl)-6-fluoro-5-hydroxy-2-methyl-1H-indol-3-yl]propanoate
butyl (2R,S)-2-[1-(4-choro-3-fluorobenzoyl)-6-fluoro-5-hydroxy-2-methyl-1H-indol-3-yl]propanoate
sec-butyl (2R,S)-2-[1-(4-choro-3-fluorobenzoyl)-6-fluoro-5-hydroxy-2-methyl-1H-indol-3-yl]propanoate
(R) sec-butyl (2R,S)-2-[1-(4-choro-3-fluorobenzoyl)-6-fluoro-5-hydroxy-2-methyl-1H-indol-3-yl]propanoate
(S) sec-butyl (2R,S)-2-[1-(4-choro-3-fluorobenzoyl)-6-fluoro-5-hydroxy-2-methyl-1H-indol-3-yl]propanoate
tert-butyl (2R,S)-2-[1-(4-choro-3-fluorobenzoyl)-6-fluoro-5-hydroxy-2-methyl-1H-indol-3-yl]propanoate
cyclobutyl (2R,S)-2-[1-(4-choro-3-fluorobenzoyl)-6-fluoro-5-hydroxy-2-methyl-1H-indol-3-yl]propanoate
cyclopentyl (2R,S)-2-[1-(4-choro-3-fluorobenzoyl)-6-fluoro-5-hydroxy-2-methyl-1H-indol-3-yl]propanoate
(2R,S)-2-[1-(4-choro-3-fluorobenzoyl)-6-fluoro-5-hydroxy-2-methyl-1H-indol-3-yl]propanamide
(2R,S)-2-[1-(4-choro-3-fluorobenzoyl)-6-fluoro-5-hydroxy-2-methyl-1H-indol-3-yl]-N-methylpropanamide
(2R,S)-2-[1-(4-choro-3-fluorobenzoyl)-6-fluoro-5-hydroxy-2-methyl-1H-indol-3-yl]-N-ethylacetamide
(2R,S)-2-[1-(4-choro-3-fluorobenzoyl)-6-fluoro-5-hydroxy-2-methyl-1H-indol-3-yl]-N-propylacetamide
(2R,S)-2-[1-(4-choro-3-fluorobenzoyl)-6-fluoro-5-hydroxy-2-methyl-1H-indol-3-yl]-N-isopropylacetamide
(2R,S)-2-[1-(4-choro-3-fluorobenzoyl)-6-fluoro-5-hydroxy-2-methyl-1H-indol-3-yl]-N-butylacetamide
(2R,S)-2-[1-(4-choro-3-fluorobenzoyl)-6-fluoro-5-hydroxy-2-methyl-1H-indol-3-yl]-N-sec-butylacetamide
(2R,S)-2-[1-(4-choro-3-fluorobenzoyl)-6-fluoro-5-hydroxy-2-methyl-1H-indol-3-yl]-N-(hydroxymethyl)propanamide
({(2R,S)-2-[1-(4-choro-3-fluorobenzoyl)-6-fluoro-5-hydroxy-2-methyl-1H-indol-3-yl]propanoyl}amino)acetic acid
({(2R,S)-2-[1-(4-choro-3-fluorobenzoyl)-6-fluoro-5-hydroxy-2-methyl-1H-indol-3-yl]propanoyl}amino)-2-methylpropionic acid
({(2R,S)-2-[1-(4-choro-3-fluorobenzoyl)-6-fluoro-5-hydroxy-2-methyl-1H-indol-3-yl]propanoyl}amino)-2,2-methylbutyric acid
(2R,S)-3-({[1-(4-choro-3-fluorobenzoyl)-6-fluoro-5-hydroxy-2-methyl-1H-indol-3-yl]propanoyl}amino)propanoic acid
(2R,S)-3-({[1-(4-choro-3-fluorobenzoyl)-6-fluoro-5-hydroxy-2-methyl-1H-indol-3-yl]propanoyl}amino)-3-methylpropanic acid
(2R,S)-3-({[1-(4-choro-3-fluorobenzoyl)-6-fluoro-5-hydroxy-2-methyl-1H-indol-3-yl]propanoyl}amino)-2-methylpropanic acid
(2R,S)-2[1-(4-choro-3-fluorobenzoyl)-4-fluoro-5-hydroxy-2-methyl-1H-indol-3-yl]propionic acid,
methyl (2R,S)-2-[1-(4-choro-3-fluorobenzoyl)-4-fluoro-5-hydroxy-2-methyl-1H-indol-3-yl]propanoate
ethyl (2R,S)-2-[1-(4-choro-3-fluorobenzoyl)-4-fluoro-5-hydroxy-2-methyl-1H-indol-3-yl]propanoate
propyl (2R,S)-2-[1-(4-choro-3-fluorobenzoyl)-4-fluoro-5-hydroxy-2-methyl-1H-indol-3-yl]propanoate
isopropyl (2R,S)-2-[1-(4-choro-3-fluorobenzoyl)-4-fluoro-5-hydroxy-2-methyl-1H-indol-3-yl]propanoate
butyl (2R,S)-2-[1-(4-choro-3-fluorobenzoyl)-4-fluoro-5-hydroxy-2-methyl-1H-indol-3-yl]propanoate
sec-butyl (2R,S)-2-[1-(4-choro-3-fluorobenzoyl)-4-fluoro-5-hydroxy-2-methyl-1H-indol-3-yl]propanoate
(R) sec-butyl (2R,S)-2-[1-(4-choro-3-fluorobenzoyl)-4-fluoro-5-hydroxy-2-methyl-1H-indol-3-yl]propanoate
(S) sec-butyl (2R,S)-2-[1-(4-choro-3-fluorobenzoyl)-4-fluoro-5-hydroxy-2-methyl-1H-indol-3-yl]propanoate
tert-butyl (2R,S)-2-[1-(4-choro-3-fluorobenzoyl)-4-fluoro-5-hydroxy-2-methyl-1H-indol-3-yl]propanoate
cyclobutyl (2R,S)-2-[1-(4-choro-3-fluorobenzoyl)-4-fluoro-5-hydroxy-2-methyl-1H-indol-3-yl]propanoate
cyclopentyl (2R,S)-2-[1-(4-choro-3-fluorobenzoyl)-4-fluoro-5-hydroxy-2-methyl-1H-indol-3-yl]propanoate
(2R,S)-2-[1-(4-choro-3-fluorobenzoyl)-4-fluoro-5-hydroxy-2-methyl-1H-indol-3-yl]propanamide
(2R,S)-2-[1-(4-choro-3-fluorobenzoyl)-4-fluoro-5-hydroxy-2-methyl-1H-indol-3-yl]-N-methylpropanamide
(2R,S)-2-[1-(4-choro-3-fluorobenzoyl)-4-fluoro-5-hydroxy-2-methyl-1H-indol-3-yl]-N-ethylacetamide
(2R,S)-2-[1-(4-choro-3-fluorobenzoyl)-4-fluoro-5-hydroxy-2-methyl-1H-indol-3-yl]-N-propylacetamide
(2R,S)-2-[1-(4-choro-3-fluorobenzoyl)-4-fluoro-5-hydroxy-2-methyl-1H-indol-3-yl]-N-isopropylacetamide
(2R,S)-2-[1-(4-choro-3-fluorobenzoyl)-4-fluoro-5-hydroxy-2-methyl-1H-indol-3-yl]-N-butylacetamide
(2R,S)-2-[1-(4-choro-3-fluorobenzoyl)-4-fluoro-5-hydroxy-2-methyl-1H-indol-3-yl]-N-sec-butylacetamide
(2R,S)-2-[1-(4-choro-3-fluorobenzoyl)-4-fluoro-5-hydroxy-2-methyl-1H-indol-3-yl]-N-(hydroxymethyl)propanamide
({(2R,S)-2-[1-(4-choro-3-fluorobenzoyl)-4-fluoro-5-hydroxy-2-methyl-1H-indol-3-yl]propanoyl}amino)acetic acid
({(2R,S)-2-[1-(4-choro-3-fluorobenzoyl)-4-fluoro-5-hydroxy-2-methyl-1H-indol-3-yl]propanoyl}amino)-2-methylpropionic acid
({(2R,S)-2-[1-(4-choro-3-fluorobenzoyl)-4-fluoro-5-hydroxy-2-methyl-1H-indol-3-yl]propanoyl}amino)-2,2-methylbutyric acid
(2R,S)-3-({[1-(4-choro-3-fluorobenzoyl)-4-fluoro-5-hydroxy-2-methyl-1H-indol-3-yl]propanoyl}amino)propanoic acid
(2R,S)-3-({[1-(4-choro-3-fluorobenzoyl)-4-fluoro-5-hydroxy-2-methyl-1H-indol-3-yl]propanoyl}amino)-3-methylpropanic acid
(2R,S)-3-({[1-(4-choro-3-fluorobenzoyl)-4-fluoro-5-hydroxy-2-methyl-1H-indol-3-yl]propanoyl}amino)-2-methylpropanic acid
(2R,S)-2[1-(4-choro-3-fluorobenzoyl)-4,6-difluoro-5-hydroxy-2-methyl-1H-indol-3-yl]propionic acid,
methyl (2R,S)-2-[1-(4-choro-3-fluorobenzoyl)-4,6-difluoro-5-hydroxy-2-methyl-1H-indol-3-yl]propanoate
ethyl (2R,S)-2-[1-(4-choro-3-fluorobenzoyl)-4,6-difluoro-5-hydroxy-2-methyl-1H-indol-3-yl]propanoate
propyl (2R,S)-2-[1-(4-choro-3-fluorobenzoyl)-4,6-difluoro-5-hydroxy-2-methyl-1H-indol-3-yl]propanoate
isopropyl (2R,S)-2-[1-(4-choro-3-fluorobenzoyl)-4,6-difluoro-5-hydroxy-2-methyl-1H-indol-3-yl]propanoate
butyl (2R,S)-2-[1-(4-choro-3-fluorobenzoyl)-4,6-difluoro-5-hydroxy-2-methyl-1H-indol-3-yl]propanoate
sec-butyl (2R,S)-2-[1-(4-choro-3-fluorobenzoyl)-4,6-difluoro-5-hydroxy-2-methyl-1H-indol-3-yl]propanoate
(R) sec-butyl (2R,S)-2-[1-(4-choro-3-fluorobenzoyl)-4,6-difluoro-5-hydroxy-2-methyl-1H-indol-3-yl]propanoate
(S) sec-butyl (2R,S)-2-[1-(4-choro-3-fluorobenzoyl)-4,6-difluoro-5-hydroxy-2-methyl-1H-indol-3-yl]propanoate
tert-butyl (2R,S)-2-[1-(4-choro-3-fluorobenzoyl)-4,6-difluoro-5-hydroxy-2-methyl-1H-indol-3-yl]propanoate
cyclobutyl (2R,S)-2-[1-(4-choro-3-fluorobenzoyl)-4,6-difluoro-5-hydroxy-2-methyl-1H-indol-3-yl]propanoate
cyclopentyl (2R,S)-2-[1-(4-choro-3-fluorobenzoyl)-4,6-difluoro-5-hydroxy-2-methyl-1H-indol-3-yl]propanoate
(2R,S)-2-[1-(4-choro-3-fluorobenzoyl)-4,6-difluoro-5-hydroxy-2-methyl-1H-indol-3-yl]propanamide
(2R,S)-2-[1-(4-choro-3-fluorobenzoyl)-4,6-difluoro-5-hydroxy-2-methyl-1H-indol-3-yl]-N-methylpropanamide
(2R,S)-2-[1-(4-choro-3-fluorobenzoyl)-4,6-difluoro-5-hydroxy-2-methyl-1H-indol-3-yl]-N-ethylacetamide
(2R,S)-2-[1-(4-choro-3-fluorobenzoyl)-4,6-difluoro-5-hydroxy-2-methyl-1H-indol-3-yl]-N-propylacetamide
(2R,S)-2-[1-(4-choro-3-fluorobenzoyl)-4,6-difluoro-5-hydroxy-2-methyl-1H-indol-3-yl]-N-isopropylacetamide
(2R,S)-2-[1-(4-choro-3-fluorobenzoyl)-4,6-difluoro-5-hydroxy-2-methyl-1H-indol-3-yl]-N-butylacetamide
(2R,S)-2-[1-(4-choro-3-fluorobenzoyl)-4,6-difluoro-5-hydroxy-2-methyl-1H-indol-3-yl]-N-sec-butylacetamide
(2R,S)-2-[1-(4-choro-3-fluorobenzoyl)-4,6-difluoro-5-hydroxy-2-methyl-1H-indol-3-yl]-N-(hydroxymethyl)propanamide
({(2R,S)-2-[1-(4-choro-3-fluorobenzoyl)-4,6-difluoro-5-hydroxy-2-methyl-1H-indol-3-yl]propanoyl}amino)acetic acid
({(2R,S)-2-[1-(4-choro-3-fluorobenzoyl)-4,6-difluoro-5-hydroxy-2-methyl-1H-indol-3-yl]propanoyl}amino)-2-methylpropionic acid
({(2R,S)-2-[1-(4-choro-3-fluorobenzoyl)-4,6-difluoro-5-hydroxy-2-methyl-1H-indol-3-yl]propanoyl}amino)-2,2-methylbutyric acid
(2R,S)-3-({[1-(4-choro-3-fluorobenzoyl)-4,6-difluoro-5-hydroxy-2-methyl-1H-indol-3-yl]propanoyl}amino)propanoic acid
(2R,S)-3-({-(4-choro-3-fluorobenzoyl)-4,6-difluoro-5-hydroxy-2-methyl-1H-indol-3-yl]propanoyl}amino)-3-methylpropanic acid
(2R,S)-3-({[1-(4-choro-3-fluorobenzoyl)-4,6-difluoro-5-hydroxy-2-methyl-1H-indol-3-yl]propanoyl}amino)-2-methylpropanic acid
(2R,S)-2[1-(3-fluorobenzoyl)-5-hydroxy-2-methyl-1H-indol-3-yl]propionic acid,
methyl (2R,S)-2-[1-(3-fluorobenzoyl)-5-hydroxy-2-methyl-1H-indol-3-yl]propanoate
ethyl (2R,S)-2-[1-(3-fluorobenzoyl)-5-hydroxy-2-methyl-1H-indol-3-yl]propanoate
propyl (2R,S)-2-[1-(3-fluorobenzoyl)-5-hydroxy-2-methyl-1H-indol-3-yl]propanoate
isopropyl (2R,S)-2-[1-(3-fluorobenzoyl)-5-hydroxy-2-methyl-1H-indol-3-yl]propanoate
butyl (2R,S)-2-[1-(3-fluorobenzoyl)-5-hydroxy-2-methyl-1H-indol-3-yl]propanoate
sec-butyl (2R,S)-2-[1-(3-fluorobenzoyl)-5-hydroxy-2-methyl-1H-indol-3-yl]propanoate
(R) sec-butyl (2R,S)-2-[1-(3-fluorobenzoyl)-5-hydroxy-2-methyl-1H-indol-3-yl]propanoate
(S) sec-butyl (2R,S)-2-[1-(3-fluorobenzoyl)-5-hydroxy-2-methyl-1H-indol-3-yl]propanoate
tert-butyl (2R,S)-2-[1-(3-fluorobenzoyl)-5-hydroxy-2-methyl-1H-indol-3-yl]propanoate
cyclobutyl (2R,S)-2-[1-(3-fluorobenzoyl)-5-hydroxy-2-methyl-1H-indol-3-yl]propanoate
cyclopentyl (2R,S)-2-[1-(3-fluorobenzoyl)-5-hydroxy-2-methyl-1H-indol-3-yl]propanoate
(2R,S)-2-[1-(3-fluorobenzoyl)-5-hydroxy-2-methyl-1H-indol-3-yl]propanamide
(2R,S)-2-[1-(3-fluorobenzoyl)-5-hydroxy-2-methyl-1H-indol-3-yl]-N-methylpropanamide
(2R,S)-2-[1-(3-fluorobenzoyl)-5-hydroxy-2-methyl-1H-indol-3-yl]-N-ethylacetamide
(2R,S)-2-[1-(3-fluorobenzoyl)-5-hydroxy-2-methyl-1H-indol-3-yl]-N-propylacetamide
(2R,S)-2-[1-(3-fluorobenzoyl)-5-hydroxy-2-methyl-1H-indol-3-yl]-N-isopropylacetamide
(2R,S)-2-[1-(3-fluorobenzoyl)-5-hydroxy-2-methyl-1H-indol-3-yl]-N-butylacetamide
(2R,S)-2-[1-(3-fluorobenzoyl)-5-hydroxy-2-methyl-1H-indol-3-yl]-N-sec-butylacetamide
(2R,S)-2-[1-(3-fluorobenzoyl)-5-hydroxy-2-methyl-1H-indol-3-yl]-N-(hydroxymethyl)propanamide
({(2R,S)-2-[1-(3-fluorobenzoyl)-5-hydroxy-2-methyl-1H-indol-3-yl]propanoyl}amino)acetic acid
({(2R,S)-2-[1-(3-fluorobenzoyl)-5-hydroxy-2-methyl-1H-indol-3-yl]propanoyl}amino)-2-methylpropionic acid
({(2R,S)-2-[1-(3-fluorobenzoyl)-5-hydroxy-2-methyl-1H-indol-3-yl]propanoyl}amino)-2,2-methylbutyric acid
(2R,S)-3-({[1-(3-fluorobenzoyl)-5-hydroxy-2-methyl-1H-indol-3-yl]propanoyl lamino)propanoic acid
(2R,S)-3-([[1-(3-fluorobenzoyl)-5-hydroxy-2-methyl-1H-indol-3-yl]propanoyl}amino)-3-methylpropanic acid
(2R,S)-3-({[1-(3-fluorobenzoyl)-5-hydroxy-2-methyl-1H-indol-3-yl]propanoyl}amino)-2-methylpropanic acid
(2R,S)-2[1-(3-fluorobenzoyl)-6-fluoro-5-hydroxy-2-methyl-1H-indol-3-yl]propionic acid,
methyl (2R,S)-2-[1-(3-fluorobenzoyl)-6-fluoro-5-hydroxy-2-methyl-1H-indol-3-yl]propanoate
ethyl (2R,S)-2-[1-(3-fluorobenzoyl)-6-fluoro-5-hydroxy-2-methyl-1H-indol-3-yl]propanoate
propyl (2R,S)-2-[1-(3-fluorobenzoyl)-6-fluoro-5-hydroxy-2-methyl-1H-indol-3-yl]propanoate
isopropyl (2R,S)-2-[1-(3-fluorobenzoyl)-6-fluoro-5-hydroxy-2-methyl-1H-indol-3-yl]propanoate
butyl (2R,S)-2-[1-(3-fluorobenzoyl)-6-fluoro-5-hydroxy-2-methyl-1H-indol-3-yl]propanoate
sec-butyl (2R,S)-2-[1-(3-fluorobenzoyl)-6-fluoro-5-hydroxy-2-methyl-1H-indol-3-yl]propanoate
(R) sec-butyl (2R,S)-2-[1-(3-fluorobenzoyl)-6-fluoro-5-hydroxy-2-methyl-1H-indol-3-yl]propanoate
(S) sec-butyl (2R,S)-2-[1-(3-fluorobenzoyl)-6-fluoro-5-hydroxy-2-methyl-1H-indol-3-yl]propanoate
tert-butyl (2R,S)-2-[1-(3-fluorobenzoyl)-6-fluoro-5-hydroxy-2-methyl-1H-indol-3-yl]propanoate
cyclobutyl (2R,S)-2-[1-(3-fluorobenzoyl)-6-fluoro-5-hydroxy-2-methyl-1H-indol-3-yl]propanoate
cyclopentyl (2R,S)-2-[1-(3-fluorobenzoyl)-6-fluoro-5-hydroxy-2-methyl-1H-indol-3-yl]propanoate
(2R,S)-2-[1-(3-fluorobenzoyl)-6-fluoro-5-hydroxy-2-methyl-1H-indol-3-yl]propanamide
(2R,S)-2-[1-(3-fluorobenzoyl)-6-fluoro-5-hydroxy-2-methyl-1H-indol-3-yl]-N-methylpropanamide
(2R,S)-2-[1-(3-fluorobenzoyl)-6-fluoro-5-hydroxy-2-methyl-1H-indol-3-yl]-N-ethylacetamide
(2R,S)-2-[1-(3-fluorobenzoyl)-6-fluoro-5-hydroxy-2-methyl-1H-indol-3-yl]-N-propylacetamide
(2R,S)-2-[1-(3-fluorobenzoyl)-6-fluoro-5-hydroxy-2-methyl-1H-indol-3-yl]-N-isopropylacetamide
(2R,S)-2-[1-(3-fluorobenzoyl)-6-fluoro-5-hydroxy-2-methyl-1H-indol-3-yl]-N-butylacetamide
(2R,S)-2-[1-(3-fluorobenzoyl)-6-fluoro-5-hydroxy-2-methyl-1H-indol-3-yl]-N-sec-butylacetamide
(2R,S)-2-[1-(3-fluorobenzoyl)-6-fluoro-5-hydroxy-2-methyl-1H-indol-3-yl]-N-(hydroxymethyl)propanamide
({(2R,S)-2-[1-(3-fluorobenzoyl)-6-fluoro-5-hydroxy-2-methyl-1H-indol-3-yl]propanoyl}amino)acetic acid
({(2R,S)-2-[1-(3-fluorobenzoyl)-6-fluoro-5-hydroxy-2-methyl-1H-indol-3-yl]propanoyl}amino)-2-methylpropionic acid
({(2R,S)-2-[1-(3-fluorobenzoyl)-6-fluoro-5-hydroxy-2-methyl-1H-indol-3-yl]propanoyl}amino)-2,2-methylbutyric acid
(2R,S)-3-({[1-(3-fluorobenzoyl)-6-fluoro-5-hydroxy-2-methyl-1H-indol-3-yl]propanoyl}amino)propanoic acid
(2R,S)-3-({[1-(3-fluorobenzoyl)-6-fluoro-5-hydroxy-2-methyl-1H-indol-3-yl]propanoyl}amino)-3-methylpropanic acid
(2R,S)-3-({[1-(3-fluorobenzoyl)-6-fluoro-5-hydroxy-2-methyl-1H-indol-3-yl]propanoyl}amino)-2-methylpropanic acid
(2R,S)-2[1-(3-fluorobenzoyl)-4-fluoro-5-hydroxy-2-methyl-1H-indol-3-yl]propionic acid,
methyl (2R,S)-2-[1-(3-fluorobenzoyl)-4-fluoro-5-hydroxy-2-methyl-1H-indol-3-yl]propanoate
ethyl (2R,S)-2-[1-(3-fluorobenzoyl)-4-fluoro-5-hydroxy-2-methyl-1H-indol-3-yl]propanoate
propyl (2R,S)-2-[1-(3-fluorobenzoyl)-4-fluoro-5-hydroxy-2-methyl-1H-indol-3-yl]propanoate
isopropyl (2R,S)-2-[1-(3-fluorobenzoyl)-4-fluoro-5-hydroxy-2-methyl-1H-indol-3-yl]propanoate
butyl (2R,S)-2-[1-(3-fluorobenzoyl)-4-fluoro-5-hydroxy-2-methyl-1H-indol-3-yl]propanoate
sec-butyl (2R,S)-2-[1-(3-fluorobenzoyl)-4-fluoro-5-hydroxy-2-methyl-1H-indol-3-yl]propanoate
(R) sec-butyl (2R,S)-2-[1-(3-fluorobenzoyl)-4-fluoro-5-hydroxy-2-methyl-1H-indol-3-yl]propanoate
(S) sec-butyl (2R,S)-2-[1-(3-fluorobenzoyl)-4-fluoro-5-hydroxy-2-methyl-1H-indol-3-yl]propanoate
tert-butyl (2R,S)-2-[1-(3-fluorobenzoyl)-4-fluoro-5-hydroxy-2-methyl-1H-indol-3-yl]propanoate
cyclobutyl (2R,S)-2-[1-(3-fluorobenzoyl)-4-fluoro-5-hydroxy-2-methyl-1H-indol-3-yl]propanoate
cyclopentyl (2R,S)-2-[1-(3-fluorobenzoyl)-4-fluoro-5-hydroxy-2-methyl-1H-indol-3-yl]propanoate
(2R,S)-2-[1-(3-fluorobenzoyl)-4-fluoro-5-hydroxy-2-methyl-1H-indol-3-yl]propanamide
(2R,S)-2-[1-(3-fluorobenzoyl)-4-fluoro-5-hydroxy-2-methyl-1H-indol-3-yl]-N-methylpropanamide
(2R,S)-2-[1-(3-fluorobenzoyl)-4-fluoro-5-hydroxy-2-methyl-1H-indol-3-yl]-N-ethylacetamide
(2R,S)-2-[1-(3-fluorobenzoyl)-4-fluoro-5-hydroxy-2-methyl-1H-indol-3-yl]-N-propylacetamide
(2R,S)-2-[1-(3-fluorobenzoyl)-4-fluoro-5-hydroxy-2-methyl-1H-indol-3-yl]-N-isopropylacetamide
(2R,S)-2-[1-(3-fluorobenzoyl)-4-fluoro-5-hydroxy-2-methyl-1H-indol-3-yl]-N-butylacetamide
(2R,S)-2-[1-(3-fluorobenzoyl)-4-fluoro-5-hydroxy-2-methyl-1H-indol-3-yl]-N-sec-butylacetamide
(2R,S)-2-[1-(3-fluorobenzoyl)-4-fluoro-5-hydroxy-2-methyl-1H-indol-3-yl]-N-(hydroxymethyl)propanamide
({(2R,S)-2-[1-(3-fluorobenzoyl)-4-fluoro-5-hydroxy-2-methyl-1H-indol-3-yl]propanoyl}amino)acetic acid
({(2R,S)-2-[1-(3-fluorobenzoyl)-4-fluoro-5-hydroxy-2-methyl-1H-indol-3-yl]propanoyl}amino)-2-methylpropionic acid
({(2R,S)-2-[1-(3-fluorobenzoyl)-4-fluoro-5-hydroxy-2-methyl-1H-indol-3-yl]propanoyl}amino)-2,2-methylbutyric acid
(2R,S)-3-({[1-(3-fluorobenzoyl)-4-fluoro-5-hydroxy-2-methyl-1H-indol-3-yl]propanoyl}amino)propanoic acid
(2R,S)-3-({[1-(3-fluorobenzoyl)-4-fluoro-5-hydroxy-2-methyl-1H-indol-3-yl]propanoyl}amino)-3-methylpropanic acid
(2R,S)-3-({[1-(3-fluorobenzoyl)-4-fluoro-5-hydroxy-2-methyl-1H-indol-3-yl]propanoyl}amino)-2-methylpropanic acid
(2R,S)-2[1-(3-fluorobenzoyl)-4,6-difluoro-5-hydroxy-2-methyl-1H-indol-3-yl]propionic acid,
methyl (2R,S)-2-[1-(3-fluorobenzoyl)-4,6-difluoro-5-hydroxy-2-methyl-1H-indol-3-yl]propanoate
ethyl (2R,S)-2-[1-(3-fluorobenzoyl)-4,6-difluoro-5-hydroxy-2-methyl-1H-indol-3-yl]propanoate
propyl (2R,S)-2-[1-(3-fluorobenzoyl)-4,6-difluoro-5-hydroxy-2-methyl-1H-indol-3-yl]propanoate
isopropyl (2R,S)-2-[1-(3-fluorobenzoyl)-4,6-difluoro-5-hydroxy-2-methyl-1H-indol-3-yl]propanoate
butyl (2R,S)-2-[1-(3-fluorobenzoyl)-4,6-difluoro-5-hydroxy-2-methyl-1H-indol-3-yl]propanoate
sec-butyl (2R,S)-2-[1-(3-fluorobenzoyl)-4,6-difluoro-5-hydroxy-2-methyl-1H-indol-3-yl]propanoate
(R) sec-butyl (2R,S)-2-[1-(3-fluorobenzoyl)-4,6-difluoro-5-hydroxy-2-methyl-1H-indol-3-yl]propanoate
(S) sec-butyl (2R,S)-2-[1-(3-fluorobenzoyl)-4,6-difluoro-5-hydroxy-2-methyl-1H-indol-3-yl]propanoate
tert-butyl (2R,S)-2-[1-(3-fluorobenzoyl)-4,6-difluoro-5-hydroxy-2-methyl-1H-indol-3-yl]propanoate
cyclobutyl (2R,S)-2-[1-(3-fluorobenzoyl)-4,6-difluoro-5-hydroxy-2-methyl-1H-indol-3-yl]propanoate
cyclopentyl (2R,S)-2-[1-(3-fluorobenzoyl)-4,6-difluoro-5-hydroxy-2-methyl-1H-indol-3-yl]propanoate
(2R,S)-2-[1-(3-fluorobenzoyl)-4,6-difluoro-5-hydroxy-2-methyl-1H-indol-3-yl]propanamide
(2R,S)-2-[1-(3-fluorobenzoyl)-4,6-difluoro-5-hydroxy-2-methyl-1H-indol-3-yl]-N-methylpropanamide
(2R,S)-2-[1-(3-fluorobenzoyl)-4,6-difluoro-5-hydroxy-2-meth yl-1H-indol-3-yl]-N-ethylacetamide
(2R,S)-2-[1-(3-fluorobenzoyl)-4,6-difluoro-5-hydroxy-2-methyl-1H-indol-3-yl]-N-propylacetamide
(2R,S)-2-[1-(3-fluorobenzoyl)-4,6-difluoro-5-hydroxy-2-methyl-1H-indol-3-yl]-N-isopropylacetamide
(2R,S)-2-[1-(3-fluorobenzoyl)-4,6-difluoro-5-hydroxy-2-methyl-1H-indol-3-yl]-N-butylacetamide
(2R,S)-2-[1-(3-fluorobenzoyl)-4,6-difluoro-5-hydroxy-2-methyl-1H-indol-3-yl]-N-sec-butylacetamide
(2R,S)-2-[1-(3-fluorobenzoyl)-4,6-difluoro-5-hydroxy-2-methyl-1H-indol-3-yl]-N-(hydroxymethyl)propanamide
({(2R,S)-2-[1-(3-fluorobenzoyl)-4,6-difluoro-5-hydroxy-2-methyl-1H-indol-3-yl]propanoyl}amino)acetic acid
({(2R,S)-2-[1-(3-fluorobenzoyl)-4,6-difluoro-5-hydroxy-2-methyl-1H-indol-3-yl]propanoyl}amino)-2-methylpropionic acid
({(2R,S)-2-[1-(3-fluorobenzoyl)-4,6-difluoro-5-hydroxy-2-methyl-1H-indol-3-yl]propanoyl}amino)-2,2-methylbutyric acid
(2R,S)-3-({[1-(3-fluorobenzoyl)-4,6-difluoro-5-hydroxy-2-methyl-1H-indol-3-yl]propanoyl}amino)propanoic acid
(2R,S)-3-({[1-(3-fluorobenzoyl)-4,6-difluoro-5-hydroxy-2-methyl-1H-indol-3-yl]propanoyl}amino)-3-methylpropanic acid
(2R,S)-3-({[1-(3-fluorobenzoyl)-4,6-difluoro-5-hydroxy-2-methyl-1H-indol-3-yl]propanoyl}amino)-2-methylpropanic acid
[1-(4-trifluoromethoxybenzoyl)-5-hydroxy-2-methyl-1H-indol-3-yl]acetic acid,
methyl [1-(4-trifluoromethoxybenzoyl)-5-hydroxy-2-methyl-1H-indol-3-yl]acetate
ethyl [1-(4-trifluoromethoxybenzoyl)-5-hydroxy-2-methyl-1H-indol-3-yl]acetate
propyl [1-(4-trifluoromethoxybenzoyl)-5-hydroxy-2-methyl-1H-indol-3-yl]acetate
butyl [1-(4-trifluoromethoxybenzoyl)-5-hydroxy-2-methyl-1H-indol-3-yl]acetate
sec-butyl [1-(4-trifluoromethoxybenzoyl)-5-hydroxy-2-methyl-1H-indol-3-yl]acetate
(R) sec-butyl [1-(4-trifluoromethoxybenzoyl)-5-hydroxy-2-methyl-1H-indol-3-yl]acetate
(S) sec-butyl [1-(4-trifluoromethoxybenzoyl)-5-hydroxy-2-methyl-1H-indol-3-yl]acetate
isopropyl [1-(4-trifluoromethoxybenzoyl)-5-hydroxy-2-methyl-1H-indol-3-yl]acetate
tert-butyl [1-(4-trifluoromethoxybenzoyl)-5-hydroxy-2-methyl-1H-indol-3-yl]acetate
cyclobutyl [1-(4-trifluoromethoxybenzoyl)-5-hydroxy-2-methyl-1H-indol-3-yl]acetate
cyclopentyl [1-(4-trifluoromethoxybenzoyl)-5-hydroxy-2-methyl-1H-indol-3-yl]acetate
2-[1-(4-trifluoromethoxybenzoyl)-5-hydroxy-2-methyl-1H-indol-3-yl]acetamide
2-[1-(4-trifluoromethoxybenzoyl)-5-hydroxy-2-methyl-1H-indol-3-yl]-N-methylacetamide
2-[1-(4-trifluoromethoxybenzoyl)-5-hydroxy-2-methyl-1H-indol-3-yl]-N-ethylacetamide
2-[1-(4-trifluoromethoxybenzoyl)-5-hydroxy-2-methyl-1H-indol-3-yl]-N-propylacetamide
2-[1-(4-trifluoromethoxybenzoyl)-5-hydroxy-2-methyl-1H-indol-3-yl]-N-isopropylacetamide
2-[1-(4-trifluoromethoxybenzoyl)-5-hydroxy-2-methyl-1H-indol-3-yl]-N-butylacetamide
2-[1-(4-trifluoromethoxybenzoyl)-5-hydroxy-2-methyl-1H-indol-3-yl]-N-sec-butylacetamide
2-[1-(4-trifluoromethoxybenzoyl)-5-hydroxy-2-methyl-1H-indol-3-yl]-N-(2-hydroxyethyl)acetamide
({[1-(4-trifluoromethoxybenzoyl)-5-hydroxy-2-methyl-1H-indol-3-yl]acetyl}amino)acetic acid
3-({[1-(4-trifluoromethoxybenzoyl)-5-hydroxy-2-methyl-1H-indol-3-yl]acetyl}amino)propanoic acid
3-({[1-(4-trifluoromethoxybenzoyl)-5-hydroxy-2-methyl-1H-indol-3-yl]acetyl}amino)-2-methylpropanoic acid
3-({[1-(4-trifluoromethoxybenzoyl)-5-hydroxy-2-methyl-1H-indol-3-yl]acetyl}amino)-3-methylpropanoic acid
2-({[1-(4-trifluoromethoxybenzoyl)-5-hydroxy-2-methyl-1H-indol-3-yl]acetyl}amino)propanoic acid
2-({[1-(4-trifluoromethoxybenzoyl)-5-hydroxy-2-methyl-1H-indol-3-yl]acetyl}amino)-2-methylpropanoic acid
(2R,S)-2[1-(4-trifluoromethoxybenzoyl)-5-hydroxy-2-methyl-1H-indol-3-yl]propionic acid,
methyl (2R,S)-2-[1-(4-trifluoromethoxybenzoyl)-5-hydroxy-2-methyl-1H-indol-3-yl]propanoate
ethyl (2R,S)-2-[1-(4-trifluoromethoxybenzoyl)-5-hydroxy-2-methyl-1H-indol-3-yl]propanoate
propyl (2R,S)-2-[1-(4-trifluoromethoxybenzoyl)-5-hydroxy-2-methyl-1H-indol-3-yl]propanoate
isopropyl (2R,S)-2-[1-(4-trifluoromethoxybenzoyl)-5-hydroxy-2-methyl-1H-indol-3-yl]propanoate
butyl (2R,S)-2-[1-(4-trifluoromethoxybenzoyl)-5-hydroxy-2-methyl-1H-indol-3-yl]propanoate
sec-butyl (2R,S)-2-[1-(4-trifluoromethoxybenzoyl)-5-hydroxy-2-methyl-1H-indol-3-yl]propanoate
(R) sec-butyl (2R,S)-2-[1-(4-trifluoromethoxybenzoyl)-5-hydroxy-2-methyl-1H-indol-3-yl]propanoate
(S) sec-butyl (2R,S)-2-[1-(4-trifluoromethoxybenzoyl)-5-hydroxy-2-methyl-1H-indol-3-yl]propanoate
tert-butyl (2R,S)-2-[1-(4-trifluoromethoxybenzoyl)-5-hydroxy-2-methyl-1H-indol-3-yl]propanoate
cyclobutyl (2R,S)-2-[1-(4-trifluoromethoxybenzoyl)-5-hydroxy-2-methyl-1H-indol-3-yl]propanoate
cyclopentyl (2R,S)-2-[1-(4-trifluoromethoxybenzoyl)-5-hydroxy-2-methyl-1H-indol-3-yl]propanoate
(2R,S)-2-[1-(4-trifluoromethoxybenzoyl)-5-hydroxy-2-methyl-1H-indol-3-yl]propanamide
(2R,S)-2-[1-(4-trifluoromethoxybenzoyl)-5-hydroxy-2-methyl-1H-indol-3-yl]-N-methylpropanamide
(2R,S)-2-[1-(4-trifluoromethoxybenzoyl)-5-hydroxy-2-methyl-1H-indol-3-yl]-N-ethylacetamide
(2R,S)-2-[1-(4-trifluoromethoxybenzoyl)-5-hydroxy-2-methyl-1H-indol-3-yl]-N-propylacetamide
(2R,S)-2-[1-(4-trifluoromethoxybenzoyl)-5-hydroxy-2-methyl-1H-indol-3-yl]-N-isopropylacetamide
(2R,S)-2-[1-(4-trifluoromethoxybenzoyl)-5-hydroxy-2-methyl-1H-indol-3-yl]-N-butylacetamide
(2R,S)-2-[1-(4-trifluoromethoxybenzoyl)-5-hydroxy-2-methyl-1H-indol-3-yl]-N-sec-butylacetamide
(2R,S)-2-[1-(4-trifluoromethoxybenzoyl)-5-hydroxy-2-methyl-1H-indol-3-yl]-N-(hydroxymethyl)propanamide
({(2R,S)-2-[1-(4-trifluoromethoxybenzoyl)-5-hydroxy-2-methyl-1H-indol-3-yl]propanoyl}amino)acetic acid
({(2R,S)-2-[1-(4-trifluoromethoxybenzoyl)-5-hydroxy-2-methyl-1H-indol-3-yl]propanoyl}amino)-2-methylpropionic acid
({(2R,S)-2-[1-(4-trifluoromethoxybenzoyl)-5-hydroxy-2-methyl-1H-indol-3-yl]propanoyl}amino)-2,2-methylbutyric acid
(2R,S)-3-({[1-(4-trifluoromethoxybenzoyl)-5-hydroxy-2-methyl-1H-indol-3-yl]propanoyl}amino)propanoic acid
(2R,S)-3-({[1-(4-trifluoromethoxybenzoyl)-5-hydroxy-2-methyl-1H-indol-3-yl]propanoyl}amino)-3-methylpropanic acid
(2R,S)-3-({[1-(4-trifluoromethoxybenzoyl)-5-hydroxy-2-methyl-1H-indol-3-yl]propanoyl}amino)-2-methylpropanic acid
[1-(4-trifluoromethoxybenzoyl)-6-fluoro-5-hydroxy-2-methyl-1H-indol-3-yl]acetic acid,
methyl [1-(4-trifluoromethoxybenzoyl)-6-fluoro-5-hydroxy-2-methyl-1H-indol-3-yl]acetate
ethyl [1-(4-trifluoromethoxybenzoyl)-6-fluoro-5-hydroxy-2-methyl-1H-indol-3-yl]acetate
propyl [1-(4-trifluoromethoxybenzoyl)-6-fluoro-5-hydroxy-2-methyl-1H-indol-3-yl]acetate
butyl [1-(4-trifluoromethoxybenzoyl)-6-fluoro-5-hydroxy-2-methyl-1H-indol-3-yl]acetate
sec-butyl [1-(4-trifluoromethoxybenzoyl)-6-fluoro-5-hydroxy-2-methyl-1H-indol-3-yl]acetate
(R) sec-butyl [1-(4-trifluoromethoxybenzoyl)-6-fluoro-5-hydroxy-2-methyl-1H-indol-3-yl]acetate
(S) sec-butyl [1-(4-trifluoromethoxybenzoyl)-6-fluoro-5-hydroxy-2-methyl-1H-indol-3-yl]acetate
isopropyl [1-(4-trifluoromethoxybenzoyl)-6-fluoro-5-hydroxy-2-methyl-1H-indol-3-yl]acetate
tert-butyl [1-(4-trifluoromethoxybenzoyl)-6-fluoro-5-hydroxy-2-methyl-1H-indol-3-yl]acetate
cyclobutyl [1-(4-trifluoromethoxybenzoyl)-6-fluoro-5-hydroxy-2-methyl-1H-indol-3-yl]acetate
cyclopentyl [1-(4-trifluoromethoxybenzoyl)-6-fluoro-5-hydroxy-2-methyl-1H-indol-3-yl]acetate
2-[1-(4-trifluoromethoxybenzoyl)-6-fluoro-5-hydroxy-2-methyl-1H-indol-3-yl]acetamide
2-[1-(4-trifluoromethoxybenzoyl)-6-fluoro-5-hydroxy-2-methyl-1H-indol-3-yl]-N-methylacetamide
2-[1-(4-trifluoromethoxybenzoyl)-6-fluoro-5-hydroxy-2-methyl-1H-indol-3-yl]-N-ethylacetamide
2-[1-(4-trifluoromethoxybenzoyl)-6-fluoro-5-hydroxy-2-methyl-1H-indol-3-yl]-N-propylacetamide
2-[1-(4-trifluoromethoxybenzoyl)-6-fluoro-5-hydroxy-2-methyl-1H-indol-3-yl]-N-isopropylacetamide
2-[1-(4-trifluoromethoxybenzoyl)-6-fluoro-5-hydroxy-2-methyl-1H-indol-3-yl]-N-butylacetamide
2-[1-(4-trifluoromethoxybenzoyl)-6-fluoro-5-hydroxy-2-methyl-1H-indol-3-yl]-N-sec-butylacetamide
2-[1-(4-trifluoromethoxybenzoyl)-6-fluoro-5-hydroxy-2-methyl-1H-indol-3-yl]-N-(2-hydroxyethyl)acetamide
({[1-(4-trifluoromethoxybenzoyl)-6-fluoro-5-hydroxy-2-methyl-1H-indol-3-yl]acetyl}amino)acetic acid
3-({[1-(4-trifluoromethoxybenzoyl)-6-fluoro-5-hydroxy-2-methyl-1H-indol-3-yl]acetyl}amino)propanoic acid
3-({[1-(4-trifluoromethoxybenzoyl)-6-fluoro-5-hydroxy-2-methyl-1H-indol-3-yl]acetyl}amino)-2-methylpropanoic acid
3-({[1-(4-trifluoromethoxybenzoyl)-6-fluoro-5-hydroxy-2-methyl-1H-indol-3-yl]acetyl}amino)-3-methylpropanoic acid
2-({[1-(4-trifluoromethoxybenzoyl)-6-fluoro-5-hydroxy-2-methyl-1H-indol-3-yl]acetyl}amino)propanoic acid
2-({[1-(4-trifluoromethoxybenzoyl)-6-fluoro-5-hydroxy-2-methyl-1H-indol-3-yl]acetyl}amino)-2-methylpropanoic acid
(2R,S)-2[1-(4-trifluoromethoxybenzoyl)-6-fluoro-5-hydroxy-2-methyl-1H-indol-3-yl]propionic acid,
methyl (2R,S)-2-[1-(4-trifluoromethoxybenzoyl)-6-fluoro-5-hydroxy-2-methyl-1H-indol-3-yl]propanoate
ethyl (2R,S)-2-[1-(4-trifluoromethoxybenzoyl)-6-fluoro-5-hydroxy-2-methyl-1H-indol-3-yl]propanoate
propyl (2R,S)-2-[1-(4-trifluoromethoxybenzoyl)-6-fluoro-5-hydroxy-2-methyl-1H-indol-3-yl]propanoate
isopropyl (2R,S)-2-[1-(4-trifluoromethoxybenzoyl)-6-fluoro-5-hydroxy-2-methyl-1H-indol-3-yl]propanoate
butyl (2R,S)-2-[1-(4-trifluoromethoxybenzoyl)-6-fluoro-5-hydroxy-2-methyl-1H-indol-3-yl]propanoate
sec-butyl (2R,S)-2-[1-(4-trifluoromethoxybenzoyl)-6-fluoro-5-hydroxy-2-methyl-1H-indol-3-yl]propanoate
(R) sec-butyl (2R,S)-2-[1-(4-trifluoromethoxybenzoyl)-6-fluoro-5-hydroxy-2-methyl-1H-indol-3-yl]propanoate
(S) sec-butyl (2R,S)-2-[1-(4-trifluoromethoxybenzoyl)-6-fluoro-5-hydroxy-2-methyl-1H-indol-3-yl]propanoate
tert-butyl (2R,S)-2-[1-(4-trifluoromethoxybenzoyl)-6-fluoro-5-hydroxy-2-methyl-1H-indol-3-yl]propanoate
cyclobutyl (2R,S)-2-[1-(4-trifluoromethoxybenzoyl)-6-fluoro-5-hydroxy-2-methyl-1H-indol-3-yl]propanoate
cyclopentyl (2R,S)-2-[1-(4-trifluoromethoxybenzoyl)-6-fluoro-5-hydroxy-2-methyl-1H-indol-3-yl]propanoate
(2R,S)-2-[1-(4-trifluoromethoxybenzoyl)-6-fluoro-5-hydroxy-2-methyl-1H-indol-3-yl]propanamide
(2R,S)-2-[1-(4-trifluoromethoxybenzoyl)-6-fluoro-5-hydroxy-2-methyl-1H-indol-3-yl]-N-methylpropanamide
(2R,S)-2-[1-(4-trifluoromethoxybenzoyl)-6-fluoro-5-hydroxy-2-methyl-1H-indol-3-yl]-N-ethylacetamide
(2R,S)-2-[1-(4-trifluoromethoxybenzoyl)-6-fluoro-5-hydroxy-2-methyl-1H-indol-3-yl]-N-propylacetamide
(2R,S)-2-[1-(4-trifluoromethoxybenzoyl)-6-fluoro-5-hydroxy-2-methyl-1H-indol-3-yl]-N-isopropylacetamide
(2R,S)-2-[1-(4-trifluoromethoxybenzoyl)-6-fluoro-5-hydroxy-2-methyl-1H-indol-3-yl]-N-butylacetamide
(2R,S)-2-[1-(4-trifluoromethoxybenzoyl)-6-fluoro-5-hydroxy-2-methyl-1H-indol-3-yl]-N-sec-butylacetamide
(2R,S)-2-[1-(4-trifluoromethoxybenzoyl)-6-fluoro-5-hydroxy-2-methyl-1H-indol-3-yl]-N-(hydroxymethyl)propanamide
({(2R,S)-2-[1-(4-trifluoromethoxybenzoyl)-6-fluoro-5-hydroxy-2-methyl-1H-indol-3-yl]propanoyl}amino)acetic acid
({(2R,S)-2-[1-(4-trifluoromethoxybenzoyl)-6-fluoro-5-hydroxy-2-methyl-1H-indol-3-yl]propanoyl}amino)-2-methylpropionic acid
({(2R,S)-2-[1-(4-trifluoromethoxybenzoyl)-6-fluoro-5-hydroxy-2-methyl-1H-indol-3-yl]propanoyl}amino)-2,2-methylbutyric acid
(2R,S)-3-({[1-(4-trifluoromethoxybenzoyl)-6-fluoro-5-hydroxy-2-methyl-1H-indol-3-yl]propanoyl}amino)propanoic acid
(2R,S)-3-({[1-(4-trifluoromethoxybenzoyl)-6-fluoro-5-hydroxy-2-methyl-1H-indol-3-yl]propanoyl}amino)-3-methylpropanic acid
(2R,S)-3-({[1-(4-trifluoromethoxybenzoyl)-6-fluoro-5-hydroxy-2-methyl-1H-indol-3-yl]propanoyl}amino)-2-methylpropanic acid
[1-(4-trifluoromethoxybenzoyl)-4-fluoro-5-hydroxy-2-methyl-1H-indol-3-yl]acetic acid,
methyl [1-(4-trifluoromethoxybenzoyl)-4-fluoro-5-hydroxy-2-methyl-1H-indol-3-yl]acetate
ethyl [1-(4-trifluoromethoxybenzoyl)-4-fluoro-5-hydroxy-2-methyl-1H-indol-3-yl]acetate
propyl [1-(4-trifluoromethoxybenzoyl)-4-fluoro-5-hydroxy-2-methyl-1H-indol-3-yl]acetate
butyl [1-(4-trifluoromethoxybenzoyl)-4-fluoro-5-hydroxy-2-methyl-1H-indol-3-yl]acetate
sec-butyl [1-(4-trifluoromethoxybenzoyl)-4-fluoro-5-hydroxy-2-methyl-1H-indol-3-yl]acetate
(R) sec-butyl [1-(4-trifluoromethoxybenzoyl)-4-fluoro-5-hydroxy-2-methyl-1H-indol-3-yl]acetate
(S) sec-butyl [1-(4-trifluoromethoxybenzoyl)-4-fluoro-5-hydroxy-2-methyl-1H-indol-3-yl]acetate
isopropyl [1-(4-trifluoromethoxybenzoyl)-4-fluoro-5-hydroxy-2-methyl-1H-indol-3-yl]acetate
tert-butyl [1-(4-trifluoromethoxybenzoyl)-4-fluoro-5-hydroxy-2-methyl-1H-indol-3-yl]acetate
cyclobutyl [1-(4-trifluoromethoxybenzoyl)-4-fluoro-5-hydroxy-2-methyl-1H-indol-3-yl]acetate
cyclopentyl [1-(4-trifluoromethoxybenzoyl)-4-fluoro-5-hydroxy-2-methyl-1H-indol-3-yl]acetate
2-[1-(4-trifluoromethoxybenzoyl)-4-fluoro-5-hydroxy-2-methyl-1H-indol-3-yl]acetamide
2-[1-(4-trifluoromethoxybenzoyl)-4-fluoro-5-hydroxy-2-methyl-1H-indol-3-yl]-N-methylacetamide
2-[1-(4-trifluoromethoxybenzoyl)-4-fluoro-5-hydroxy-2-methyl-1H-indol-3-yl]-N-ethylacetamide
2-[1-(4-trifluoromethoxybenzoyl)-4-fluoro-5-hydroxy-2-methyl-1H-indol-3-yl]-N-propylacetamide
2-[1-(4-trifluoromethoxybenzoyl)-4-fluoro-5-hydroxy-2-methyl-1H-indol-3-yl]-N-isopropylacetamide
2-[1-(4-trifluoromethoxybenzoyl)-4-fluoro-5-hydroxy-2-methyl-1H-indol-3-yl]-N-butylacetamide
2-[1-(4-trifluoromethoxybenzoyl)-4-fluoro-5-hydroxy-2-methyl-1H-indol-3-yl]-N-sec-butylacetamide
2-[1-(4-trifluoromethoxybenzoyl)-4-fluoro-5-hydroxy-2-methyl-1H-indol-3-yl]-N-(2-hydroxyethyl)acetamide
({[1-(4-trifluoromethoxybenzoyl)-4-fluoro-5-hydroxy-2-methyl-1H-indol-3-yl]acetyl}amino)acetic acid
3-({[1-(4-trifluoromethoxybenzoyl)-4-fluoro-5-hydroxy-2-methyl-1H-indol-3-yl]acetyl}amino)propanoic acid
3-({[1-(4-trifluoromethoxybenzoyl)-4-fluoro-5-hydroxy-2-methyl-1H-indol-3-yl]acetyl}amino)-2-methylpropanoic acid
3-({[1-(4-trifluoromethoxybenzoyl)-4-fluoro-5-hydroxy-2-methyl-1H-indol-3-yl]acetyl}amino)-3-methylpropanoic acid
2-({[1-(4-trifluoromethoxybenzoyl)-4-fluoro-5-hydroxy-2-methyl-1H-indol-3-yl]acetyl}amino)propanoic acid
2-({[1-(4-trifluoromethoxybenzoyl)-4-fluoro-5-hydroxy-2-methyl-1H-indol-3-yl]acetyl}amino)-2-methylpropanoic acid
2-[1-(4-trifluoromethoxybenzoyl)-4-fluoro-5-hydroxy-2-methyl-1H-indol-3-yl]propionic acid,
(2R,S)-2[1-(4-trifluoromethoxybenzoyl)-4-fluoro-5-hydroxy-2-methyl-1H-indol-3-yl]propionic acid,
methyl (2R,S)-2-[1-(4-trifluoromethoxybenzoyl)-4-fluoro-5-hydroxy-2-methyl-1H-indol-3-yl]propanoate
ethyl (2R,S)-2-[1-(4-trifluoromethoxybenzoyl)-4-fluoro-5-hydroxy-2-methyl-1H-indol-3-yl]propanoate
propyl (2R,S)-2-[1-(4-trifluoromethoxybenzoyl)-4-fluoro-5-hydroxy-2-methyl-1H-indol-3-yl]propanoate
isopropyl (2R,S)-2-[1-(4-trifluoromethoxybenzoyl)-4-fluoro-5-hydroxy-2-methyl-1H-indol-3-yl]propanoate
butyl (2R,S)-2-[1-(4-trifluoromethoxybenzoyl)-4-fluoro-5-hydroxy-2-methyl-1H-indol-3-yl]propanoate
sec-butyl (2R,S)-2-[1-(4-trifluoromethoxybenzoyl)-4-fluoro-5-hydroxy-2-methyl-1H-indol-3-yl]propanoate
(R) sec-butyl (2R,S)-2-[1-(4-trifluoromethoxybenzoyl)-4-fluoro-5-hydroxy-2-methyl-1H-indol-3-yl]propanoate
(S) sec-butyl (2R,S)-2-[1-(4-trifluoromethoxybenzoyl)-4-fluoro-5-hydroxy-2-methyl-1H-indol-3-yl]propanoate
tert-butyl (2R,S)-2-[1-(4-trifluoromethoxybenzoyl)-4-fluoro-5-hydroxy-2-methyl-1H-indol-3-yl]propanoate
cyclobutyl (2R,S)-2-[1-(4-trifluoromethoxybenzoyl)-4-fluoro-5-hydroxy-2-methyl-1H-indol-3-yl]propanoate
cyclopentyl (2R,S)-2-[1-(4-trifluoromethoxybenzoyl)-4-fluoro-5-hydroxy-2-methyl-1H-indol-3-yl]propanoate
(2R,S)-2-[1-(4-trifluoromethoxybenzoyl)-4-fluoro-5-hydroxy-2-methyl-1H-indol-3-yl]propanamide
(2R,S)-2-[1-(4-trifluoromethoxybenzoyl)-4-fluoro-5-hydroxy-2-methyl-1H-indol-3-yl]-N-methylpropanamide
(2R,S)-2-[1-(4-trifluoromethoxybenzoyl)-4-fluoro-5-hydroxy-2-methyl-1H-indol-3-yl]-N-ethylacetamide
(2R,S)-2-[1-(4-trifluoromethoxybenzoyl)-4-fluoro-5-hydroxy-2-methyl-1H-indol-3-yl]-N-propylacetamide
(2R,S)-2-[1-(4-trifluoromethoxybenzoyl)-4-fluoro-5-hydroxy-2-methyl-1H-indol-3-yl]-N-isopropylacetamide
(2R,S)-2-[1-(4-trifluoromethoxybenzoyl)-4-fluoro-5-hydroxy-2-methyl-1H-indol-3-yl]-N-butylacetamide
(2R,S)-2-[1-(4-trifluoromethoxybenzoyl)-4-fluoro-5-hydroxy-2-methyl-1H-indol-3-yl]-N-sec-butylacetamide
(2R,S)-2-[1-(4-trifluoromethoxybenzoyl)-4-fluoro-5-hydroxy-2-methyl-1H-indol-3-yl]-N-(hydroxymethyl)propanamide
({(2R,S)-2-[1-(4-trifluoromethoxybenzoyl)-4-fluoro-5-hydroxy-2-methyl-1H-indol-3-yl]propanoyl}amino)acetic acid
({(2R,S)-2-[1-(4-trifluoromethoxybenzoyl)-4-fluoro-5-hydroxy-2-methyl-1H-indol-3-yl]propanoyl}amino)-2-methylpropionic acid
({(2R,S)-2-[1-(4-trifluoromethoxybenzoyl)-4-fluoro-5-hydroxy-2-methyl-1H-indol-3-yl]propanoyl}amino)-2,2-methylbutyric acid
(2R,S)-3-({[1-(4-trifluoromethoxybenzoyl)-4-fluoro-5-hydroxy-2-methyl-1H-indol-3-yl]propanoyl}amino)propanoic acid
(2R,S)-3-({[1-(4-trifluoromethoxybenzoyl)-4-fluoro-5-hydroxy-2-methyl-1H-indol-3-yl]propanoyl}amino)-3-methylpropanic acid
(2R,S)-3-({[1-(4-trifluoromethoxybenzoyl)-4-fluoro-5-hydroxy-2-methyl-1H-indol-3-yl]propanoyl}amino)-2-methylpropanic acid
[1-(4-trifluoromethoxybenzoyl)-4,6-difluoro-5-hydroxy-2-methyl-1H-indol-3-yl]acetic acid,
methyl [1-(4-trifluoromethoxybenzoyl)-4,6-difluoro-5-hydroxy-2-methyl-1H-indol-3-yl]acetate
ethyl [1-(4-trifluoromethoxybenzoyl)-4,6-difluoro-5-hydroxy-2-methyl-1H-indol-3-yl]acetate
propyl [1-(4-trifluoromethoxybenzoyl)-4,6-difluoro-5-hydroxy-2-methyl-1H-indol-3-yl]acetate
butyl [1-(4-trifluoromethoxybenzoyl)-4,6-difluoro-5-hydroxy-2-methyl-1H-indol-3-yl]acetate
sec-butyl [1-(4-trifluoromethoxybenzoyl)-4,6-difluoro-5-hydroxy-2-methyl-1H-indol-3-yl]acetate
(R) sec-butyl [1-(4-trifluoromethoxybenzoyl)-4,6-difluoro-5-hydroxy-2-methyl-1H-indol-3-yl]acetate
(S) sec-butyl [1-(4-trifluoromethoxybenzoyl)-4,6-difluoro-5-hydroxy-2-methyl-1H-indol-3-yl]acetate
isopropyl [1-(4-trifluoromethoxybenzoyl)-4,6-difluoro-5-hydroxy-2-methyl-1H-indol-3-yl]acetate
tert-butyl [1-(4-trifluoromethoxybenzoyl)-4,6-difluoro-5-hydroxy-2-methyl-1H-indol-3-yl]acetate
cyclobutyl [1-(4-trifluoromethoxybenzoyl)-4,6-difluoro-5-hydroxy-2-methyl-1H-indol-3-yl]acetate
cyclopentyl [1-(4-trifluoromethoxybenzoyl)-4,6-difluoro-5-hydroxy-2-methyl-1H-indol-3-yl]acetate
2-[1-(4-trifluoromethoxybenzoyl)-4,6-difluoro-5-hydroxy-2-methyl-1H-indol-3-yl]acetamide
2-[1-(4-trifluoromethoxybenzoyl)-4,6-difluoro-5-hydroxy-2-methyl-1H-indol-3-yl]-N-methylacetamide
2-[1-(4-trifluoromethoxybenzoyl)-4,6-difluoro-5-hydroxy-2-methyl-1H-indol-3-yl]-N-ethylacetamide
2-[1-(4-trifluoromethoxybenzoyl)-4,6-difluoro-5-hydroxy-2-methyl-1H-indol-3-yl]-N-propylacetamide
2-[1-(4-trifluoromethoxybenzoyl)-4,6-difluoro-5-hydroxy-2-methyl-1H-indol-3-yl]-N-isopropylacetamide
2-[1-(4-trifluoromethoxybenzoyl)-4,6-difluoro-5-hydroxy-2-methyl-1H-indol-3-yl]-N-butylacetamide
2-[1-(4-trifluoromethoxybenzoyl)-4,6-difluoro-5-hydroxy-2-methyl-1H-indol-3-yl]-N-sec-butylacetamide
2-[1-(4-trifluoromethoxybenzoyl)-4,6-difluoro-5-hydroxy-2-methyl-1H-indol-3-yl]-N-(2-hydroxyethyl)acetamide
({[1-(4-trifluoromethoxybenzoyl)-4,6-difluoro-5-hydroxy-2-methyl-1H-indol-3-yl]acetyl}amino)acetic acid
3-({[1-(4-trifluoromethoxybenzoyl)-4,6-difluoro-5-hydroxy-2-methyl-1H-indol-3-yl]acetyl}amino)propanoic acid
3-({[1-(4-trifluoromethoxybenzoyl)-4,6-difluoro-5-hydroxy-2-methyl-1H-indol-3-yl]acetyl}amino)-2-methylpropanoic acid
3-({[1-(4-trifluoromethoxybenzoyl)-4,6-difluoro-5-hydroxy-2-methyl-1H-indol-3-yl]acetyl}amino)-3-methylpropanoic acid
2-({[1-(4-trifluoromethoxybenzoyl)-4,6-difluoro-5-hydroxy-2-methyl-1H-indol-3-yl]acetyl}amino)propanoic acid
2-({[1-(4-trifluoromethoxybenzoyl)-4,6-difluoro-5-hydroxy-2-methyl-1H-indol-3-yl]acetyl}amino)-2-methylpropanoic acid
(2R,S)-2[1-(4-trifluoromethoxybenzoyl)-4,6-difluoro-5-hydroxy-2-methyl-1H-indol-3-yl]propionic acid,
methyl (2R,S)-2-[1-(4-trifluoromethoxybenzoyl)-4,6-difluoro-5-hydroxy-2-methyl-[H-indol-3-yl]propanoate
ethyl (2R,S)-2-[1-(4-trifluoromethoxybenzoyl)-4,6-difluoro-5-hydroxy-2-methyl-1H-indol-3-yl]propanoate
propyl (2R,S)-2-[1-(4-trifluoromethoxybenzoyl)-4,6-difluoro-5-hydroxy-2-methyl-1H-indol-3-yl]propanoate
isopropyl (2R,S)-2-[1-(4-trifluoromethoxybenzoyl)-4,6-difluoro-5-hydroxy-2-methyl-1H-indol-3-yl]propanoate
butyl (2R,S)-2-[1-(4-trifluoromethoxybenzoyl)-4,6-difluoro-5-hydroxy-2-methyl-1H-indol-3-yl]propanoate
sec-butyl (2R,S)-2-[1-(4-trifluoromethoxybenzoyl)-4,6-difluoro-5-hydroxy-2-methyl-1H-indol-3-yl]propanoate
(R) sec-butyl (2R,S)-2-[1-(4-trifluoromethoxybenzoyl)-4,6-difluoro-5-hydroxy-2-methyl-1H-indol-3-yl]propanoate
(S) sec-butyl (2R,S)-2-[1-(4-trifluoromethoxybenzoyl)-4,6-difluoro-5-hydroxy-2-methyl-1H-indol-3-yl]propanoate
tert-butyl (2R,S)-2-[1-(4-trifluoromethoxybenzoyl)-4,6-difluoro-5-hydroxy-2-methyl-1H-indol-3-yl]propanoate
cyclobutyl (2R,S)-2-[1-(4-trifluoromethoxybenzoyl)-4,6-difluoro-5-hydroxy-2-methyl-1H-indol-3-yl]propanoate
cyclopentyl (2R,S)-2-[1-(4-trifluoromethoxybenzoyl)-4,6-difluoro-5-hydroxy-2-methyl-1H-indol-3-yl]propanoate
(2R,S)-2-[1-(4-trifluoromethoxybenzoyl)-4,6-difluoro-5-hydroxy-2-methyl-1H-indol-3-yl]propanamide
(2R,S)-2-[1-(4-trifluoromethoxybenzoyl)-4,6-difluoro-5-hydroxy-2-methyl-1H-indol-3-yl]-N-methylpropanamide
(2R,S)-2-[1-(4-trifluoromethoxybenzoyl)-4,6-difluoro-5-hydroxy-2-methyl-1H-indol-3-yl]-N-ethylacetamide
(2R,S)-2-[1-(4-trifluoromethoxybenzoyl)-4,6-difluoro-5-hydroxy-2-methyl-1H-indol-3-yl]-N-propylacetamide
(2R,S)-2-[1-(4-trifluoromethoxybenzoyl)-4,6-difluoro-5-hydroxy-2-methyl-1H-indol-3-yl]-N-isopropylacetamide
(2R,S)-2-[1-(4-trifluoromethoxybenzoyl)-4,6-difluoro-5-hydroxy-2-methyl-1H-indol-3-yl]-N-butylacetamide
(2R,S)-2-[1-(4-trifluoromethoxybenzoyl)-4,6-difluoro-5-hydroxy-2-methyl-1H-indol-3-yl]-N-sec-butylacetamide
(2R,S)-2-[1-(4-trifluoromethoxybenzoyl)-4,6-difluoro-5-hydroxy-2-methyl-1H-indol-3-yl]-N-(hydroxymethyl)propanamide
({(2R,S)-2-[1-(4-trifluoromethoxybenzoyl)-4,6-difluoro-5-hydroxy-2-methyl-1H-indol-3-yl]propanoyl}amino)acetic acid
({(2R,S)-2-[1-(4-trifluoromethoxybenzoyl)-4,6-difluoro-5-hydroxy-2-methyl-1H-indol-3-yl]propanoyl}amino)-2-methylpropionic acid
({(2R,S)-2-[1-(4-trifluoromethoxybenzoyl)-4,6-difluoro-5-hydroxy-2-methyl-1H-indol-3-yl]propanoyl}amino)-2,2-methylbutyric acid
(2R,S)-3-({[1-(4-trifluoromethoxybenzoyl)-4,6-difluoro-5-hydroxy-2-methyl-1H-indol-3-yl]propanoyl}amino)propanoic acid
(2R,S)-3-({[1-(4-trifluoromethoxybenzoyl)-4,6-difluoro-5-hydroxy-2-methyl-1H-indol-3-yl]propanoyl}amino)-3-methylpropanic acid
(2R,S)-3-({[1-(4-trifluoromethoxybenzoyl)-4,6-difluoro-5-hydroxy-2-methyl-1H-indol-3-yl]propanoyl}amino)-2-methylpropanic acid
{1-[4-(difluoromethoxy)benzoyl]-6-fluoro-5-methoxy-2-methyl-1H-indol-3-yl]acetic acid,
methyl {1-[4-(difluoromethoxy)benzoyl]-6-fluoro-5-methoxy-2-methyl-1H-indol-3-yl]acetate
ethyl {1-[4-(difluoromethoxy)benzoyl]-6-fluoro-5-methoxy-2-methyl-1H-indol-3-yl]acetate
propyl {1-[4-(difluoromethoxy)benzoyl]-6-fluoro-5-methoxy-2-methyl-1H-indol-3-yl]acetate
butyl {1-[4-(difluoromethoxy)benzoyl]-6-fluoro-5-methoxy-2-methyl-1H-indol-3-yl]acetate
sec-butyl {1-[4-(difluoromethoxy)benzoyl]-6-fluoro-5-methoxy-2-methyl-1H-indol-3-yl]acetate
(R) sec-butyl {1-[4-(difluoromethoxy)benzoyl]-6-fluoro-5-methoxy-2-methyl-1H-indol-3-yl]acetate
(S) sec-butyl {1-[4-(difluoromethoxy)benzoyl]-6-fluoro-5-methoxy-2-methyl-1H-indol-3-yl]acetate
isopropyl {1-[4-(difluoromethoxy)benzoyl]-6-fluoro-5-methoxy-2-methyl-1H-indol-3-yl]acetate
tert-butyl {1-[4-(difluoromethoxy)benzoyl]-6-fluoro-5-methoxy-2-methyl-1H-indol-3-yl]acetate
cyclobutyl {1-[4-(difluoromethoxy)benzoyl]-6-fluoro-5-methoxy-2-methyl-1H-indol-3-yl]acetate
cyclopentyl {1-[4-(difluoromethoxy)benzoyl]-6-fluoro-5-methoxy-2-methyl-1H-indol-3-yl]acetate
2-{1-[4-(difluoromethoxy)benzoyl]-6-fluoro-5-methoxy-2-methyl-1H-indol-3-yl]acetamide
2-{1-[4-(difluoromethoxy)benzoyl]-6-fluoro-5-methoxy-2-methyl-1H-indol-3-yl]-N-methylacetamide
2-{1-[4-(difluoromethoxy)benzoyl]-6-fluoro-5-methoxy-2-methyl-1H-indol-3-yl]-N-ethylacetamide
2-{1-[4-(difluoromethoxy)benzoyl]-6-fluoro-5-methoxy-2-methyl-1H-indol-3-yl]-N-propylacetamide
2-{1-[4-(difluoromethoxy)benzoyl]-6-fluoro-5-methoxy-2-methyl-1H-indol-3-yl]-N-isopropylacetamide
2-{1-[4-(difluoromethoxy)benzoyl]-6-fluoro-5-methoxy-2-methyl-1H-indol-3-yl]-N-butylacetamide
2-{1-[4-(difluoromethoxy)benzoyl]-6-fluoro-5-methoxy-2-methyl-1H-indol-3-yl]-N-sec-butylacetamide
2-{1-[4-(difluoromethoxy)benzoyl]-6-fluoro-5-methoxy-2-methyl-1H-indol-3-yl]-N-(2-hydroxyethyl)acetamide
({{1-[4-(difluoromethoxy)benzoyl]-6-fluoro-5-methoxy-2-methyl-1H-indol-3-yl]acetyl}amino)acetic acid
3-({{1-[4-(difluoromethoxy)benzoyl]-6-fluoro-5-methoxy-2-methyl-1H-indol-3-yl]acetyl}amino)propanoic acid
3-({{1-[4-(difluoromethoxy)benzoyl]-6-fluoro-5-methoxy-2-methyl-1H-indol-3-yl]acetyl}amino)-2-methylpropanoic acid
3-({{1-[4-(difluoromethoxy)benzoyl]-6-fluoro-5-methoxy-2-methyl-1H-indol-3-yl]acetyl}amino)-3-methylpropanoic acid
2-({{1-[4-(difluoromethoxy)benzoyl]-6-fluoro-5-methoxy-2-methyl-1H-indol-3-yl]acetyl}amino)propanoic acid
2-({{1-[4-(difluoromethoxy)benzoyl]-6-fluoro-5-methoxy-2-methyl-1H-indol-3-yl]acetyl}amino)-2-methylpropanoic acid
1-[4-(difluoromethoxy)benzoyl]-6-fluoro-5-hydroxy-2-methyl-1H-indol-3-yl]acetic acid,
methyl {1-[4-(difluoromethoxy)benzoyl]-6-fluoro-5-hydroxy-2-methyl-1H-indol-3-yl]acetate
ethyl {1-[4-(difluoromethoxy)benzoyl]-6-fluoro-5-hydroxy-2-methyl-1H-indol-3-yl]acetate
propyl {1-[4-(difluoromethoxy)benzoyl]-6-fluoro-5-hydroxy-2-methyl-1H-indol-3-yl]acetate
butyl {1-[4-(difluoromethoxy)benzoyl]-6-fluoro-5-hydroxy-2-methyl-1H-indol-3-yl]acetate
sec-butyl {1-[4-(difluoromethoxy)benzoyl]-6-fluoro-5-hydroxy-2-methyl-1H-indol-3-yl]acetate
(R) sec-butyl {1-[4-(difluoromethoxy)benzoyl]-6-fluoro-5-hydroxy-2-methyl-1H-indol-3-yl]acetate
(S) sec-butyl {1-[4-(difluoromethoxy)benzoyl]-6-fluoro-5-hydroxy-2-methyl-1H-indol-3-yl]acetate
isopropyl {1-[4-(difluoromethoxy)benzoyl]-6-fluoro-5-hydroxy-2-methyl-1H-indol-3-yl]acetate
tert-butyl {1-[4-(difluoromethoxy)benzoyl]-6-fluoro-5-hydroxy-2-methyl-1H-indol-3-yl]acetate
cyclobutyl {1-[4-(difluoromethoxy)benzoyl]-6-fluoro-5-hydroxy-2-methyl-1H-indol-3-yl]acetate
cyclopentyl {1-[4-(difluoromethoxy)benzoyl]-6-fluoro-5-hydroxy-2-methyl-1H-indol-3-yl]acetate
2-{1-[4-(difluoromethoxy)benzoyl]-6-fluoro-5-hydroxy-2-methyl-1H-indol-3-yl]acetamide
2-{1-[4-(difluoromethoxy)benzoyl]-6-fluoro-5-hydroxy-2-methyl-1H-indol-3-yl]-N-methylacetamide
2-{1-[4-(difluoromethoxy)benzoyl]-6-fluoro-5-hydroxy-2-methyl-1H-indol-3-yl]-N-ethylacetamide
2-{1-[4-(difluoromethoxy)benzoyl]-6-fluoro-5-hydroxy-2-methyl-1H-indol-3-yl]-N-propylacetamide
2-{1-[4-(difluoromethoxy)benzoyl]-6-fluoro-5-hydroxy-2-methyl-1H-indol-3-yl]-N-isopropylacetamide
2-{1-[4-(difluoromethoxy)benzoyl]-6-fluoro-5-hydroxy-2-methyl-1H-indol-3-yl]-N-butylacetamide
2-{1-[4-(difluoromethoxy)benzoyl]-6-fluoro-5-hydroxy-2-methyl-1H-indol-3-yl]-N-sec-butylacetamide
2-{1-[4-(difluoromethoxy)benzoyl]-6-fluoro-5-hydroxy-2-methyl-1H-indol-3-yl]-N-(2-hydroxyethyl)acetamide
({{1-[4-(difluoromethoxy)benzoyl]-6-fluoro-5-hydroxy-2-methyl-1H-indol-3-yl]acetyl}amino)acetic acid
3-({{1-[4-(difluoromethoxy)benzoyl]-6-fluoro-5-hydroxy-2-methyl-1H-indol-3-yl]acetyl}amino)propanoic acid
3-({{1-[4-(difluoromethoxy)benzoyl]-6-fluoro-5-hydroxy-2-methyl-1H-indol-3-yl]acetyl}amino)-2-methylpropanoic acid
3-({{1-[4-(difluoromethoxy)benzoyl]-6-fluoro-5-hydroxy-2-methyl-1H-indol-3-yl]acetyl}amino)-3-methylpropanoic acid
2-({{1-[4-(difluoromethoxy)benzoyl]-6-fluoro-5-hydroxy-2-methyl-1H-indol-3-yl]acetyl}amino)propanoic acid
2-({{1-[4-(difluoromethoxy)benzoyl]-6-fluoro-5-hydroxy-2-methyl-1H-indol-3-yl]acetyl}amino)-2-methylpropanoic acid
1-[4-(difluoromethoxy)benzoyl]-5-methoxy-2-methyl-1H-indol-3-yl]acetic acid,
methyl {1-[4-(difluoromethoxy)benzoyl]-5-methoxy-2-methyl-1H-indol-3-yl]acetate
ethyl {1-[4-(difluoromethoxy)benzoyl]-5-methoxy-2-methyl-1H-indol-3-yl]acetate
propyl {1-[4-(difluoromethoxy)benzoyl]-5-methoxy-2-methyl-1H-indol-3-yl]acetate
butyl {1-[4-(difluoromethoxy)benzoyl]-5-methoxy-2-methyl-1H-indol-3-yl]acetate
sec-butyl {1-[4-(difluoromethoxy)benzoyl]-5-methoxy-2-methyl-1H-indol-3-yl]acetate
(R) sec-butyl {1-[4-(difluoromethoxy)benzoyl]-5-methoxy-2-methyl-1H-indol-3-yl]acetate
(S) sec-butyl {1-[4-(difluoromethoxy)benzoyl]-5-methoxy-2-methyl-1H-indol-3-yl]acetate
isopropyl {1-[4-(difluoromethoxy)benzoyl]-5-methoxy-2-methyl-1H-indol-3-yl]acetate
tert-butyl {1-[4-(difluoromethoxy)benzoyl]-5-methoxy-2-methyl-1H-indol-3-yl]acetate
cyclobutyl {1-[4-(difluoromethoxy)benzoyl]-5-methoxy-2-methyl-1H-indol-3-yl]acetate
cyclopentyl {1-[4-(difluoromethoxy)benzoyl]-5-methoxy-2-methyl-1H-indol-3-yl]acetate
2-{1-[4-(difluoromethoxy)benzoyl]-5-methoxy-2-methyl-1H-indol-3-yl]acetamide
2-{1-[4-(difluoromethoxy)benzoyl]-5-methoxy-2-methyl-1H-indol-3-yl]-N-methylacetamide
2-{1-[4-(difluoromethoxy)benzoyl]-5-methoxy-2-methyl-1H-indol-3-yl]-N-ethylacetamide
2-{1-[4-(difluoromethoxy)benzoyl]-5-methoxy-2-methyl-1H-indol-3-yl]-N-propylacetamide
2-{1-[4-(difluoromethoxy)benzoyl]-5-methoxy-2-methyl-1H-indol-3-yl]-N-isopropylacetamide
2-{1-[4-(difluoromethoxy)benzoyl]-5-methoxy-2-methyl-1H-indol-3-yl]-N-butylacetamide
2-{1-[4-(difluoromethoxy)benzoyl]-5-methoxy-2-methyl-1H-indol-3-yl]-N-sec-butylacetamide
2-{1-[4-(difluoromethoxy)benzoyl]-5-methoxy-2-methyl-1H-indol-3-yl]-N-(2-hydroxyethyl)acetamide
({{1-[4-(difluoromethoxy)benzoyl]-5-methoxy-2-methyl-1H-indol-3-yl]acetyl}amino)acetic acid
3-({{1-[4-(difluoromethoxy)benzoyl]-5-methoxy-2-methyl-1H-indol-3-yl]acetyl}amino)propanoic acid
3-({{1-[4-(difluoromethoxy)benzoyl]-5-methoxy-2-methyl-1H-indol-3-yl]acetyl}amino)-2-methylpropanoic acid
3-({{1-[4-(difluoromethoxy)benzoyl]-5-methoxy-2-methyl-1H-indol-3-yl]acetyl}amino)-3-methylpropanoic acid
2-({{1-[4-(difluoromethoxy)benzoyl]-5-methoxy-2-methyl-1H-indol-3-yl]acetyl}amino)propanoic acid
2-({{1-[4-(difluoromethoxy)benzoyl]-5-methoxy-2-methyl-1H-indol-3-yl]acetyl}amino)-2-methylpropanoic acid
1-[4-(difluoromethoxy)benzoyl]-5-hydroxy-2-methyl-1H-indol-3-yl]acetic acid,
methyl {1-[4-(difluoromethoxy)benzoyl]-5-hydroxy-2-methyl-1H-indol-3-yl]acetate
ethyl {1-[4-(difluoromethoxy)benzoyl]-5-hydroxy-2-methyl-1H-indol-3-yl]acetate
propyl {1-[4-(difluoromethoxy)benzoyl]-5-hydroxy-2-methyl-1H-indol-3-yl]acetate
butyl {1-[4-(difluoromethoxy)benzoyl]-5-hydroxy-2-methyl-1H-indol-3-yl]acetate
sec-butyl {1-[4-(difluoromethoxy)benzoyl]-5-hydroxy-2-methyl-1H-indol-3-yl]acetate
(R) sec-butyl {1-[4-(difluoromethoxy)benzoyl]-5-hydroxy-2-methyl-1H-indol-3-yl]acetate
(S) sec-butyl {1-[4-(difluoromethoxy)benzoyl]-5-hydroxy-2-methyl-1H-indol-3-yl]acetate
isopropyl {1-[4-(difluoromethoxy)benzoyl]-5-hydroxy-2-methyl-1H-indol-3-yl]acetate
tert-butyl {1-[4-(difluoromethoxy)benzoyl]-5-hydroxy-2-methyl-1H-indol-3-yl]acetate
cyclobutyl {1-[4-(difluoromethoxy)benzoyl]-5-hydroxy-2-methyl-1H-indol-3-yl]acetate
cyclopentyl {1-[4-(difluoromethoxy)benzoyl]-5-hydroxy-2-methyl-1H-indol-3-yl]acetate
2-{1-[4-(difluoromethoxy)benzoyl]-5-hydroxy-2-methyl-1H-indol-3-yl]acetamide
2-{1-[4-(difluoromethoxy)benzoyl]-5-hydroxy-2-methyl-1H-indol-3-yl]-N-methylacetamide
2-{1-[4-(difluoromethoxy)benzoyl]-5-hydroxy-2-methyl-1H-indol-3-yl]-N-ethylacetamide
2-{1-[4-(difluoromethoxy)benzoyl]-5-hydroxy-2-methyl-1H-indol-3-yl]-N-propylacetamide
2-{1-[4-(difluoromethoxy)benzoyl]-5-hydroxy-2-methyl-1H-indol-3-yl]-N-isopropylacetamide
2-{1-[4-(difluoromethoxy)benzoyl]-5-hydroxy-2-methyl-1H-indol-3-yl]-N-butylacetamide
2-{1-[4-(difluoromethoxy)benzoyl]-5-hydroxy-2-methyl-1H-indol-3-yl]-N-sec-butylacetamide
2-{1-[4-(difluoromethoxy)benzoyl]-5-hydroxy-2-methyl-1H-indol-3-yl]-N-(2-hydroxyethyl)acetamide
({{1-[4-(difluoromethoxy)benzoyl]-5-hydroxy-2-methyl-1H-indol-3-yl]acetyl}amino)acetic acid
3-({{1-[4-(difluoromethoxy)benzoyl]-5-hydroxy-2-methyl-1H-indol-3-yl]acetyl}amino)propanoic acid
3-({{1-[4-(difluoromethoxy)benzoyl]-5-hydroxy-2-methyl-1H-indol-3-yl]acetyl}amino)-2-methylpropanoic acid
3-({{1-[4-(difluoromethoxy)benzoyl]-5-hydroxy-2-methyl-1H-indol-3-yl]acetyl}amino)-3-methylpropanoic acid
2-({{1-[4-(difluoromethoxy)benzoyl]-5-hydroxy-2-methyl-1H-indol-3-yl]acetyl}amino)propanoic acid
2-({{1-[4-(difluoromethoxy)benzoyl]-5-hydroxy-2-methyl-1H-indol-3-yl]acetyl}amino)-2-methylpropanoic acid
1-[4-chlorobenzoyl]-6-chloro-5-methoxy-2-methyl-1H-indol-3-yl]acetic acid,
methyl {1-[4-chlorobenzoyl]-6-chloro-5-methoxy-2-methyl-1H-indol-3-yl]acetate
ethyl {1-[4-chlorobenzoyl]-6-chloro-5-methoxy-2-methyl-1H-indol-3-yl]acetate
propyl {1-[4-chlorobenzoyl]-6-chloro-5-methoxy-2-methyl-1H-indol-3-yl]acetate
butyl {[1-[4-chlorobenzoyl]-6-chloro-5-methoxy-2-methyl-1H-indol-3-yl]acetate
sec-butyl {1-[4-chlorobenzoyl]-6-chloro-5-methoxy-2-methyl-1H-indol-3-yl]acetate
(R) sec-butyl {1-[4-chlorobenzoyl]-6-chloro-5-methoxy-2-methyl-1H-indol-3-yl]acetate
(S) sec-butyl {1-[4-chlorobenzoyl]-6-chloro-5-methoxy-2-methyl-1H-indol-3-yl]acetate
isopropyl {1-[4-chlorobenzoyl]-6-chloro-5-methoxy-2-methyl-1H-indol-3-yl]acetate
tert-butyl {1-[4-chlorobenzoyl]-6-chloro-5-methoxy-2-methyl-1H-indol-3-yl]acetate
cyclobutyl {1-[4-chlorobenzoyl]-6-chloro-5-methoxy-2-methyl-1H-indol-3-yl]acetate
cyclopentyl {1-[4-chlorobenzoyl]-6-chloro-5-methoxy-2-methyl-1H-indol-3-yl]acetate
2-{1-[4-chlorobenzoyl]-6-chloro-5-methoxy-2-methyl-1H-indol-3-yl]acetamide
2-{1-[4-chlorobenzoyl]-6-chloro-5-methoxy-2-methyl-1H-indol-3-yl]-N-methylacetamide
2-{1-[4-chlorobenzoyl]-6-chloro-5-methoxy-2-methyl-1H-indol-3-yl]-N-ethylacetamide
2-{1-[4-chlorobenzoyl]-6-chloro-5-methoxy-2-methyl-1H-indol-3-yl]-N-propylacetamide
2-{1-[4-chlorobenzoyl]-6-chloro-5-methoxy-2-methyl-1H-indol-3-yl]-N-isopropylacetamide
2-{1-[4-chlorobenzoyl]-6-chloro-5-methoxy-2-methyl-1H-indol-3-yl]-N-butylacetamide
2-{1-[4-chlorobenzoyl]-6-chloro-5-methoxy-2-methyl-1H-indol-3-yl]-N-sec-butylacetamide
2-{1-[4-chlorobenzoyl]-6-chloro-5-methoxy-2-methyl-1H-indol-3-yl]-N-(2-hydroxyethyl)acetamide
({{1-[4-chlorobenzoyl]-6-chloro-5-methoxy-2-methyl-1H-indol-3-yl]acetyl}amino)acetic acid
3-({{1-[4-chlorobenzoyl]-6-chloro-5-methoxy-2-methyl-1H-indol-3-yl]acetyl}amino)propanoic acid
3-({{1-[4-chlorobenzoyl]-6-chloro-5-methoxy-2-methyl-1H-indol-3-yl]acetyl}amino)-2-methylpropanoic acid
3-({{1-[4-chlorobenzoyl]-6-chloro-5-methoxy-2-methyl-1H-indol-3-yl]acetyl}amino)-3-methylpropanoic acid
2-({{1-[4-chlorobenzoyl]-6-chloro-5-methoxy-2-methyl-1H-indol-3-yl]acetyl}amino)propanoic acid
2-({{1-[4-chlorobenzoyl]-6-chloro-5-methoxy-2-methyl-1H-indol-3-yl]acetyl}amino)-2-methylpropanoic acid
1-[4-chlorobenzoyl]-6-chloro-5-hydroxy-2-methyl-1H-indol-3-yl]acetic acid,
methyl {1-[4-chlorobenzoyl]-6-chloro-5-hydroxy-2-methyl-1H-indol-3-yl]acetate
ethyl {1-[4-chlorobenzoyl]-6-chloro-5-hydroxy-2-methyl-1H-indol-3-yl]acetate
propyl {1-[4-chlorobenzoyl]-6-chloro-5-hydroxy-2-methyl-1H-indol-3-yl]acetate
butyl {1-[4-chlorobenzoyl]-6-chloro-5-hydroxy-2-methyl-1H-indol-3-yl]acetate
sec-butyl {1-[4-chlorobenzoyl]-6-chloro-5-hydroxy-2-methyl-1H-indol-3-yl]acetate
(R) sec-butyl {1-[4-chlorobenzoyl]-6-chloro-5-hydroxy-2-methyl-1H-indol-3-yl]acetate
(S) sec-butyl {1-[4-chlorobenzoyl]-6-chloro-5-hydroxy-2-methyl-1H-indol-3-yl]acetate
isopropyl {1-[4-chlorobenzoyl]-6-chloro-5-hydroxy-2-methyl-1H-indol-3-yl]acetate
tert-butyl {1-[4-chlorobenzoyl]-6-chloro-5-hydroxy-2-methyl-1H-indol-3-yl]acetate
cyclobutyl {1-[4-chlorobenzoyl]-6-chloro-5-hydroxy-2-methyl-1H-indol-3-yl]acetate
cyclopentyl {1-[4-chlorobenzoyl]-6-chloro-5-hydroxy-2-methyl-1H-indol-3-yl]acetate
2-{1-[4-chlorobenzoyl]-6-chloro-5-hydroxy-2-methyl-1H-indol-3-yl]acetamide
2-{1-[4-chlorobenzoyl]-6-chloro-5-hydroxy-2-methyl-1H-indol-3-yl]-N-methylacetamide
2-{1-[4-chlorobenzoyl]-6-chloro-5-hydroxy-2-methyl-1H-indol-3-yl]-N-ethylacetamide
2-{1-[4-chlorobenzoyl]-6-chloro-5-hydroxy-2-methyl-1H-indol-3-yl]-N-propylacetamide
2-{1-[4-chlorobenzoyl]-6-chloro-5-hydroxy-2-methyl-1H-indol-3-yl]-N-isopropylacetamide
2-{1-[4-chlorobenzoyl]-6-chloro-5-hydroxy-2-methyl-1H-indol-3-yl]-N-butylacetamide
2-{1-[4-chlorobenzoyl]-6-chloro-5-hydroxy-2-methyl-1H-indol-3-yl]-N-sec-butylacetamide
2-{1-[4-chlorobenzoyl]-6-chloro-5-hydroxy-2-methyl-1H-indol-3-yl]-N-(2-hydroxyethyl)acetamide
({{1-[4-chlorobenzoyl]-6-chloro-5-hydroxy-2-methyl-1H-indol-3-yl]acetyl}amino)acetic acid
3-({{1-[4-chlorobenzoyl]-6-chloro-5-hydroxy-2-methyl-1H-indol-3-yl]acetyl}amino)propanoic acid
3-({{1-[4-chlorobenzoyl]-6-chloro-5-hydroxy-2-methyl-1H-indol-3-yl]acetyl}amino)-2-methylpropanoic acid
3-({{1-[4-chlorobenzoyl]-6-chloro-5-hydroxy-2-methyl-1H-indol-3-yl]acetyl}amino)-3-methylpropanoic acid
2-({{1-[4-chlorobenzoyl]-6-chloro-5-hydroxy-2-methyl-1H-indol-3-yl]acetyl}amino)propanoic acid
2-({{1-[4-chlorobenzoyl]-6-chloro-5-hydroxy-2-methyl-1H-indol-3-yl]acetyl}amino)-2-methylpropanoic acid
1-[4-chlorobenzoyl]-4-chloro-5-methoxy-2-methyl-1H-indol-3-yl]acetic acid,
methyl {1-[4-chlorobenzoyl]-4-chloro-5-methoxy-2-methyl-1H-indol-3-yl]acetate
ethyl {1-[4-chlorobenzoyl]-4-chloro-5-methoxy-2-methyl-1H-indol-3-yl]acetate
propyl {1-[4-chlorobenzoyl]-4-chloro-5-methoxy-2-methyl-1H-indol-3-yl]acetate
butyl {1-[4-chlorobenzoyl]-4-chloro-5-methoxy-2-methyl-1H-indol-3-yl]acetate
sec-butyl {1-[4-chlorobenzoyl]-4-chloro-5-methoxy-2-methyl-1H-indol-3-yl]acetate
(R) sec-butyl {1-[4-chlorobenzoyl]-4-chloro-5-methoxy-2-methyl-1H-indol-3-yl]acetate
(S) sec-butyl {1-[4-chlorobenzoyl]-4-chloro-5-methoxy-2-methyl-1H-indol-3-yl]acetate
isopropyl {1-[4-chlorobenzoyl]-4-chloro-5-methoxy-2-methyl-1H-indol-3-yl]acetate
tert-butyl {1-[4-chlorobenzoyl]-4-chloro-5-methoxy-2-methyl-1H-indol-3-yl]acetate
cyclobutyl {1-[4-chlorobenzoyl]-4-chloro-5-methoxy-2-methyl-1H-indol-3-yl]acetate
cyclopentyl {1-[4-chlorobenzoyl]-4-chloro-5-methoxy-2-methyl-1H-indol-3-yl]acetate
2-{1-[4-chlorobenzoyl]-4-chloro-5-methoxy-2-methyl-1H-indol-3-yl]acetamide
2-{1-[4-chlorobenzoyl]-4-chloro-5-methoxy-2-methyl-1H-indol-3-yl]-N-methylacetamide
2-{1-[4-chlorobenzoyl]-4-chloro-5-methoxy-2-methyl-1H-indol-3-yl]-N-ethylacetamide
2-{1-[4-chlorobenzoyl]-4-chloro-5-methoxy-2-methyl-1H-indol-3-yl]-N-propylacetamide
2-{1-[4-chlorobenzoyl]-4-chloro-5-methoxy-2-methyl-1H-indol-3-yl]-N-isopropylacetamide
2-{1-[4-chlorobenzoyl]-4-chloro-5-methoxy-2-methyl-1H-indol-3-yl]-N-butylacetamide
2-{1-[4-chlorobenzoyl]-4-chloro-5-methoxy-2-methyl-1H-indol-3-yl]-N-sec-butylacetamide
2-{1-[4-chlorobenzoyl]-4-chloro-5-methoxy-2-methyl-1H-indol-3-yl]-N-(2-hydroxyethyl)acetamide
({{1-[4-chlorobenzoyl]-4-chloro-5-methoxy-2-methyl-1H-indol-3-yl]acetyl}amino)acetic acid
3-({{1-[4-chlorobenzoyl]-4-chloro-5-methoxy-2-methyl-1H-indol-3-yl]acetyl}amino)propanoic acid
3-({{1-[4-chlorobenzoyl]-4-chloro-5-methoxy-2-methyl-1H-indol-3-yl]acetyl}amino)-2-methylpropanoic acid
3-({{1-[4-chlorobenzoyl]-4-chloro-5-methoxy-2-methyl-1H-indol-3-yl]acetyl}amino)-3-methylpropanoic acid
2-({{1-[4-chlorobenzoyl]-4-chloro-5-methoxy-2-methyl-1H-indol-3-yl]acetyl}amino)propanoic acid
2-({{1-[4-chlorobenzoyl]-4-chloro-5-methoxy-2-methyl-1H-indol-3-yl]acetyl}amino)-2-methylpropanoic acid
1-[4-chlorobenzoyl]-4-chloro-5-hydroxy-2-methyl-1H-indol-3-yl]acetic acid,
methyl {1-[4-chlorobenzoyl]-4-chloro-5-hydroxy-2-methyl-1H-indol-3-yl]acetate
ethyl {1-[4-chlorobenzoyl]-4-chloro-5-hydroxy-2-methyl-1H-indol-3-yl]acetate
propyl {1-[4-chlorobenzoyl]-4-chloro-5-hydroxy-2-methyl-1H-indol-3-yl]acetate
butyl {1-[4-chlorobenzoyl]-4-chloro-5-hydroxy-2-methyl-1H-indol-3-yl]acetate
sec-butyl {1-[4-chlorobenzoyl]-4-chloro-5-hydroxy-2-methyl-1H-indol-3-yl]acetate
(R) sec-butyl {1-[4-chlorobenzoyl]-4-chloro-5-hydroxy-2-methyl-1H-indol-3-yl]acetate
(S) sec-butyl {1-[4-chlorobenzoyl]-4-chloro-5-hydroxy-2-methyl-1H-indol-3-yl]acetate
isopropyl {1-[4-chlorobenzoyl]-4-chloro-5-hydroxy-2-methyl-1H-indol-3-yl]acetate
tert-butyl {1-[4-chlorobenzoyl]-4-chloro-5-hydroxy-2-methyl-1H-indol-3-yl]acetate
cyclobutyl {1-[4-chlorobenzoyl]-4-chloro-5-hydroxy-2-methyl-1H-indol-3-yl]acetate
cyclopentyl {1-[4-chlorobenzoyl]-4-chloro-5-hydroxy-2-methyl-1H-indol-3-yl]acetate
2-{1-[4-chlorobenzoyl]-4-chloro-5-hydroxy-2-methyl-1H-indol-3-yl]acetamide
2-{1-[4-chlorobenzoyl]-4-chloro-5-hydroxy-2-methyl-1H-indol-3-yl]-N-methylacetamide
2-{1-[4-chlorobenzoyl]-4-chloro-5-hydroxy-2-methyl-1H-indol-3-yl]-N-ethylacetamide
2-{1-[4-chlorobenzoyl]-4-chloro-5-hydroxy-2-methyl-1H-indol-3-yl]-N-propylacetamide
2-{1-[4-chlorobenzoyl]-4-chloro-5-hydroxy-2-methyl-1H-indol-3-yl]-N-isopropylacetamide
2-{1-[4-chlorobenzoyl]-4-chloro-5-hydroxy-2-methyl-1H-indol-3-yl]-N-butylacetamide
2-{1-[4-chlorobenzoyl]-4-chloro-5-hydroxy-2-methyl-1H-indol-3-yl]-N-sec-butylacetamide
2-{1-[4-chlorobenzoyl]-4-chloro-5-hydroxy-2-methyl-1H-indol-3-yl]-N-(2-hydroxyethyl)acetamide
({{1-[4-chlorobenzoyl]-4-chloro-5-hydroxy-2-methyl-1H-indol-3-yl]acetyl}amino)acetic acid
3-({{1-[4-chlorobenzoyl]-4-chloro-5-hydroxy-2-methyl-1H-indol-3-yl]acetyl}amino)propanoic acid
3-({{1-[4-chlorobenzoyl]-4-chloro-5-hydroxy-2-methyl-1H-indol-3-yl]acetyl}amino)-2-methylpropanoic acid
3-({{1-[4-chlorobenzoyl]-4-chloro-5-hydroxy-2-methyl-1H-indol-3-yl]acetyl}amino)-3-methylpropanoic acid
2-({{1-[4-chlorobenzoyl]-4-chloro-5-hydroxy-2-methyl-1H-indol-3-yl]acetyl}amino)propanoic acid
2-({{1-[4-chlorobenzoyl]-4-chloro-5-hydroxy-2-methyl-1H-indol-3-yl]acetyl}amino)-2-methylpropanoic acid
[1-(4-trifluoromethoxybenzoyl)-5-methoxy-2-methyl-1H-indol-3-yl]acetic acid,
methyl [1-(4-trifluoromethoxybenzoyl)-5-methoxy-2-methyl-1H-indol-3-yl]acetate
ethyl [1-(4-trifluoromethoxybenzoyl)-5-methoxy-2-methyl-1H-indol-3-yl]acetate
propyl [1-(4-trifluoromethoxybenzoyl)-5-methoxy-2-methyl-1H-indol-3-yl]acetate
butyl [1-(4-trifluoromethoxybenzoyl)-5-methoxy-2-methyl-1H-indol-3-yl]acetate
sec-butyl [1-(4-trifluoromethoxybenzoyl)-5-methoxy-2-methyl-1H-indol-3-yl]acetate
(R) sec-butyl [1-(4-trifluoromethoxybenzoyl)-5-methoxy-2-methyl-1H-indol-3-yl]acetate
(S) sec-butyl [1-(4-trifluoromethoxybenzoyl)-5-methoxy-2-methyl-1H-indol-3-yl]acetate
isopropyl [1-(4-trifluoromethoxybenzoyl)-5-methoxy-2-methyl-1H-indol-3-yl]acetate
tert-butyl [1-(4-trifluoromethoxybenzoyl)-5-methoxy-2-methyl-1H-indol-3-yl]acetate
cyclobutyl [1-(4-trifluoromethoxybenzoyl)-5-methoxy-2-methyl-1H-indol-3-yl]acetate
cyclopentyl [1-(4-trifluoromethoxybenzoyl)-5-methoxy-2-methyl-1H-indol-3-yl]acetate
2-[1-(4-trifluoromethoxybenzoyl)-5-methoxy-2-methyl-1H-indol-3-yl]acetamide
2-[1-(4-trifluoromethoxybenzoyl)-5-methoxy-2-methyl-1H-indol-3-yl]-N-methylacetamide
2-[1-(4-trifluoromethoxybenzoyl)-5-methoxy-2-methyl-1H-indol-3-yl]-N-ethylacetamide
2-[1-(4-trifluoromethoxybenzoyl)-5-methoxy-2-methyl-1H-indol-3-yl]-N-propylacetamide
2-[1-(4-trifluoromethoxybenzoyl)-5-methoxy-2-methyl-1H-indol-3-yl]-N-isopropylacetamide
2-[1-(4-trifluoromethoxybenzoyl)-5-methoxy-2-methyl-1H-indol-3-yl]-N-butylacetamide
2-[1-(4-trifluoromethoxybenzoyl)-5-methoxy-2-methyl-1H-indol-3-yl]-N-sec-butylacetamide
2-[1-(4-trifluoromethoxybenzoyl)-5-methoxy-2-methyl-1H-indol-3-yl]-N-(2-hydroxyethyl)acetamide
({[1-(4-trifluoromethoxybenzoyl)-5-methoxy-2-methyl-1H-indol-3-yl]acetyl}amino)acetic acid
3-({[1-(4-trifluoromethoxybenzoyl)-5-methoxy-2-methyl-1H-indol-3-yl]acetyl}amino)propanoic acid
3-({[1-(4-trifluoromethoxybenzoyl)-5-methoxy-2-methyl-1H-indol-3-yl]acetyl}amino)-2-methylpropanoic acid
3-({[1-(4-trifluoromethoxybenzoyl)-5-methoxy-2-methyl-1H-indol-3-yl]acetyl}amino)-3-methylpropanoic acid
2-({[1-(4-trifluoromethoxybenzoyl)-5-methoxy-2-methyl-1H-indol-3-yl]acetyl}amino)propanoic acid
2-({[1-(4-trifluoromethoxybenzoyl)-5-methoxy-2-methyl-1H-indol-3-yl]acetyl}amino)-2-methylpropanoic acid
[1-(4-trifluoromethoxybenzoyl)-5-methoxy-6-fluoro-2-methyl-1H-indol-3-yl]acetic acid,
methyl [1-(4-trifluoromethoxybenzoyl)-5-methoxy-6-fluoro-2-methyl-1H-indol-3-yl]acetate
ethyl [1-(4-trifluoromethoxybenzoyl)-5-methoxy-6-fluoro-2-methyl-1H-indol-3-yl]acetate
propyl [1-(4-trifluoromethoxybenzoyl)-5-methoxy-6-fluoro-2-methyl-1H-indol-3-yl]acetate
butyl [1-(4-trifluoromethoxybenzoyl)-5-methoxy-6-fluoro-2-methyl-1H-indol-3-yl]acetate
sec-butyl [1-(4-trifluoromethoxybenzoyl)-5-methoxy-6-fluoro-2-methyl-1H-indol-3-yl]acetate
(R) sec-butyl [1-(4-trifluoromethoxybenzoyl)-5-methoxy-6-fluoro-2-methyl-1H-indol-3-yl]acetate
(S) sec-butyl [1-(4-trifluoromethoxybenzoyl)-5-methoxy-6-fluoro-2-methyl-1H-indol-3-yl]acetate
isopropyl [1-(4-trifluoromethoxybenzoyl)-5-methoxy-6-fluoro-2-methyl-1H-indol-3-yl]acetate
tert-butyl [1-(4-trifluoromethoxybenzoyl)-5-methoxy-6-fluoro-2-methyl-1H-indol-3-yl]acetate
cyclobutyl [1-(4-trifluoromethoxybenzoyl)-5-methoxy-6-fluoro-2-methyl-1H-indol-3-yl]acetate
cyclopentyl [1-(4-trifluoromethoxybenzoyl)-5-methoxy-6-fluoro-2-methyl-1H-indol-3-yl]acetate
2-[1-(4-trifluoromethoxybenzoyl)-5-methoxy-6-fluoro-2-methyl-1H-indol-3-yl]acetamide
2-[1-(4-trifluoromethoxybenzoyl)-5-methoxy-6-fluoro-2-methyl-1H-indol-3-yl]-N-methylacetamide
2-[1-(4-trifluoromethoxybenzoyl)-5-methoxy-6-fluoro-2-methyl-1H-indol-3-yl]-N-ethylacetamide
2-[1-(4-trifluoromethoxybenzoyl)-5-methoxy-6-fluoro-2-methyl-1H-indol-3-yl]-N-propylacetamide
2-[1-(4-trifluoromethoxybenzoyl)-5-methoxy-6-fluoro-2-methyl-1H-indol-3-yl]-N-isopropylacetamide
2-[1-(4-trifluoromethoxybenzoyl)-5-methoxy-6-fluoro-2-methyl-1H-indol-3-yl]-N-butylacetamide
2-[1-(4-trifluoromethoxybenzoyl)-5-methoxy-6-fluoro-2-methyl-1H-indol-3-yl]-N-sec-butylacetamide
2-[1-(4-trifluoromethoxybenzoyl)-5-methoxy-6-fluoro-2-methyl 1H-indol-3-yl]-N-(2-hydroxyethyl)acetamide
({[1-(4-trifluoromethoxybenzoyl)-5-methoxy-6-fluoro-2-methyl-1H-indol-3-yl]acetyl}amino)acetic acid
3-({[1-(4-trifluoromethoxybenzoyl)-5-methoxy-6-fluoro-2-methyl-1H-indol-3-yl]acetyl}amino)propanoic acid
3-({[1-(4-trifluoromethoxybenzoyl)-5-methoxy-6-fluoro-2-methyl-1H-indol-3-yl]acetyl}amino)-2-methylpropanoic acid
3-({[1-(4-trifluoromethoxybenzoyl)-5-methoxy-6-fluoro-2-methyl-1H-indol-3-yl]acetyl}amino)-3-methylpropanoic acid
2-({[1-(4-trifluoromethoxybenzoyl)-5-methoxy-6-fluoro-2-methyl-1H-indol-3-yl]acetyl}amino)propanoic acid
2-({[1-(4-trifluoromethoxybenzoyl)-5-methoxy-6-fluoro-2-methyl-1H-indol-3-yl]acetyl}amino)-2-methylpropanoic acid
[1-(4-trifluoromethylbenzoyl)-5-hydroxy-6-fluoro-2-methyl-1H-indol-3-yl]acetic acid,
methyl [1-(4-trifluoromethylbenzoyl)-5-hydroxy-6-fluoro-2-methyl-1H-indol-3-yl]acetate
ethyl [1-(4-trifluoromethylbenzoyl)-5-hydroxy-6-fluoro-2-methyl-1H-indol-3-yl]acetate
propyl [1-(4-trifluoromethylbenzoyl)-5-hydroxy-6-fluoro-2-methyl-1H-indol-3-yl]acetate
butyl [1-(4-trifluoromethylbenzoyl)-5-hydroxy-6-fluoro-2-methyl-1H-indol-3-yl]acetate
sec-butyl [1-(4-trifluoromethylbenzoyl)-5-hydroxy-6-fluoro-2-methyl-1H-indol-3-yl]acetate
(R) sec-butyl [1-(4-trifluoromethylbenzoyl)-5-hydroxy-6-fluoro-2-methyl-1H-indol-3-yl]acetate
(S) sec-butyl [1-(4-trifluoromethylbenzoyl)-5-hydroxy-6-fluoro-2-methyl-1H-indol-3-yl]acetate
isopropyl [1-(4-trifluoromethylbenzoyl)-5-hydroxy-6-fluoro-2-methyl-1H-indol-3-yl]acetate
tert-butyl [1-(4-trifluoromethylbenzoyl)-5-hydroxy-6-fluoro-2-methyl-1H-indol-3-yl]acetate
cyclobutyl [1-(4-trifluoromethylbenzoyl)-5-hydroxy-6-fluoro-2-methyl-1H-indol-3-yl]acetate
cyclopentyl [1-(4-trifluoromethylbenzoyl)-5-hydroxy-6-fluoro-2-methyl-1H-indol-3-yl]acetate
2-[1-(4-trifluoromethylbenzoyl)-5-hydroxy-6-fluoro-2-methyl-1H-indol-3-yl]acetamide
2-[1-(4-trifluoromethylbenzoyl)-5-hydroxy-6-fluoro-2-methyl-1H-indol-3-yl]-N-methylacetamide
2-[1-(4-trifluoromethylbenzoyl)-5-hydroxy-6-fluoro-2-methyl-1H-indol-3-yl]-N-ethylacetamide
2-[1-(4-trifluoromethylbenzoyl)-5-hydroxy-6-fluoro-2-methyl-1H-indol-3-yl]-N-propylacetamide
2-[1-(4-trifluoromethylbenzoyl)-5-hydroxy-6-fluoro-2-methyl-1H-indol-3-yl]-N-isopropylacetamide
2-[1-(4-trifluoromethylbenzoyl)-5-hydroxy-6-fluoro-2-methyl-1H-indol-3-yl]-N-butylacetamide
2-[1-(4-trifluoromethylbenzoyl)-5-hydroxy-6-fluoro-2-methyl-1H-indol-3-yl]-N-sec-butylacetamide
2-[1-(4-trifluoromethylbenzoyl)-5-hydroxy-6-fluoro-2-methyl-1H-indol-3-yl]-N-(2-hydroxyethyl)acetamide
({[1-(4-trifluoromethylbenzoyl)-5-hydroxy-6-fluoro-2-methyl-1H-indol-3-yl]acetyl}amino)acetic acid
3-({[1-(4-trifluoromethylbenzoyl)-5-hydroxy-6-fluoro-2-methyl-1H-indol-3-yl]acetyl}amino)propanoic acid
3-({[1-(4-trifluoromethylbenzoyl)-5-hydroxy-6-fluoro-2-methyl-1H-indol-3-yl]acetyl}amino)-2-methylpropanoic acid
3-({[1-(4-trifluoromethylbenzoyl)-5-hydroxy-6-fluoro-2-methyl-1H-indol-3-yl]acetyl}amino)-3-methylpropanoic acid
2-({[1-(4-trifluoromethylbenzoyl)-5-hydroxy-6-fluoro-2-methyl-1H-indol-3-yl]acetyl}amino)propanoic acid
2-({[1-(4-trifluoromethylbenzoyl)-5-hydroxy-6-fluoro-2-methyl-1H-indol-3-yl]acetyl}amino)-2-methylpropanoic acid
[1-(4-trifluoromethylbenzoyl)-5-methoxy-6-fluoro-2-methyl-1H-indol-3-yl]acetic acid,
methyl [1-(4-trifluoromethylbenzoyl)-5-methoxy-6-fluoro-2-methyl-1H-indol-3-yl]acetate
ethyl [1-(4-trifluoromethylbenzoyl)-5-methoxy-6-fluoro-2-methyl-1H-indol-3-yl]acetate
propyl [1-(4-trifluoromethylbenzoyl)-5-methoxy-6-fluoro-2-methyl-1H-indol-3-yl]acetate
butyl [1-(4-trifluoromethylbenzoyl)-5-methoxy-6-fluoro-2-methyl-1H-indol-3-yl]acetate
sec-butyl [1-(4-trifluoromethylbenzoyl)-5-methoxy-6-fluoro-2-methyl-1H-indol-3-yl]acetate
(R) sec-butyl [1-(4-trifluoromethylbenzoyl)-5-methoxy-6-fluoro-2-methyl-1H-indol-3-yl]acetate
(S) sec-butyl [1-(4-trifluoromethylbenzoyl)-5-methoxy-6-fluoro-2-methyl-1H-indol-3-yl]acetate
isopropyl [1-(4-trifluoromethylbenzoyl)-5-methoxy-6-fluoro-2-methyl-1H-indol-3-yl]acetate
tert-butyl [1-(4-trifluoromethylbenzoyl)-5-methoxy-6-fluoro-2-methyl-1H-indol-3-yl]acetate
cyclobutyl [1-(4-trifluoromethylbenzoyl)-5-methoxy-6-fluoro-2-methyl-1H-indol-3-yl]acetate
cyclopentyl [1-(4-trifluoromethylbenzoyl)-5-methoxy-6-fluoro-2-methyl-1H-indol-3-yl]acetate
2-[1-(4-trifluoromethylbenzoyl)-5-methoxy-6-fluoro-2-methyl-1H-indol-3-yl]acetamide
2-[1-(4-trifluoromethylbenzoyl)-5-methoxy-6-fluoro-2-methyl-1H-indol-3-yl]-N-methylacetamide
2-[1-(4-trifluoromethylbenzoyl)-5-methoxy-6-fluoro-2-methyl-1H-indol-3-yl]-N-ethylacetamide
2-[1-(4-trifluoromethylbenzoyl)-5-methoxy-6-fluoro-2-methyl-1H-indol-3-yl]-N-propylacetamide
2-[1-(4-trifluoromethylbenzoyl)-5-methoxy-6-fluoro-2-methyl-1H-indol-3-yl]-N-isopropylacetamide
2-[1-(4-trifluoromethylbenzoyl)-5-methoxy-6-fluoro-2-methyl-1H-indol-3-yl]-N-butylacetamide
2-[1-(4-trifluoromethylbenzoyl)-5-methoxy-6-fluoro-2-methyl-1H-indol-3-yl]-N-sec-butylacetamide
2-[1-(4-trifluoromethylbenzoyl)-5-methoxy-6-fluoro-2-methyl-1H-indol-3-yl]-N-(2-hydroxyethyl)acetamide
({[1-(4-trifluoromethylbenzoyl)-5-methoxy-6-fluoro-2-methyl-1H-indol-3-yl]acetyl}amino)acetic acid
3-({[1-(4-trifluoromethylbenzoyl)-5-methoxy-6-fluoro-2-methyl-1H-indol-3-yl]acetyl}amino)propanoic acid
3-({[1-(4-trifluoromethylbenzoyl)-5-methoxy-6-fluoro-2-methyl-1H-indol-3-yl]acetyl}amino)-2-methylpropanoic acid
3-({[1-(4-trifluoromethylbenzoyl)-5-methoxy-6-fluoro-2-methyl-1H-indol-3-yl]acetyl}amino)-3-methylpropanoic acid
2-({[1-(4-trifluoromethylbenzoyl)-5-methoxy-6-fluoro-2-methyl-1H-indol-3-yl]acetyl}amino)propanoic acid
2-({[1-(4-trifluoromethylbenzoyl)-5-methoxy-6-fluoro-2-methyl-1H-indol-3-yl]acetyl}amino)-2-methylpropanoic acid
[1-(4-chlorobenzoyl)-5-hydroxy-6-fluoro-2-methyl-1H-indol-3-yl]acetic acid,
methyl [1-(4-chlorobenzoyl)-5-hydroxy-6-fluoro-2-methyl-1H-indol-3-yl]acetate
ethyl [1-(4-chlorobenzoyl)-5-hydroxy-6-fluoro-2-methyl-1H-indol-3-yl]acetate
propyl [1-(4-chlorobenzoyl)-5-hydroxy-6-fluoro-2-methyl-1H-indol-3-yl]acetate
butyl [1-(4-chlorobenzoyl)-5-hydroxy-6-fluoro-2-methyl-1H-indol-3-yl]acetate
sec-butyl [1-(4-chlorobenzoyl)-5-hydroxy-6-fluoro-2-methyl-1H-indol-3-yl]acetate
(R) sec-butyl [1-(4-chlorobenzoyl)-5-hydroxy-6-fluoro-2-methyl-1H-indol-3-yl]acetate
(S) sec-butyl [1-(4-chlorobenzoyl)-5-hydroxy-6-fluoro-2-methyl-1H-indol-3-yl]acetate
isopropyl [1-(4-chlorobenzoyl)-5-hydroxy-6-fluoro-2-methyl-1H-indol-3-yl]acetate
tert-butyl [1-(4-chlorobenzoyl)-5-hydroxy-6-fluoro-2-methyl-1H-indol-3-yl]acetate
cyclobutyl [1-(4-chlorobenzoyl)-5-hydroxy-6-fluoro-2-methyl-1H-indol-3-yl]acetate
cyclopentyl [1-(4-chlorobenzoyl)-5-hydroxy-6-fluoro-2-methyl-1H-indol-3-yl]acetate
2-[1-(4-chlorobenzoyl)-5-hydroxy-6-fluoro-2-methyl-1H-indol-3-yl]acetamide
2-[1-(4-chlorobenzoyl)-5-hydroxy-6-fluoro-2-methyl-1H-indol-3-yl]-N-methylacetamide
2-[1-(4-chlorobenzoyl)-5-hydroxy-6-fluoro-2-methyl-1H-indol-3-yl]-N-ethylacetamide
2-[1-(4-chlorobenzoyl)-5-hydroxy-6-fluoro-2-methyl-1H-indol-3-yl]-N-propylacetamide
2-[1-(4-chlorobenzoyl)-5-hydroxy-6-fluoro-2-methyl-1H-indol-3-yl]-N-isopropylacetamide
2-[1-(4-chlorobenzoyl)-5-hydroxy-6-fluoro-2-methyl-1H-indol-3-yl]-N-butylacetamide
2-[1-(4-chlorobenzoyl)-5-hydroxy-6-fluoro-2-methyl-1H-indol-3-yl]-N-sec-butylacetamide
2-[1-(4-chlorobenzoyl)-5-hydroxy-6-fluoro-2-methyl-1H-indol-3-yl]-N-(2-hydroxyethyl)acetamide
({[1-(4-chlorobenzoyl)-5-hydroxy-6-fluoro-2-methyl-1H-indol-3-yl]acetyl}amino)acetic acid
3-({[1-(4-chlorobenzoyl)-5-hydroxy-6-fluoro-2-methyl-1H-indol-3-yl]acetyl}amino)propanoic acid
3-({[1-(4-chlorobenzoyl)-5-hydroxy-6-fluoro-2-methyl-1H-indol-3-yl]acetyl}amino)-2-methylpropanoic acid
3-({[1-(4-chlorobenzoyl)-5-hydroxy-6-fluoro-2-methyl-1H-indol-3-yl]acetyl}amino)-3-methylpropanoic acid
2-({[1-(4-chlorobenzoyl)-5-hydroxy-6-fluoro-2-methyl-1H-indol-3-yl]acetyl}amino)propanoic acid
2-({[1-(4-chlorobenzoyl)-5-hydroxy-6-fluoro-2-methyl-1H-indol-3-yl]acetyl}amino)-2-methylpropanoic acid
[1-(4-fluorobenzoyl)-5-methoxy-6-fluoro-2-methyl-1H-indol-3-yl]acetic acid,
methyl [1-(4-fluorobenzoyl)-5-methoxy-6-fluoro-2-methyl-1H-indol-3-yl]acetate
ethyl [1-(4-fluorobenzoyl)-5-methoxy-6-fluoro-2-methyl-1H-indol-3-yl]acetate
propyl [1-(4-fluorobenzoyl)-5-methoxy-6-fluoro-2-methyl-1H-indol-3-yl]acetate
butyl [1-(4-fluorobenzoyl)-5-methoxy-6-fluoro-2-methyl-1H-indol-3-yl]acetate
sec-butyl [1-(4-fluorobenzoyl)-5-methoxy-6-fluoro-2-methyl-1H-indol-3-yl]acetate
(R) sec-butyl [1-(4-fluorobenzoyl)-5-methoxy-6-fluoro-2-methyl-1H-indol-3-yl]acetate
(S) sec-butyl [1-(4-fluorobenzoyl)-5-methoxy-6-fluoro-2-methyl-1H-indol-3-yl]acetate
isopropyl [1-(4-fluorobenzoyl)-5-methoxy-6-fluoro-2-methyl-1H-indol-3-yl]acetate
tert-butyl [1-(4-fluorobenzoyl)-5-methoxy-6-fluoro-2-methyl-1H-indol-3-yl]acetate
cyclobutyl [1-(4-fluorobenzoyl)-5-methoxy-6-fluoro-2-methyl-1H-indol-3-yl]acetate
cyclopentyl [1-(4-fluorobenzoyl)-5-methoxy-6-fluoro-2-methyl-1H-indol-3-yl]acetate
2-[1-(4-fluorobenzoyl)-5-methoxy-6-fluoro-2-methyl-1H-indol-3-yl]acetamide
2-[1-(4-fluorobenzoyl)-5-methoxy-6-fluoro-2-methyl-1H-indol-3-yl]-N-methylacetamide
2-[1-(4-fluorobenzoyl)-5-methoxy-6-fluoro-2-methyl-1H-indol-3-yl]-N-ethylacetamide
2-[1-(4-fluorobenzoyl)-5-methoxy-6-fluoro-2-methyl-1H-indol-3-yl]-N-propylacetamide
2-[1-(4-fluorobenzoyl)-5-methoxy-6-fluoro-2-methyl-1H-indol-3-yl]-N-isopropylacetamide
2-[1-(4-fluorobenzoyl)-5-methoxy-6-fluoro-2-methyl-1H-indol-3-yl]-N-butylacetamide
2-[1-(4-fluorobenzoyl)-5-methoxy-6-fluoro-2-methyl-1H-indol-3-yl]-N-sec-butylacetamide
2-[1-(4-fluorobenzoyl)-5-methoxy-6-fluoro-2-methyl-1H-indol-3-yl]-N-(2-hydroxyethyl)acetamide
({[1-(4-fluorobenzoyl)-5-methoxy-6-fluoro-2-methyl-1H-indol-3-yl]acetyl}amino)acetic acid
3-({[1-(4-fluorobenzoyl)-5-methoxy-6-fluoro-2-methyl-1H-indol-3-yl]acetyl}amino)propanoic acid
3-({[1-(4-fluorobenzoyl)-5-methoxy-6-fluoro-2-methyl-1H-indol-3-yl]acetyl}amino)-2-methylpropanoic acid
3-({[1-(4-fluorobenzoyl)-5-methoxy-6-fluoro-2-methyl-1H-indol-3-yl]acetyl amino)-3-methylpropanoic acid
2-({[1-(4-fluorobenzoyl)-5-methoxy-6-fluoro-2-methyl-1H-indol-3-yl]acetyl}amino)propanoic acid
2-({[1-(4-fluorobenzoyl)-5-methoxy-6-fluoro-2-methyl-1H-indol-3-yl]acetyl}amino)-2-methylpropanoic acid
[1-(4-fluorobenzoyl)-5-hydroxy-6-fluoro-2-methyl-1H-indol-3-yl]acetic acid,
methyl [1-(4-fluorobenzoyl)-5-hydroxy-6-fluoro-2-methyl-1H-indol-3-yl]acetate
ethyl [1-(4-fluorobenzoyl)-5-hydroxy-6-fluoro-2-methyl-1H-indol-3-yl]acetate
propyl [1-(4-fluorobenzoyl)-5-hydroxy-6-fluoro-2-methyl-1H-indol-3-yl]acetate
butyl [1-(4-fluorobenzoyl)-5-hydroxy-6-fluoro-2-methyl-1H-indol-3-yl]acetate
sec-butyl [1-(4-fluorobenzoyl)-5-hydroxy-6-fluoro-2-methyl-1H-indol-3-yl]acetate
(R) sec-butyl [1-(4-fluorobenzoyl)-5-hydroxy-6-fluoro-2-methyl-1H-indol-3-yl]acetate
(S) sec-butyl [1-(4-fluorobenzoyl)-5-hydroxy-6-fluoro-2-methyl-1H-indol-3-yl]acetate
isopropyl [1-(4-fluorobenzoyl)-5-hydroxy-6-fluoro-2-methyl-1H-indol-3-yl]acetate
tert-butyl [1-(4-fluorobenzoyl)-5-hydroxy-6-fluoro-2-methyl-1H-indol-3-yl]acetate
cyclobutyl [1-(4-fluorobenzoyl)-5-hydroxy-6-fluoro-2-methyl-1H-indol-3-yl]acetate
cyclopentyl [1-(4-fluorobenzoyl)-5-hydroxy-6-fluoro-2-methyl-1H-indol-3-yl]acetate
2-[1-(4-fluorobenzoyl)-5-hydroxy-6-fluoro-2-methyl-1H-indol-3-yl]acetamide
2-[1-(4-fluorobenzoyl)-5-hydroxy-6-fluoro-2-methyl-1H-indol-3-yl]-N-methylacetamide
2-[1-(4-fluorobenzoyl)-5-hydroxy-6-fluoro-2-methyl-1H-indol-3-yl]-N-ethylacetamide
2-[1-(4-fluorobenzoyl)-5-hydroxy-6-fluoro-2-methyl-1H-indol-3-yl]-N-propylacetamide
2-[1-(4-fluorobenzoyl)-5-hydroxy-6-fluoro-2-methyl-1H-indol-3-yl]-N-isopropylacetamide
2-[1-(4-fluorobenzoyl)-5-hydroxy-6-fluoro-2-methyl-1H-indol-3-yl]-N-butylacetamide
2-[1-(4-fluorobenzoyl)-5-hydroxy-6-fluoro-2-methyl-1H-indol-3-yl]-N-sec-butylacetamide
2-[1-(4-fluorobenzoyl)-5-hydroxy-6-fluoro-2-methyl-1H-indol-3-yl]-N-(2-hydroxyethyl)acetamide
({[1-(4-fluorobenzoyl)-5-hydroxy-6-fluoro-2-methyl-1H-indol-3-yl]acetyl}amino)acetic acid
3-({[1-(4-fluorobenzoyl)-5-hydroxy-6-fluoro-2-methyl-1H-indol-3-yl]acetyl}amino)propanoic acid
3-({[1-(4-fluorobenzoyl)-5-hydroxy-6-fluoro-2-methyl-1H-indol-3-yl]acetyl}amino)-2-methylpropanoic acid
3-({[1-(4-fluorobenzoyl)-5-hydroxy-6-fluoro-2-methyl-1H-indol-3-yl]acetyl}amino)-3-methylpropanoic acid
2-({[1-(4-fluorobenzoyl)-5-hydroxy-6-fluoro-2-methyl-1H-indol-3-yl]acetyl}amino)propanoic acid
2-({[1-(4-fluorobenzoyl)-5-hydroxy-6-fluoro-2-methyl-1H-indol-3-yl]acetyl}amino)-2-methylpropanoic acid
[1-(4-bromobenzoyl)-5-hydroxy-6-fluoro-2-methyl-1H-indol-3-yl]acetic acid,
methyl [1-(4-bromobenzoyl)-5-hydroxy-6-fluoro-2-methyl-1H-indol-3-yl]acetate
ethyl [1-(4-bromobenzoyl)-5-hydroxy-6-fluoro-2-methyl-1H-indol-3-yl]acetate
propyl [1-(4-bromobenzoyl)-5-hydroxy-6-fluoro-2-methyl-1H-indol-3-yl]acetate
butyl [1-(4-bromobenzoyl)-5-hydroxy-6-fluoro-2-methyl-1H-indol-3-yl]acetate
sec-butyl [1-(4-bromobenzoyl)-5-hydroxy-6-fluoro-2-methyl-1H-indol-3-yl]acetate
(R) sec-butyl [1-(4-bromobenzoyl)-5-hydroxy-6-fluoro-2-methyl-1H-indol-3-yl]acetate
(S) sec-butyl [1-(4-bromobenzoyl)-5-hydroxy-6-fluoro-2-methyl-1H-indol-3-yl]acetate
isopropyl [1-(4-bromobenzoyl)-5-hydroxy-6-fluoro-2-methyl-1H-indol-3-yl]acetate
tert-butyl [1-(4-bromobenzoyl)-5-hydroxy-6-fluoro-2-methyl-1H-indol-3-yl]acetate
cyclobutyl [1-(4-bromobenzoyl)-5-hydroxy-6-fluoro-2-methyl-1H-indol-3-yl]acetate
cyclopentyl [1-(4-bromobenzoyl)-5-hydroxy-6-fluoro-2-methyl-1H-indol-3-yl]acetate
2-[1-(4-bromobenzoyl)-5-hydroxy-6-fluoro-2-methyl-1H-indol-3-yl]acetamide
2-[1-(4-bromobenzoyl)-5-hydroxy-6-fluoro-2-methyl-1H-indol-3-yl]-N-methylacetamide
2-[1-(4-bromobenzoyl)-5-hydroxy-6-fluoro-2-methyl-1H-indol-3-yl]-N-ethylacetamide
2-[1-(4-bromobenzoyl)-5-hydroxy-6-fluoro-2-methyl-1H-indol-3-yl]-N-propylacetamide
2-[1-(4-bromobenzoyl)-5-hydroxy-6-fluoro-2-methyl-1H-indol-3-yl]-N-isopropylacetamide
2-[1-(4-bromobenzoyl)-5-hydroxy-6-fluoro-2-methyl-1H-indol-3-yl]-N-butylacetamide
2-[1-(4-bromobenzoyl)-5-hydroxy-6-fluoro-2-methyl-1H-indol-3-yl]-N-sec-butylacetamide
2-[1-(4-bromobenzoyl)-5-hydroxy-6-fluoro-2-methyl-1H-indol-3-yl]-N-(2-hydroxyethyl)acetamide
({[1-(4-bromobenzoyl)-5-hydroxy-6-fluoro-2-methyl-1H-indol-3-yl]acetyl}amino)acetic acid
3-({[1-(4-bromobenzoyl)-5-hydroxy-6-fluoro-2-methyl-1H-indol-3-yl]acetyl}amino)propanoic acid
3-({[1-(4-bromobenzoyl)-5-hydroxy-6-fluoro-2-methyl-1H-indol-3-yl]acetyl}amino)-2-methylpropanoic acid
3-({[1-(4-bromobenzoyl)-5-hydroxy-6-fluoro-2-methyl-1H-indol-3-yl]acetyl}amino)-3-methylpropanoic acid
2-({[1-(4-bromobenzoyl)-5-hydroxy-6-fluoro-2-methyl-1H-indol-3-yl]acetyl}amino)propanoic acid
2-({[1-(4-bromobenzoyl)-5-hydroxy-6-fluoro-2-methyl-1H-indol-3-yl]acetyl}amino)-2-methylpropanoic acid
[1-(4-bromobenzoyl)-5-methoxy-6-fluoro-2-methyl-1H-indol-3-yl]acetic acid,
methyl [1-(4-bromobenzoyl)-5-methoxy-6-fluoro-2-methyl-1H-indol-3-yl]acetate
ethyl [1-(4-bromobenzoyl)-5-methoxy-6-fluoro-2-methyl-1H-indol-3-yl]acetate
propyl [1-(4-bromobenzoyl)-5-methoxy-6-fluoro-2-methyl-1H-indol-3-yl]acetate
butyl [1-(4-bromobenzoyl)-5-methoxy-6-fluoro-2-methyl-1H-indol-3-yl]acetate
sec-butyl [1-(4-bromobenzoyl)-5-methoxy-6-fluoro-2-methyl-1H-indol-3-yl]acetate
(R) sec-butyl [1-(4-bromobenzoyl)-5-methoxy-6-fluoro-2-methyl-1H-indol-3-yl]acetate
(S) sec-butyl [1-(4-bromobenzoyl)-5-methoxy-6-fluoro-2-methyl-1H-indol-3-yl]acetate
isopropyl [1-(4-bromobenzoyl)-5-methoxy-6-fluoro-2-methyl-1H-indol-3-yl]acetate
tert-butyl [1-(4-bromobenzoyl)-5-methoxy-6-fluoro-2-methyl-1H-indol-3-yl]acetate
cyclobutyl [1-(4-bromobenzoyl)-5-methoxy-6-fluoro-2-methyl-1H-indol-3-yl]acetate
cyclopentyl [1-(4-bromobenzoyl)-5-methoxy-6-fluoro-2-methyl-1H-indol-3-yl]acetate
2-[1-(4-bromobenzoyl)-5-methoxy-6-fluoro-2-methyl-1H-indol-3-yl]acetamide
2-[1-(4-bromobenzoyl)-5-methoxy-6-fluoro-2-methyl-1H-indol-3-yl]-N-methylacetamide
2-[1-(4-bromobenzoyl)-5-methoxy-6-fluoro-2-methyl-1H-indol-3-yl]-N-ethylacetamide
2-[1-(4-bromobenzoyl)-5-methoxy-6-fluoro-2-methyl-1H-indol-3-yl]-N-propylacetamide
2-[1-(4-bromobenzoyl)-5-methoxy-6-fluoro-2-methyl-1H-indol-3-yl]-N-isopropylacetamide
2-[1-(4-bromobenzoyl)-5-methoxy-6-fluoro-2-methyl-1H-indol-3-yl]-N-butylacetamide
2-[1-(4-bromobenzoyl)-5-methoxy-6-fluoro-2-methyl-1H-indol-3-yl]-N-sec-butylacetamide
2-[1-(4-bromobenzoyl)-5-methoxy-6-fluoro-2-methyl-1H-indol-3-yl]-N-(2-hydroxyethyl)acetamide
({[1-(4-bromobenzoyl)-5-methoxy-6-fluoro-2-methyl-1H-indol-3-yl]acetyl}amino)acetic acid
3-({[1-(4-bromobenzoyl)-5-methoxy-6-fluoro-2-methyl-1H-indol-3-yl]acetyl}amino)propanoic acid
3-({[1-(4-bromobenzoyl)-5-methoxy-6-fluoro-2-methyl-1H-indol-3-yl]acetyl}amino)-2-methylpropanoic acid
3-({[1-(4-bromobenzoyl)-5-methoxy-6-fluoro-2-methyl-1H-indol-3-yl]acetyl}amino)-3-methylpropanoic acid
2-({[1-(4-bromobenzoyl)-5-methoxy-6-fluoro-2-methyl-1H-indol-3-yl]acetyl}amino)propanoic acid
2-({[1-(4-bromobenzoyl)-5-methoxy-6-fluoro-2-methyl-1H-indol-3-yl]acetyl}amino)-2-methylpropanoic acid
[1-(4-bromobenzoyl)-5-hydroxy-2-methyl-1H-indol-3-yl]acetic acid,
methyl [1-(4-bromobenzoyl)-5-hydroxy-2-methyl-1H-indol-3-yl]acetate
ethyl [1-(4-bromobenzoyl)-5-hydroxy-2-methyl-1H-indol-3-yl]acetate
propyl [1-(4-bromobenzoyl)-5-hydroxy-2-methyl-1H-indol-3-yl]acetate
butyl [1-(4-bromobenzoyl)-5-hydroxy-2-methyl-1H-indol-3-yl]acetate
sec-butyl [1-(4-bromobenzoyl)-5-hydroxy-2-methyl-1H-indol-3-yl]acetate
(R) sec-butyl [1-(4-bromobenzoyl)-5-hydroxy-2-methyl-1H-indol-3-yl]acetate
(S) sec-butyl [1-(4-bromobenzoyl)-5-hydroxy-2-methyl-1H-indol-3-yl]acetate
isopropyl [1-(4-bromobenzoyl)-5-hydroxy-2-methyl-1H-indol-3-yl]acetate
tert-butyl [1-(4-bromobenzoyl)-5-hydroxy-2-methyl-1H-indol-3-yl]acetate
cyclobutyl [1-(4-bromobenzoyl)-5-hydroxy-2-methyl-1H-indol-3-yl]acetate
cyclopentyl [1-(4-bromobenzoyl)-5-hydroxy-2-methyl-1H-indol-3-yl]acetate
2-[1-(4-bromobenzoyl)-5-hydroxy-2-methyl-1H-indol-3-yl]acetamide
2-[1-(4-bromobenzoyl)-5-hydroxy-2-methyl-1H-indol-3-yl]-N-methylacetamide
2-[1-(4-bromobenzoyl)-5-hydroxy-2-methyl-1H-indol-3-yl]-N-ethylacetamide
2-[1-(4-bromobenzoyl)-5-hydroxy-2-methyl-1H-indol-3-yl]-N-propylacetamide
2-[1-(4-bromobenzoyl)-5-hydroxy-2-methyl-1H-indol-3-yl]-N-isopropylacetamide
2-[1-(4-bromobenzoyl)-5-hydroxy-2-methyl-1H-indol-3-yl]-N-butylacetamide
2-[1-(4-bromobenzoyl)-5-hydroxy-2-methyl-1H-indol-3-yl]-N-sec-butylacetamide
2-[1-(4-bromobenzoyl)-5-hydroxy-2-methyl-1H-indol-3-yl]-N-(2-hydroxyethyl)acetamide
({[1-(4-bromobenzoyl)-5-hydroxy-2-methyl-1H-indol-3-yl]acetyl}amino)acetic acid
3-({[1-(4-bromobenzoyl)-5-hydroxy-2-methyl-1H-indol-3-yl]acetyl}amino)propanoic acid
3-({[1-(4-bromobenzoyl)-5-hydroxy-2-methyl-1H-indol-3-yl]acetyl}amino)-2-methylpropanoic acid
3-({[1-(4-bromobenzoyl)-5-hydroxy-2-methyl-1H-indol-3-yl]acetyl}amino)-3-methylpropanoic acid
2-({[1-(4-bromobenzoyl)-5-hydroxy-2-methyl-1H-indol-3-yl]acetyl}amino)propanoic acid
2-({[1-(4-bromobenzoyl)-5-hydroxy-2-methyl-1H-indol-3-yl]acetyl}amino)-2-methylpropanoic acid
[1-(4-methylbenzoyl)-5-methoxy-6-fluoro-2-methyl-1H-indol-3-yl]acetic acid,
methyl [1-(4-methylbenzoyl)-5-methoxy-6-fluoro-2-methyl-1H-indol-3-yl]acetate
ethyl [1-(4-methylbenzoyl)-5-methoxy-6-fluoro-2-methyl-1H-indol-3-yl]acetate
propyl [1-(4-methylbenzoyl)-5-methoxy-6-fluoro-2-methyl-1H-indol-3-yl]acetate
butyl [1-(4-methylbenzoyl)-5-methoxy-6-fluoro-2-methyl-1H-indol-3-yl]acetate
sec-butyl [1-(4-methylbenzoyl)-5-methoxy-6-fluoro-2-methyl-1H-indol-3-yl]acetate
(R) sec-butyl [1-(4-methylbenzoyl)-5-methoxy-6-fluoro-2-methyl-1H-indol-3-yl]acetate
(S) sec-butyl [1-(4-methylbenzoyl)-5-methoxy-6-fluoro-2-methyl-1H-indol-3-yl]acetate
isopropyl [1-(4-methylbenzoyl)-5-methoxy-6-fluoro-2-methyl-1H-indol-3-yl]acetate
tert-butyl [1-(4-methylbenzoyl)-5-methoxy-6-fluoro-2-methyl-1H-indol-3-yl]acetate
cyclobutyl [1-(4-methylbenzoyl)-5-methoxy-6-fluoro-2-methyl-1H-indol-3-yl]acetate
cyclopentyl [1-(4-methylbenzoyl)-5-methoxy-6-fluoro-2-methyl-1H-indol-3-yl]acetate
2-[1-(4-methylbenzoyl)-5-methoxy-6-fluoro-2-methyl-1H-indol-3-yl]acetamide
2-[1-(4-methylbenzoyl)-5-methoxy-6-fluoro-2-methyl-1H-indol-3-yl]-N-methylacetamide
2-[1-(4-methylbenzoyl)-5-methoxy-6-fluoro-2-methyl-1H-indol-3-yl]-N-ethylacetamide
2-[1-(4-methylbenzoyl)-5-methoxy-6-fluoro-2-methyl-1H-indol-3-yl]-N-propylacetamide
2-[1-(4-methylbenzoyl)-5-methoxy-6-fluoro-2-methyl-1H-indol-3-yl]-N-isopropylacetamide
2-[1-(4-methylbenzoyl)-5-methoxy-6-fluoro-2-methyl-1H-indol-3-yl]-N-butylacetamide
2-[1-(4-methylbenzoyl)-5-methoxy-6-fluoro-2-methyl-1H-indol-3-yl]-N-sec-butylacetamide
2-[1-(4-methylbenzoyl)-5-methoxy-6-fluoro-2-methyl-1H-indol-3-yl]-N-(2-hydroxyethyl)acetamide
({[1-(4-methylbenzoyl)-5-methoxy-6-fluoro-2-methyl-1H-indol-3-yl]acetyl}amino)acetic acid
3-({[1-(4-methylbenzoyl)-5-methoxy-6-fluoro-2-methyl-1H-indol-3-yl]acetyl}amino)propanoic acid
3-({[1-(4-methylbenzoyl)-5-methoxy-6-fluoro-2-methyl-1H-indol-3-yl]acetyl}amino)-2-methylpropanoic acid
3-({[1-(4-methylbenzoyl)-5-methoxy-6-fluoro-2-methyl-1H-indol-3-yl]acetyl}amino)-3-methylpropanoic acid
2-({[1-(4-methylbenzoyl)-5-methoxy-6-fluoro-2-methyl-1H-indol-3-yl]acetyl}amino)propanoic acid
2-({[1-(4-methylbenzoyl)-5-methoxy-6-fluoro-2-methyl-1H-indol-3-yl]acetyl}amino)-2-methylpropanoic acid
[1-(4-methylbenzoyl)-5-hydroxy-6-fluoro-2-methyl-1H-indol-3-yl]acetic acid,
methyl [1-(4-methylbenzoyl)-5-hydroxy-6-fluoro-2-methyl-1H-indol-3-yl]acetate
ethyl [1-(4-methylbenzoyl)-5-hydroxy-6-fluoro-2-methyl-1H-indol-3-yl]acetate
propyl [1-(4-methylbenzoyl)-5-hydroxy-6-fluoro-2-methyl-1H-indol-3-yl]acetate
butyl [1-(4-methylbenzoyl)-5-hydroxy-6-fluoro-2-methyl-1H-indol-3-yl]acetate
sec-butyl [1-(4-methylbenzoyl)-5-hydroxy-6-fluoro-2-methyl-1H-indol-3-yl]acetate
(R) sec-butyl [1-(4-methylbenzoyl)-5-hydroxy-6-fluoro-2-methyl-1H-indol-3-yl]acetate
(S) sec-butyl [1-(4-methylbenzoyl)-5-hydroxy-6-fluoro-2-methyl-1H-indol-3-yl]acetate
isopropyl [1-(4-methylbenzoyl)-5-hydroxy-6-fluoro-2-methyl-1H-indol-3-yl]acetate
tert-butyl [1-(4-methylbenzoyl)-5-hydroxy-6-fluoro-2-methyl-1H-indol-3-yl]acetate
cyclobutyl [1-(4-methylbenzoyl)-5-hydroxy-6-fluoro-2-methyl-1H-indol-3-yl]acetate
cyclopentyl [1-(4-methylbenzoyl)-5-hydroxy-6-fluoro-2-methyl-1H-indol-3-yl]acetate
2-[1-(4-methylbenzoyl)-5-hydroxy-6-fluoro-2-methyl-1H-indol-3-yl]acetamide
2-[1-(4-methylbenzoyl)-5-hydroxy-6-fluoro-2-methyl-1H-indol-3-yl]-N-methylacetamide
2-[1-(4-methylbenzoyl)-5-hydroxy-6-fluoro-2-methyl-1H-indol-3-yl]-N-ethylacetamide
2-[1-(4-methylbenzoyl)-5-hydroxy-6-fluoro-2-methyl-1H-indol-3-yl]-N-propylacetamide
2-[1-(4-methylbenzoyl)-5-hydroxy-6-fluoro-2-methyl-1H-indol-3-yl]-N-isopropylacetamide
2-[1-(4-methylbenzoyl)-5-hydroxy-6-fluoro-2-methyl-1H-indol-3-yl]-N-butylacetamide
2-[1-(4-methylbenzoyl)-5-hydroxy-6-fluoro-2-methyl-1H-indol-3-yl]-N-sec-butylacetamide
2-[1-(4-methylbenzoyl)-5-hydroxy-6-fluoro-2-methyl-1H-indol-3-yl]-N-(2-hydroxyethyl)acetamide
({[1-(4-methylbenzoyl)-5-hydroxy-6-fluoro-2-methyl-1H-indol-3-yl]acetyl}amino)acetic acid
3-({[1-(4-methylbenzoyl)-5-hydroxy-6-fluoro-2-methyl-1H-indol-3-yl]acetyl}amino)propanoic acid
3-({[1-(4-methylbenzoyl)-5-hydroxy-6-fluoro-2-methyl-1H-indol-3-yl]acetyl}amino)-2-methylpropanoic acid
3-({[1-(4-methylbenzoyl)-5-hydroxy-6-fluoro-2-methyl-1H-indol-3-yl]acetyl}amino)-3-methylpropanoic acid
2-({[1-(4-methylbenzoyl)-5-hydroxy-6-fluoro-2-methyl-1H-indol-3-yl]acetyl}amino)propanoic acid
2-({[1-(4-methylbenzoyl)-5-hydroxy-6-fluoro-2-methyl-1H-indol-3-yl]acetyl}amino)-2-methylpropanoic acid
[1-(4-methylthiobenzoyl)-5-methoxy-6-fluoro-2-methyl-1H-indol-3-yl]acetic acid,
methyl [1-(4-methylthiobenzoyl)-5-methoxy-6-fluoro-2-methyl-1H-indol-3-yl]acetate
ethyl [1-(4-methylthiobenzoyl)-5-methoxy-6-fluoro-2-methyl-1H-indol-3-yl]acetate
propyl [1-(4-methylthiobenzoyl)-5-methoxy-6-fluoro-2-methyl-1H-indol-3-yl]acetate
butyl [1-(4-methylthiobenzoyl)-5-methoxy-6-fluoro-2-methyl-1H-indol-3-yl]acetate
sec-butyl [1-(4-methylthiobenzoyl)-5-methoxy-6-fluoro-2-methyl-1H-indol-3-yl]acetate
(R) sec-butyl [1-(4-methylthiobenzoyl)-5-methoxy-6-fluoro-2-methyl-1H-indol-3-yl]acetate
(S) sec-butyl [1-(4-methylthiobenzoyl)-5-methoxy-6-fluoro-2-methyl-1H-indol-3-yl]acetate
isopropyl [1-(4-methylthiobenzoyl)-5-methoxy-6-fluoro-2-methyl-1H-indol-3-yl]acetate
tert-butyl [1-(4-methylthiobenzoyl)-5-methoxy-6-fluoro-2-methyl-1H-indol-3-yl]acetate
cyclobutyl [1-(4-methylthiobenzoyl)-5-methoxy-6-fluoro-2-methyl-1H-indol-3-yl]acetate
cyclopentyl [1-(4-methylthiobenzoyl)-5-methoxy-6-fluoro-2-methyl-1H-indol-3-yl]acetate
2-[1-(4-methylthiobenzoyl)-5-methoxy-6-fluoro-2-methyl-1H-indol-3-yl]acetamide
2-[1-(4-methylthiobenzoyl)-5-methoxy-6-fluoro-2-methyl-1H-indol-3-yl]-N-methylacetamide
2-[1-(4-methylthiobenzoyl)-5-methoxy-6-fluoro-2-methyl-1H-indol-3-yl]-N-ethylacetamide
2-[1-(4-methylthiobenzoyl)-5-methoxy-6-fluoro-2-methyl-1H-indol-3-yl]-N-propylacetamide
2-[1-(4-methylthiobenzoyl)-5-methoxy-6-fluoro-2-methyl-1H-indol-3-yl]-N-isopropylacetamide
2-[1-(4-methylthiobenzoyl)-5-methoxy-6-fluoro-2-methyl-1H-indol-3-yl]-N-butylacetamide
2-[1-(4-methylthiobenzoyl)-5-methoxy-6-fluoro-2-methyl-1H-indol-3-yl]-N-sec-butylacetamide
2-[1-(4-methylthiobenzoyl)-5-methoxy-6-fluoro-2-methyl-1H-indol-3-yl]-N-(2-hydroxyethyl)acetamide
({[1-(4-methylthiobenzoyl)-5-methoxy-6-fluoro-2-methyl-1H-indol-3-yl]acetyl}amino)acetic acid
3-({[1-(4-methylthiobenzoyl)-5-methoxy-6-fluoro-2-methyl-1H-indol-3-yl]acetyl}amino)propanoic acid
3-({[1-(4-methylthiobenzoyl)-5-methoxy-6-fluoro-2-methyl-1H-indol-3-yl]acetyl}amino)-2-methylpropanoic acid
3-({[1-(4-methylthiobenzoyl)-5-methoxy-6-fluoro-2-methyl-1H-indol-3-yl]acetyl}amino)-3-methylpropanoic acid
2-({[1-(4-methylthiobenzoyl)-5-methoxy-6-fluoro-2-methyl-1H-indol-3-yl]acetyl}amino)propanoic acid
2-({[1-(4-methylthiobenzoyl)-5-methoxy-6-fluoro-2-methyl-1H-indol-3-yl]acetyl}amino)-2-methylpropanoic acid
[1-(4-methylthiobenzoyl)-5-hydroxy-6-fluoro-2-methyl-1H-indol-3-yl]acetic acid,
methyl [1-(4-methylthiobenzoyl)-5-hydroxy-6-fluoro-2-methyl-1H-indol-3-yl]acetate
ethyl [1-(4-methylthiobenzoyl)-5-hydroxy-6-fluoro-2-methyl-1H-indol-3-yl]acetate
propyl [1-(4-methylthiobenzoyl)-5-hydroxy-6-fluoro-2-methyl-1H-indol-3-yl]acetate
butyl [1-(4-methylthiobenzoyl)-5-hydroxy-6-fluoro-2-methyl-1H-indol-3-yl]acetate
sec-butyl [1-(4-methylthiobenzoyl)-5-hydroxy-6-fluoro-2-methyl-1H-indol-3-yl]acetate
(R) sec-butyl [1-(4-methylthiobenzoyl)-5-hydroxy-6-fluoro-2-methyl-1H-indol-3-yl]acetate
(S) sec-butyl [1-(4-methylthiobenzoyl)-5-hydroxy-6-fluoro-2-methyl-1H-indol-3-yl]acetate
isopropyl [1-(4-methylthiobenzoyl)-5-hydroxy-6-fluoro-2-methyl-1H-indol-3-yl]acetate
tert-butyl [1-(4-methylthiobenzoyl)-5-hydroxy-6-fluoro-2-methyl-1H-indol-3-yl]acetate
cyclobutyl [1-(4-methylthiobenzoyl)-5-hydroxy-6-fluoro-2-methyl-1H-indol-3-yl]acetate
cyclopentyl [1-(4-methylthiobenzoyl)-5-hydroxy-6-fluoro-2-methyl-1H-indol-3-yl]acetate
2-[1-(4-methylthiobenzoyl)-5-hydroxy-6-fluoro-2-methyl-1H-indol-3-yl]acetamide
2-[1-(4-methylthiobenzoyl)-5-hydroxy-6-fluoro-2-methyl-1H-indol-3-yl]-N-methylacetamide
2-[1-(4-methylthiobenzoyl)-5-hydroxy-6-fluoro-2-methyl-1H-indol-3-yl]-N-ethylacetamide
2-[1-(4-methylthiobenzoyl)-5-hydroxy-6-fluoro-2-methyl-1H-indol-3-yl]-N-propylacetamide
2-[1-(4-methylthiobenzoyl)-5-hydroxy-6-fluoro-2-methyl-1H-indol-3-yl]-N-isopropylacetamide
2-[1-(4-methylthiobenzoyl)-5-hydroxy-6-fluoro-2-methyl-1H-indol-3-yl]-N-butylacetamide
2-[1-(4-methylthiobenzoyl)-5-hydroxy-6-fluoro-2-methyl-1H-indol-3-yl]-N-sec-butylacetamide
2-[1-(4-methylthiobenzoyl)-5-hydroxy-6-fluoro-2-methyl-1H-indol-3-yl]-N-(2-hydroxyethyl)acetamide
({[1-(4-methylthiobenzoyl)-5-hydroxy-6-fluoro-2-methyl-1H-indol-3-yl]acetyl}amino)acetic acid
3-({[1-(4-methylthiobenzoyl)-5-hydroxy-6-fluoro-2-methyl-1H-indol-3-yl]acetyl}amino)propanoic acid
3-({[1-(4-methylthiobenzoyl)-5-hydroxy-6-fluoro-2-methyl-1H-indol-3-yl]acetyl}amino)-2-methylpropanoic acid
3-({[1-(4-methylthiobenzoyl)-5-hydroxy-6-fluoro-2-methyl-1H-indol-3-yl]acetyl}amino)-3-methylpropanoic acid
2-({[1-(4-methylthiobenzoyl)-5-hydroxy-6-fluoro-2-methyl-1H-indol-3-yl]acetyl}amino)propanoic acid
2-({[1-(4-methylthiobenzoyl)-5-hydroxy-6-fluoro-2-methyl-1H-indol-3-yl]acetyl}amino)-2-methylpropanoic acid
[1-(4-chlorobenzoyl)-5-hydroxy-2-methyl-1H-indol-3-yl]acetic acid,
methyl [1-(4-chlorobenzoyl)-5-hydroxy-2-methyl-1H-indol-3-yl]acetate
ethyl [1-(4-chlorobenzoyl)-5-hydroxy-2-methyl-1H-indol-3-yl]acetate
propyl [1-(4-chlorobenzoyl)-5-hydroxy-2-methyl-1H-indol-3-yl]acetate
butyl [1-(4-chlorobenzoyl)-5-hydroxy-2-methyl-1H-indol-3-yl]acetate
sec-butyl [1-(4-chlorobenzoyl)-5-hydroxy-2-methyl-1H-indol-3-yl]acetate
(R) sec-butyl [1-(4-chlorobenzoyl)-5-hydroxy-2-methyl-1H-indol-3-yl]acetate
(S) sec-butyl [1-(4-chlorobenzoyl)-5-hydroxy-2-methyl-1H-indol-3-yl]acetate
isopropyl [1-(4-chlorobenzoyl)-5-hydroxy-2-methyl-1H-indol-3-yl]acetate
tert-butyl [1-(4-chlorobenzoyl)-5-hydroxy-2-methyl-1H-indol-3-yl]acetate
cyclobutyl [1-(4-chlorobenzoyl)-5-hydroxy-2-methyl-1H-indol-3-yl]acetate
cyclopentyl [1-(4-chlorobenzoyl)-5-hydroxy-2-methyl-1H-indol-3-yl]acetate
2-[1-(4-chlorobenzoyl)-5-hydroxy-2-methyl-1H-indol-3-yl]acetamide
2-[1-(4-chlorobenzoyl)-5-hydroxy-2-methyl-1H-indol-3-yl]-N-methylacetamide
2-[1-(4-chlorobenzoyl)-5-hydroxy-2-methyl-1H-indol-3-yl]-N-ethylacetamide
2-[1-(4-chlorobenzoyl)-5-hydroxy-2-methyl-1H-indol-3-yl]-N-propylacetamide
2-[1-(4-chlorobenzoyl)-5-hydroxy-2-methyl-1H-indol-3-yl]-N-isopropylacetamide
2-[1-(4-chlorobenzoyl)-5-hydroxy-2-methyl-1H-indol-3-yl]-N-butylacetamide
2-[1-(4-chlorobenzoyl)-5-hydroxy-2-methyl-1H-indol-3-yl]-N-sec-butylacetamide
2-[1-(4-chlorobenzoyl)-5-hydroxy-2-methyl-1H-indol-3-yl]-N-(2-hydroxyethyl)acetamide
({[1-(4-chlorobenzoyl)-5-hydroxy-2-methyl-1H-indol-3-yl]acetyl lamino)acetic acid
3-({[1-(4-chlorobenzoyl)-5-hydroxy-2-methyl-1H-indol-3-yl]acetyl}amino)propanoic acid
3-({[1-(4-chlorobenzoyl)-5-hydroxy-2-methyl-1H-indol-3-yl]acetyl}amino)-2-methylpropanoic acid
3-({[1-(4-chlorobenzoyl)-5-hydroxy-2-methyl-1H-indol-3-yl]acetyl}amino)-3-methylpropanoic acid
2-({[1-(4-chlorobenzoyl)-5-hydroxy-2-methyl-1H-indol-3-yl]acetyl}amino)propanoic acid
2-({[1-(4-chlorobenzoyl)-5-hydroxy-2-methyl-1H-indol-3-yl]acetyl}amino)-2-methylpropanoic acid
1-[4-fluorobenzoyl]-6-chloro-5-methoxy-2-methyl-1H-indol-3-yl]acetic acid,
methyl {1-[4-fluorobenzoyl]-6-chloro-5-methoxy-2-methyl-1H-indol-3-yl]acetate
ethyl {1-[4-fluorobenzoyl]-6-chloro-5-methoxy-2-methyl-1H-indol-3-yl]acetate
propyl {1-[4-fluorobenzoyl]-6-chloro-5-methoxy-2-methyl-1H-indol-3-yl]acetate
butyl {1-[4-fluorobenzoyl]-6-chloro-5-methoxy-2-methyl-1H-indol-3-yl]acetate
sec-butyl {1-[4-fluorobenzoyl]-6-chloro-5-methoxy-2-methyl-1H-indol-3-yl]acetate
(R) sec-butyl {1-[4-fluorobenzoyl]-6-chloro-5-methoxy-2-methyl-1H-indol-3-yl]acetate
(S) sec-butyl {1-[4-fluorobenzoyl]-6-chloro-5-methoxy-2-methyl-1H-indol-3-yl]acetate
isopropyl {1-[4-fluorobenzoyl]-6-chloro-5-methoxy-2-methyl-1H-indol-3-yl]acetate
tert-butyl {1-[4-fluorobenzoyl]-6-chloro-5-methoxy-2-methyl-1H-indol-3-yl]acetate
cyclobutyl {1-[4-fluorobenzoyl]-6-chloro-5-methoxy-2-methyl-1H-indol-3-yl]acetate
cyclopentyl {1-[4-fluorobenzoyl]-6-chloro-5-methoxy-2-methyl-1H-indol-3-yl]acetate
2-{1-[4-fluorobenzoyl]-6-chloro-5-methoxy-2-methyl-1H-indol-3-yl]acetamide
2-{1-[4-fluorobenzoyl]-6-chloro-5-methoxy-2-methyl-1H-indol-3-yl]-N-methylacetamide
2-{1-[4-fluorobenzoyl]-6-chloro-5-methoxy-2-methyl-1H-indol-3-yl]-N-ethylacetamide
2-{1-[4-fluorobenzoyl]-6-chloro-5-methoxy-2-methyl-1H-indol-3-yl]-N-propylacetamide
2-{1-[4-fluorobenzoyl]-6-chloro-5-methoxy-2-methyl-1H-indol-3-yl]-N-isopropylacetamide
2-{1-[4-fluorobenzoyl]-6-chloro-5-methoxy-2-methyl-1H-indol-3-yl]-N-butylacetamide
2-{1-[4-fluorobenzoyl]-6-chloro-5-methoxy-2-methyl-1H-indol-3-yl]-N-sec-butylacetamide
2-{1-[4-fluorobenzoyl]-6-chloro-5-methoxy-2-methyl-1H-indol-3-yl]-N-(2-hydroxyethyl)acetamide
({{1-[4-fluorobenzoyl]-6-chloro-5-methoxy-2-methyl-1H-indol-3-yl]acetyl}amino)acetic acid
3-({{1-[4-fluorobenzoyl]-6-chloro-5-methoxy-2-methyl-1H-indol-3-yl]acetyl}amino)propanoic acid
3-({{1-[4-fluorobenzoyl]-6-chloro-5-methoxy-2-methyl-1H-indol-3-yl]acetyl}amino)-2-methylpropanoic acid
3-({{1-[4-fluorobenzoyl]-6-chloro-5-methoxy-2-methyl-1H-indol-3-yl]acetyl}amino)-3-methylpropanoic acid
2-({{1-[4-fluorobenzoyl]-6-chloro-5-methoxy-2-methyl-1H-indol-3-yl]acetyl}amino)propanoic acid
2-({{1-[4-fluorobenzoyl]-6-chloro-5-methoxy-2-methyl-1H-indol-3-yl]acetyl}amino)-2-methylpropanoic acid
1-[4-fluorobenzoyl]-6-chloro-5-hydroxy-2-methyl-1H-indol-3-yl]acetic acid,
methyl {1-[4-fluorobenzoyl]-6-chloro-5-hydroxy-2-methyl-1H-indol-3-yl]acetate
ethyl {1-[4-fluorobenzoyl]-6-chloro-5-hydroxy-2-methyl-1H-indol-3-yl]acetate
propyl {1-[4-fluorobenzoyl]-6-chloro-5-hydroxy-2-methyl-1H-indol-3-yl]acetate
butyl {1-[4-fluorobenzoyl]-6-chloro-5-hydroxy-2-methyl-1H-indol-3-yl]acetate
sec-butyl {1-[4-fluorobenzoyl]-6-chloro-5-hydroxy-2-methyl-1H-indol-3-yl]acetate
(R) sec-butyl {1-[4-fluorobenzoyl]-6-chloro-5-hydroxy-2-methyl-1H-indol-3-yl]acetate
(S) sec-butyl {1-[4-fluorobenzoyl]-6-chloro-5-hydroxy-2-methyl-1H-indol-3-yl]acetate
isopropyl {1-[4-fluorobenzoyl]-6-chloro-5-hydroxy-2-methyl-1H-indol-3-yl]acetate
tert-butyl {1-[4-fluorobenzoyl]-6-chloro-5-hydroxy-2-methyl-1H-indol-3-yl]acetate
cyclobutyl {1-[4-fluorobenzoyl]-6-chloro-5-hydroxy-2-methyl-1H-indol-3-yl]acetate
cyclopentyl {1-[4-fluorobenzoyl]-6-chloro-5-hydroxy-2-methyl-1H-indol-3-yl]acetate
2-{1-[4-fluorobenzoyl]-6-chloro-5-hydroxy-2-methyl-1H-indol-3-yl]acetamide
2-{1-[4-fluorobenzoyl]-6-chloro-5-hydroxy-2-methyl-1H-indol-3-yl]-N-methylacetamide
2-{1-[4-fluorobenzoyl]-6-chloro-5-hydroxy-2-methyl-1H-indol-3-yl]-N-ethylacetamide
2-{1-[4-fluorobenzoyl]-6-chloro-5-hydroxy-2-methyl-1H-indol-3-yl]-N-propylacetamide
2-{1-[4-fluorobenzoyl]-6-chloro-5-hydroxy-2-methyl-1H-indol-3-yl]-N-isopropylacetamide
2-{1-[4-fluorobenzoyl]-6-chloro-5-hydroxy-2-methyl-1H-indol-3-yl]-N-butylacetamide
2-{1-[4-fluorobenzoyl]-6-chloro-5-hydroxy-2-methyl-1H-indol-3-yl]-N-sec-butylacetamide
2-{1-[4-fluorobenzoyl]-6-chloro-5-hydroxy-2-methyl-1H-indol-3-yl]-N-(2-hydroxyethyl)acetamide
({{1-[4-fluorobenzoyl]-6-chloro-5-hydroxy-2-methyl-1H-indol-3-yl]acetyl}amino)acetic acid
3-({{1-[4-fluorobenzoyl]-6-chloro-5-hydroxy-2-methyl-1H-indol-3-yl]acetyl}amino)propanoic acid
3-({{1-[4-fluorobenzoyl]-6-chloro-5-hydroxy-2-methyl-1H-indol-3-yl]acetyl}amino)-2-methylpropanoic acid
3-({{1-[4-fluorobenzoyl]-6-chloro-5-hydroxy-2-methyl-1H-indol-3-yl]acetyl}amino)-3-methylpropanoic acid
2-({{1-[4-fluorobenzoyl]-6-chloro-5-hydroxy-2-methyl-1H-indol-3-yl]acetyl}amino)propanoic acid
2-({{1-[4-fluorobenzoyl]-6-chloro-5-hydroxy-2-methyl-1H-indol-3-yl]acetyl}amino)-2-methylpropanoic acid
1-[4-bromobenzoyl]-6-chloro-5-methoxy-2-methyl-1H-indol-3-yl]acetic acid,
methyl {1-[4-bromobenzoyl]-6-chloro-5-methoxy-2-methyl-1H-indol-3-yl]acetate
ethyl {1-[4-bromobenzoyl]-6-chloro-5-methoxy-2-methyl-1H-indol-3-yl]acetate
propyl {1-[4-bromobenzoyl]-6-chloro-5-methoxy-2-methyl-1H-indol-3-yl]acetate
butyl {1-[4-bromobenzoyl]-6-chloro-5-methoxy-2-methyl-1H-indol-3-yl]acetate
sec-butyl {1-[4-bromobenzoyl]-6-chloro-5-methoxy-2-methyl-1H-indol-3-yl]acetate
(R) sec-butyl {1-[4-bromobenzoyl]-6-chloro-5-methoxy-2-methyl-1H-indol-3-yl]acetate
(S) sec-butyl {1-[4-bromobenzoyl]-6-chloro-5-methoxy-2-methyl-1H-indol-3-yl]acetate
isopropyl {1-[4-bromobenzoyl]-6-chloro-5-methoxy-2-methyl-1H-indol-3-yl]acetate
tert-butyl {1-[4-bromobenzoyl]-6-chloro-5-methoxy-2-methyl-1H-indol-3-yl]acetate
cyclobutyl {1-[4-bromobenzoyl]-6-chloro-5-methoxy-2-methyl-1H-indol-3-yl]acetate
cyclopentyl {1-[4-bromobenzoyl]-6-chloro-5-methoxy-2-methyl-1H-indol-3-yl]acetate
2-{1-[4-bromobenzoyl]-6-chloro-5-methoxy-2-methyl-1H-indol-3-yl]acetamide
2-{1-[4-bromobenzoyl]-6-chloro-5-methoxy-2-methyl-1H-indol-3-yl]-N-methylacetamide
2-{1-[4-bromobenzoyl]-6-chloro-5-methoxy-2-methyl-1H-indol-3-yl]-N-ethylacetamide
2-{1-[4-bromobenzoyl]-6-chloro-5-methoxy-2-methyl-1H-indol-3-yl]-N-propylacetamide
2-{1-[4-bromobenzoyl]-6-chloro-5-methoxy-2-methyl-1H-indol-3-yl]-N-isopropylacetamide
2-{1-[4-bromobenzoyl]-6-chloro-5-methoxy-2-methyl-1H-indol-3-yl]-N-butylacetamide
2-{1-[4-bromobenzoyl]-6-chloro-5-methoxy-2-methyl-1H-indol-3-yl]-N-sec-butylacetamide
2-{1-[4-bromobenzoyl]-6-chloro-5-methoxy-2-methyl-1H-indol-3-yl]-N-(2-hydroxyethyl)acetamide
({{1-[4-bromobenzoyl]-6-chloro-5-methoxy-2-methyl-1H-indol-3-yl]acetyl}amino)acetic acid
3-({{1-[4-bromobenzoyl]-6-chloro-5-methoxy-2-methyl-1H-indol-3-yl]acetyl}amino)propanoic acid
3-({{1-[4-bromobenzoyl]-6-chloro-5-methoxy-2-methyl-1H-indol-3-yl]acetyl}amino)-2-methylpropanoic acid
3-({{1-[4-bromobenzoyl]-6-chloro-5-methoxy-2-methyl-1H-indol-3-yl]acetyl}amino)-3-methylpropanoic acid
2-({{1-[4-bromobenzoyl]-6-chloro-5-methoxy-2-methyl-1H-indol-3-yl]acetyl}amino)propanoic acid
2-({{1-[4-bromobenzoyl]-6-chloro-5-methoxy-2-methyl-1H-indol-3-yl]acetyl}amino)-2-methylpropanoic acid
1-[4-bromobenzoyl]-6-chloro-5-hydroxy-2-methyl-1H-indol-3-yl]acetic acid,
methyl {1-[4-bromobenzoyl]-6-chloro-5-hydroxy-2-methyl-1H-indol-3-yl]acetate
ethyl {1-[4-bromobenzoyl]-6-chloro-5-hydroxy-2-methyl-1H-indol-3-yl]acetate
propyl {1-[4-bromobenzoyl]-6-chloro-5-hydroxy-2-methyl-1H-indol-3-yl]acetate
butyl {1-[4-bromobenzoyl]-6-chloro-5-hydroxy-2-methyl-1H-indol-3-yl]acetate
sec-butyl {1-[4-bromobenzoyl]-6-chloro-5-hydroxy-2-methyl-1H-indol-3-yl]acetate
(R) sec-butyl {1-[4-bromobenzoyl]-6-chloro-5-hydroxy-2-methyl-1H-indol-3-yl]acetate
(S) sec-butyl {1-[4-bromobenzoyl]-6-chloro-5-hydroxy-2-methyl-1H-indol-3-yl]acetate
isopropyl {1-[4-bromobenzoyl]-6-chloro-5-hydroxy-2-methyl-1H-indol-3-yl]acetate
tert-butyl {1-[4-bromobenzoyl]-6-chloro-5-hydroxy-2-methyl-1H-indol-3-yl]acetate
cyclobutyl {1-[4-bromobenzoyl]-6-chloro-5-hydroxy-2-methyl-1H-indol-3-yl]acetate
cyclopentyl {1-[4-bromobenzoyl]-6-chloro-5-hydroxy-2-methyl-1H-indol-3-yl]acetate
2-{1-[4-bromobenzoyl]-6-chloro-5-hydroxy-2-methyl-1H-indol-3-yl]acetamide
2-{1-[4-bromobenzoyl]-6-chloro-5-hydroxy-2-methyl-1H-indol-3-yl]-N-methylacetamide
2-{1-[4-bromobenzoyl]-6-chloro-5-hydroxy-2-methyl-1H-indol-3-yl]-N-ethylacetamide
2-{1-[4-bromobenzoyl]-6-chloro-5-hydroxy-2-methyl-1H-indol-3-yl]-N-propylacetamide
2-{1-[4-bromobenzoyl]-6-chloro-5-hydroxy-2-methyl-1H-indol-3-yl]-N-isopropylacetamide
2-{1-[4-bromobenzoyl]-6-chloro-5-hydroxy-2-methyl-1H-indol-3-yl]-N-butylacetamide
2-{1-[4-bromobenzoyl]-6-chloro-5-hydroxy-2-methyl-1H-indol-3-yl]-N-sec-butylacetamide
2-{1-[4-bromobenzoyl]-6-chloro-5-hydroxy-2-methyl-1H-indol-3-yl]-N-(2-hydroxyethyl)acetamide
({{1-[4-bromobenzoyl]-6-chloro-5-hydroxy-2-methyl-1H-indol-3-yl]acetyl}amino)acetic acid
3-({{1-[4-bromobenzoyl]-6-chloro-5-hydroxy-2-methyl-1H-indol-3-yl]acetyl}amino)propanoic acid
3-({{1-[4-bromobenzoyl]-6-chloro-5-hydroxy-2-methyl-1H-indol-3-yl]acetyl}amino)-2-methylpropanoic acid
3-({{1-[4-bromobenzoyl]-6-chloro-5-hydroxy-2-methyl-1H-indol-3-yl]acetyl}amino)-3-methylpropanoic acid
2-({{1-[4-bromobenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-trifluoromethylbenzoyl]-6-chloro-5-methoxy-2-methyl-1H-indol-3-yl]acetic acid,
methyl {1-[4-trifluoromethylbenzoyl]-6-chloro-5-methoxy-2-methyl-1H-indol-3-yl]acetate
ethyl {1-[4-trifluoromethylbenzoyl]-6-chloro-5-methoxy-2-methyl-1H-indol-3-yl]acetate
propyl {1-[4-trifluoromethylbenzoyl]-6-chloro-5-methoxy-2-methyl-1H-indol-3-yl]acetate
butyl {1-[4-trifluoromethylbenzoyl]-6-chloro-5-methoxy-2-methyl-1H-indol-3-yl]acetate
sec-butyl {1-[4-trifluoromethylbenzoyl]-6-chloro-5-methoxy-2-methyl-1H-indol-3-yl]acetate
(R) sec-butyl {1-[4-trifluoromethylbenzoyl]-6-chloro-5-methoxy-2-methyl-1H-indol-3-yl]acetate
(S) sec-butyl {1-[4-trifluoromethylbenzoyl]-6-chloro-5-methoxy-2-methyl-1H-indol-3-yl]acetate
isopropyl {1-[4-trifluoromethylbenzoyl]-6-chloro-5-methoxy-2-methyl-1H-indol-3-yl]acetate
tert-butyl {1-[4-trifluoromethylbenzoyl]-6-chloro-5-methoxy-2-methyl-1H-indol-3-yl]acetate
cyclobutyl {1-[4-trifluoromethylbenzoyl]-6-chloro-5-methoxy-2-methyl-1H-indol-3-yl]acetate
cyclopentyl {1-[4-trifluoromethylbenzoyl]-6-chloro-5-methoxy-2-methyl-1H-indol-3-yl]acetate
2-{1-[4-trifluoromethylbenzoyl]-6-chloro-5-methoxy-2-methyl-1H-indol-3-yl]acetamide
2-{1-[4-trifluoromethylbenzoyl]-6-chloro-5-methoxy-2-methyl-1H-indol-3-yl]-N-methylacetamide
2-{1-[4-trifluoromethylbenzoyl]-6-chloro-5-methoxy-2-methyl-1H-indol-3-yl]-N-ethylacetamide
2-{1-[4-trifluoromethylbenzoyl]-6-chloro-5-methoxy-2-methyl-1H-indol-3-yl]-N-propylacetamide
2-{1-[4-trifluoromethylbenzoyl]-6-chloro-5-methoxy-2-methyl-1H-indol-3-yl]-N-isopropylacetamide
2-{1-[4-trifluoromethylbenzoyl]-6-chloro-5-methoxy-2-methyl-1H-indol-3-yl]-N-butylacetamide
2-{1-[4-trifluoromethylbenzoyl]-6-chloro-5-methoxy-2-methyl-1H-indol-3-yl]-N-sec-butylacetamide
2-{1-[4-trifluoromethylbenzoyl]-6-chloro-5-methoxy-2-methyl-1H-indol-3-yl]-N-(2-hydroxyethyl)acetamide
({{1-[4-trifluoromethylbenzoyl]-6-chloro-5-methoxy-2-methyl-1H-indol-3-yl]acetyl}amino)acetic acid
3-({{1-[4-trifluoromethylbenzoyl]-6-chloro-5-methoxy-2-methyl-1H-indol-3-yl]acetyl}amino)propanoic acid
3-({{1-[4-trifluoromethylbenzoyl]-6-chloro-5-methoxy-2-methyl-1H-indol-3-yl]acetyl}amino)-2-methylpropanoic acid
3-({{1-[4-trifluoromethylbenzoyl]-6-chloro-5-methoxy-2-methyl-1H-indol-3-yl]acetyl}amino)-3-methylpropanoic acid
2-({{1-[4-trifluoromethylbenzoyl]-6-chloro-5-methoxy-2-methyl-1H-indol-3-yl]acetyl}amino)propanoic acid
2-({{1-[4-trifluoromethylbenzoyl]-6-chloro-5-methoxy-2-methyl-1H-indol-3-yl]acetyl}amino)-2-methylpropanoic acid
1-[4-trifluoromethylbenzoyl]-6-chloro-5-hydroxy-2-methyl-1H-indol-3-yl]acetic acid,
methyl {1-[4-trifluoromethylbenzoyl]-6-chloro-5-hydroxy-2-methyl-1H-indol-3-yl]acetate
ethyl {1-[4-trifluoromethylbenzoyl]-6-chloro-5-hydroxy-2-methyl-1H-indol-3-yl]acetate
propyl {1-[4-trifluoromethylbenzoyl]-6-chloro-5-hydroxy-2-methyl-1H-indol-3-yl]acetate
butyl {1-[4-trifluoromethylbenzoyl]-6-chloro-5-hydroxy-2-methyl-1H-indol-3-yl]acetate
sec-butyl {1-[4-trifluoromethylbenzoyl]-6-chloro-5-hydroxy-2-methyl-1H-indol-3-yl]acetate
(R) sec-butyl {1-[4-trifluoromethylbenzoyl]-6-chloro-5-hydroxy-2-methyl-1H-indol-3-yl]acetate
(S) sec-butyl {1-[4-trifluoromethylbenzoyl]-6-chloro-5-hydroxy-2-methyl-1H-indol-3-yl]acetate
isopropyl {1-[4-trifluoromethylbenzoyl]-6-chloro-5-hydroxy-2-methyl-1H-indol-3-yl]acetate
tert-butyl {1-[4-trifluoromethylbenzoyl]-6-chloro-5-hydroxy-2-methyl-1H-indol-3-yl]acetate
cyclobutyl {1-[4-trifluoromethylbenzoyl]-6-chloro-5-hydroxy-2-methyl-1H-indol-3-yl]acetate
cyclopentyl {1-[4-trifluoromethylbenzoyl]-6-chloro-5-hydroxy-2-methyl-1H-indol-3-yl]acetate
2-{1-[4-trifluoromethylbenzoyl]-6-chloro-5-hydroxy-2-methyl-1H-indol-3-yl]acetamide
2-{1-[4-trifluoromethylbenzoyl]-6-chloro-5-hydroxy-2-methyl-1H-indol-3-yl]-N-methylacetamide
2-{1-[4-trifluoromethylbenzoyl]-6-chloro-5-hydroxy-2-methyl-1H-indol-3-yl]-N-ethylacetamide
2-{1-[4-trifluoromethylbenzoyl]-6-chloro-5-hydroxy-2-methyl-1H-indol-3-yl]-N-propylacetamide
2-{1-[4-trifluoromethylbenzoyl]-6-chloro-5-hydroxy-2-methyl-1H-indol-3-yl]-N-isopropylacetamide
2-{1-[4-trifluoromethylbenzoyl]-6-chloro-5-hydroxy-2-methyl-1H-indol-3-yl]-N-butylacetamide
2-{1-[4-trifluoromethylbenzoyl]-6-chloro-5-hydroxy-2-methyl-1H-indol-3-yl]-N-sec-butylacetamide
2-{1-[4-trifluoromethylbenzoyl]-6-chloro-5-hydroxy-2-methyl-1H-indol-3-yl]-N-(2-hydroxyethyl)acetamide
({{1-[4-trifluoromethylbenzoyl]-6-chloro-5-hydroxy-2-methyl-1H-indol-3-yl]acetyl}amino)acetic acid
3-({{1-[4-trifluoromethylbenzoyl]-6-chloro-5-hydroxy-2-methyl-1H-indol-3-yl]acetyl}amino)propanoic acid
3-({{1-[4-trifluoromethylbenzoyl]-6-chloro-5-hydroxy-2-methyl-1H-indol-3-yl]acetyl}amino)-2-methylpropanoic acid
3-({{1-[4-trifluoromethylbenzoyl]-6-chloro-5-hydroxy-2-methyl-1H-indol-3-yl]acetyl}amino)-3-methylpropanoic acid
2-({{1-[4-trifluoromethylbenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-trifluoromethoxybenzoyl]-6-chloro-5-methoxy-2-methyl-1H-indol-3-yl]acetic acid,
methyl {1-[4-trifluoromethoxybenzoyl]-6-chloro-5-methoxy-2-methyl-1H-indol-3-yl]acetate
ethyl {1-[4-trifluoromethoxybenzoyl]-6-chloro-5-methoxy-2-methyl-1H-indol-3-yl]acetate
propyl {1-[4-trifluoromethoxybenzoyl]-6-chloro-5-methoxy-2-methyl-1H-indol-3-yl]acetate
butyl {1-[4-trifluoromethoxybenzoyl]-6-chloro-5-methoxy-2-methyl-1H-indol-3-yl]acetate
sec-butyl {1-[4-trifluoromethoxybenzoyl]-6-chloro-5-methoxy-2-methyl-1H-indol-3-yl]acetate
(R) sec-butyl {1-[4-trifluoromethoxybenzoyl]-6-chloro-5-methoxy-2-methyl-1H-indol-3-yl]acetate
(S) sec-butyl {1-[4-trifluoromethoxybenzoyl]-6-chloro-5-methoxy-2-methyl-1H-indol-3-yl]acetate
isopropyl {1-[4-trifluoromethoxybenzoyl]-6-chloro-5-methoxy-2-methyl-1H-indol-3-yl]acetate
tert-butyl {1-[4-trifluoromethoxybenzoyl]-6-chloro-5-methoxy-2-methyl-1H-indol-3-yl]acetate
cyclobutyl {1-[4-trifluoromethoxybenzoyl]-6-chloro-5-methoxy-2-methyl-1H-indol-3-yl]acetate
cyclopentyl {1-[4-trifluoromethoxybenzoyl]-6-chloro-5-methoxy-2-methyl-1H-indol-3-yl]acetate
2-{1-[4-trifluoromethoxybenzoyl]-6-chloro-5-methoxy-2-methyl-1H-indol-3-yl]acetamide
2-{1-[4-trifluoromethoxybenzoyl]-6-chloro-5-methoxy-2-methyl-1H-indol-3-yl]-N-methylacetamide
2-{1-[4-trifluoromethoxybenzoyl]-6-chloro-5-methoxy-2-methyl-1H-indol-3-yl]-N-ethylacetamide
2-{1-[4-trifluoromethoxybenzoyl]-6-chloro-5-methoxy-2-methyl-1H-indol-3-yl]-N-propylacetamide
2-{1-[4-trifluoromethoxybenzoyl]-6-chloro-5-methoxy-2-methyl-1H-indol-3-yl]-N-isopropylacetamide
2-{1-[4-trifluoromethoxybenzoyl]-6-chloro-5-methoxy-2-methyl-1H-indol-3-yl]-N-butylacetamide
2-{1-[4-trifluoromethoxybenzoyl]-6-chloro-5-methoxy-2-methyl-1H-indol-3-yl]-N-sec-butylacetamide
2-{1-[4-trifluoromethoxybenzoyl]-6-chloro-5-methoxy-2-methyl-1H-indol-3-yl]-N-(2-hydroxyethyl)acetamide
({{1-[4-trifluoromethoxybenzoyl]-6-chloro-5-methoxy-2-methyl-1H-indol-3-yl]acetyl}amino)acetic acid
3-({{1-[4-trifluoromethoxybenzoyl]-6-chloro-5-methoxy-2-methyl-1H-indol-3-yl]acetyl}amino)propanoic acid
3-({{1-[4-trifluoromethoxybenzoyl]-6-chloro-5-methoxy-2-methyl-1H-indol-3-yl]acetyl}amino)-2-methylpropanoic acid
3-({{1-[4-trifluoromethoxybenzoyl]-6-chloro-5-methoxy-2-methyl-1H-indol-3-yl]acetyl}amino)-3-methylpropanoic acid
2-({{1-[4-trifluoromethoxybenzoyl]-6-chloro-5-methoxy-2-methyl-1H-indol-3-yl]acetyl}amino)propanoic acid
2-({{1-[4-trifluoromethoxybenzoyl]-6-chloro-5-methoxy-2-methyl-1H-indol-3-yl]acetyl}amino)-2-methylpropanoic acid
1-[4-trifluoromethoxybenzoyl]-6-chloro-5-hydroxy-2-methyl-1H-indol-3-yl]acetic acid,
methyl {1-[4-trifluoromethoxybenzoyl]-6-chloro-5-hydroxy-2-methyl-1H-indol-3-yl]acetate
ethyl {1-[4-trifluoromethoxybenzoyl]-6-chloro-5-hydroxy-2-methyl-1H-indol-3-yl]acetate
propyl {1-[4-trifluoromethoxybenzoyl]-6-chloro-5-hydroxy-2-methyl-1H-indol-3-yl]acetate
butyl {1-[4-trifluoromethoxybenzoyl]-6-chloro-5-hydroxy-2-methyl-1H-indol-3-yl]acetate
sec-butyl {1-[4-trifluoromethoxybenzoyl]-6-chloro-5-hydroxy-2-methyl-1H-indol-3-yl]acetate
(R) sec-butyl {1-[4-trifluoromethoxybenzoyl]-6-chloro-5-hydroxy-2-methyl-1H-indol-3-yl]acetate
(S) sec-butyl {1-[4-trifluoromethoxybenzoyl]-6-chloro-5-hydroxy-2-methyl-1H-indol-3-yl]acetate
isopropyl {1-[4-trifluoromethoxybenzoyl]-6-chloro-5-hydroxy-2-methyl-1H-indol-3-yl]acetate
tert-butyl {1-[4-trifluoromethoxybenzoyl]-6-chloro-5-hydroxy-2-methyl-1H-indol-3-yl]acetate
cyclobutyl {1-[4-trifluoromethoxybenzoyl]-6-chloro-5-hydroxy-2-methyl-1H-indol-3-yl]acetate
cyclopentyl {1-[4-trifluoromethoxybenzoyl]-6-chloro-5-hydroxy-2-methyl-1H-indol-3-yl]acetate
2-{1-[4-trifluoromethoxybenzoyl]-6-chloro-5-hydroxy-2-methyl-1H-indol-3-yl]acetamide
2-{1-[4-trifluoromethoxybenzoyl]-6-chloro-5-hydroxy-2-methyl-1H-indol-3-yl]-N-methylacetamide
2-{1-[4-trifluoromethoxybenzoyl]-6-chloro-5-hydroxy-2-methyl-1H-indol-3-yl]-N-ethylacetamide
2-{1-[4-trifluoromethoxybenzoyl]-6-chloro-5-hydroxy-2-methyl-1H-indol-3-yl]-N-propylacetamide
2-{1-[4-trifluoromethoxybenzoyl]-6-chloro-5-hydroxy-2-methyl-1H-indol-3-yl]-N-isopropylacetamide
2-{1-[4-trifluoromethoxybenzoyl]-6-chloro-5-hydroxy-2-methyl-1H-indol-3-yl]-N-butylacetamide
2-{1-[4-trifluoromethoxybenzoyl]-6-chloro-5-hydroxy-2-methyl-1H-indol-3-yl]-N-sec-butylacetamide
2-{1-[4-trifluoromethoxybenzoyl]-6-chloro-5-hydroxy-2-methyl-1H-indol-3-yl]-N-(2-hydroxyethyl)acetamide
({{1-[4-trifluoromethoxybenzoyl]-6-chloro-5-hydroxy-2-methyl-1H-indol-3-yl]acetyl}amino)acetic acid
3-({{1-[4-trifluoromethoxybenzoyl]-6-chloro-5-hydroxy-2-methyl-1H-indol-3-yl]acetyl}amino)propanoic acid
3-({{1-[4-trifluoromethoxybenzoyl]-6-chloro-5-hydroxy-2-methyl-1H-indol-3-yl]acetyl}amino)-2-methylpropanoic acid
3-({{1-[4-trifluoromethoxybenzoyl]-6-chloro-5-hydroxy-2-methyl-1H-indol-3-yl]acetyl}amino)-3-methylpropanoic acid
2-({{1-[4-trifluoromethoxybenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-difluoromethoxybenzoyl]-6-chloro-5-methoxy-2-methyl-1H-indol-3-yl]acetic acid,
methyl {1-[4-difluoromethoxybenzoyl]-6-chloro-5-methoxy-2-methyl-1H-indol-3-yl]acetate
ethyl {1-[4-difluoromethoxybenzoyl]-6-chloro-5-methoxy-2-methyl-1H-indol-3-yl]acetate
propyl {1-[4-difluoromethoxybenzoyl]-6-chloro-5-methoxy-2-methyl-1H-indol-3-yl]acetate
butyl {1-[4-difluoromethoxybenzoyl]-6-chloro-5-methoxy-2-methyl-1H-indol-3-yl]acetate
sec-butyl {1-[4-difluoromethoxybenzoyl]-6-chloro-5-methoxy-2-methyl-1H-indol-3-yl]acetate
(R) sec-butyl {1-[4-difluoromethoxybenzoyl]-6-chloro-5-methoxy-2-methyl-1H-indol-3-yl]acetate
(S) sec-butyl {1-[4-difluoromethoxybenzoyl]-6-chloro-5-methoxy-2-methyl-1H-indol-3-yl]acetate
isopropyl {1-[4-difluoromethoxybenzoyl]-6-chloro-5-methoxy-2-methyl-1H-indol-3-yl]acetate
tert-butyl {1-[4-difluoromethoxybenzoyl]-6-chloro-5-methoxy-2-methyl-1H-indol-3-yl]acetate
cyclobutyl {1-[4-difluoromethoxybenzoyl]-6-chloro-5-methoxy-2-methyl-1H-indol-3-yl]acetate
cyclopentyl {1-[4-difluoromethoxybenzoyl]-6-chloro-5-methoxy-2-methyl-1H-indol-3-yl]acetate
2-{1-[4-difluoromethoxybenzoyl]-6-chloro-5-methoxy-2-methyl-1H-indol-3-yl]acetamide
2-{1-[4-difluoromethoxybenzoyl]-6-chloro-5-methoxy-2-methyl-1H-indol-3-yl]-N-methylacetamide
2-{1-[4-difluoromethoxybenzoyl]-6-chloro-5-methoxy-2-methyl-1H-indol-3-yl]-N-ethylacetamide
2-{1-[4-difluoromethoxybenzoyl]-6-chloro-5-methoxy-2-methyl-1H-indol-3-yl]-N-propylacetamide
2-{1-[4-difluoromethoxybenzoyl]-6-chloro-5-methoxy-2-methyl-1H-indol-3-yl]-N-isopropylacetamide
2-{1-[4-difluoromethoxybenzoyl]-6-chloro-5-methoxy-2-methyl-1H-indol-3-yl]-N-butylacetamide
2-{1-[4-difluoromethoxybenzoyl]-6-chloro-5-methoxy-2-methyl-1H-indol-3-yl]-N-sec-butylacetamide
2-{1-[4-difluoromethoxybenzoyl]-6-chloro-5-methoxy-2-methyl-1H-indol-3-yl]-N-(2-hydroxyethyl)acetamide
({{1-[4-difluoromethoxybenzoyl]-6-chloro-5-methoxy-2-methyl 1H-indol-3-yl]acetyl}amino)acetic acid
3-({{1-[4-difluoromethoxybenzoyl]-6-chloro-5-methoxy-2-methyl-1H-indol-3-yl]acetyl}amino)propanoic acid
3-({{1-[4-difluoromethoxybenzoyl]-6-chloro-5-methoxy-2-methyl-1H-indol-3-yl]acetyl}amino)-2-methylpropanoic acid
3-({{1-[4-difluoromethoxybenzoyl]-6-chloro-5-methoxy-2-methyl-1H-indol-3-yl]acetyl}amino)-3-methylpropanoic acid
2-({{1-[4-difluoromethoxybenzoyl]-6-chloro-5-methoxy-2-methyl-1H-indol-3-yl]acetyl}amino)propanoic acid
2-({{1-[4-difluoromethoxybenzoyl]-6-chloro-5-methoxy-2-methyl-1H-indol-3-yl]acetyl}amino)-2-methylpropanoic acid
1-[4-difluoromethoxybenzoyl]-6-chloro-5-hydroxy-2-methyl-1H-indol-3-yl]acetic acid,
methyl {1-[4-difluoromethoxybenzoyl]-6-chloro-5-hydroxy-2-methyl-1H-indol-3-yl]acetate
ethyl {1-[4-difluoromethoxybenzoyl]-6-chloro-5-hydroxy-2-methyl-1H-indol-3-yl]acetate
propyl {1-[4-difluoromethoxybenzoyl]-6-chloro-5-hydroxy-2-methyl-1H-indol-3-yl]acetate
butyl {1-[4-difluoromethoxybenzoyl]-6-chloro-5-hydroxy-2-methyl-1H-indol-3-yl]acetate
sec-butyl {1-[4-difluoromethoxybenzoyl]-6-chloro-5-hydroxy-2-methyl-1H-indol-3-yl]acetate
(R) sec-butyl {1-[4-difluoromethoxybenzoyl]-6-chloro-5-hydroxy-2-methyl-1H-indol-3-yl]acetate
(S) sec-butyl {1-[4-difluoromethoxybenzoyl]-6-chloro-5-hydroxy-2-methyl-1H-indol-3-yl]acetate
isopropyl {1-[4-difluoromethoxybenzoyl]-6-chloro-5-hydroxy-2-methyl-1H-indol-3-yl]acetate
tert-butyl {1-[4-difluoromethoxybenzoyl]-6-chloro-5-hydroxy-2-methyl-1H-indol-3-yl]acetate
cyclobutyl {1-[4-difluoromethoxybenzoyl]-6-chloro-5-hydroxy-2-methyl-1H-indol-3-yl]acetate
cyclopentyl {1-[4-difluoromethoxybenzoyl]-6-chloro-5-hydroxy-2-methyl-1H-indol-3-yl]acetate
2-{1-[4-difluoromethoxybenzoyl]-6-chloro-5-hydroxy-2-methyl-1H-indol-3-yl]acetamide
2-{1-[4-difluoromethoxybenzoyl]-6-chloro-5-hydroxy-2-methyl-1H-indol-3-yl]-N-methylacetamide
2-{1-[4-difluoromethoxybenzoyl]-6-chloro-5-hydroxy-2-methyl-1H-indol-3-yl]-N-ethylacetamide
2-{1-[4-difluoromethoxybenzoyl]-6-chloro-5-hydroxy-2-methyl-1H-indol-3-yl]-N-propylacetamide
2-{1-[4-difluoromethoxybenzoyl]-6-chloro-5-hydroxy-2-methyl-1H-indol-3-yl]-N-isopropylacetamide
2-{1-[4-difluoromethoxybenzoyl]-6-chloro-5-hydroxy-2-methyl-1H-indol-3-yl]-N-butylacetamide
2-{1-[4-difluoromethoxybenzoyl]-6-chloro-5-hydroxy-2-methyl-1H-indol-3-yl]-N-sec-butylacetamide
2-{1-[4-difluoromethoxybenzoyl]-6-chloro-5-hydroxy-2-methyl-1H-indol-3-yl]-N-(2-hydroxyethyl)acetamide
({{1-[4-difluoromethoxybenzoyl]-6-chloro-5-hydroxy-2-methyl-1H-indol-3-yl]acetyl}amino)acetic acid
3-({{1-[4-difluoromethoxybenzoyl]-6-chloro-5-hydroxy-2-methyl-1H-indol-3-yl]acetyl}amino)propanoic acid
3-({{1-[4-difluoromethoxybenzoyl]-6-chloro-5-hydroxy-2-methyl-1H-indol-3-yl]acetyl}amino)-2-methylpropanoic acid
3-({{1-[4-difluoromethoxybenzoyl]-6-chloro-5-hydroxy-2-methyl-1H-indol-3-yl]acetyl}amino)-3-methylpropanoic acid
2-({{1-[4-difluoromethoxybenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-methylthiobenzoyl]-6-chloro-5-methoxy-2-methyl-1H-indol-3-yl]acetic acid,
methyl {1-[4-methylthiobenzoyl]-6-chloro-5-methoxy-2-methyl-1H-indol-3-yl]acetate
ethyl {1-[4-methylthiobenzoyl]-6-chloro-5-methoxy-2-methyl-1H-indol-3-yl]acetate
propyl {1-[4-methylthiobenzoyl]-6-chloro-5-methoxy-2-methyl-1H-indol-3-yl]acetate
butyl {1-[4-methylthiobenzoyl]-6-chloro-5-methoxy-2-methyl-1H-indol-3-yl]acetate
sec-butyl {1-[4-methylthiobenzoyl]-6-chloro-5-methoxy-2-methyl-1H-indol-3-yl]acetate
(R) sec-butyl {1-[4-methylthiobenzoyl]-6-chloro-5-methoxy-2-methyl-1H-indol-3-yl]acetate
(S) sec-butyl {1-[4-methylthiobenzoyl]-6-chloro-5-methoxy-2-methyl-1H-indol-3-yl]acetate
isopropyl {1-[4-methylthiobenzoyl]-6-chloro-5-methoxy-2-methyl-1H-indol-3-yl]acetate
tert-butyl {1-[4-methylthiobenzoyl]-6-chloro-5-methoxy-2-methyl-1H-indol-3-yl]acetate
cyclobutyl {1-[4-methylthiobenzoyl]-6-chloro-5-methoxy-2-methyl-1H-indol-3-yl]acetate
cyclopentyl {1-[4-methylthiobenzoyl]-6-chloro-5-methoxy-2-methyl-1H-indol-3-yl]acetate
2-{1-[4-methylthiobenzoyl]-6-chloro-5-methoxy-2-methyl-1H-indol-3-yl]acetamide
2-{1-[4-methylthiobenzoyl]-6-chloro-5-methoxy-2-methyl-1H-indol-3-yl]-N-methylacetamide
2-{1-[4-methylthiobenzoyl]-6-chloro-5-methoxy-2-methyl-1H-indol-3-yl]-N-ethylacetamide
2-{1-[4-methylthiobenzoyl]-6-chloro-5-methoxy-2-methyl-1H-indol-3-yl]-N-propylacetamide
2-{1-[4-methylthiobenzoyl]-6-chloro-5-methoxy-2-methyl-1H-indol-3-yl]-N-isopropylacetamide
2-{1-[4-methylthiobenzoyl]-6-chloro-5-methoxy-2-methyl-1H-indol-3-yl]-N-butylacetamide
2-{1-[4-methylthiobenzoyl]-6-chloro-5-methoxy-2-methyl-1H-indol-3-yl]-N-sec-butylacetamide
2-{1-[4-methylthiobenzoyl]-6-chloro-5-methoxy-2-methyl-1H-indol-3-yl]-N-(2-hydroxyethyl)acetamide
({{1-[4-methylthiobenzoyl]-6-chloro-5-methoxy-2-methyl-1H-indol-3-yl]acetyl}amino)acetic acid
3-({{1-[4-methylthiobenzoyl]-6-chloro-5-methoxy-2-methyl-1H-indol-3-yl]acetyl}amino)propanoic acid
3-({{1-[4-methylthiobenzoyl]-6-chloro-5-methoxy-2-methyl-1H-indol-3-yl]acetyl}amino)-2-methylpropanoic acid
3-({{1-[4-methylthiobenzoyl]-6-chloro-5-methoxy-2-methyl-1H-indol-3-yl]acetyl}amino)-3-methylpropanoic acid
2-({{1-[4-methylthiobenzoyl]-6-chloro-5-methoxy-2-methyl-1H-indol-3-yl]acetyl}amino)propanoic acid
2-({{1-[4-methylthiobenzoyl]-6-chloro-5-methoxy-2-methyl-1H-indol-3-yl]acetyl}amino)-2-methylpropanoic acid
1-[4-methylthiobenzoyl]-6-chloro-5-hydroxy-2-methyl-1H-indol-3-yl]acetic acid,
methyl {1-[4-methylthiobenzoyl]-6-chloro-5-hydroxy-2-methyl-1H-indol-3-yl]acetate
ethyl {1-[4-methylthiobenzoyl]-6-chloro-5-hydroxy-2-methyl-1H-indol-3-yl]acetate
propyl {1-[4-methylthiobenzoyl]-6-chloro-5-hydroxy-2-methyl-1H-indol-3-yl]acetate
butyl {1-[4-methylthiobenzoyl]-6-chloro-5-hydroxy-2-methyl-1H-indol-3-yl]acetate
sec-butyl {1-[4-methylthiobenzoyl]-6-chloro-5-hydroxy-2-methyl-1H-indol-3-yl]acetate
(R) sec-butyl {1-[4-methylthiobenzoyl]-6-chloro-5-hydroxy-2-methyl-1H-indol-3-yl]acetate
(S) sec-butyl {1-[4-methylthiobenzoyl]-6-chloro-5-hydroxy-2-methyl-1H-indol-3-yl]acetate
isopropyl {1-[4-methylthiobenzoyl]-6-chloro-5-hydroxy-2-methyl-1H-indol-3-yl]acetate
tert-butyl {1-[4-methylthiobenzoyl]-6-chloro-5-hydroxy-2-methyl-1H-indol-3-yl]acetate
cyclobutyl {1-[4-methylthiobenzoyl]-6-chloro-5-hydroxy-2-methyl-1H-indol-3-yl]acetate
cyclopentyl {1-[4-methylthiobenzoyl]-6-chloro-5-hydroxy-2-methyl-1H-indol-3-yl]acetate
2-{1-[4-methylthiobenzoyl]-6-chloro-5-hydroxy-2-methyl-1H-indol-3-yl]acetamide
2-{1-[4-methylthiobenzoyl]-6-chloro-5-hydroxy-2-methyl-1H-indol-3-yl]-N-methylacetamide
2-{1-[4-methylthiobenzoyl]-6-chloro-5-hydroxy-2-methyl-1H-indol-3-yl]-N-ethylacetamide
2-{1-[4-methylthiobenzoyl]-6-chloro-5-hydroxy-2-methyl-1H-indol-3-yl]-N-propylacetamide
2-{1-[4-methylthiobenzoyl]-6-chloro-5-hydroxy-2-methyl-1H-indol-3-yl]-N-isopropylacetamide
2-{1-[4-methylthiobenzoyl]-6-chloro-5-hydroxy-2-methyl-1H-indol-3-yl]-N-butylacetamide
2-{1-[4-methylthiobenzoyl]-6-chloro-5-hydroxy-2-methyl-1H-indol-3-yl]-N-sec-butylacetamide
2-{1-[4-methylthiobenzoyl]-6-chloro-5-hydroxy-2-methyl-1H-indol-3-yl]-N-(2-hydroxyethyl)acetamide
({{1-[4-methylthiobenzoyl]-6-chloro-5-hydroxy-2-methyl-1H-indol-3-yl]acetyl}amino)acetic acid
3-({{1-[4-methylthiobenzoyl]-6-chloro-5-hydroxy-2-methyl-1H-indol-3-yl]acetyl}amino)propanoic acid
3-({{1-[4-methylthiobenzoyl]-6-chloro-5-hydroxy-2-methyl-1H-indol-3-yl]acetyl}amino)-2-methylpropanoic acid
3-({{1-[4-methylthiobenzoyl]-6-chloro-5-hydroxy-2-methyl-1H-indol-3-yl]acetyl}amino)-3-methylpropanoic acid
2-({{1-[4-methylthiobenzoyl]-6-chloro-5-hydroxy-2-methyl-1H-indol-3-yl]acetyl}amino)propanoic acid
2-({{1-[4-methylthiobenzoyl]-6-chloro-5-hydroxy-2-methyl-1H-indol-3-yl]acetyl}amino)-2-methylpropanoic acid
[1-(4-methylbenzoyl)-5-methoxy-6-chloro-2-methyl-1H-indol-3-yl]acetic acid,
methyl [1-(4-methylbenzoyl)-5-methoxy-6-chloro-2-methyl-1H-indol-3-yl]acetate
ethyl [1-(4-methylbenzoyl)-5-methoxy-6-chloro-2-methyl-1H-indol-3-yl]acetate
propyl [1-(4-methylbenzoyl)-5-methoxy-6-chloro-2-methyl-1H-indol-3-yl]acetate
butyl [1-(4-methylbenzoyl)-5-methoxy-6-chloro-2-methyl-1H-indol-3-yl]acetate
sec-butyl [1-(4-methylbenzoyl)-5-methoxy-6-chloro-2-methyl-1H-indol-3-yl]acetate
(R) sec-butyl [1-(4-methylbenzoyl)-5-methoxy-6-chloro-2-methyl-1H-indol-3-yl]acetate
(S) sec-butyl [1-(4-methylbenzoyl)-5-methoxy-6-chloro-2-methyl-1H-indol-3-yl]acetate
isopropyl [1-(4-methylbenzoyl)-5-methoxy-6-chloro-2-methyl-1H-indol-3-yl]acetate
tert-butyl [1-(4-methylbenzoyl)-5-methoxy-6-chloro-2-methyl-1H-indol-3-yl]acetate
cyclobutyl [1-(4-methylbenzoyl)-5-methoxy-6-chloro-2-methyl-1H-indol-3-yl]acetate
cyclopentyl [1-(4-methylbenzoyl)-5-methoxy-6-chloro-2-methyl-1H-indol-3-yl]acetate
2-[1-(4-methylbenzoyl)-5-methoxy-6-chloro-2-methyl-1H-indol-3-yl]acetamide
2-[1-(4-methylbenzoyl)-5-methoxy-6-chloro-2-methyl-1H-indol-3-yl]-N-methylacetamide
2-[1-(4-methylbenzoyl)-5-methoxy-6-chloro-2-methyl-1H-indol-3-yl]-N-ethylacetamide
2-[1-(4-methylbenzoyl)-5-methoxy-6-chloro-2-methyl-1H-indol-3-yl]-N-propylacetamide
2-[1-(4-methylbenzoyl)-5-methoxy-6-chloro-2-methyl-1H-indol-3-yl]-N-isopropylacetamide
2-[1-(4-methylbenzoyl)-5-methoxy-6-chloro-2-methyl-1H-indol-3-yl]-N-butylacetamide
2-[1-(4-methylbenzoyl)-5-methoxy-6-chloro-2-methyl-1H-indol-3-yl]-N-sec-butylacetamide
2-[1-(4-methylbenzoyl)-5-methoxy-6-chloro-2-methyl-1H-indol-3-yl]-N-(2-hydroxyethyl)acetamide
({[1-(4-methylbenzoyl)-5-methoxy-6-chloro-2-methyl-1H-indol-3-yl]acetyl}amino)acetic acid
3-({[1-(4-methylbenzoyl)-5-methoxy-6-chloro-2-methyl 1H-indol-3-yl]acetyl}amino)propanoic acid
3-({[1-(4-methylbenzoyl)-5-methoxy-6-chloro-2-methyl-1H-indol-3-yl]acetyl}amino)-2-methylpropanoic acid
3-({[1-(4-methylbenzoyl)-5-methoxy-6-chloro-2-methyl-1H-indol-3-yl]acetyl}amino)-3-methylpropanoic acid
2-({[1-(4-methylbenzoyl)-5-methoxy-6-chloro-2-methyl-1H-indol-3-yl]acetyl}amino)propanoic acid
2-({[1-(4-methylbenzoyl)-5-methoxy-6-chloro-2-methyl-1H-indol-3-yl]acetyl}amino)-2-methylpropanoic acid
[1-(4-methylbenzoyl)-5-hydroxy-6-chloro-2-methyl-1H-indol-3-yl]acetic acid,
methyl [1-(4-methylbenzoyl)-5-hydroxy-6-chloro-2-methyl-1H-indol-3-yl]acetate
ethyl [1-(4-methylbenzoyl)-5-hydroxy-6-chloro-2-methyl-1H-indol-3-yl]acetate
propyl [1-(4-methylbenzoyl)-5-hydroxy-6-chloro-2-methyl-1H-indol-3-yl]acetate
butyl [1-(4-methylbenzoyl)-5-hydroxy-6-chloro-2-methyl-1H-indol-3-yl]acetate
sec-butyl [1-(4-methylbenzoyl)-5-hydroxy-6-chloro-2-methyl-1H-indol-3-yl]acetate
(R) sec-butyl [1-(4-methylbenzoyl)-5-hydroxy-6-chloro-2-methyl-1H-indol-3-yl]acetate
(S) sec-butyl [1-(4-methylbenzoyl)-5-hydroxy-6-chloro-2-methyl-1H-indol-3-yl]acetate
isopropyl [1-(4-methylbenzoyl)-5-hydroxy-6-chloro-2-methyl-1H-indol-3-yl]acetate
tert-butyl [1-(4-methylbenzoyl)-5-hydroxy-6-chloro-2-methyl-1H-indol-3-yl]acetate
cyclobutyl [1-(4-methylbenzoyl)-5-hydroxy-6-chloro-2-methyl-1H-indol-3-yl]acetate
cyclopentyl [1-(4-methylbenzoyl)-5-hydroxy-6-chloro-2-methyl-1H-indol-3-yl]acetate
2-[1-(4-methylbenzoyl)-5-hydroxy-6-chloro-2-methyl-1H-indol-3-yl]acetamide
2-[1-(4-methylbenzoyl)-5-hydroxy-6-chloro-2-methyl-1H-indol-3-yl]-N-methylacetamide
2-[1-(4-methylbenzoyl)-5-hydroxy-6-chloro-2-methyl-1H-indol-3-yl]-N-ethylacetamide
2-[1-(4-methylbenzoyl)-5-hydroxy-6-chloro-2-methyl-1H-indol-3-yl]-N-propylacetamide
2-[1-(4-methylbenzoyl)-5-hydroxy-6-chloro-2-methyl-1H-indol-3-yl]-N-isopropylacetamide
2-[1-(4-methylbenzoyl)-5-hydroxy-6-chloro-2-methyl-1H-indol-3-yl]-N-butylacetamide
2-[1-(4-methylbenzoyl)-5-hydroxy-6-chloro-2-methyl-1H-indol-3-yl]-N-sec-butylacetamide
2-[1-(4-methylbenzoyl)-5-hydroxy-6-chloro-2-methyl-1H-indol-3-yl]-N-(2-hydroxyethyl)acetamide
({[1-(4-methylbenzoyl)-5-hydroxy-6-chloro-2-methyl-1H-indol-3-yl]acetyl}amino)acetic acid
3-({[1-(4-methylbenzoyl)-5-hydroxy-6-chloro-2-methyl-1H-indol-3-yl]acetyl}amino)propanoic acid
3-({[1-(4-methylbenzoyl)-5-hydroxy-6-chloro-2-methyl-1H-indol-3-yl]acetyl}amino)-2-methylpropanoic acid
3-({[1-(4-methylbenzoyl)-5-hydroxy-6-chloro-2-methyl-1H-indol-3-yl]acetyl}amino)-3-methylpropanoic acid
2-({[1-(4-methylbenzoyl)-5-hydroxy-6-chloro-2-methyl-1H-indol-3-yl]acetyl}amino)propanoic acid
2-({[1-(4-methylbenzoyl)-5-hydroxy-6-chloro-2-methyl-1H-indol-3-yl]acetyl}amino)-2-methylpropanoic acid
{5-methoxy-2-methyl-1-[4-(trifluoromethoxy)benzoyl]-1H-indol-3-yl}acetic acid
{6-chloro-5-methoxy-2-methyl-1-[4-(trifluoromethoxy)benzoyl]-1H-indol-3-yl}acetic acid.
{6-chloro-5-hydroxy-2-methyl-1-[4-(trifluoromethoxy)benzoyl]-1H-indol-3-yl}acetic acid
{5-hydroxy-2-methyl-1-[4-(trifluoromethoxy)benzoyl]-1H-indol-3-yl}acetic acid
{1-[4-(difluoromethoxy)benzoyl]-5-hydroxy-2-methyl-1H-indol-3-yl}acetic acid
{6-chloro-1-[4-(difluoromethoxy)benzoyl]-5-hydroxy-2-methyl-1H-indol-3-yl}acetic acid
{6-chloro-1-[4-(difluoromethoxy)benzoyl]-5-methoxy-2-methyl-1H-indol-3-yl}acetic acid
{1-[4-(difluoromethoxy)benzoyl]-5-methoxy-2-methyl-1H-indol-3-yl}acetic acid

The invention also features compositions comprising a compound having Formula I, wherein the composition contains no more than 0.0001%, 0.001%, 0.01%, 0.1%, 0.3%, 0.5%, 0.9%. 1.9%, 5.0%, or 10% by weight other compounds.

The invention also features a method of treating a disorder associated with unwanted COX-2 activity or unwanted FAAH activity or both unwanted COX-2 activity and unwanted FAAH activity. In certain embodiments, the method comprises providing a patient with a therapeutically effective serum concentration of a compound having Formula I in the absence of measurable serum indomethacin. In some embodiments of the method: the disorder is an inflammatory disorder; and R²O— is a hydroxy group or a group that is metabolized to a hydroxy group, i.e., R²O— is a prodrug of a hydroxy group. In certain embodiments R²O— is an alkoxy group that is not rapidly metabolically converted to a hydroxy group or is not significantly metabolically converted to a hydroxy group. In other embodiments, the method comprises administering to the patient an agent for the treatment of inflammation, pain or fever, e.g., a NSAID other than indomethacin.

The invention also features a compound having Formula I wherein the prodrug of a hydroxy moiety is selected from: (a) an ester having a C₁ to C₆ branched or straight chain alkyl group, (b) phosphate ester having C₁₋₆ branched or straight chain alkyl groups, (c) a carbamate having C₁ to C₆ branched or straight chain alkyl groups, and (d) a carbonate group having a C₁ to C₆ branched or straight chain alkyl group.

In some embodiments of the invention, the composition is administered to a patient that is not being treated with a non-selective NSAID, e.g., a patient that is not being treated with indomethacin.

In certain embodiments the compounds are administered in combination with a second compound useful for reducing inflammation or pain.

The subject can be a mammal, preferably a human. Identifying a subject in need of such treatment can be in the judgment of a subject or a health care professional and can be subjective (e.g., opinion) or objective (e.g., measurable by a test or diagnostic method).

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.05 mg/Kg to about 500 mg/Kg, alternatively from about 1 to about 50 mg/Kg. Effective doses will also vary depending on route of administration, as well as the possibility of co-usage with other agents.

The term “mammal” includes, for example, mice, hamsters, rats, cows, sheep, pigs, goats, and horses, monkeys, dogs (e.g., Canis familiaris), cats, rabbits, guinea pigs, and primates, including humans.

The term “prodrug” refers to compounds which are drug precursors which, following administration and absorption, release the drug in vivo through a metabolic process. Exemplary prodrugs include acyl amides of the amino compounds of this invention such as amides of alkanoic (C₁ to C₆)acids, amides of aryl acids (e.g., benzoic acid) and alkane (C₁ to C₆)dioic acids.

The invention includes prodrugs that are converted in vivo so that R²O— becomes a hydroxyl group. Thus, in the prodrug form of the compounds of the invention R²O— is a group that is converted to a hydroxyl group. For example, in a prodrug from of the compounds of the invention, R²O— can be a carbonate, ester, carbamate, or phosphate ester or a similar group.

Thus, R² can be, for example, 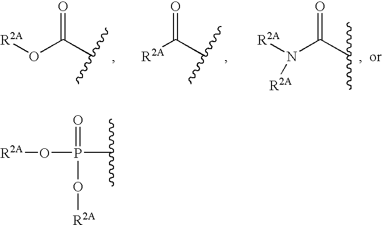
wherein each R^{2A} is independently: H or a C₁ to C₆ alkyl, alkenyl, alkynyl, aryl, cycloalkyl, or arylalkyl optionally independently substituted with one or more halogen.

Particularly useful are compound in which R^{2A} is selected from: H and a substituted or unsubstituted C₁ alkyl, a C₂ alkyl, a C₃ alkyl or a C₄ alkyl.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it (i.e., 1, 2, 3, 4, 5, 6, 7, 8, 9, 10, 11, 12). The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups.

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbornyl, and adamantyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents. The details of one or more embodiments of the invention are set forth in the accompanying drawings and the description below. Other features, objects, and advantages of the invention will be apparent from the description and drawings, and from the claims. The patents, patent applications, and publications referenced herein are hereby incorporated by reference in their entirety.

### DESCRIPTION OF DRAWINGS

FIG. 1 is a graph depicting the results of assays measuring the influence of indomethacin on COX-1 activity and COX-2 activity.

FIG. 2 is a graph depicting the results of assays measuring the influence of desmethylindomethacin on COX-1 activity and COX-2 activity.

FIG. 3 is a table that provides COX-1 IC₅₀ (purified enzyme assay), COX-2 IC₅₀ (purified enzyme assay), COX-1 IC₅₀ (human whole blood (HWB) assay), COX-2 IC₅₀ (human whole blood (HWB) assay), FAAH IC₅₀ (rat brain assay), FAAH IC₅₀ (human brain assay) and FAAH IC₅₀ (human MCF7 cell assay) for a number of compounds. All numbers are in μm units. Each assay was performed from 1-6 times. Most reported data points reflect the average of at least 2 repetitions of the assay.

### DETAILED DESCRIPTION

The invention features compounds that inhibit COX-2 and/or FAAH. The COX-2 inhibitors are selective COX-2 inhibitors in that they are selective for inhibition of COX-2 as compared to COX-1. Certain of the FAAH inhibitors are selective for inhibition of FAAH relative to both COX-2 and COX-1. Certain of the COX-2 inhibitors, in addition to being selective for COX-2 relative to COX-1, are selective for COX-2 relative to FAAH.

Certain compounds of the invention are expected to have an increased half-life in the human body compared to certain structurally related compounds. Certain compounds of the invention are expected to have reduced renal toxicity compared to certain structurally related compounds.

Useful selective COX-2 inhibitors are those which inhibit COX-2 activity at physiological concentrations where COX-1 activity is not significantly inhibited. Thus, the compounds have an IC₅₀ for COX-1 that is at least 2-, 5-, 10-, 15-, 20-, 100-, 500-, 1,000-fold greater than the IC₅₀ for COX-2. Particularly desirable are compounds that do not significantly inhibit COX-1 at a therapeutically effective concentration, e.g., a concentration effective to reduce pain or inflammation attributable to COX-2 associated prostaglandin production. Useful compounds include those having an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for COX-1 of greater than about 1, 5, 10, 15, 20, 40 or 100 μM. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 IC₅₀ for indomethacin in the same assay. In certain embodiments, the selectivity for COX-2 over COX-1 for a compound is greater than 3, 5, 10, 50, 100, 200, 500 or 1000 times the selectivity of indomethacin in the same assays.

Certain useful selective FAAH inhibitors include those which inhibit FAAH activity at a physiological concentration at which COX-1 and COX-2 activity is not significantly inhibited. Thus, the compounds have an IC₅₀ for COX-1 and COX-2 that is at least 2-, 5-, 10-, 15-, 20-, 100-, 500-, 1,000-fold greater than the IC₅₀ for FAAH. Particularly desirable are compounds that do not measurably inhibit COX-1 and COX-2 at a therapeutically effective concentration, e.g., a concentration effective to reduce pain. Useful compounds include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, and have an IC₅₀ for COX-1 and COX-2 of greater than about 1, 5, 10, 15, 20, 50, 100, 200, or 400 μM. In certain embodiments, the IC₅₀ for FAAH for a compound is no more than about 5, 1, 0.1, 0.05, 0.01 or 0.001 times the IC₅₀ for FAAH of indomethacin in the same assay.

Of course, other useful FAAH inhibitors also inhibit COX-2 at physiological concentrations at which COX-1 activity is not significantly inhibited. Particularly desirable are compounds that do not measurably inhibit COX-1 at a therapeutically effective concentration, e.g., a concentration effective to reduce pain. Useful compounds include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, an IC50 for COX-2 of less than about 2.0, 1.5, 1.0, 0.5, 0.4, 0.3, 0.2, 0.1, 0.08, 0.06, 0.04, 0.02, or 0.01 μM, and an IC₅₀ for COX-1 of greater than about 1, 5, 10, 15, or 20 μM. In certain embodiments the COX-2 IC₅₀ for such a FAAH inhibitor is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for such a FAAH inhibitor is at least 2, 5, 10, 25, 50, 100, 500, 1000 or more times the COX-1 IC₅₀ for indomethacin in the same assay.

Certain useful selective COX-2 inhibitors include those which inhibit COX-2 activity at physiological concentrations where FAAH activity is not significantly inhibited; Particularly desirable are compounds that do not significantly inhibit FAAH at a therapeutically effective concentration, e.g., a concentration effective to reduce pain. Useful compounds include those having an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for FAAH of greater than about 5, 10, 15, 20, 50, 100, 200 or 400 μM. Of course, other useful COX-2 inhibitors also inhibit FAAH at therapeutically relevant doses, i.e., they are not particularly selective for COX-2 over FAAH. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 IC₅₀ for indomethacin in the same assay.

Certain compounds having Formula I, e.g., those in which R⁶ is a C₁ to C₆ alkyl are effective FAAH inhibitors, but are not particularly effective COX-2 inhibitors. However, many such compounds are metabolized to a form in which R⁶ is H. Many of these metabolites are effective COX-2 inhibitors, but are not highly effective FAAH inhibitors, although they can inhibit FAAH to some extent. Thus, compounds having Formula I which are FAAH inhibitors and in which R⁶ is a C₁ to C₆ alkyl can exhibit two different phases of activity when administered to a patient—an initial, relatively high FAAH inhibition phase characterized by little or no significant COX-2 inhibition followed by a COX-2 inhibition phase characterized by reduced FAAH inhibition.

### EXAMPLES

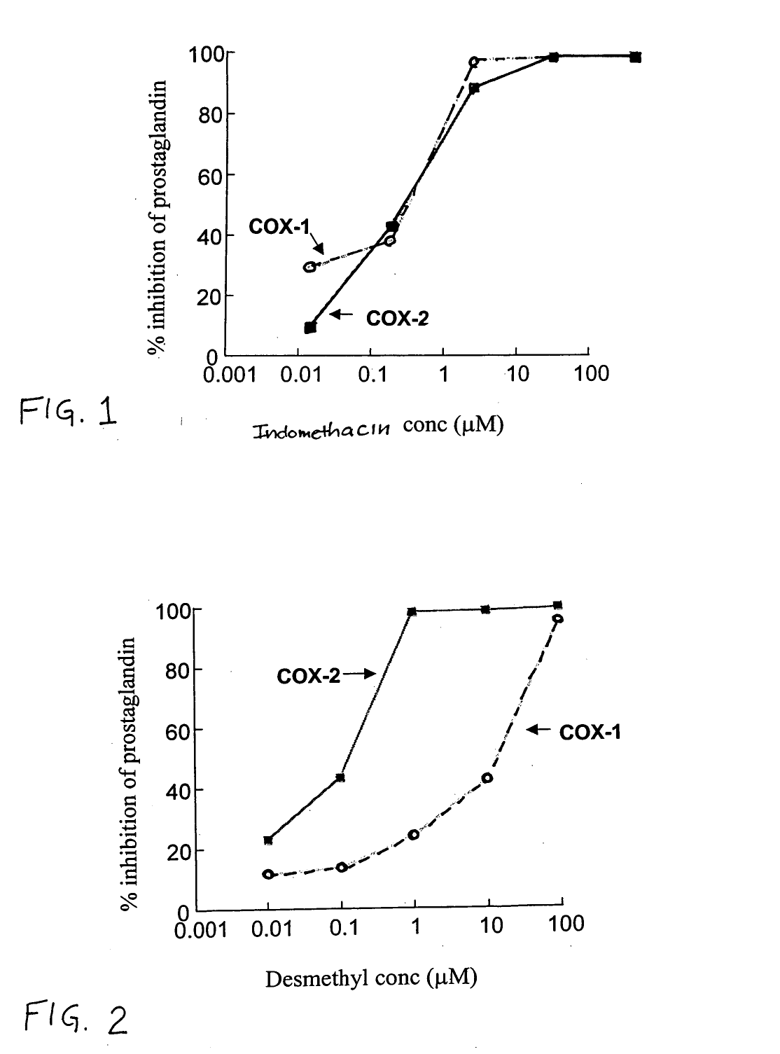

Using the Cox purified enzyme assay methods described below, the inhibition of human COX-2 and ovine COX-1 by indomethacin was measured. As shown in FIG. 1, the IC₅₀ for inhibition of COX-1 by indomethacin (0.13 μM) was nearly identical to the IC₅₀ for inhibition of COX-2 by indomethacin (0.1 μM). In contrast (as shown in FIG. 2), the IC₅₀ for inhibition of COX-1 by desmethylindomethacin (15 μM) was 50- to 150-fold greater than the IC₅₀ for inhibition of COX-2 by desmethylindomethacin (0.1 to 0.3 μM). Thus, the COX-2 selectivity (IC₅₀ for COX-1/IC₅₀ for COX-2) of indomethacin is only 1.3, while the COX-2 selectivity of desmethylindomethacin is 50-150.

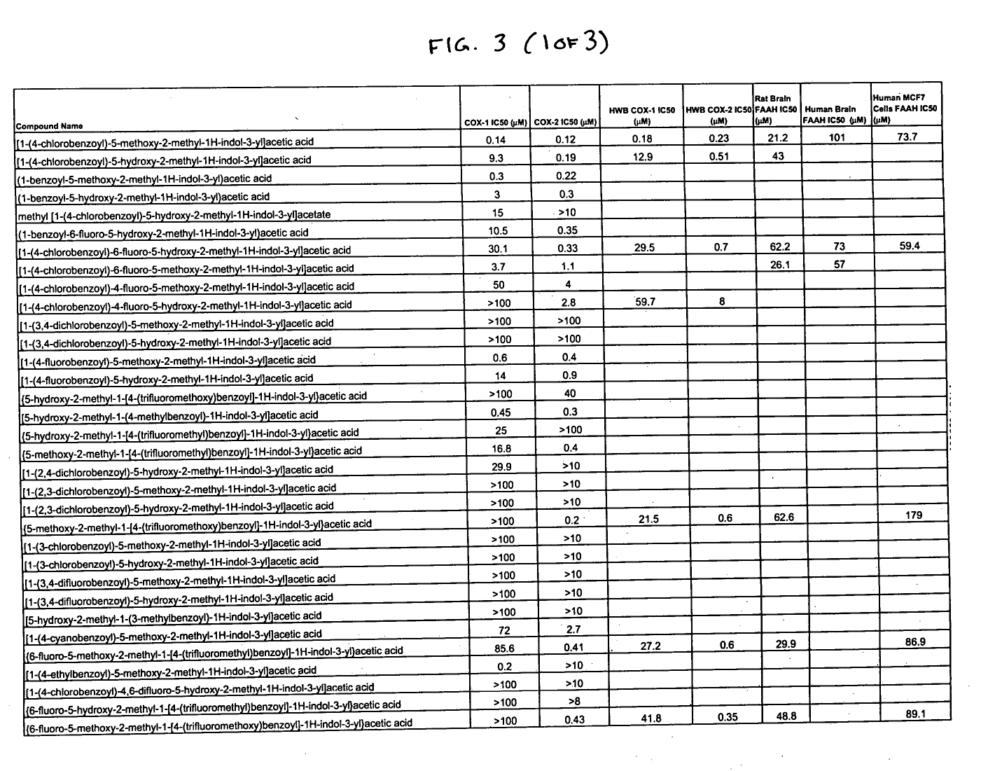

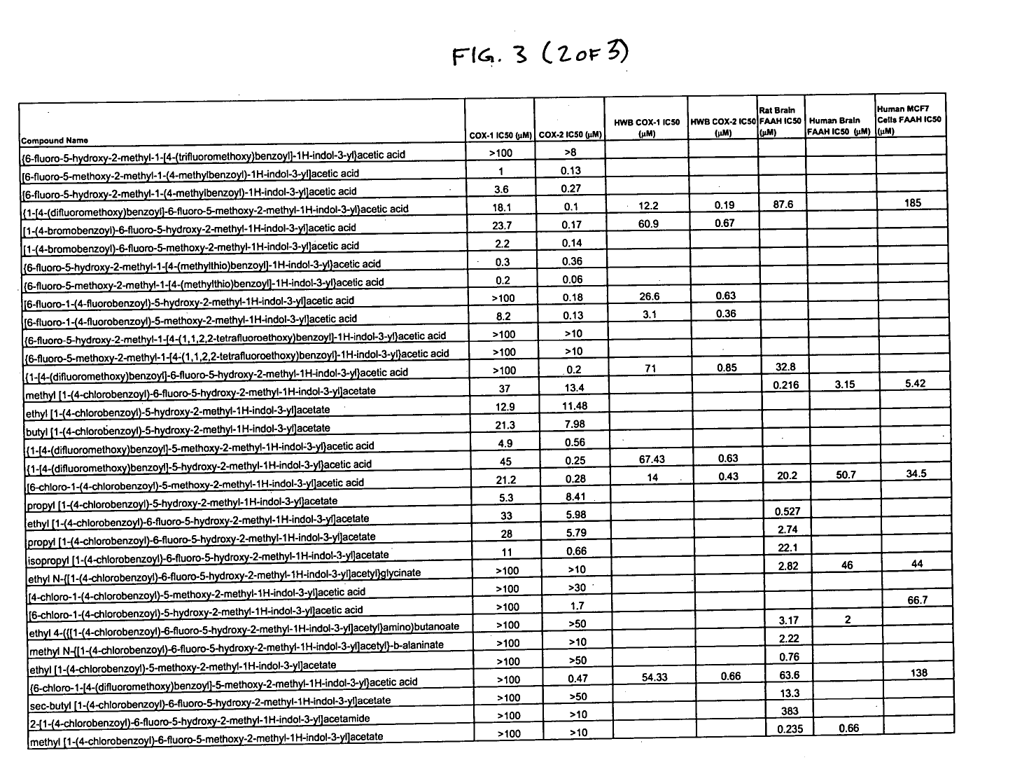

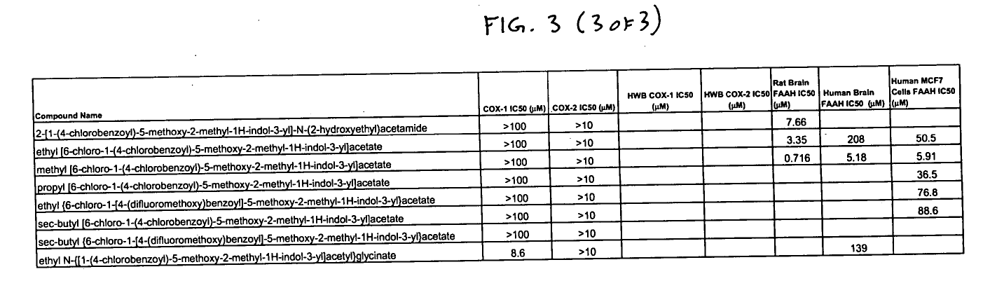

Certain compounds were synthesized and the inhibitory activity of each of these compounds on COX-1, COX-2 and FAAH was measured using the methods described below. The results of this analysis are presented in FIG. 3 which provides COX-1 IC₅₀ (purified enzyme assay), COX-2 IC₅₀ (purified enzyme assay), COX-1 IC₅₀ (human whole blood (HWB) assay), COX-2 IC₅₀ (human whole blood (HWB) assay), FAAH IC₅₀ (rat brain assay), FAAH IC₅₀ (human brain assay) and FAAH IC₅₀ (human MCF7 cell assay) for a number of compounds.

COX-1 and COX-2 Inhibition: Purified Enzyme Assays

The inhibitory activity of each compound on purified COX-1 and purified COX-2 was measured using a test kit available from Cayman Chemical (Ann Arbor, Mich.). Because COX-1 and COX-2 convert arachidonic acid to prostaglandin H₂ (PGH₂), one can assess COX inhibitory activity of a test compound by measuring the effect of the compound on PGH₂ production in the presence of purified COX-1 enzyme and in the presence of purified COX-2 enzyme. In this assay, the production of PGH₂ can be measured by reducing PGH₂ to prostaglandin F_{2α} (PGF_{2α}) with SnCl₂ and then detecting PGF_{2α} by enzyme immunoassay (EIA) using a suitable antibody. The results of this analysis are presented in FIGS. 1-3.

COX-1 and COX-2 Inhibition: Human Whole Blood Assay

A human whole blood assay was also used to measure the inhibitory activity of each compound on COX-1 and COX-2. Briefly, human whole blood was drawn from 3-6 healthy volunteers who had not taken NSAIDS the previous 2 weeks. To measure COX-1 activity in whole blood, 100 μl of whole blood was combined with a 2 μl aliquot of test compound in vehicle or vehicle alone and incubated for 1 hr at 37° C. as described by Berg et al. (1999 Inflamm. Res. 48, 369-379). Serum was isolated from the sample by centrifugation at 12,000 g for 5 min at 4° C. and then assayed for thromboxane B2 (TXB2) levels using an ELISA assay (e.g., Cayman EIA Kit, Catalog Number 519031). To measure COX-2 activity in whole blood, 100 μl of heparinized whole blood was combined with a 1 μl aliquot of 10 mg/ml LPS (lipopolysaccharide) and a 2 μl aliquot of test compound in vehicle or vehicle alone and incubated for 24 h at 37° C. as described by Berg et al. (supra). Serum was isolated from the sample by centrifugation at 12,000 g for 5 min at 4° C. and assayed for prostaglandin E₂ (PGE₂) using an ELISA assay (e.g., Cayman EIA Kit, Catalog Number 514010). The results of this analysis are presented in FIG. 3.

FAAH Inhibition: Human Whole Cell Assay and Rat and Human Brain Homogenate Assays

The ability of the compounds to inhibit FAAH was measured in human whole cell and human and rat brain homogenates as described below

FAAH Rat Brain Membrane (RBM) Homogenate Preparation

9 adult rats (Charles River CD strain, female, 200 g) were anaesthetized with isofluorane and rapidly decapitated, respectively. Each brain was quickly removed and chilled in tubes (3 brains per tube) on ice. Total wet weight of the 9 brains was ˜18 g. 25 mL of “homogenization buffer” (20 mM HEPES buffer, pH 7.0, with 1 mM MgCl₂) was added to each tube. The brains were homogenized on ice for 1 minute using an Omni GLH homogenizer (Omni International, Marietta, Ga.). The homogenates were transferred to three centrifuge tubes and centrifuged at 36,500 g for 20 minutes at 4° C. The supernatant was discarded and each pellet was re-suspended in 25 ml “homogenization buffer”. The re-suspended material was again centrifuged (36,500 g, 20 min at 4° C.). Pellets were combined by resuspension in 10 mL of “homogenization buffer” and incubated in a 37° C. water bath for 15 min. The tubes were then placed on ice for 5 min followed by centrifugation at 36,500 g for 20 minutes at 4° C. The supernatant was discarded and the membrane pellets were then re-suspended in 40 mL of “resuspension buffer” (50 mM Tris-HCl buffer, pH 7.4, containing 1 mM EDTA and 3 mM MgCl₂). A Bradford Protein assay was performed to determine protein concentration. The protein was aliquotted into screw cap Cryo tubes each containing ˜400 μL, flash frozen in liquid nitrogen and stored at −80° C. until used for the assay.

FAAH Human Brain Membrane (HBM) Homogenate Preparation

10 g of normal human brain cortex tissue (pooled from n=3 donors) was purchased from Analytical Biological Services (ABS), Inc. (Wilmington, Del.). The tissue had been collected by ABS, Inc. ˜4 hrs post mortem and was flash frozen and stored at −80° C. The brain tissue was thawed and transferred to a large ceramic mortar on ice. 50 mL of ice-cold “homogenization buffer” (20 mM HEPES buffer, pH 7.0, with 1 mM MgCl₂) was added to the mortar and the tissue was homogenized with a pestle. The homogenate was centrifuged at 36,500 g for 20 minutes at 4° C. The supernatants were discarded and the pellets re-suspended in “homogenization buffer” and centrifuged as before. The supernatants were again discarded and the pellets were re-suspended in 30 ml homogenization buffer and incubated in a 37° C. water bath for 20 min. The homogenate was then centrifuged as before. The supernatant was discarded and the membrane pellets were re-suspended in 30 ml “resuspension buffer” (50 mM Tris-HCl buffer, pH 7.4, containing 1 mM EDTA and 3 mM MgCl₂). A Bradford Protein assay was performed to determine protein concentration. The protein was aliquotted into screw cap Cryo tubes each containing ˜200 μL, flash frozen in liquid nitrogen and stored at −80° C. until used for the assay.

FAAH Human Carcinoma Cell Membrane (HCM) Homogenate Preparation

Human breast epithelial carcinoma MCF7 cells were obtained from the American Type Culture Collection (ATCC Number HTB-22, Manassas, Va.) and cultured as essentially as described by ATCC. Briefly, cells were grown in Eagle's Minimum Essential Medium (ATCC catalog no. 30-2003) supplemented with 4 mM L-glutamine, 10% final volume of fetal bovine serum (ATCC catalog no. 30-2020), and 0.1 mg/ml human recombinant insulin (Sigma, St. Louis, Mo.). The cells were grown in 5% carbon dioxide in air. When cells reached ˜80% confluency, adherent cells were rinsed with Hanks Balanced Salts Solution (ATCC catalog no. 30-2213), scraped into suspension and collected by centrifugation in a clinical centrifuge at room temperature. Cell pellets were then washed by resuspension in Hanks Balanced Salts Solution followed by centrifugation. Cell pellets were then flash frozen in a dry ice and ethanol bath and stored at −80° C. Cell pellets were thawed and 25 ml of homogenization buffer was added. Membrane homogenates of MCF7 cells were then prepared as described above for rat brain homogenates. A Bradford Protein assay was performed to determine the protein concentration. The protein was aliquotted into screw cap Cryo tubes each containing ˜200 μL, flash frozen in liquid nitrogen and stored at −80° C. until used for the assay.

Determination of FAAH Activity

FAAH activity was assayed in the respective homogenates (Rat brain, Human brain, or Human breast cell carcinoma MCF7 cell) using a modification of the method of Omeir et al. (1995 Life Sci 56:1999) and Fowler et al (1997 J. Pharmacol Exp Ther 283:729). For assay of FAAH in rat brain membrane homogenates (RBM), RBM homogenates (7 μg protein in 20 μl final volume of 10 mM Tris pH 6.5) were mixed with 180 μl of a mixture of the following: 2.0 μM unlabelled anandamide. 0.03 μCi radiolabeled anandamide [ethanolamine 1-³H] (40-60 Ci/mmol, product number ART-626, American Radiolabelled Chemicals, St. Louis, Mo.), 1 mg/ml Bovine Serum Albumin (fatty acid-free BSA, electrophoresis grade, Sigma, St. Louis Mo.), 10 mM Tris-HCl (pH 6.5), and 1 mM EDTA in the presence and absence of inhibitors (vehicle was DMSO at a final concentration of 1%) and incubated for 10 min at 37° C. Samples were placed on ice to terminate the reactions. ³H-ethanolamine product and un-reacted ³H-anandamide substrate were separated by either of 2 respective methods (1) using chloroform/methanol extraction or (2) by passing the reaction mixture through a glass fiber filter containing activated charcoal. Samples were extracted with chloroform/methanol by adding 0.4 ml of chloroform/methanol (1:1 v/v), vigorously mixing the samples, and separation of the aqueous and organic phases by centrifugation. Radioactivity (corresponding to FAAH-catalyzed breakdown of ³H-anandamide) found in aliquots (0.2 ml) of the aqueous phase was determined by liquid scintillation counting with quench correction. IC₅₀ values are determined as described by Jonsson et al. (2001 Br J Pharmacol 133:1263). Alternatively, reactions were purified using a modification of the solid-phase extraction method described by Wilson et al (2003 Anal Biochem 318:270). This method was modified as follows: after reactions were incubated at 37° C. for 10 min and chilled on ice, the reaction mixtures were acidified by adding 10 μl of sodium phosphate solution [0.5M (pH 2.0)]. 90 μl aliquots of the acidified reaction mixtures were applied to activated charcoal (that was previously washed with methanol as described by Wilson et al.) containing 80 μl of water on top of a glass fiber filter, centrifuged, and the radioactivity in the eluate was counted as described previously by Wilson et al.

Measurement of Pharmacokinetic Parameters

To determine the various pharmacokinetic parameters, serum samples from animals dosed with a test compound are collected and analyzed by LC/MS. Briefly, samples are injected (10 μL) into a flow of 10% methanol in water onto a sample extraction cartridge (Waters Oasis HLB Direct Connect). The sample is washed for 0.5 min followed by a column switch that places the sample extraction cartridge into the path of the HPLC. The sample is eluted onto a reverse phase HPLC column (Waters Xterra IS C₈ 2.1×20 mm, 5 um particle size) and is eluted with a gradient (Mobile Phase A:10 mM NH₄OH in dH₂O; Mobile Phase B: 20% methanol in Acetonitrile). Initial condition of 20% B, ramping to 90% B over 1.5 minutes, and holding for 0.5 minutes, then returning to initial conditions to re-equilibrate the column for 1 min, all at a flow rate of 0.4 ml/min.). A Micromass Quattro Micro (Waters Corp.; Milford, Mass.) triple quadrupole mass spectrometer operated in MRM mode is used to detect test compound as it elutes from the HPLC column. Concentrations are determined by relative response to an internal standard and calculated based on a standard concentration curve of the test compound. MassLynx software (Waters, Corp.; Milford, Mass.) is used to calculate the absolute concentration of test compound in each serum sample. A concentration versus time plot is generated from the data in Microsoft Excel, Summit Software PK Solutions 2.0 or Graph Pad Prism (GraphPad Software, Inc., San Diego, Calif.) to generate PK curves. From the generated curve, the AUCₙ (Area Under the Curve, n=length of experiment in hours) is calculated by the software for both intravenous (IV) and orally dosed animals. Oral Bioavailability (Fₙ) is calculated using the equation: F=AUCₒᵣₐₗ/AUC_{IV}. Cₘₐₓ and Tₘₐₓ are determined by visual inspection of the oral concentration curve. Cₘₐₓ is the maximum concentration of the test compound circulating in the blood through the duration of the experiment reported at time, T (Tₘₐₓ). The terminal half life, t_{1/2}, is calculated using at least two data points on the IV curve representing the elimination phase. Thus, the t_{1/2} is calculated by inserting the slope (β) of the line generated by plotting the natural log of the test article concentration versus time (during the elimination phase) into the equation t_{1/2}=0.693/β. The volume of distribution (Vd) is calculated using the equation Vd=Cls/β (Cls=systemic clearance, β=slope from t_{1/2} equation). Cls are determined by dividing the dose by the AUC_{IV}.

Any of a variety of animal models can be used to test the compounds of the invention for their effectiveness in reducing inflammation and treating pain. Useful compounds can exhibit effectiveness in reducing inflammation or pain in one or more animal models.

Animal Models for Assessing Anti-inflammatory Activity

Carrageenan-Induced Foot Pad Edema Model

The model is described, for example, by Winter et al. (1962 Proc Soc Exp Biol Med 111:544). Briefly, rats are fasted with free access to water for 17 to 19 hours before oral treatment with up to three doses of a test compound, indomethacin or celecoxib, or a control vehicle (1% methylcellulose in deionized water). One hour after the last treatment, paw edema is induced by injecting 0.05 ml of a 2% carrageenan solution into the left hindpaw.) The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

Complete Freund's Adjuvant (CFA) Induced Arthritis Model

In this model arthritis is induced in groups of eight Lewis derived male rats weighing 160±10 g by injecting a well-ground suspension of killed Mycobacterium tuberculosis (0.3 mg in 0.1 ml of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through day 5) beginning one hour before injection of CFA. For CFA-injected vehicle control rats, the increase in paw volume on Day 5 relative to Day 1 (Acute Phase of inflammation) is generally between 0.7 and 0.9 ml; and, that on Day 18 relative to day 14 (Delayed Phase of inflammation) is generally between 0.2 and 0.4 ml. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean±SEM for each treatment group is determined and a Dunnett test is applied for comparison between vehicle and treated groups. Differences are considered significant at P<0.05. Polyarthritis of fore paw, tail, nose and ear can be scored visually and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds (Bertorelli et al. 1999 Brit Journ Pharmacol 128:1252)

Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 ml of sterile air into the intrascapular area of the back to create air cavities. An additional 10 ml of air is injected into the cavity every 3 days to keep the space open. Seven days after the initial air injection, 2 ml of a 1% solution of carrageenan dissolved in sterile saline is injected directly into the pouch to produce an inflammatory response. In treated and untreated animals the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

Animal Models for Assessing Analgesic Activity

Carrageenan-Induced Thermal Hyperalgesia

This model is described by Hargreaves et al. (1988 Pain 32:77). Briefly, inflammation is induced by subplantar injection of a 2% carrageenan suspension (0.1 ml) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 hour after the oral treatment with the test compound or a control. The results can be expressed as the nociceptive threshold in seconds (sec) for each hindpaw and the percentage of variation of the nociceptive threshold (mean±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

Phenylbenzoquinone-Induced Writhing Model

This model is described by Siegmund et al. (1957 Proc Soc Exp Bio Med 95:729). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th minute after PBQ injection, and can also be counted between the 35^{th} and 40^{th} minute and between the 60^{th} and 65^{th} minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

Kaolin-Induced Arthritis Model.

This model is described by Hertz et al. (1980 Arzneim Forsch 30:1549). Briefly, arthritis is induced by injection of 0.1 ml of kaolin suspension into the knee joint of the right hind leg of a rat. Test compounds are administered subcutaneously after 15 minutes and again after two hours. Reference compounds can be administered orally or subcutaneously. Gait is assessed every hour from 1.5 hours to 5.5 hours after treatment and is scored as follows: normal gait (0), mild disability (1), intermittent raising of paw (2), and elevated paw (3). Results are expressed as the mean gait score (mean±SEM) calculated from individual values at each time point and the percentage of variation of the mean score calculated from the mean value of the vehicle-treated group at 4.5 hours and 5.5 hours after treatment. The statistical significance of differences between the treated groups and the vehicle-treated group is determined by a Dunnett's test using the residual variance after a one-way analysis of variance (P<0.05) at each time point.

Peripheral Mononeuropathy Model

This model is described by Bennett et al. (1988 Pain 33:87) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is be induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 ml/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 minutes after treatment. The results are expressed as: the nociceptive threshold (mean±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat® v. 2.0.3 (SPSS Science Software, Erkrath GmbH)).

Diabetic Neuropathy Paw Pressure Test

Complete protocol details can be found in Rakieten et al. (1963 Cancer Chemother Rep 29:91). Briefly, diabetes is induced by intraperitoneal injection of streptozotocin in rats. Three weeks later, the nociceptive threshold is measured using the paw pressure test to assess hyperalgesia. Test compound or controls are administered intraperitoneally 30 minutes prior to pain measurement.

Acetic Acid Writhing Test

Briefly, a test compound is administered orally one hour before intraperitoneal injection of acetic acid (0.5%, 10 ml/kg) in rats. Reduction in the number of writhes by 50 percent or more (≧50) per group of animals observed during the 5 to 11 minute period after acetic acid administration, relative to a vehicle treated control group, indicates possible analgesic activity. This assay is based on that described in Inoue, K. et al. (1991 Arzneim. Forsch./Drug Res. 41: 235).

Formalin Test

Complete protocol details can be found in Hunskaar et al. (1985 Neurosci. Meth. 14:69). Briefly, 30 minutes after intraperitoneal administration of a test compound or a control, 20 μl of a 5% formalin solution is injected by subplantar route into the right hindpaw of the rat. Hindpaw licking time is recorded during the early phase and the later phase after formalin injection.

Tail Flick Test

Complete protocol details can be found in D'Amour and Smith (1941 J Pharmacol. Exp Ther. 72:74). Briefly, 30 minutes after intraperitoneal administration of a test compound or a control, a light beam is focused onto the tail of the rat. The nociceptive reaction latency, characterized by tail withdrawal, is recorded. The cutoff time is set to 15 seconds.

Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

Hot Plate Test

Complete protocol details can be found in Eddy et al. (1950 J. Pharmacol. Exp. Ther. 98:121). Briefly, 30 minutes after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

Assays for Assessing Anxiolytic Activity

Compounds of the invention that modulate FAAH activity, and thus fatty acid amide levels, may also have anxiolytic activity. Animal models to assess anxiolytic activity include:

Elevated Plus Maze

The elevated plus maze consists of four maze arms that originate from a central platform, effectively forming a plus sign shape as described in van Gaalen and Steckler (2000 Behavioural Brain Research 115:95). The maze can be made of plexiglas and is generally elevated. Two of the maze arms are unwalled (open) and two are walled (closed). The two open arms are well lit and the two enclosed arms are dark (Crawley 2000 What's Wrong With My Mouse?: Behavioral Phenotyping of Transgenic and Knockout Mice. Wiley-Liss, New York). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 minutes following intraperitoneal administration of test compound or control, an animal is placed individually on the central platform, facing one of the open arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-minute test session. A greater amount of time spent or entries made by the animal in the open versus the closed arms is an indicator of anxiolytic activity.

Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9: 76). Briefly, 30 minutes following intraperitoneal administration of test compound or control, an animal is placed on one open sector in front of an enclosed sector. Time in a new sector is recorded as entry with all four paws. Behavior will be scored by an observer and/or via a video camera over a 5-minute test session. A greater amount of time spent or entries made by the animal in the open versus the walled sector is an indicator of anxiolytic activity.

Assays for Assessing Antinociception Mechanism

Compounds can be tested to determine if they influence pathways involved in nociception. The results of such assays can be used to investigate the mechanism by which a test compound mediates its antinociceptive effect.

Elevation of 3α,5α-THP

3α-hydroxy-5α-pregan-20-one (3α,5α-THP or allopregnanolone) is a pregnane steroid that acts as an agonist of the inhibitory GABAA receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (1982 J Neuroscience 20:200) as follows. Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 ml aliquots of 10% (v/v) ethyl acetate in heptane. The aliquots are combined and diluted with 4 ml of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 μl of the redissolved extract by liquid scintillation spectroscopy and the remaining sample is used in the determination of 3α,5α-THP by radioimmunoassay. Reconstituted sample extracts (75 μl) and 3α,5α-THP standards (5-40,000 pg in 6.25% v/v ethanol, 31% v/v isopropyl alcohol in RIA buffer) are assayed in duplicate by the addition of 725 μl of RIA buffer, 100 μl of [³H]3α,5α-THP (20,000 dpm), and 100 μl of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μl of cold dextran coated charcoal (DCC; 0.04% dextran, 0.4% powdered charcoal in double-distilled H₂O) for 20 min. DCC is removed by centrifugation at 2000×g for 10 min. Bound radioactivity in the supernatant is determined by liquid scintillation spectroscopy. Sample values are compared to a concurrently run 3α,5α-THP standard curve and corrected for extraction efficiency.

Inhibition of Fatty Acid Amide Hydrolase

The endogenous cannabinoid system is involved in the regulation of nociception, among other physiological effects. One component of this system is fatty acid amide hydrolase (FAAH), which inactivates the fatty acid amide anandamide. Inhibitors of FAAH could thus inhibit anandamide degradation and result in increased levels of anandamide, with resulting analgesic effects. The effect of test compounds on FAAH activity can be assayed in human whole cell and in human and rat brain homogenates as described above.

Whole Cell Anandamide Hydrolysis Assay

FAAH activity can be assayed in whole cells using methods disclosed previously (Maccarone et al. 1998 J Biol Chem 273:32332 and Bisogno et al. 1997 J Biol Chem 272:3315). In addition to the cell lines described in Maccarone et al. and Bisogno et al., MCF7 (ATCC designation HTB-22) and T84 (ATCC designation CCL-248) cell lines may be used in these assays.

Determination of Endogenous and Exogenous Anandamide Levels in Rat Plasma and Brain Tissue

The effects of test compounds on endogenous and exogenously dosed anandamide levels can be measured. Rats dosed with test article are sacrificed at various time points to determine the levels of anandamide both circulating and within the brain tissue. For experiments measuring exogenous levels of anandamide, the anandamide (Cayman Chemical, Ann Arbor, Mich. or Sigma Chemical, St. Louis, Mo.) is dosed (in the range of 3-30 mg/kg) intraperitoneally (IP) 30 minutes post dosing of test compound. Animals are sacrificed at either 15, 30, or 60 minutes after anandamide administration upon anesthesia administration followed by decapitation. Brains are immediately extracted and the plasma is recovered from the blood.

Anandamide is extracted from the plasma by first precipitating the proteins by adding an equal volume of cold acetone with 10 ng of d8-anadamide (Cayman Chemicals, Ann Arbor, Mich.) as an internal standard. The acetone is evaporated from the supernatant followed by an extraction with chloroform:methanol (2:1). The chloroform layer is collected and evaporated to dryness. The pellet containing the anandamide is resuspended into methanol:chloroform (3:1) and injected onto an Xterra IS 2.1×20 mm C8 column (Waters Corporation, Milford, Mass.) and followed by detection by a Waters Quattro Micro LCMSMS (Waters Corporation, Milford, Mass.). The HPLC method consists of a step gradient (mobile phase A: 10 mM ammonium hydroxide in water, mobile phase B: 20% methanol in Acetonitrile) starting at 25% B and stepping up to 90% B at 2.2 minutes and holding for 2 minutes. Quantities are measured against known standards spiked into blank plasma using MassLynx v.4.0 software (Waters Corporation, Milford, Mass.).

Levels of anandamide from brain tissue are determined as follows. Brain tissue is homogenized in ethyl acetate and water (3:1) with 10 ng of d8-anadamide (Cayman Chemicals, Ann Arbor, Mich.) as an internal standard. The ethyl acetate layer is collected and evaporated to dryness. The pellet containing anandamide is resuspended in methanol:chloroform (3:1) and analyzed by the same method as plasma and normalized against the fresh tissue weight.

Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561), and CB₂ is expressed by macrophages and in the spleen (Munro et al. 1993 Nature 365:61). Both of these receptors have been implicated in mediating analgesic effects through binding of agonists (see, for example, Clayton et al. 2002 Pain 96:253). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁ ligand, e.g., [³H]WIN 55212-2 (2 nM for CB₁ and 0.8 nM for CB₂) to membranes isolated from HEK-293 cells expressing recombinant CB₁ is measured in the presence and absence of a compound. Non-specific binding is separately determined in the presence of several-fold excess of unlabelled WIN 55212-2 (5 μM for CB₁ and 10 μM for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(1+(L/K_{D})), where L=concentration of radioligand in the assay, and K_{D}=affinity of the radioligand for the receptor).

Therapeutic Methods

The compounds of the invention can be used, for example, to treat conditions or disorders in which it is considered desirable to reduce or eliminate COX-2 activity and/or FAAH activity. Thus, they can be used in any situation in which a COX-2 inhibitor or FAAH inhibitor is used as well as in other situations. For example, compounds of Formula I and related prodrugs can be used to treat an inflammatory disorder, including both disorders in which inflammation is considered a significant component of the disorder and those in which inflammation is considered a relatively minor component of the disorder, to treat acute and chronic pain (analgesic) and to treat fever (antipyretic). Among the inflammatory disorders that can be treated are auto-immune disorders.

Disorders that can be treated with a composition comprising a compound having Formula I and related prodrugs include: arthritis (including rheumatoid arthritis, spondyloarthopathies, gouty arthritis, degenerative joint diseases (i.e. osteoarthritis), systemic lupus erythematosus, ankylosing spondylitis, acute painful shoulder, psoriatic, and juvenile arthritis), asthma, atherosclerosis, osteoporosis, bronchitis, tendonitis, bursitis, skin inflammation disorders (i.e. psoriasis, eczema, burns, dermatitis), enuresis, eosinophilic disease, gastrointestinal disorders (including inflammatory bowel disease, peptic ulcers, regional enteritis, diverticulitis, gastrointestinal bleeding, Crohn's disease, gastritis, irritable bowel syndrome and ulcerative colitis), and disorders ameliorated by a gastroprokinetic agent (i.e. ileus, for example post-operative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP)).

The compounds of the invention can also be used in the treatment of symptoms associated with influenza or other viral infections, common cold, sprains and strains, myositis, neuralgia, synovitis, injuries such as sports injuries and those following surgical and dental procedures, coagulation disorders, kidney disease (e.g., impaired renal function), ophthalmic disorders (including glaucoma, retinitis, retinopathies, uveitis and acute injury to the eye tissue), liver diseases (i.e., inflammatory liver disease including chronic viral hepatitis B, chronic viral hepatitis C, alcoholic liver injury, primary biliary cirrhosis, autoimmune hepatitis, nonalcoholic steatohepatitis and liver transplant rejection), and pulmonary inflammatory diseases (e.g., including asthma, allergic rhinitis, respiratory distress syndrome chronic bronchitis, and emphysema). Compositions comprising a compound having Formula I or and related prodrugs can also be used to treat, for example, inflammation associated with: vascular diseases, migraine headaches, tension headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, multiple sclerosis, and ischemia (e.g., myocardial ischemia), and the like. The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis and inflammatory bowel disease). The compounds may also be useful in treating inflammation associated with trauma and non-inflammatory myalgia. The compounds can also be administered to those prior to surgery or taking anticoagulants. The compounds of the invention may reduce the risk of a thrombotic cardiovascular event which is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fagax), reversible ischemic neurologic deficits, and any similar thrombotic event in any vascular bed (splanchnic, renal, aortic, peripheral, etc.).

The compounds of the invention may inhibit uterus contraction caused by hormones and prostanoid-induced smooth muscle contraction. The compounds of the invention may be useful in treating premature labor, menstrual cramps, menstrual irregularity, and dysmenorrhea.

The compounds of the invention may inhibit cellular neoplastic transformations and metastatic tumor growth. The compounds of the invention may be associated with reducing the number of adenomatous colorectal polyps. Thus, compounds and prodrugs may also be useful in reducing the risk of certain cancers, e.g., solid tumor cancers such as colon or colorectal cancer. The compounds and prodrugs may also be used in the treatment of prevention of all cancers including cancers of the bladder, cancers associated with overexpression of HER-2/neu cervix, skin, esophagus, head and neck, lung including non small-cell lung cancers, kidney, pancreas, prostate, gall bladder and bile duct and endometrial cancers, gastric cancers, gliomas, hepatocellular carcinomas, colonic adenomas, mammary cancers, ovarian cancers and salivary cancers. In addition, the compounds and prodrugs may be useful in treating large intestine cancer and prostate cancer. The compounds may also be useful in cases where the patient is at risk for cancer including oral premalignant lesions, cervical intraepithelial neoplasia, chronic hepatitis, bile duct hyperplasia, atypical adenomatous hyperplasia of lung, prostatic, intraepithelial neoplasia, bladder dysplasia, actinic keratoses of skin, colorectal adenomas, gastric metaplasia, and Barrett's esophagus.

Compounds of the invention are also useful for the treatment of cognitive disorders such as dementia, particularly degenerative dementia (including senile dementia, Alzheimer's disease (and precursors thereof), Pick's disease, Huntington's chorea, Parkinson's disease and Creutzfeldt-Jakob disease), and vascular dementia (including multiinfarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

Compounds of the invention may also prevent neuronal injury by inhibiting the generation of neuronal free radicals (and hence oxidative stress) and therefore are of use in the treatment of stroke; epilepsy; and epileptic seizures (including grand mal, petit mal, myoclonic epilepsy and partial seizures). The compounds of the invention may be useful to control or suppress seizures (including those that are chemically induced).

The compounds of the invention can be used in treatment of all varieties of pain including pain associated with a cough condition, pain associated with cancer, preoperative pain, arthritic pain and other forms of chronic pain such as post-operative pain, lumbosacral pain, musculo-skeletal pain, headache, migraine, muscle ache, lower back and neck pain, toothache and the like. The compounds of the invention are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; neuralgia, such as post-herpetic neuralgia and trigeminal neuralgia; and pain resulting from physical trauma, amputation, cancer, toxins or chronic inflammatory conditions. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of the invention may also be of use in the treatment and/or prevention of cyclooxygenase-mediated proliferative disorders such as may occur in diabetic retinopathy and tumor angiogenesis. The compounds of the invention may be used to inhibit angiogenesis, such as occurs in wet macular degeneration.

The compounds of the invention may also be used for treating sexual behavior problems and/or improving sexual performances.

The compounds useful in the prevention and/or treatment of pain, in particular acute or chronic neurogenic pain, migraine, neuropathic pains including the forms associated with herpes virus and diabetes, acute or chronic pain associated with the inflammatory diseases: arthritis, rheumatoid arthritis, osteoarthritis, spondylitis, gout, vascularitis, Crohn's disease, irritable bowel syndrome and acute/sharp or chronic pains at the periphery. The compounds of the invention can also be used to prevent and/or treat emesis, dizziness, vomiting, and nausea, especially after chemotherapy, food behavioral problems/feeding disorders (i.e. eating disorders, in particular anorexias and cachexias of various natures, weight loss associated with cancer and other wasting conditions), neurological pathologies, psychiatric tremors (e.g., dyskinesias, dystonia, spasticity, obsessive compulsive behavior, Tourette's syndrome, all forms of depression and anxiety of any nature and origin, mood disturbances, psychoses), acute or chronic neurodegenerative diseases (e.g., Parkinson's disease, Alzheimer's disease, senile insanity, Huntington's chorea, lesions related to cerebral ischemia and cranial and medullary traumas, epilepsy, sleep disorders (sleep apnea), cardiovascular diseases (in particular hypertension, cardiac arrhythmias, arteriosclerosis, heart attacks, cardiac ischemias, renal ischemia), cancers (benign tumors of the skin, papillomas and cerebral tumors, prostate tumors, cerebral tumors (glioblastomas, medullary epitheliomas, medullary blastomas, neuroblastomas, tumors of origin, astrocytomas, astroblastomas, ependymomas, oligodendrogliomas, plexus tumor, neuroepithelioma, epiphysis tumor, ependyblastomas, malignant meningiomas, sarcomatosis, malignant melanomas, schwan cell cancers), disorders of the immune system (in particular autoimmune diseases including psoriasis, erythematous lupus), diseases of conjunctive or connective tissue, Sjogren's syndrome, spondylarthritis anchylosis, undifferentiated spondylarthritis undifferentiated, Behcet's disease, autoimmune hemolytic anaemias, multiple sclerosis, amyotrophic side sclerosis, amyloses, graft rejection, and illnesses affecting the blastocytes, allergic diseases (i.e., immediate or delayed hypersensitivity, allergic rhinitis or conjunctivitis, contact dermatitis), viral or bacterial parasitic infectious diseases (i.e. AIDS, meningitis), inflammatory diseases (in particular arthritic diseases: arthritis, rheumatoid arthritis osteoarthritis, spondylitis, gout, vascularitis, Crohn's disease, irritable bowel syndrome, osteoporosis, psoriasis, ocular infections and disorders (i.e. ocular hypertension, glaucoma, wet macular degeneration), lung diseases (i.e. diseases of the respiratory tracts, bronchyospasms, cough, asthma, chronic bronchitis, chronic obstruction of the respiratory tracts, emphysema), gastrointestinal disorders (i.e. irritable bowel syndrome, intestinal inflammatory disorders, ulcers, diarrheas, acid reflux), urinary incontinence, vesical inflammation, movement disorders, psychomotor disorders, hypertension, and AIDS-related complex. The compounds of the invention can be used as a sleep aid, to treat insomnia or to induce sleep. The compounds may be used to reduce or control body weight (or fat) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). The compounds of the invention may be used to prevent, control or treat schizophrenia, paranoia or other related disorders, or other disorders of dopamine transmission.

The compounds of the invention can also be used to treat anxiety (including generalized anxiety disorder, panic disorder, and social anxiety Disorder) and depression.

Administration of Compounds

The compounds of the invention can be used alone or in combination with other compounds used to treat inflammatory disorders. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Thus, the compounds of the invention can be used in a co-therapy with a second agent, e.g., an anti-inflammatory agent. Anti-inflammatory agents which can be used in co-therapy include: NSAIDs, 5-lipoxygenase(LO) inhibitors (e.g., masoprocol, tenidap, zileuton, pranlukast, tepoxalin, rilopirox, and flezelastine hydrochloride, enazadrem phosphate, and bunaprolast), p38 inhibitors (e.g. SB203580 and Vertex compound VX745), LTB₄ antagonists and LTA₄ hydrolase inhibitors, CRTH2 modulators (e.g. ramatroban), steroids, corticosteroids (e.g. betamethasone, budesonide, cortisone, prednisone, triamcinolone, methylprednisone, prednisone, and dexamethasone, hydrocortisone), Bayer Bay-x-1005, Ciba Geigy compound CGS-25019C, Leo Denmark compound ETH-615, Lilly compound LY-293111, Ono compound ONO-4057, Terumo compound TMK-688, Lilly compounds LY-213024, 264086 and 292728, ONO compound ONO-LB457, Searle compound SC-53228, calcitrol, Lilly compounds LY-210073, LY-223982, LY-233469, and LY-255283, ONO compound ONO-LB-448, Searle compounds SC-41930, SC-50605 and SC-51146, and SmithKline SKF-104493.

The compounds of the invention can be used in combination with selective COX-2 inhibitors, e.g., Celecoxib®, Valdecoxib®, Parecoxib®, Rofecoxib®, Etoricoxib®, and Lumaricoxib®.

The compounds of the invention can be used in a co-therapy with a an agent used to treat an anxiety disorders, including: benzodiazepines (e.g., Xanax®, Librium®) and SSRIs (e.g., Prozac®, Zoloft®), monoamine oxidase inhibitors (MAOIs) and tricyclic antidepressants (TCAs, e.g., amitryptilline).

The compounds of the invention can be used in a co-therapy with a an agent used to treat rheumatoid arthritis including etanercept (Enbrel®) and infliximab (Remicade®).

The compounds of the invention can also be used in a co-therapy with a second agent that has analgesic activity. Analgesics which can be used in co-therapy include, but are not limited to: NSAIDs (e.g., aspirin, ibuprofen, fenoprofen, acetaminophen, phenacetin, diclofenac, etodolac, ketoprofen, ketorolac, flurbiprofen, indomethacin, mefenamic acid, diflusinal, fenbufen, meclofenamic acid, sulindac, flufenisal, piroxicam, phenylbutazone, tolmetin, zomepirac, nabumetone oxaprozin and naproxen), a non-narcotic analgesic such as tramadol, or a narcotic analgesic (e.g., codeine, oxycodone, dihydrocodeine, hydrocodone, loperamide, fedotozine, and fentanyl, naloxone, naltrexone, methyl nalozone, nalmefene, cypridime, beta funaltrexamine, naloxonazine, naltrindole, and nor-binaltorphimine, morphine, diphenyloxylate, enkephalin pentapeptide, and trimebutine), levorphanol, APF112, mepivacaine), NK1 receptor antagonists (e.g., ezlopitant and SR-14033, SSR-241585), CCK receptor agonists (e.g., loxiglumide), NK1 receptor antagonists, NK3 receptor antagonists (e.g., talnetant, osanetant SR-142801, SSR-241585), norepinephrine-serotonin reuptake inhibitors (NSRI; e.g., milnacipran), vanilloid receptor agonists and antagonists, cannabinoid receptor agonists (e.g., arvanil), sialorphin, compounds or peptides that are inhibitors of neprilysin, frakefamide (H-Tyr-D-Ala-Phe(F)-Phe-NH₂; WO 01/019849 A1), loperamide, Tyr-Arg (kyotorphin), CCK receptor agonists (caerulein), conotoxin peptides, peptide analogs of thymulin, loxiglumide, dexloxiglumide (the R-isomer of loxiglumide) (WO 88/05774), and analgesic peptides (e.g. endomorphin-1, endomorphin-2, nocistatin, dalargin, lupron, and substance P). In addition, certain antidepressants can be used in co-therapy either because they have analgesic activity or are otherwise beneficial to use in combination with an analgesic. Examples of such anti-depressants include: selective serotonin reuptake inhibitors (e.g., fluoxetine, paroxetine, sertraline), serotonin-norepinephrine dual uptake inhibitors, venlafaxine and nefazadone. Certain anti-convulsants have analgesic activity and are useful in co-therapy. Such anti-convulsants include: gabapentin, carbamazepine, phenyloin, valproate, clonazepam, topiramate and lamotrigine. Such agents are considered particularly useful for treatment of neuropathic pain, e.g., treatment of trigeminal neuralgia, postherpetic neuralgia, and painful diabetic neuropathy. Additional compounds useful in co-therapy include: alpha-2-adrenergic receptor agonists (e.g., tizanidine and clonidine), mexiletine, corticosteroids, compounds that block the NMDA (N-methyl-Daspartate) receptor (e.g, dextromethorphan, ketamine, and amantadine), glycine antagonists, carisoprodol, cyclobenzaprine, various opiates, nonopioid antitussive such as dextromethorphan, carmiphen, caramiphen or carbetapentane, opioid antitussives such as codeine or hydrocodone and metaxolone. The compounds of the invention can also be combined with inhalable gaseous nitric oxide (for treating pulmonary vasoconstriction or airway constriction), a thromboxane A2 receptor antagonist, a stimulant (i.e. caffeine), an H₂-antagonist (e.g. ranitidine), an antacid (i.e. aluminum or magnesium hydroxide), an antiflatulent (i.e. simethicone), a decongestant (including phenylephrine, phenylpropanolamine, pseudophedrine, oxymetazoline, ephinephrine, naphazoline, xylometazoline, propylhexedrine, or levodesoxyephedrine), a prostaglandin (i.e. misoprostol, enprostil, rioprostil, ornoprostol or rosaprostol), a diuretic, a sedating or non-sedating antihistamine, a 5HT1 agonist, such as a triptan (e.g. sumatriptan or naratriptan)., an adenosine Al agonist, an EP ligand, a sodium channel blocker (e.g. lamotrigine), a substance P antagonist (e.g. an NK, antagonist), a cannabinoid, a 5-lipoxygenase inhibitor, a leukotriene receptor antagonist, a DMARD (e.g. methotrexate), a neurone stabilising antiepileptic drug, a mono-aminergic uptake inhibitor (e.g. venlafaxine), a matrix metalloproteinase inhibitor, a nitric oxide synthase (NOS) inhibitor, such as an iNOS or an nNOS inhibitor, an inhibitor of the release, or action, of tumor necrosis factor, an antibody therapy, such as a monoclonal antibody therapy, an antiviral agent, such as a nucleoside inhibitor (e.g. lamivudine) or an immune system modulator (e.g. interferon), a local anaesthetic, a known FAAH inhibitor (e.g., PMSF, URB532, URB597, or BMS-1, as well as those described in those described in WO04033652, U.S. Pat. No. 6,462,054, US20030092734, US20020188009, US20030195226, and WO04033422), an antidepressant (e.g., VPI-013), a fatty acid amide (e.g. anandamide, N-palmitoyl ethanolamine, N-oleoyl ethanolamide, 2-arachidonoylglycerol, or oleamide), arvanil, analogs of anadamide and arvanil as described in US 20040122089, and a proton pump inhibitor (e.g. omeprazole).

The compound of the invention can also be used in a co-therapy with a second agent that is a cannabanoid receptor antagonist to prevent and/or treat obesity and other appetite related disorders.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include arecoline, arginine, barium, betaine, bismuth, chloroprocaine, choline, clemizole, deanol, imidazole, and morpholineethanol. In one embodiment are tris salts.

The agents of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:
BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-10™, -103™ and -105™, sold by FMC Corporation, Marcus Hook, Pa., USA), or mixtures thereof,
FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,
DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,
LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,
ANTI-CAKING AGENTS: calcium silicate, magnesium silicate, silicon dioxide, colloidal silicon dioxide, talc, or mixtures thereof,
ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and
COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1,U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in U.S. 20030198619, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract. The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

A number of embodiments of the invention have been described. Nevertheless, it will be understood that various modifications may be made without departing from the spirit and scope of the invention.

1. A compound having the formula: 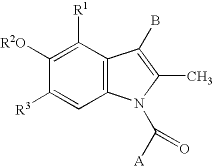
wherein:
R¹ is H or a halogen;
R² is H; a C₁ to C₆ alkyl, optionally independently substituted with one or more halogen; 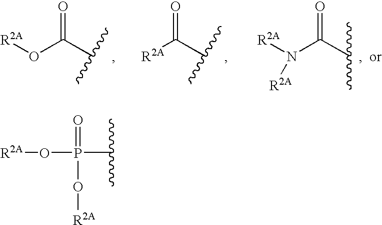
wherein each R^{2A} is independently: H or a C₁ to C₆ alkyl, alkenyl, alkynyl, aryl, cycloalkyl, or arylalkyl optionally independently substituted with one or more halogen —OH, —C(O)OH, —NH₃;
R³ is H or a halogen
A is 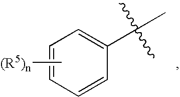
wherein
each R⁵ is independently H, a halogen, —CH₃, —CN, —OCH₃, —SCH₃, OCF₂H, OCF₃, or —CH₂CH₃, optionally independently substituted with one or more halogen; n=1, 2, 3, 4, or 5;
B is 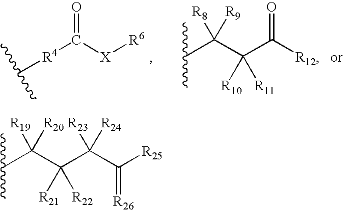
wherein
R⁴ is an optionally independently substituted C₁ to C₁₀ alkyl (wherein the substituents are selected from halogen, —OH, —C(O)OH, —CH₃, and —CH₂CH₃, optionally independently substituted with one or more halogen;
X is —O—, —S—, —N(H)— or —N(H)S(O₂)—;
R⁶ is: H; an optionally independently substituted C₁ to C₁₀ alkyl; an optionally independently substituted C₄ to C₈ cycloalkyl; a C₁ to C₆ hydroxyalkyl; a hydroxyl substituted C₄ to C₈ aryl; a primary, secondary or tertiary C₁ to C₆ alkylamino; a primary, secondary or tertiary branched C₁ to C₆ alkylamino; primary, secondary or tertiary C₄ to C₈ arylamino; C₂ to C₆ alkylcarboxylic acid; a branched C₃ to C₆ alkylcarboxylic acid; a C₁ to C₆ alkylester; a branched C₁ to C₆ alkylester; a C₄ to C₈ aryl; a C₄ to C₈ arylcarboxylic acid; a C₄ to C₈ arylester; a C₄ to C₈ aryl substituted C₁ to C₆ alkyl; a C₄ to C₈ heterocyclic alkyl or aryl; an alkyl-substituted or aryl-substituted C₄ to C₈ heterocyclic alkyl or aryl, wherein one or more H can be independently substituted by halogen, —OH, and —C(O)OH;
R⁸, R⁹, R¹⁰, and R¹¹ are independently selected from:
(a) H,
(b) F,
(c) methyl or ethyl,
(d) —CF₃, —CF₂H or —CFH₂,
(e) hydroxy, —OR¹⁴, SR¹⁴, S(O)R¹⁴, or S(O)₂R¹⁴,
(f) naphthylmethyl, and
(g) mono-substituted or di-substituted benzyl, wherein the substituents are selected from:
(i) H,
(ii) —CF₃,
(iii) —CN,
(iv) F, Cl, Br, or I,
(v) C_{1 to} C₆ alkyl, and
(vi) SR¹⁴, S(O)R¹⁴, or S(O)₂R¹⁴;
or R⁸ and R⁹ together form an oxo group,
or R¹⁰ and R¹¹ together form an oxo group,
or R⁸ and R⁹ are joined so that together with the carbon atom to which they are attached there is formed a cycloalkyl having 3, 4, 5, 6 or 7 ring carbons;
or R⁸ and R¹⁰ are joined so that together with the carbon atom to which they are attached there is formed a cycloalkyl having 3, 4, 5, 6 or 7 ring carbons;
or R⁸ and R¹ are joined so that together with the carbon atom to which they are attached there is formed a cycloalkyl having 3, 4, 5, 6 or 7 ring carbons;
or R⁹ and R¹⁰ are joined so that together with the carbon atom to which they are attached there is formed a cycloalkyl having 3, 4, 5, 6 or 7 ring carbons;
or R⁹ and R¹⁰ are joined so that together with the carbon atom to which they are attached there is formed a cycloalkyl having 3, 4, 5, 6 or 7 ring carbons;
or R¹⁰ and R¹¹ are joined so that together with the carbon atom to which they are attached there is formed a cycloalkyl having 3, 4, 5, 6 or 7 ring carbons;
R¹² is selected from:
(a) —OR₁₃, and
(b) —NR₁₅R₁₆;
R¹³ is selected from:
(a) H, and
(b) C₁ to C₄ alkyl;
R¹⁴ is selected from methyl, ethyl, mono-substituted benzyl or di-substituted benzyl, wherein the substituents are selected from:
(a) H,
(b) CF₃,
(c) CN,
(d) F, Cl, Br, I, and
(e) C₁ to C₆ alkyl;
R¹⁵ and R¹⁶ are independently selected from:
(a) H,
(b) C Ito C₃ alkyl,
(c) —OR₁₃,
(d) —C(O)R₁₇
(e) —S(O)₂R₁₈,
(f) a mono-substituted C₂ to C₄ alkyl, wherein the substituent is selected from:
(i) hydroxy,
(ii) amino,
(iii) methylamino,
(iv) dimethyl amino, and
(g) an unsubstituted, mono-substituted, or disubstituted phenyl, benzyl or pyridyl group, wherein the substituents are selected from:
(i) H,
(ii) CF₃,
(iii) CN,
(iv) F, Cl, Br, I, and
(v) C₁₋₆ alkyl;
or R¹⁵ and R¹⁶ are joined so that together with the nitrogen atom to which they are attached there is formed a 3, 4, 5, 6, or 7-membered ring, optionally including one or two additional heteroatoms, wherein the additional heteroatoms are selected from N, O, and S, the ring optionally including one or two carbonyl or sulfonyl groups;
R¹⁷ is selected from:
(a) H,
(b) C₁ to C₄ alkyl,
(c) CF₃, and
(d) an unsubstituted, mono-substituted, or disubstituted phenyl or benzyl group, wherein the substituents are selected from:
(i) H,
(ii) CF₃,
(iii) CN,
(iv) F, Cl, Br, I, and
(v) C₁ to C₆ alkyl;
R¹⁸ is selected from:
(a) C₁ to C₄ alkyl,
(b) CF₃, and
(c) an unsubstituted, mono-substituted, or disubstituted phenyl or benzyl group, wherein the substituents are selected from:
(i) H,
(ii) CF₃,
(iii) CN,
(iv) F, Cl, Br, I, and
(v) C₁ to C₆ alkyl;
R¹⁹R²⁰, R²¹, R²², R²³ and R²⁴ are independently selected from:
(a) H,
(b) F or Cl,
(c) C₁ to C₅ alkyl or haloalkyl,
(d) C₃ to C₆ cycloalkyl,
(e) —CF₃, —CF₂H or —CFH₂,
(f) hydroxy, —OR₂₇, SR₂₇, S(O)R₂₇, or S(O)₂R₂₇,
(g) mono-substituted or di-substituted benzyl, wherein the substituents are selected from:
(i) —CF₃,
(ii) —CN,
(iii) F, Cl, Br, or I,
(iv) C₁ to C₆ alkyl, and
(v) SR₂₇, S(O)R₂₇, or S(O)₂R₂₇,
(h) mono-substituted or di-substituted phenyl, wherein the substituents are selected from:
(i) —CF₃,
(ii) —CN,
(iii) F, Cl, Br, or I,
(iv) C₁ to C₆ alkyl, and
(v) SR₂₇, S(O)R₂₇, or S(O)₂R₂₇,
or R¹⁹ and R²⁰ together form an oxo group,
or R²¹ and R²² together form an oxo group,
or R²³ and R²⁴ together form an oxo group,
or R¹⁹ and R²⁰ are joined so that together with the carbon atom to which they are attached there is formed a cycloalkyl having saturated 3, 4, 5, 6 or 7 ring carbons;
or R²⁰ and R²¹ are joined so that together with the carbon atom to which they are attached there is formed a cycloalkyl having saturated 3, 4, 5, 6 or 7 ring carbons;
or R²⁰ and R²³ are joined so that together with the carbon atom to which they are attached there is formed cycloalkyl having saturated 3, 4, 5, 6 or 7 ring carbons;
or R²¹ and R²² are joined so that together with the carbon atom to which they are attached there is formed a cycloalkyl having saturated 3, 4, 5, 6 or 7 ring carbons;
or R²¹ and R²³ are joined so that together with the carbon atom to which they are attached there is formed a cycloalkyl having saturated 3, 4, 5, 6 or 7 ring carbons;
or R²⁴ and R²⁵ are joined so that together with the carbon atom to which they are attached there is formed a cycloalkyl having saturated 3, 4, 5, 6 or 7 ring carbons;
R²⁶ is O or S;
R²⁷ is methyl, ethyl, mono-substituted benzyl or di-substituted benzyl, wherein the substituents are selected from:
(a) CF₃,
(b) CN,
(c) F, Cl, Br, I, and
(d) C₁ to C₆ alkyl;
R²⁸ and R²⁹ are independently selected from:
(a) H,
(b) C₁₋₃ alkyl,
(c) C₃₋₆ cycloalkyl
(d) —OR³²,
(e) —C(O)R³⁰
(f) —S(O)₂R³¹,
(g) a mono-substituted C₂ to C₄ alkyl, wherein the substituent is selected from:
(i) hydroxy,
(ii) amino,
(iii) methylamino,
(iv) dimethyl amino
(h) an unsubstituted, mono-substituted, or disubstituted phenyl, benzyl or pyridyl group, wherein the substituents are selected from:
(i) H,
(ii) CF₃,
(iii) CN,
(iv) F, Cl, Br, I, and
(v) C₁ to C₆ alkyl,
or R²⁸ and R²⁹ are joined so that together with the nitrogen atom to which they are attached there is formed a 3, 4, 5, 6, or 7-membered ring, optionally including one or two additional heteroatoms, wherein the additional heteroatoms are selected from N, O, and S, wherein a carbon atom may optionally be substituted with an oxo group and a sulfur atom of the ring may optionally substituted with two oxo groups;
R³⁰ is selected from:
(a) H,
(b) C₁ to C₄ alkyl,
(c) CF₃,
(d) an unsubstituted, mono-substituted, or disubstituted phenyl or benzyl group, wherein the substituents are selected from:
(i) CF₃,
(ii) CN,
(iii) F, Cl, Br, I, and
(iv) C₁ to C₆ alkyl;
R³¹ is selected from:
(a) C₁ to C₄ alkyl,
(b) CF₃,
(c) an unsubstituted, mono-substituted, or disubstituted phenyl or benzyl group, wherein the substituents are selected from:
(i) CF₃,
(ii) CN,
(iii) F, Cl, Br, I, and
(iv) C₁ to C₆ alkyl;
provided that:
(a) when R¹ is H, R² is CH₃, R³ is H, and A is 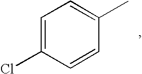
then B is other than —CH₂C(O)NHCH₂C(O)OCH₂CH₃, —CH₂C(O)NHCH₂CH₂OH, —CH₂C(O)CH₂CH₃, CH₂CH₂C(O)OH, —CH₂C(O)OCH₂CH₃ and —CH(CH₃)C(O)OCH₂CH₃;
(b) when R¹ is H, R² is CH₃, R³ is H, B is —CH₂C(O)OH and A is 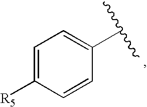
then R⁵ is other than H, F or Cl;
(c) when R¹ is H, R² is H, R³ is H, and A is 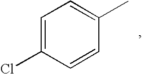
then B is other than —CH₂C(O)OH, —CH₂C(O)OCH₂CH₃;
(d) when R¹ is F, R² is —CH₃, R³ is H, A is 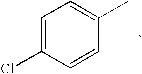
B is other than —CH₂C(O)OH;
(e) when R¹ is H, R² is —CH₃, R³ is H, A is 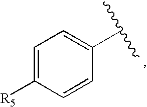
R⁵ is —CF₃, then B is other than —CH₂C(O)OH;
(f) when R¹ is H, R² is —CH₃, R³ is H, A is 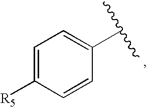
R⁵ is H, then B is other than —CH₂C(O)OH;
(g) when R¹ is H, R² is —CH₃, R³ is H, A is 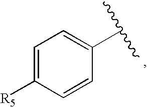
R⁵ is H, then B is other than —CH₂C(O)OCH₃ or CH₂C(O)OC(CH₃)₃; and
(h) when R¹ is H, R² is —CH₃, R³ is H, A is 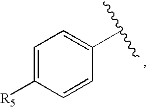
R⁵ is F, then B is other than —CH₂C(O)OCH₃.

2. The compound of claim 1 wherein R³ is F, Cl or Br.

3. The compound of claim 1 wherein B is

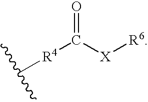

4. The compound of claim 1 wherein the compound is in the form of a TRIS salt.

5. A pharmaceutical composition comprising the compound of claim land a pharmaceutically acceptable carrier.

6. The compound of claim 1 wherein X is —O—.

7. The compound of claim 1 wherein X is —N—.

8. The compound of claim 1 wherein R⁴ is an optionally independently substituted C₁ to C₆ alkyl.

9. The compound of claim 8 wherein R⁴ is substituted.

10. The compound of claim 8 wherein R⁴ is not substituted.

11. The compound of claim 1 wherein R² is H or a C₁ to C₃ alkyl optionally substituted with a halogen.

12. The compound of claim 1 wherein R² is H or a C₁ to C₃ alkyl optionally substituted with F.

13. The compound of claim 1 wherein R² is selected from —CH₃, —CF₂H, —CF₃.

14. The compound of claim 1 wherein R^{2A} is an unsubstituted C₁ to C₃ alkyl.

15. The compound of claim 1 wherein R^{2A} is a substituted C₁ to C₃ alkyl.

16. The compound of claim 1 wherein R^{2A} is an optionally independently substituted C₁ to C₆ alkyl, alkenyl, alkynyl or an optionally independently substituted C₆ to C₁₀ aryl or cycloalkyl.

17. The compound of claim 1, 13 or 14 wherein R⁶ is an optionally independently substituted C₁ to C₆ alkyl, alkenyl, alkynyl or an optionally independently substituted C₆ to C₁₀ aryl or cycloalkyl.

18. The compound of claim 17 wherein R⁶ is substituted.

19. The compound of claim 17 wherein R⁶ is not substituted.

20. The compound of claim 1 wherein the compound exhibits an IC₅₀ for COX-1 that is at least 10-fold greater than for COX-2.

21. The compound of claim 1 wherein the compound exhibits an IC₅₀ for COX-2 that is less than 1.0 μM.

22. The compound of claim 1 wherein the compound exhibits an IC₅₀ for FAAH that is less than 50 μM.

23. A method for treating pain and inflammation comprising administering the compound of claim 1.

24. A method for treating anxiety comprising administering the compound of claim 1.

25. A method for treating pain and inflammation comprising administering the pharmaceutical composition of claim 5.

26. A method for treating anxiety comprising administering the pharmaceutical composition of claim 5.

27. A compound having the formula: 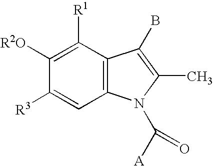
wherein:
R¹ is H or a halogen;
R² is H; a C₁ to C₆ alkyl, optionally independently substituted with one or more halogen; 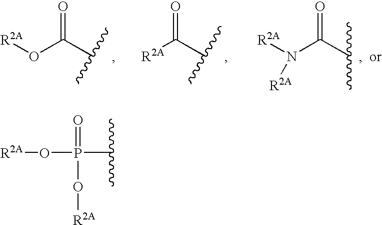
wherein each R^{2A} is independently: H or a C₁ to C₆ alkyl, alkenyl, alkynyl, aryl, cycloalkyl, or arylalkyl optionally independently substituted with one or more halogen-;
R³ is H or a halogen;
A is 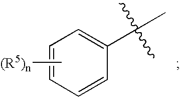
wherein
each R⁵ is independently H, a halogen, —CH₃, —CN, —OCH₃, —SCH₃, or —CH₂CH₃, optionally independently substituted with one or more halogen; n=1, 2, 3, 4, or 5;
provided that R³ is H only when R⁵ is —OCH₃, optionally independently substituted with one or more halogen;
B is 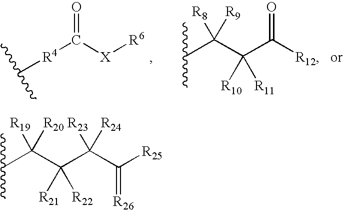
wherein
R⁴ is an optionally independently substituted C₁ to C₁₀ (, wherein the substituents are selected from halogen, —OH, —C(O)OH, —CH₃, and —CH₂CH₃, optionally independently substituted with one or more halogen;
X is —O—, —S—, —N(H)— or —N(H)S(O₂)—;
R⁶ is: H; an optionally independently substituted C₁ to Cloalkyl; an optionally independently substituted C₄ to C₈ cycloalkyl; a C₁ to C₆ hydroxyalkyl; a hydroxyl substituted C₄ to C₈ aryl; a primary, secondary or tertiary C₁ to C₆ alkylamino; a primary, secondary or tertiary branched C₁ to C₆ alkylamino; primary, secondary or tertiary C₄ to C₈ arylamino; C₂ to C₆ alkylcarboxylic acid; a branched C₃ to C₆ alkylcarboxylic acid; a C₁ to C₆ alkylester; a branched C₁ to C₆ alkylester; a C₄ to C₈ aryl; a C₄ to C₈ arylcarboxylic acid; a C₄ to C₈ arylester; a C₄ to C₈ aryl substituted C₁ to C₆ alkyl; a C₄ to C₈ heterocyclic alkyl or aryl; an alkyl-substituted or aryl-substituted C₄ to C₈ heterocyclic alkyl or aryl, wherein one or more H can be independently substituted by halogen, —OH, and —C(O)OH;
R⁸, R⁹, R¹⁰, and R¹¹ are independently selected from:
(a) H,
(b) F,
(c) methyl or ethyl,
(d) —CF₃, —CF₂H or —CFH₂,
(e) hydroxy, —OR¹⁴, SR¹⁴, S(O)R¹⁴, or S(O)₂R¹⁴,
(f) naphthylmethyl, and
(g) mono-substituted or di-substituted benzyl, wherein the substituents are selected from:
(i) H,
(ii) —CF₃,
(iii) —CN,
(iv) F, Cl, Br, or I,
(v) C₁ to C₆alkyl, and
(vi) SR¹⁴, S(O)R¹⁴, or S(O)₂R¹⁴;
or R⁸ and R⁹ together form an oxo group,
or R¹⁰ and R¹¹ together form an oxo group,
or R⁸ and R⁹ are joined so that together with the carbon atom to which they are attached there is formed a cycloalkyl having 3, 4, 5, 6 or 7 ring carbons;
or R⁸ and R¹⁰ are joined so that together with the carbon atom to which they are attached there is formed a cycloalkyl having 3, 4, 5, 6 or 7 ring carbons;
or R⁸ and R¹¹ are joined so that together with the carbon atom to which they are attached there is formed a cycloalkyl having 3, 4, 5, 6 or 7 ring carbons;
or R⁹ and R¹⁰ are joined so that together with the carbon atom to which they are attached there is formed a cycloalkyl having 3, 4, 5, 6 or 7 ring carbons;
or R⁹ and R¹¹ are joined so that together with the carbon atom to which they are attached there is formed a cycloalkyl having 3, 4, 5, 6 or 7 ring carbons;
or R¹⁰ and R¹¹ are joined so that together with the carbon atom to which they are attached there is formed a cycloalkyl having 3, 4, 5, 6 or 7 ring carbons;
R¹² is selected from:
(a) —OR₁₃, and
(b) —NR₁₅R₁₆;
R¹³ is selected from:
(a) H, and
(b) C₁ to C₄alkyl;
R¹⁴ is selected from methyl, ethyl, mono-substituted benzyl or di-substituted benzyl, wherein the substituents are selected from:
(a) H,
(b) CF₃,
(c) CN,
(d) F, Cl, Br, I, and
(e) C₁ to C₆ alkyl;
R¹⁵ and R¹⁶ are independently selected from:
(a) H,
(b) C₁ to C₃ alkyl,
(c) —OR₁₃,
(d) —C(O)R₁₇
(e) —S(O)₂R₁₈,
(f) a mono-substituted C₂ to C₄ alkyl, wherein the substituent is selected from:
(i) hydroxy,
(ii) amino,
(iii) methylamino,
(iv) dimethyl amino, and
(g) an unsubstituted, mono-substituted, or disubstituted phenyl, benzyl or pyridyl group, wherein the substituents are selected from:
(i) H,
(ii) CF₃,
(iii) CN,
(iv) F, Cl, Br, I, and
(v) C₁₋₆ alkyl;
or R¹⁵ and R¹⁶ are joined so that together with the nitrogen atom to which they are attached there is formed a 3, 4, 5, 6, or 7-membered ring, optionally including one or two additional heteroatoms, wherein the additional heteroatoms are selected from N, O, and S, the ring optionally including one or two carbonyl or sulfonyl groups;
R¹⁷ is selected from:
(a) H,
(b) C₁ to C₄ alkyl,
(c) CF₃, and
(d) an unsubstituted, mono-substituted, or disubstituted phenyl or benzyl group, wherein the substituents are selected from:
(i) H,
(ii) CF₃,
(iii) CN,
(iv) F, Cl, Br, I, and
(v) C₁ to C₆ alkyl;
R¹⁸ is selected from:
(a) C₁ to C₄ alkyl,
(b) CF₃, and
(c) an unsubstituted, mono-substituted, or disubstituted phenyl or benzyl group, wherein the substituents are selected from:
(i) H,
(ii) CF₃,
(iii) CN,
(iv) F, Cl, Br, I, and
(v) C₁ to C₆ alkyl;
R¹⁹, R²⁰, R²¹, R²², R²³ and R²⁴ are independently selected from:
(a) H,
(b) F or Cl,
(c) C₁ to C₅ alkyl or haloalkyl,
(d) C₃ to C₆ cycloalkyl,
(e) —CF₃, —CF₂H or —CFH₂,
(f) hydroxy, —OR₂₇, SR₂₇, S(O)R₂₇, or S(O)₂R₂₇,
(g) mono-substituted or di-substituted benzyl, wherein the substituents are selected from:
(i) —CF₃,
(ii) —CN,
(iii) F, Cl, Br, or I,
(iv) C₁ to C₆ alkyl, and
(v) SR₂₇, S(O)R₂₇, or S(O)₂R₂₇,
(h) mono-substituted or di-substituted phenyl, wherein the substituents are selected from:
(i) —CF₃,
(ii) —CN,
(iii) F, Cl, Br, or I,
(iv) C₁ to C₆ alkyl, and
(v) SR₂₇, S(O)R₂₇, or S(O)₂R₂₇,
or R¹⁹ and R²⁰ together form an oxo group,
or R²¹ and R²² together form an oxo group,
or R²³ and R²⁴ together form an oxo group,
or R¹⁹ and R²⁰ are joined so that together with the carbon atom to which they are attached there is formed a cycloalkyl having saturated 3, 4, 5, 6 or 7 ring carbons;
or R²⁰ and R²¹ are joined so that together with the carbon atom to which they are attached there is formed a cycloalkyl having saturated 3, 4, 5, 6 or 7 ring carbons;
or R²⁰ and R²³ are joined so that together with the carbon atom to which they are attached there is formed cycloalkyl having saturated 3, 4, 5, 6 or 7 ring carbons;
or R²¹ and R²² are joined so that together with the carbon atom to which they are attached there is formed a cycloalkyl having saturated 3, 4, 5, 6 or 7 ring carbons;
or R²¹ and R²³ are joined so that together with the carbon atom to which they are attached there is formed a cycloalkyl having saturated 3, 4, 5, 6 or 7 ring carbons;
or R²⁴ and R²⁵ are joined so that together with the carbon atom to which they are attached there is formed a cycloalkyl having saturated 3, 4, 5, 6 or 7 ring carbons;
R²⁶ is O or S;
R²⁷ is methyl, ethyl, mono-substituted benzyl or di-substituted benzyl, wherein the substituents are selected from:
(a) CF₃,
(b) CN,
(c) F, Cl, Br, I, and
(d) C₁ to C₆ alkyl;
R²⁸ and R²⁹ are independently selected from:
(a) H,
(b) C₁₋₃ alkyl,
(c) C₃₋₆ cycloalkyl
(d) —OR³²
(e) —C(O)R³⁰
(f) —S(O)₂R³¹,
(g) a mono-substituted C₂ to C₄ alkyl, wherein the substituent is selected from:
(i) hydroxy,
(ii) amino,
(iii) methylamino,
(iv) dimethyl amino
(h) an unsubstituted, mono-substituted, or disubstituted phenyl, benzyl or pyridyl group, wherein the substituents are selected from:
(i) H,
(ii) CF₃,
(iii) CN,
(iv) F, Cl, Br, I, and
(v) C₁ to C₆ alkyl,
or R²⁸ and R²⁹ are joined so that together with the nitrogen atom to which they are attached there is formed a 3, 4, 5, 6, or 7-membered ring, optionally including one or two additional heteroatoms, wherein the additional heteroatoms are selected from N, O, and S, wherein a carbon atom may optionally be substituted with an oxo group and a sulfur atom of the ring may optionally substituted with two oxo groups;
R³⁰ is selected from:
(a) H,
(b) C₁ to C₄ alkyl,
(c) CF₃,
(d) an unsubstituted, mono-substituted, or disubstituted phenyl or benzyl group, wherein the substituents are selected from:
(i) CF₃,
(ii) CN,
(iii) F, Cl, Br, I, and
(iv) C₁ to C₆ alkyl;
R³¹ is selected from:
(a) C₁ to C₄ alkyl,
(b) CF₃,
(c) an unsubstituted, mono-substituted, or disubstituted phenyl or benzyl group, wherein the substituents are selected from:
(i) CF₃,
(ii) CN,
(iii) F, Cl, Br, I, and
(iv) C₁ to C₆ alkyl;
